(19) United States
(12) Patent Application Publication
(10) Pub. No.: US 2024/0246996 A1
(43) Pub. Date: Jul. 25, 2024

(54) ORGANIC COMPOUND, ORGANIC LIGHT EMITTING DIODE AND ORGANIC LIGHT EMITTING DEVICE INCLUDING THE SAME

(71) Applicants: LG Display Co., Ltd., Seoul (KR); LT Materials Co., Ltd., Yongin-si (KR)

(72) Inventors: Seon-Keun YOO, Paju-si (KR); Young-Jun YU, Paju-si (KR); Seong-Su JEON, Paju-si (KR); Sang-Beom KIM, Paju-si (KR); Gi-Baek LEE, Yongin-si (KR); Dae-Hyuk CHOI, Yongin-si (KR); Dong-Jun KIM, Yongin-si (KR); Jun-Tae MO, Yongin-si (KR)

(21) Appl. No.: 18/480,435

(22) Filed: Oct. 3, 2023

(30) Foreign Application Priority Data
Dec. 27, 2022 (KR) 10-2022-0185637

(51) Int. Cl. C07D 498/14; C07D 513/14; H10K 50/11; H10K 85/30; H10K 85/60

(52) U.S. Cl. CPC C07D 498/14; C07D 513/14; H10K 50/11; H10K 85/342; H10K 85/656; H10K 85/6572

(57) ABSTRACT

The present disclosure relates to an organic compound and an organic light emitting diode and an organic light emitting device each including the same, and more specifically, relates to an emitting compound of following and an organic light emitting diode and an organic light emitting device each including the organometallic compound.

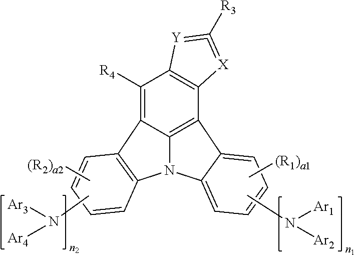

### CROSS-REFERENCE TO RELATED APPLICATIONS

The present application claims the benefit of Republic of Korea Patent Application No. 10-2022-0185637 filed in the Republic of Korea on Dec. 27, 2022, which is hereby incorporated by reference in its entirety.

### TECHNOLOGY FIELD

The present disclosure relates to an organic compound, and more specifically, to an organic compound being capable of providing low driving voltage, improved emitting efficiency and long lifespan, and an organic light emitting diode (OLED) and an organic light emitting device including the same.

### BACKGROUND ART

Recently, requirement for flat panel display devices having small occupied area is increased. Among the flat panel display devices, a technology of an organic light emitting display device, which includes an organic light emitting diode (OLED) and may be called to as an organic electroluminescent device, is rapidly developed.

The OLED includes a cathode as an electron injection electrode, an anode as a hole injection electrode and an organic light emitting layer, which is disposed between the cathode and the anode and includes a host and a dopant. When electrons from the cathode and holes from the anode enter into the organic light emitting layer, the electrons and holes are combined to generate an exciton, and the exciton is transformed from an excited state to a ground state. As a result, the light is emitted from the OLED. The OLED can be formed on a flexible transparent substrate, e.g., a plastic substrate, and can be driven by low voltage. In addition, the OLED has low power consumption and high color purity.

For example, an emitting material layer of the organic light emitting layer may include a host and a dopant, and an exciton generated in the host is transferred into the dopant so that the light is emitted from the dopant.

The emitting efficiency and the lifespan of the OLED may depend on an efficiency of generating the exciton in the host and/or an energy transfer efficiency from the host into the dopant.

Accordingly, new host material being capable of improving emitting efficiency and the lifespan of the OLED is needed.

### SUMMARY

The present disclosure is directed to an organic compound, an OLED and an organic light emitting device including the organic compound that substantially obviate one or more of the problems associated with the limitations and disadvantages of the related conventional art.

An object of the present disclosure is provide an organic compound being capable of providing low driving voltage, improved emitting efficiency and long lifespan.

Another object of the present disclosure is provide an OLED and an organic light emitting device including the organic compound.

Additional features and advantages of the present disclosure are set forth in the description which follows, and will be apparent from the description, or evident by practice of the present disclosure. The objectives and other advantages of the present disclosure are realized and attained by the features described herein as well as in the appended drawings.

To achieve these and other advantages in accordance with the purpose of the embodiments of the present disclosure, as described herein, an aspect of the present disclosure is an organic compound represented by Formula 1: [Formula 1]

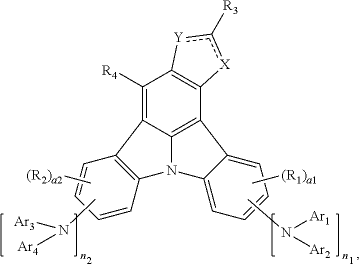

wherein one of X and Y is nitrogen (N), and the other one of X and Y is oxygen (O) or sulfur (S), each of R₁ and R₂ is independently selected from the group consisting of hydrogen, deuterium, a substituted or unsubstituted C1 to C10 alkyl group and a substituted or unsubstituted C6 to C30 aryl group, each of a1 and a2 is independently an integer of 0 to 4, each of R₃ and R₄ is independently selected from the group consisting of hydrogen, deuterium, a substituted or unsubstituted C1 to C10 alkyl group and a substituted or unsubstituted C6 to C30 aryl group, each of Ar₁ to Ar₄ is independently selected from the group consisting of a substituted or unsubstituted C6 to C30 aryl group, a substituted or unsubstituted C3 to C30 cycloalkyl group, a substituted or unsubstituted C6 to C30 aryloxy group, a substituted or unsubstituted C6 to C30 arylthioxy group and a substituted or unsubstituted C5 to C30 heteroaryl group, each of n1 and n2 is independently 0 or 1, n1 and n2 are not 0 simultaneously, the sum of a1 and n1 is less than or equal to 4, and the sum of a2 and n2 is less than or equal to 4.

Another aspect of the present disclosure is an organic light emitting diode comprising a first electrode; a second electrode facing the first electrode; and a first emitting part including a first red emitting material layer and positioned between the first and second electrodes, wherein the first red emitting material layer includes the above organic compound.

Another aspect of the present disclosure is an organic light emitting device comprising a substrate; an organic light emitting diode disposed on the substrate and including a first electrode, a second electrode facing the first electrode, a first emitting part positioned between the first and second electrodes and including a first red emitting material layer; and an encapsulation covering the organic light emitting diode, wherein the first red emitting material layer includes the above organic compound.

It is to be understood that both the foregoing general description and the following detailed description are exemplary and explanatory and are intended to further explain the present disclosure as claimed.

### BRIEF DESCRIPTION OF THE DRAWINGS

The accompanying drawings, which are included to provide a further understanding of the present disclosure and are incorporated in and constitute a part of this specification, illustrate embodiments of the present disclosure and together with the description serve to explain the principles of the present disclosure.

FIG. 1 is a schematic circuit diagram illustrating an organic light emitting display device of the present disclosure.

FIG. 2 is a schematic cross-sectional view illustrating an organic light emitting display device according to a first embodiment of the present disclosure.

FIG. 3 is a schematic cross-sectional view illustrating an OLED according to a second embodiment.

FIG. 4 is a schematic cross-sectional view illustrating an OLED according to a third embodiment.

FIG. 5 is a schematic cross-sectional view illustrating an organic light emitting display device according to a fourth embodiment of the present disclosure.

FIG. 6 is a schematic cross-sectional view illustrating an OLED according to a fifth embodiment of the present disclosure.

FIG. 7 is a schematic cross-sectional view illustrating an OLED according to a sixth embodiment.

FIG. 8 is a schematic cross-sectional view illustrating an OLED according to a seventh embodiment.

### DETAILED DESCRIPTION

Reference will now be made in detail to aspects of the present disclosure, examples of which may be illustrated in the accompanying drawings. In the following description, when a detailed description of well-known functions or configurations related to this document is determined to unnecessarily cloud a gist of the inventive concept, the detailed description thereof will be omitted. The progression of processing steps and/or operations described is an example; however, the sequence of steps and/or operations is not limited to that set forth herein and may be changed as is known in the art, with the exception of steps and/or operations necessarily occurring in a particular order. Like reference numerals designate like elements throughout. Names of the respective elements used in the following explanations are selected only for convenience of writing the specification and may be thus different from those used in actual products.

Advantages and features of the present disclosure and methods of achieving them will be apparent with reference to the aspects described below in detail with the accompanying drawings. However, the present disclosure is not limited to the aspects disclosed below, but can be realized in a variety of different forms, and only these aspects allow the disclosure of the present disclosure to be complete. The present disclosure is provided to fully inform the scope of the disclosure to the skilled in the art of the present disclosure.

The shapes, sizes, proportions, angles, numbers, and the like disclosed in the drawings for explaining the aspects of the present disclosure are illustrative, and the present disclosure is not limited to the illustrated matters. The same reference numerals refer to the same elements throughout the specification. In addition, in describing the present disclosure, if it is determined that a detailed description of the related known technology unnecessarily obscure the subject matter of the present disclosure, the detailed description thereof can be omitted. When ‘including’. ‘having’. ‘consisting’, and the like are used in this specification, other parts may be added unless ‘only’ is used. When a component is expressed in the singular, cases including the plural are included unless specific statement is described.

In construing an element, the element is construed as including an error or tolerance range although there is no explicit description of such an error or tolerance range.

In describing a position relationship, for example, when a position relation between two parts is described as, for example, “on,” “over,” “under,” and “next,” one or more other parts may be disposed between the two parts unless a more limiting term, such as “just” or “direct(ly)” is used.

In describing a time relationship, for example, when the temporal order is described as, for example, “after,” “subsequent,” “next,” and “before,” a case that is not continuous may be included unless a more limiting term, such as “just.” “immediate(ly),” or “direct(ly)” is used.

It will be understood that, although the terms “first,” “second,” etc. may be used herein to describe various elements, these elements should not be limited by these terms. These terms are only used to distinguish one element from another. For example, a first element could be termed a second element, and, similarly, a second element could be termed a first element, without departing from the scope of the present disclosure.

Features of various aspects of the present disclosure may be partially or overall coupled to or combined with each other, and may be variously inter-operated with each other and driven technically as those skilled in the art can sufficiently understand. The aspects of the present disclosure may be carried out independently from each other, or may be carried out together in co-dependent relationship.

Reference will now be made in detail to some of the examples and preferred embodiments, which are illustrated in the accompanying drawings.

In the present disclosure, an organic compound as a host is included in an emitting material layer of an OLED, and the OLED has advantages in the driving voltage, the emitting efficiency and the lifespan. For example, an organic light emitting device including the OLED may be an organic light emitting display device or an organic lightening device. As an example, an organic light emitting display device, which is a display device including the OLED of the present disclosure, will be mainly described.

The organic compound of the present disclosure provides improved emitting efficiency and lifespan. The organic compound of the present disclosure is represented by Formula 1.

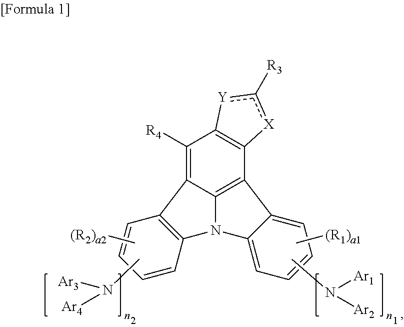

In Formula 1,

one of X and Y is nitrogen (N), and the other one of X and Y is oxygen (O) or sulfur (S),

each of R₁ and R₂ is independently selected from the group consisting of hydrogen, deuterium, a substituted or unsubstituted C1 to C10 alkyl group and a substituted or unsubstituted C6 to C30 aryl group,

each of a1 and a2 is independently an integer of 0 to 4,

each of R₃ and R₄ is independently selected from the group consisting of hydrogen, deuterium, a substituted or unsubstituted C1 to C10 alkyl group and a substituted or unsubstituted C6 to C30 aryl group,

each of Ar₁ to Ar₄ is independently selected from the group consisting of a substituted or unsubstituted C6 to C30 aryl group, a substituted or unsubstituted C3 to C30 cycloalkyl group, a substituted or unsubstituted C6 to C30 aryloxy group, a substituted or unsubstituted C6 to C30 arylthioxy group and a substituted or unsubstituted C5 to C30 heteroaryl group, and

each of n1 and n2 is independently 0 or 1, n1 and n2 are not 0 simultaneously, the sum of a1 and n1 is less than or equal to 4, and the sum of a2 and n2 is less than or equal to 4.

In the present disclosure, without specific definition, when an alkyl group, a cycloalkyl group, an aryloxy group, an arylthioxy group, an aryl group and a heteroaryl group are substituted, a substituent may be selected from the group consisting of deuterium, a C1 to C10 alkyl group unsubstituted or substituted with deuterium, a C3 to C30 cycloalkyl group unsubstituted or substituted with deuterium, a C6 to C30 aryl group unsubstituted or substituted with deuterium and a C5 to C30 heteroaryl group unsubstituted or substituted with deuterium.

In the present disclosure, without specific definition, a C1 to C10 alkyl group may be selected from the group consisting of methyl, ethyl, propyl and butyl, e.g., tert-butyl.

In the present disclosure, without specific definition, a C6 to C30 aryl group may be selected from the group consisting of phenyl, biphenyl, terphenyl, naphthyl, anthracenyl, pentanenyl, indenyl, indenoindenyl, heptalenyl, biphenylenyl, indacenyl, phenanthrenyl, benzophenanthrenyl, dibenzophenanthrenyl, azulenyl, pyrenyl, fluoranthenyl, triphenylenyl, chrysenyl, tetraphenyl, tetrasenyl, picenyl, pentaphenyl, pentacenyl, fluorenyl, indenofluorenyl and spiro-fluorenyl.

In the present disclosure, without specific definition, a C5 to C30 heteroaryl group may be selected from the group consisting of pyrrolyl, pyridinyl, pyrimidinyl, pyrazinyl, pyridazinyl, triazinyl, tetrazinyl, imidazolyl, pyrazolyl, indolyl, isoindolyl, indazolyl, indolizinyl, pyrrolizinyl, carbazolyl, benzocarbazolyl, dibenzocarbazolyl, indolocarbazolyl, indenocarbazolyl, benzofurocarbazolyl, benzothienocarbazolyl, quinolinyl, isoquinolinyl, phthalazinyl, quinoxalinyl, cinnolinyl, quinazolinyl, quinozolinyl, purinyl, benzoquinolinyl, benzoisoquinolinyl, benzoquinazolinyl, benzoquinoxalinyl, acridinyl, phenanthrolinyl, perimidinyl, phenanthridinyl, pteridinyl, cinnolinyl, naphtharidinyl, furanyl, oxazinyl, oxazolyl, oxadiazolyl, triazolyl, dioxynyl, benzofuranyl, dibenzofuranyl, thiopyranyl, xanthenyl, chromanyl, isochromanyl, thioazinyl, thiophenyl, benzothiophenyl, dibenzothiophenyl, difuropyrazinyl, benzofurodibenzofuranyl, benzothienobenzothiophenyl, benzothienodibenzothiophenyl, benzothienobenzofuranyl, and benzothienodibenzofuranyl.

In an aspect of the present disclosure, each of a1 and a2 may be 0.

In an aspect of the present disclosure, one of n1 and n2 may be 0, and the other one of n1 and n2 may be 1.

In an aspect of the present disclosure, R₃ may be hydrogen or a substituted or unsubstituted C6 to C30 aryl group, e.g., phenyl.

In an aspect of the present disclosure. R₄ may be hydrogen.

In an aspect of the present disclosure, each of Ar₁ to Ar₄ may be independently selected from the group consisting of a C6 to C30 aryl group, e.g., phenyl, biphenyl, naphthyl, fluorenyl or terphenyl, unsubstituted or substituted with at least one of a C1 to C10 alkyl group, a C6 to C30 aryl group and a C5 to C30 heteroaryl group and a C5 to C30 heteroaryl group, e.g., dibenzofuranyl or dibenzothiophenyl, unsubstituted or substituted with a C6 to C30 aryl group.

In an aspect of the present disclosure, R₃ in Formula 1 may be a substituted or unsubstituted C6 to C30 aryl group, e.g., phenyl, at least one of Ar₁ and Ar₂ or at least one of Ar₃ and Ar₄ may a C6 to C30 aryl group substituted with at least one of a C1 to C10 alkyl group, e.g., dimethylfluorenyl.

The organic compound in Formula 1 has a structure, where an amine group is combined to an indolo[2,3,1-jk]carbazole moiety to which an oxazole or thiazole is fused, so that the exciton generation efficiency and the energy transfer efficiency are improved. Accordingly, when the organic compound as a host is included in the emitting material layer of the OLED, the emitting efficiency and the lifespan of the OLED are improved.

In Formula 1, Y may be N, one of n1 and n2 may be 0, and the other one of n1 and n2 may be 1. Namely, Formula 1 may be represented by Formula 1-1 or Formula 1-2.

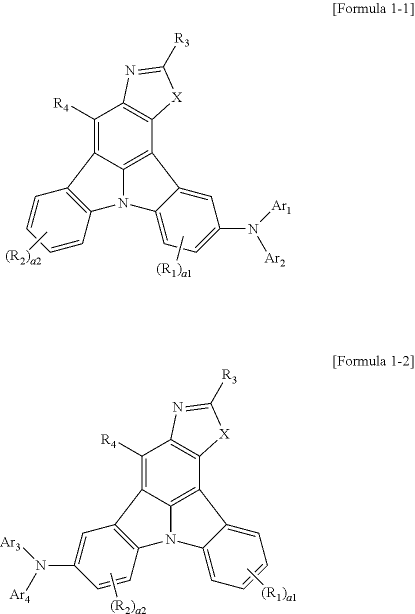

In Formula 1-1, the definitions of X, R₁ to R₄, a1, a2, Ar₁ and Ar₂ are same as those in Formula 1, and

in Formula 1-2, the definitions of X, R₁ to R₄, a1, a2, Ar₃ and Ar₄ are same as those in Formula 1.

For example, the organic compound of the present disclosure may be one of compounds in Formula 2.

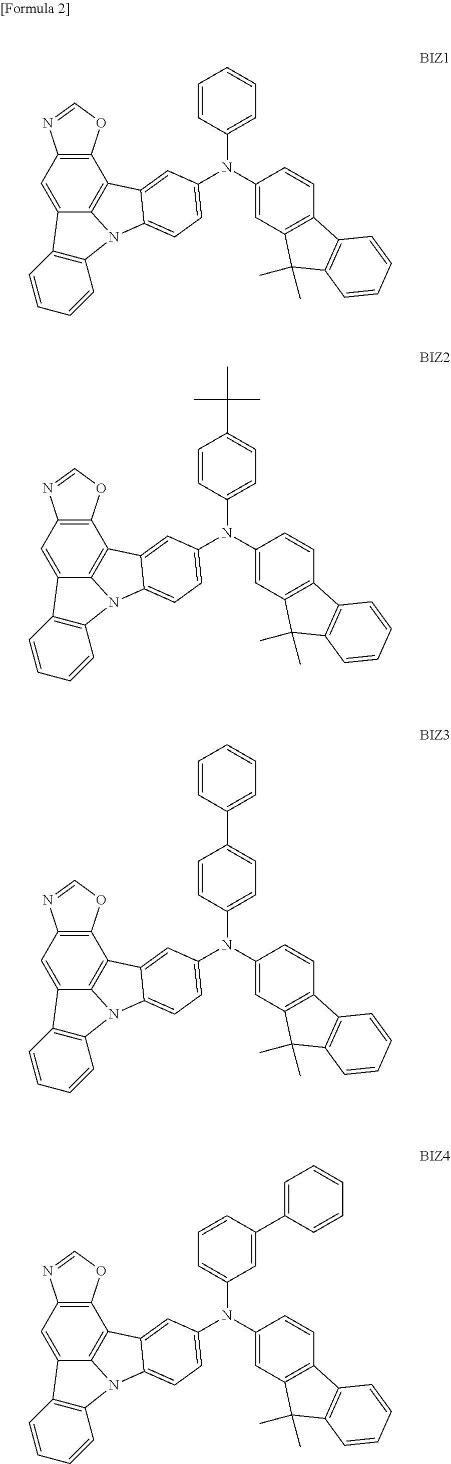 [SMILES: CC1(C)c2ccccc2-c2ccc(N(c3ccccc3)c3ccc4c(c3)c3c5ocnc5cc5c6ccccc6n4c53)cc21 ; CC(C)(C)c1ccc(N(c2ccc3c(c2)C(C)(C)c2ccccc2-3)c2ccc3c(c2)c2c4ocnc4cc4c5ccccc5n3c42)cc1 ; CC1(C)c2ccccc2-c2ccc(N(c3ccc(-c4ccccc4)cc3)c3ccc4c(c3)c3c5ocnc5cc5c6ccccc6n4c53)cc21 ; CC1(C)c2ccccc2-c2ccc(N(c3cccc(-c4ccccc4)c3)c3ccc4c(c3)c3c5ocnc5cc5c6ccccc6n4c53)cc21]

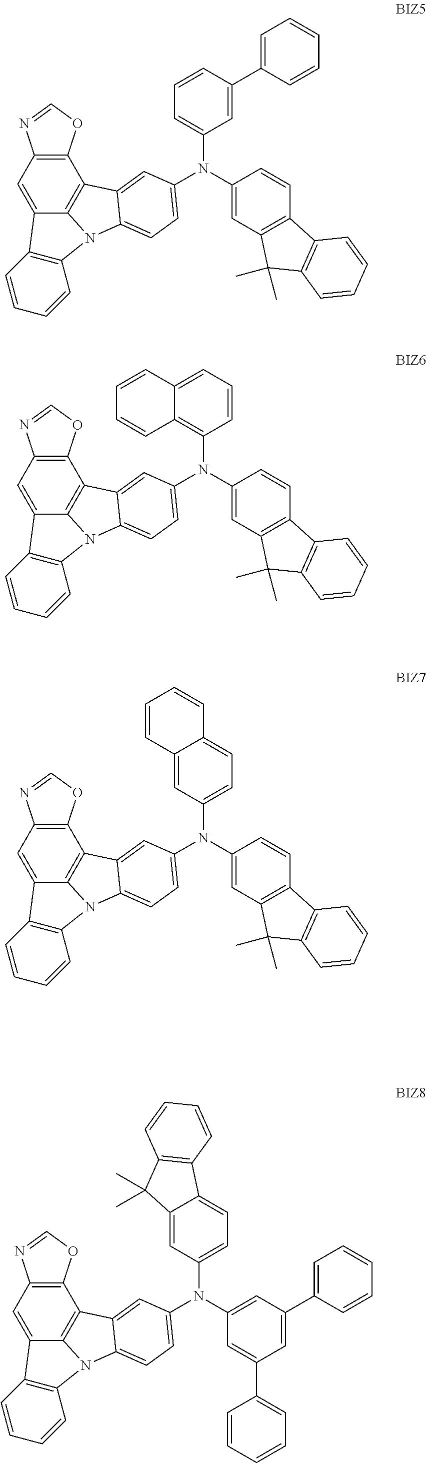 [SMILES: CC1(C)c2ccccc2-c2ccc(N(c3cccc(-c4ccccc4)c3)c3ccc4c(c3)c3c5ocnc5cc5c6ccccc6n4c53)cc21 ; CC1(C)c2ccccc2-c2ccc(N(c3ccc4c(c3)c3c5ocnc5cc5c6ccccc6n4c53)c3cccc4ccccc34)cc21 ; CC1(C)c2ccccc2-c2ccc(N(c3ccc4ccccc4c3)c3ccc4c(c3)c3c5ocnc5cc5c6ccccc6n4c53)cc21 ; CC1(C)c2ccccc2-c2ccc(N(c3cc(-c4ccccc4)cc(-c4ccccc4)c3)c3ccc4c(c3)c3c5ocnc5cc5c6ccccc6n4c53)cc21]

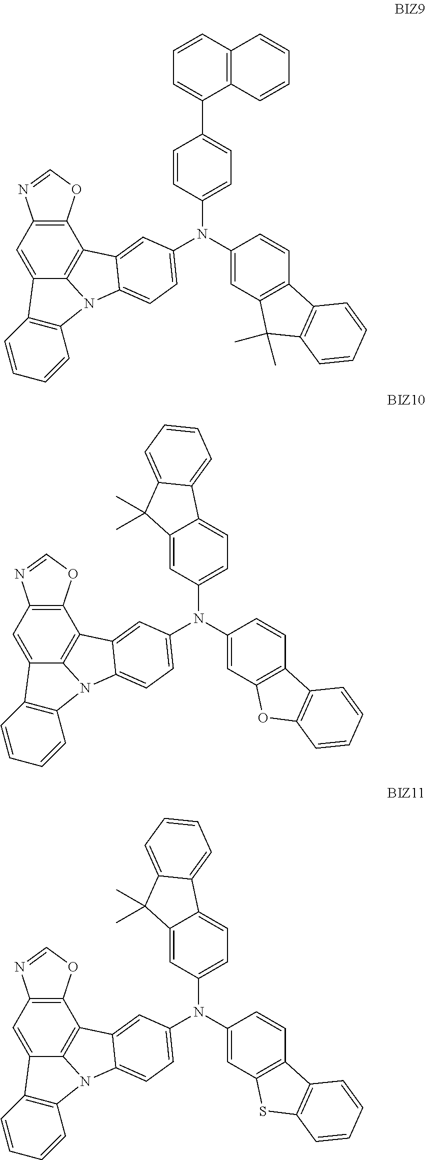 [SMILES: CC1(C)c2ccccc2-c2ccc(N(c3ccc(-c4cccc5ccccc45)cc3)c3ccc4c(c3)c3c5ocnc5cc5c6ccccc6n4c53)cc21 ; CC1(C)c2ccccc2-c2ccc(N(c3ccc4c(c3)oc3ccccc34)c3ccc4c(c3)c3c5ocnc5cc5c6ccccc6n4c53)cc21 ; CC1(C)c2ccccc2-c2ccc(N(c3ccc4c(c3)sc3ccccc34)c3ccc4c(c3)c3c5ocnc5cc5c6ccccc6n4c53)cc21]

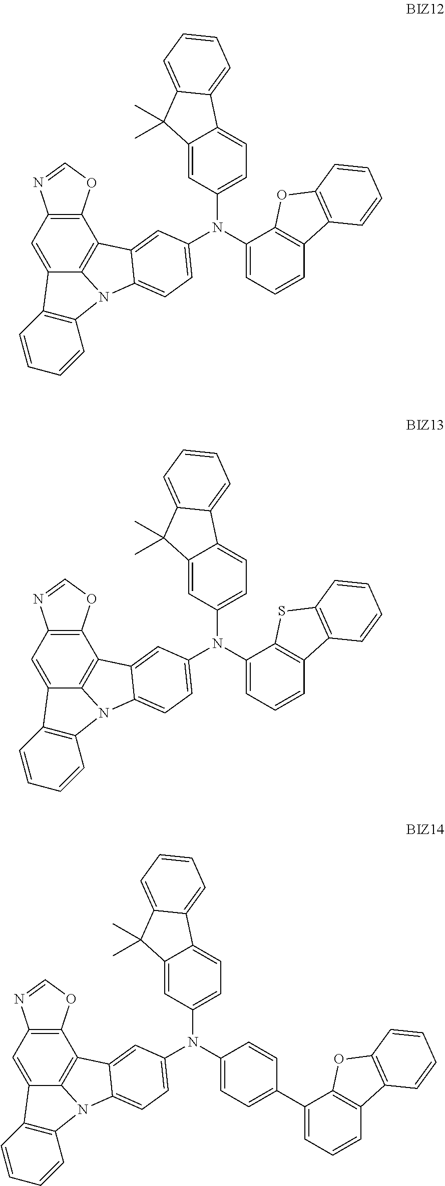 [SMILES: CC1(C)c2ccccc2-c2ccc(N(c3ccc4c(c3)c3c5ocnc5cc5c6ccccc6n4c53)c3cccc4c3oc3ccccc34)cc21 ; CC1(C)c2ccccc2-c2ccc(N(c3ccc4c(c3)c3c5ocnc5cc5c6ccccc6n4c53)c3cccc4c3sc3ccccc34)cc21 ; CC1(C)c2ccccc2-c2ccc(N(c3ccc(-c4cccc5c4oc4ccccc45)cc3)c3ccc4c(c3)c3c5ocnc5cc5c6ccccc6n4c53)cc21]

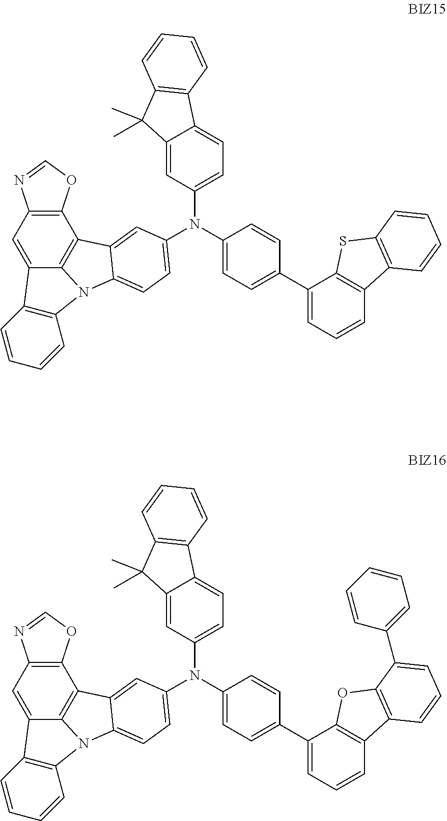 [SMILES: CC1(C)c2ccccc2-c2ccc(N(c3ccc(-c4cccc5c4sc4ccccc45)cc3)c3ccc4c(c3)c3c5ocnc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5ocnc5cc5c6ccccc6n4c53)cc21]

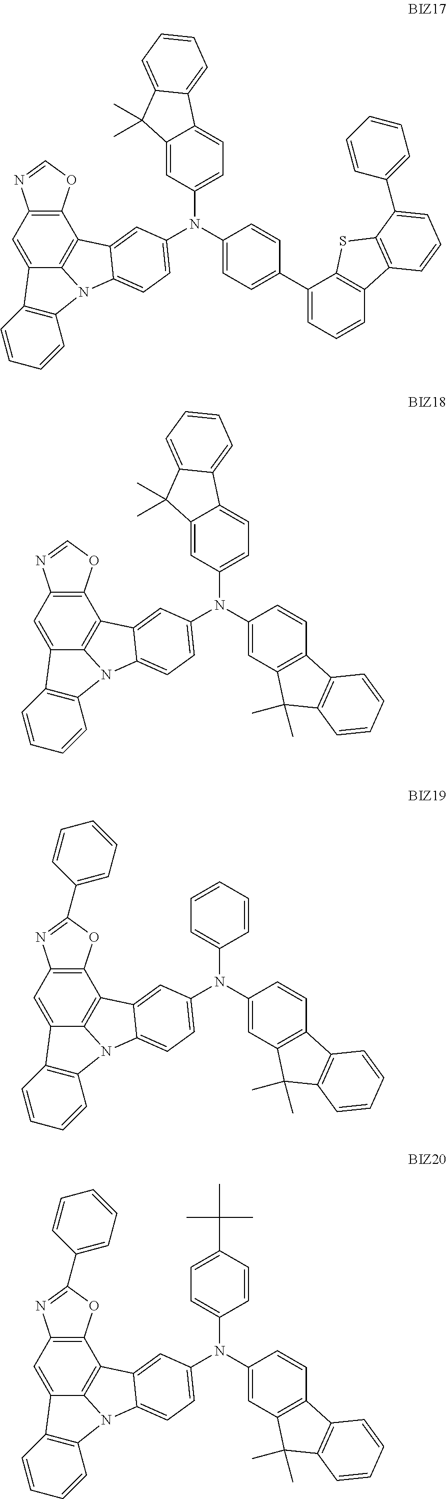 [SMILES: CC1(C)c2ccccc2-c2ccc(N(c3ccc(-c4cccc5c4sc4c(-c6ccccc6)cccc45)cc3)c3ccc4c(c3)c3c5ocnc5cc5c6ccccc6n4c53)cc21 ; CC1(C)c2ccccc2-c2ccc(N(c3ccc4c(c3)C(C)(C)c3ccccc3-4)c3ccc4c(c3)c3c5ocnc5cc5c6ccccc6n4c53)cc21 ; CC1(C)c2ccccc2-c2ccc(N(c3ccccc3)c3ccc4c(c3)c3c5oc(-c6ccccc6)nc5cc5c6ccccc6n4c53)cc21 ; CC(C)(C)c1ccc(N(c2ccc3c(c2)C(C)(C)c2ccccc2-3)c2ccc3c(c2)c2c4oc(-c5ccccc5)nc4cc4c5ccccc5n3c42)cc1]

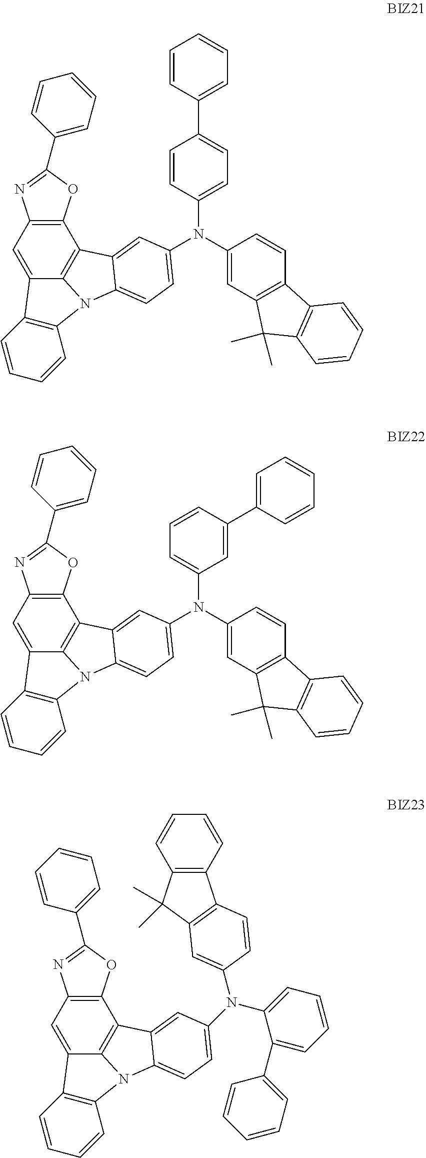 [SMILES: CC1(C)c2ccccc2-c2ccc(N(c3ccc(-c4ccccc4)cc3)c3ccc4c(c3)c3c5oc(-c6ccccc6)nc5cc5c6ccccc6n4c53)cc21 ; CC1(C)c2ccccc2-c2ccc(N(c3cccc(-c4ccccc4)c3)c3ccc4c(c3)c3c5oc(-c6ccccc6)nc5cc5c6ccccc6n4c53)cc21 ; CC1(C)c2ccccc2-c2ccc(N(c3ccc4c(c3)c3c5oc(-c6ccccc6)nc5cc5c6ccccc6n4c53)c3ccccc3-c3ccccc3)cc21]

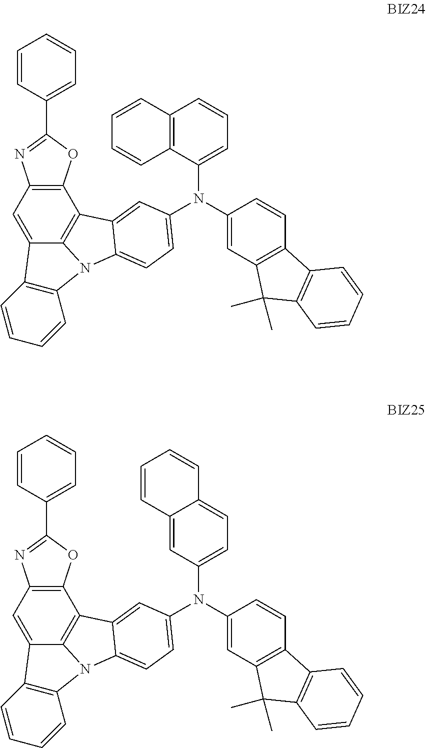 [SMILES: CC1(C)c2ccccc2-c2ccc(N(c3ccc4c(c3)c3c5oc(-c6ccccc6)nc5cc5c6ccccc6n4c53)c3cccc4ccccc34)cc21 ; CC1(C)c2ccccc2-c2ccc(N(c3ccc4ccccc4c3)c3ccc4c(c3)c3c5oc(-c6ccccc6)nc5cc5c6ccccc6n4c53)cc21]

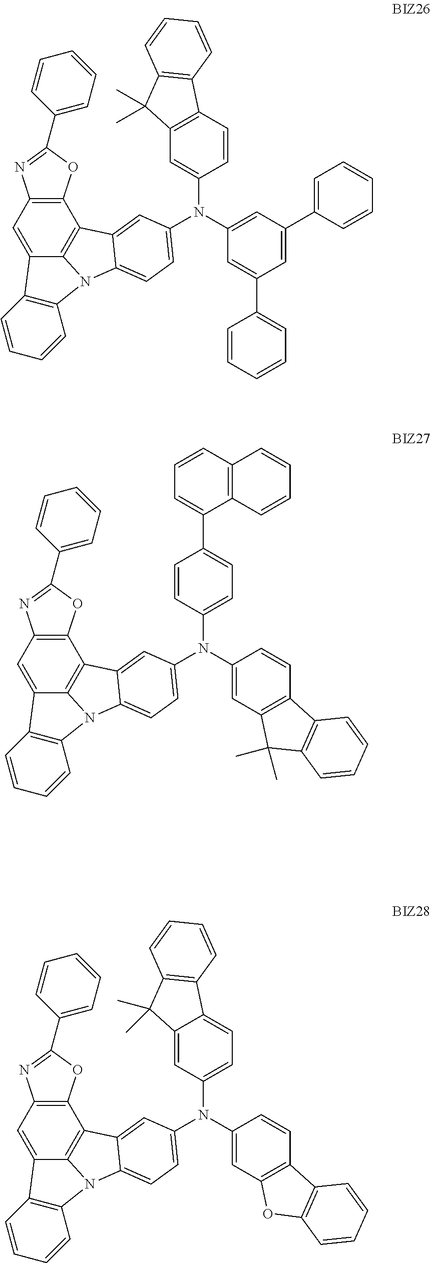 [SMILES: CC1(C)c2ccccc2-c2ccc(N(c3cc(-c4ccccc4)cc(-c4ccccc4)c3)c3ccc4c(c3)c3c5oc(-c6ccccc6)nc5cc5c6ccccc6n4c53)cc21 ; CC1(C)c2ccccc2-c2ccc(N(c3ccc(-c4cccc5ccccc45)cc3)c3ccc4c(c3)c3c5oc(-c6ccccc6)nc5cc5c6ccccc6n4c53)cc21 ; CC1(C)c2ccccc2-c2ccc(N(c3ccc4c(c3)oc3ccccc34)c3ccc4c(c3)c3c5oc(-c6ccccc6)nc5cc5c6ccccc6n4c53)cc21]

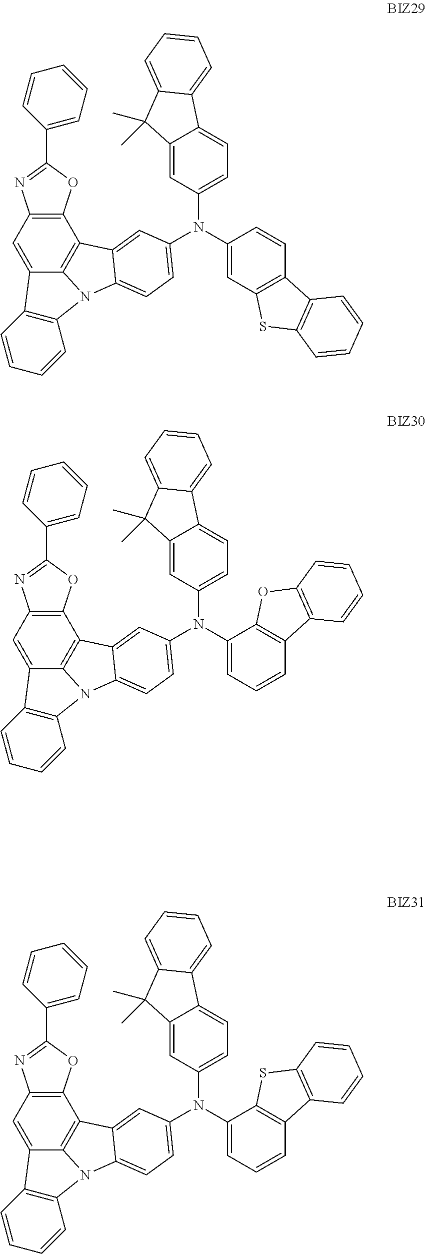 [SMILES: CC1(C)c2ccccc2-c2ccc(N(c3ccc4c(c3)sc3ccccc34)c3ccc4c(c3)c3c5oc(-c6ccccc6)nc5cc5c6ccccc6n4c53)cc21 ; CC1(C)c2ccccc2-c2ccc(N(c3ccc4c(c3)c3c5oc(-c6ccccc6)nc5cc5c6ccccc6n4c53)c3cccc4c3oc3ccccc34)cc21 ; CC1(C)c2ccccc2-c2ccc(N(c3ccc4c(c3)c3c5oc(-c6ccccc6)nc5cc5c6ccccc6n4c53)c3cccc4c3sc3ccccc34)cc21]

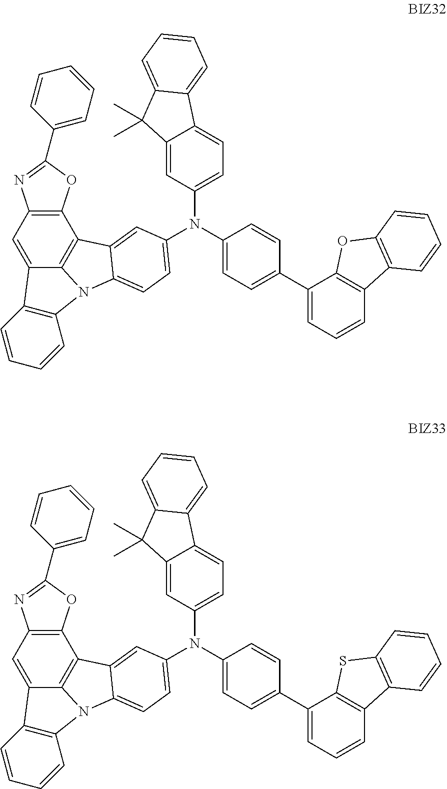 [SMILES: CC1(C)c2ccccc2-c2ccc(N(c3ccc(-c4cccc5c4oc4ccccc45)cc3)c3ccc4c(c3)c3c5oc(-c6ccccc6)nc5cc5c6ccccc6n4c53)cc21 ; CC1(C)c2ccccc2-c2ccc(N(c3ccc(-c4cccc5c4sc4ccccc45)cc3)c3ccc4c(c3)c3c5oc(-c6ccccc6)nc5cc5c6ccccc6n4c53)cc21]

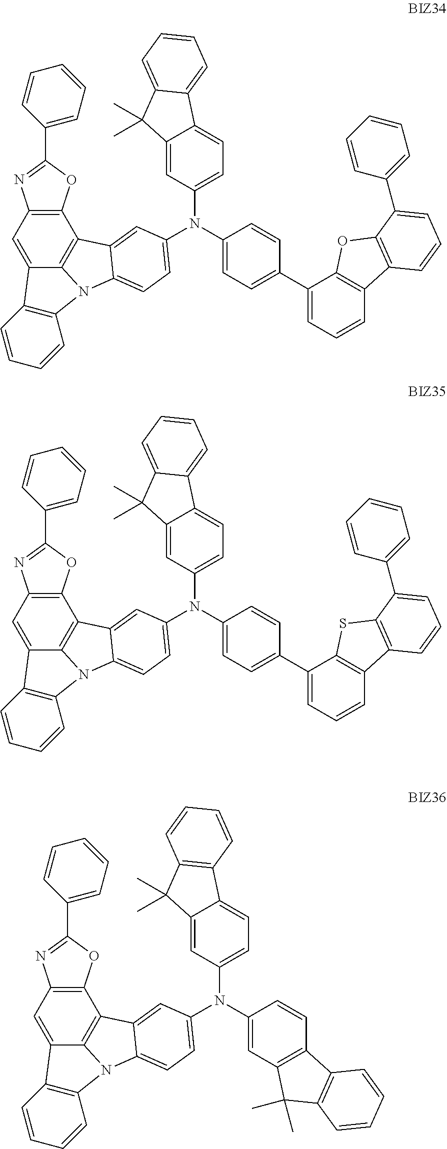 [SMILES: CC1(C)c2ccccc2-c2ccc(N(c3ccc(-c4cccc5c4oc4c(-c6ccccc6)cccc45)cc3)c3ccc4c(c3)c3c5oc(-c6ccccc6)nc5cc5c6ccccc6n4c53)cc21 ; CC1(C)c2ccccc2-c2ccc(N(c3ccc(-c4cccc5c4sc4c(-c6ccccc6)cccc45)cc3)c3ccc4c(c3)c3c5oc(-c6ccccc6)nc5cc5c6ccccc6n4c53)cc21 ; CC1(C)c2ccccc2-c2ccc(N(c3ccc4c(c3)C(C)(C)c3ccccc3-4)c3ccc4c(c3)c3c5oc(-c6ccccc6)nc5cc5c6ccccc6n4c53)cc21]

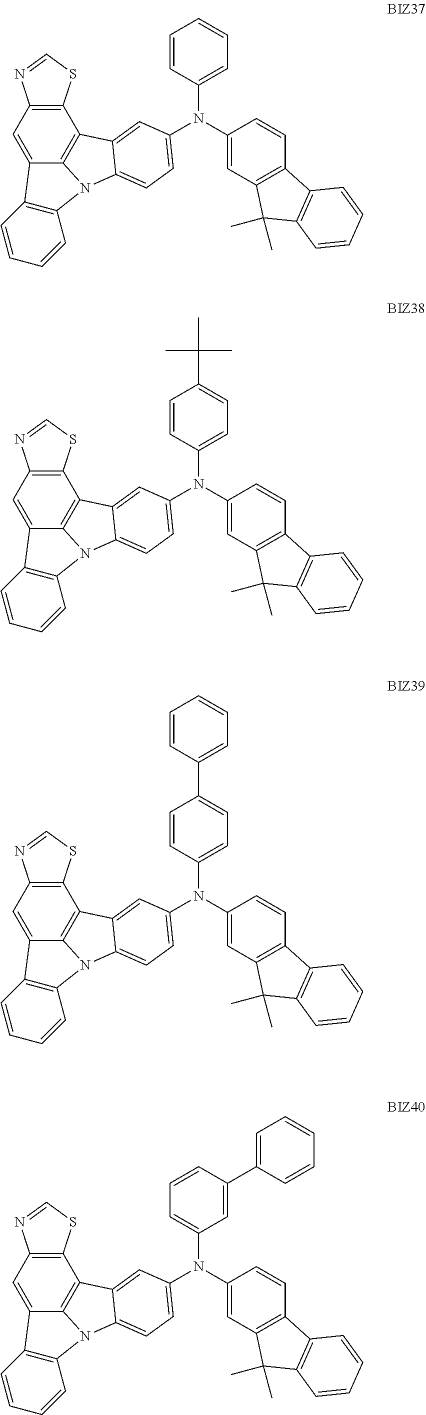 [SMILES: CC1(C)c2ccccc2-c2ccc(N(c3ccccc3)c3ccc4c(c3)c3c5scnc5cc5c6ccccc6n4c53)cc21 ; CC(C)(C)c1ccc(N(c2ccc3c(c2)C(C)(C)c2ccccc2-3)c2ccc3c(c2)c2c4scnc4cc4c5ccccc5n3c42)cc1 ; CC1(C)c2ccccc2-c2ccc(N(c3ccc(-c4ccccc4)cc3)c3ccc4c(c3)c3c5scnc5cc5c6ccccc6n4c53)cc21 ; CC1(C)c2ccccc2-c2ccc(N(c3cccc(-c4ccccc4)c3)c3ccc4c(c3)c3c5scnc5cc5c6ccccc6n4c53)cc21]

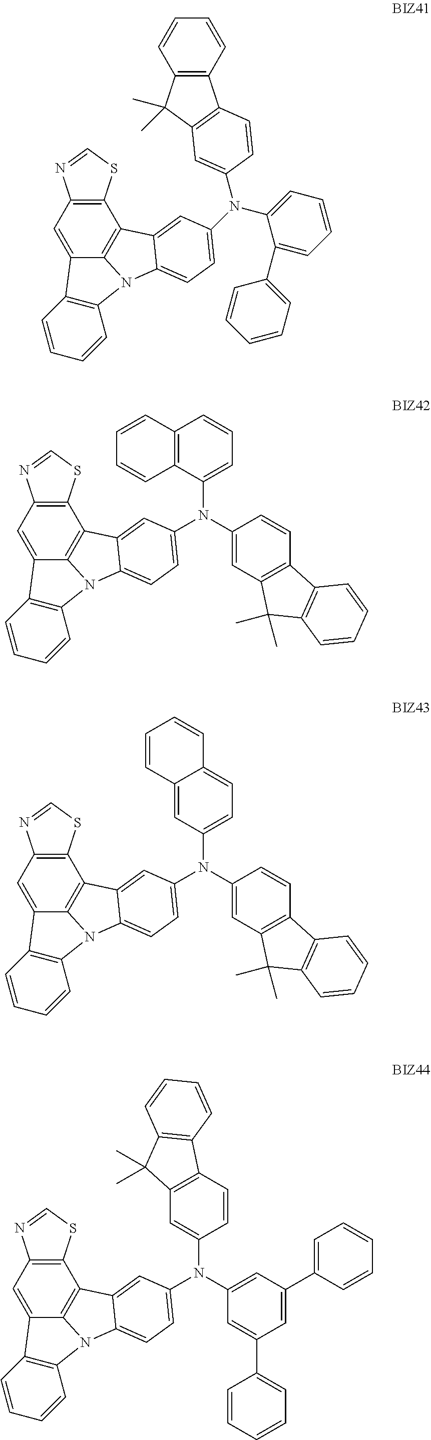 [SMILES: CC1(C)c2ccccc2-c2ccc(N(c3ccc4c(c3)c3c5scnc5cc5c6ccccc6n4c53)c3ccccc3-c3ccccc3)cc21 ; CC1(C)c2ccccc2-c2ccc(N(c3ccc4c(c3)c3c5scnc5cc5c6ccccc6n4c53)c3cccc4ccccc34)cc21 ; CC1(C)c2ccccc2-c2ccc(N(c3ccc4ccccc4c3)c3ccc4c(c3)c3c5scnc5cc5c6ccccc6n4c53)cc21 ; CC1(C)c2ccccc2-c2ccc(N(c3cc(-c4ccccc4)cc(-c4ccccc4)c3)c3ccc4c(c3)c3c5scnc5cc5c6ccccc6n4c53)cc21]

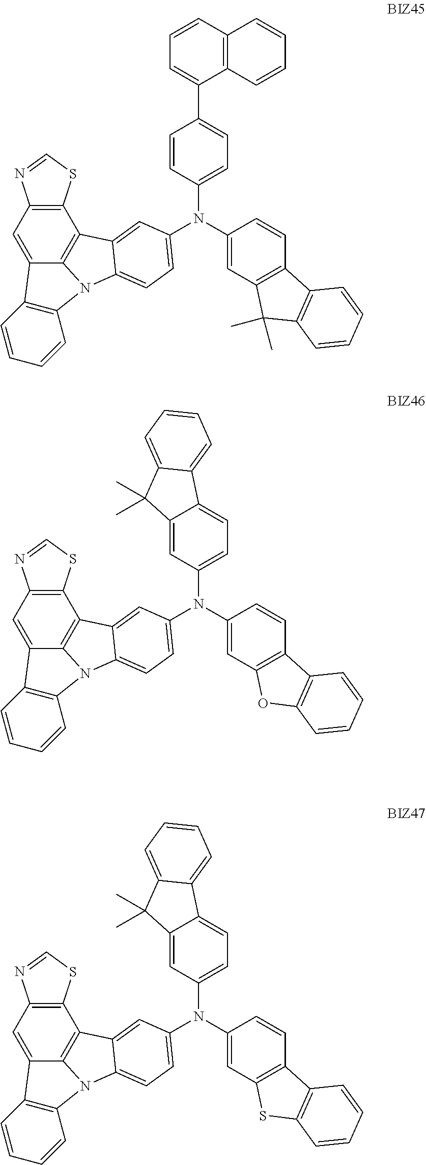 [SMILES: CC1(C)c2ccccc2-c2ccc(N(c3ccc(-c4cccc5ccccc45)cc3)c3ccc4c(c3)c3c5scnc5cc5c6ccccc6n4c53)cc21 ; CC1(C)c2ccccc2-c2ccc(N(c3ccc4c(c3)oc3ccccc34)c3ccc4c(c3)c3c5scnc5cc5c6ccccc6n4c53)cc21 ; CC1(C)c2ccccc2-c2ccc(N(c3ccc4c(c3)sc3ccccc34)c3ccc4c(c3)c3c5scnc5cc5c6ccccc6n4c53)cc21]

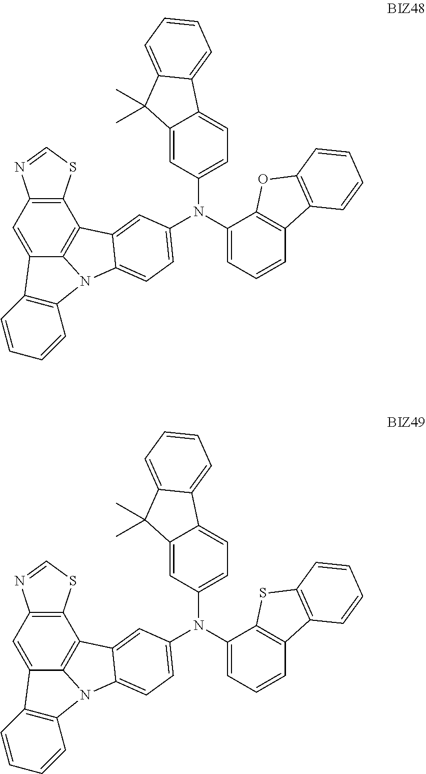 [SMILES: CC1(C)c2ccccc2-c2ccc(N(c3ccc4c(c3)c3c5scnc5cc5c6ccccc6n4c53)c3cccc4c3oc3ccccc34)cc21 ; CC1(C)c2ccccc2-c2ccc(N(c3ccc4c(c3)c3c5scnc5cc5c6ccccc6n4c53)c3cccc4c3sc3ccccc34)cc21]

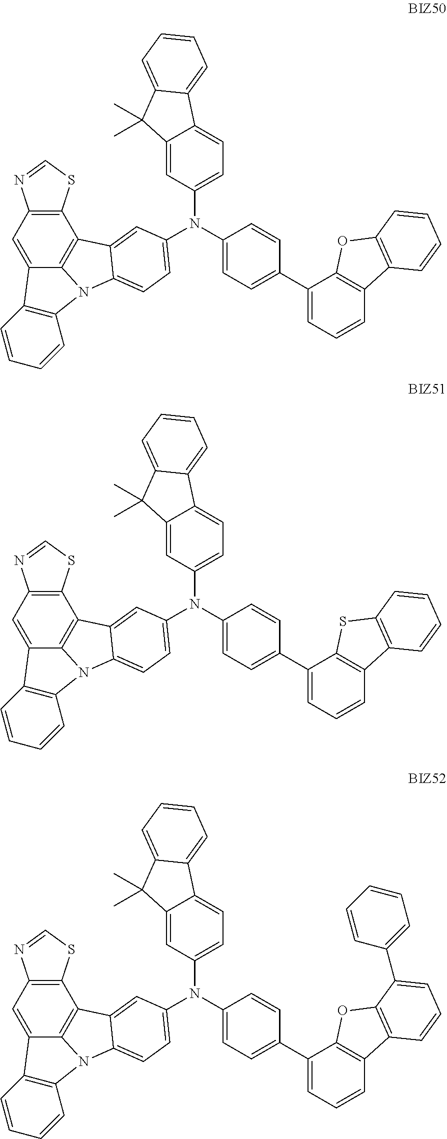 [SMILES: CC1(C)c2ccccc2-c2ccc(N(c3ccc(-c4cccc5c4oc4ccccc45)cc3)c3ccc4c(c3)c3c5scnc5cc5c6ccccc6n4c53)cc21 ; CC1(C)c2ccccc2-c2ccc(N(c3ccc(-c4cccc5c4sc4ccccc45)cc3)c3ccc4c(c3)c3c5scnc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5scnc5cc5c6ccccc6n4c53)cc21]

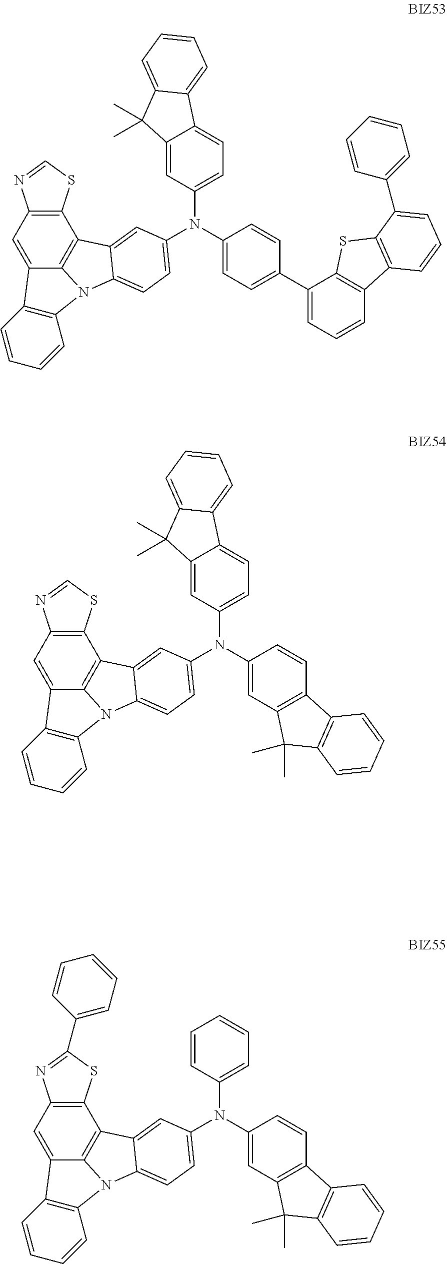 [SMILES: CC1(C)c2ccccc2-c2ccc(N(c3ccc(-c4cccc5c4sc4c(-c6ccccc6)cccc45)cc3)c3ccc4c(c3)c3c5scnc5cc5c6ccccc6n4c53)cc21 ; CC1(C)c2ccccc2-c2ccc(N(c3ccc4c(c3)C(C)(C)c3ccccc3-4)c3ccc4c(c3)c3c5scnc5cc5c6ccccc6n4c53)cc21 ; CC1(C)c2ccccc2-c2ccc(N(c3ccccc3)c3ccc4c(c3)c3c5sc(-c6ccccc6)nc5cc5c6ccccc6n4c53)cc21]

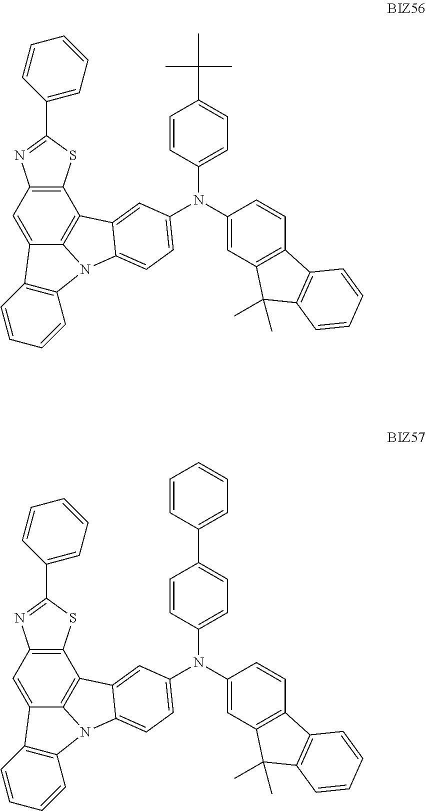 [SMILES: CC(C)(C)c1ccc(N(c2ccc3c(c2)C(C)(C)c2ccccc2-3)c2ccc3c(c2)c2c4sc(-c5ccccc5)nc4cc4c5ccccc5n3c42)cc1 ; CC1(C)c2ccccc2-c2ccc(N(c3ccc(-c4ccccc4)cc3)c3ccc4c(c3)c3c5sc(-c6ccccc6)nc5cc5c6ccccc6n4c53)cc21]

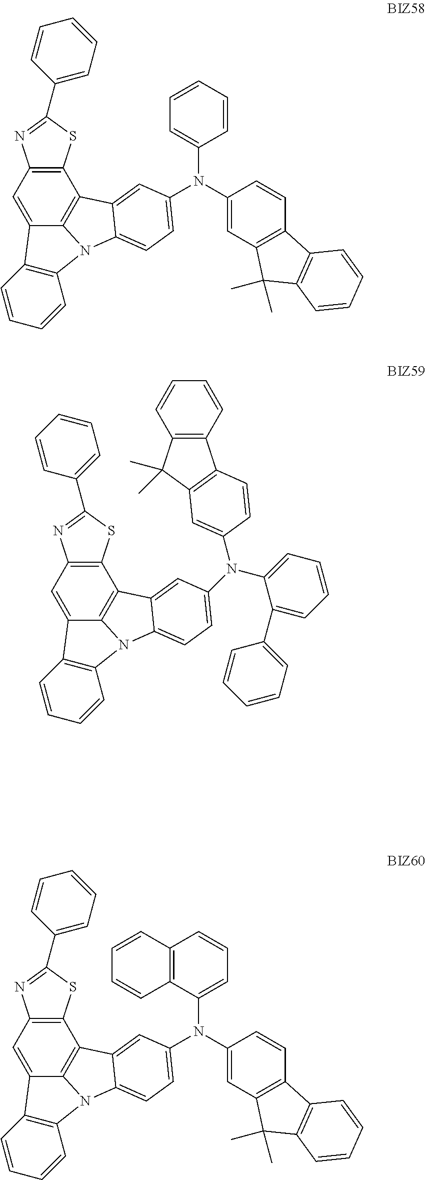 [SMILES: CC1(C)c2ccccc2-c2ccc(N(c3ccccc3)c3ccc4c(c3)c3c5sc(-c6ccccc6)nc5cc5c6ccccc6n4c53)cc21 ; CC1(C)c2ccccc2-c2ccc(N(c3ccc4c(c3)c3c5sc(-c6ccccc6)nc5cc5c6ccccc6n4c53)c3ccccc3-c3ccccc3)cc21 ; CC1(C)c2ccccc2-c2ccc(N(c3ccc4c(c3)c3c5sc(-c6ccccc6)nc5cc5c6ccccc6n4c53)c3cccc4ccccc34)cc21]

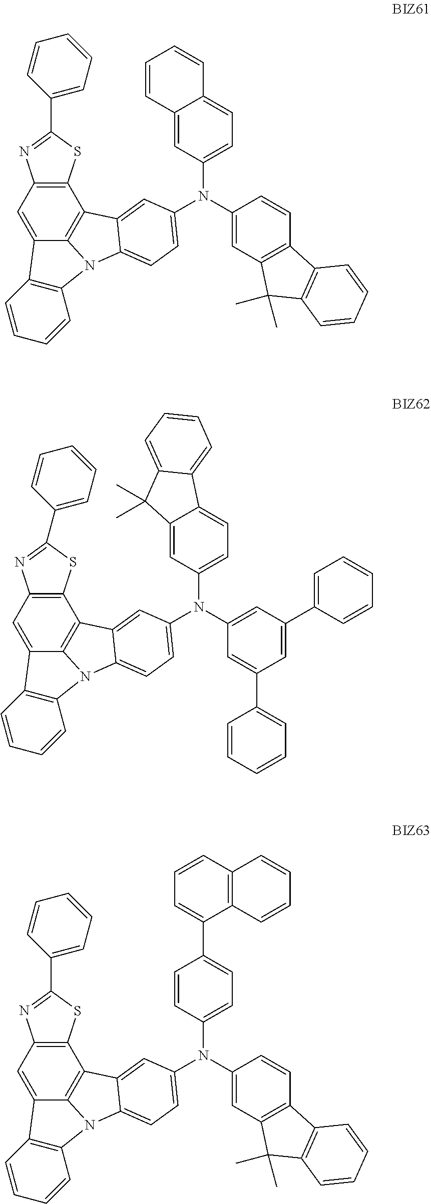 [SMILES: CC1(C)c2ccccc2-c2ccc(N(c3ccc4ccccc4c3)c3ccc4c(c3)c3c5sc(-c6ccccc6)nc5cc5c6ccccc6n4c53)cc21 ; CC1(C)c2ccccc2-c2ccc(N(c3cc(-c4ccccc4)cc(-c4ccccc4)c3)c3ccc4c(c3)c3c5sc(-c6ccccc6)nc5cc5c6ccccc6n4c53)cc21 ; CC1(C)c2ccccc2-c2ccc(N(c3ccc(-c4cccc5ccccc45)cc3)c3ccc4c(c3)c3c5sc(-c6ccccc6)nc5cc5c6ccccc6n4c53)cc21]

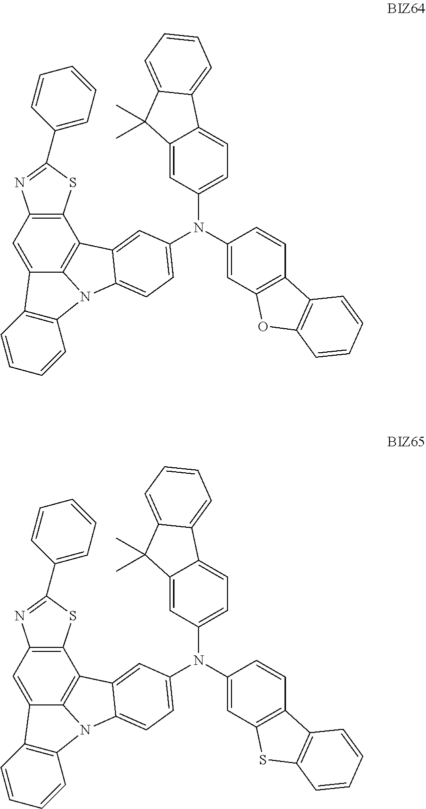 [SMILES: CC1(C)c2ccccc2-c2ccc(N(c3ccc4c(c3)oc3ccccc34)c3ccc4c(c3)c3c5sc(-c6ccccc6)nc5cc5c6ccccc6n4c53)cc21 ; CC1(C)c2ccccc2-c2ccc(N(c3ccc4c(c3)sc3ccccc34)c3ccc4c(c3)c3c5sc(-c6ccccc6)nc5cc5c6ccccc6n4c53)cc21]

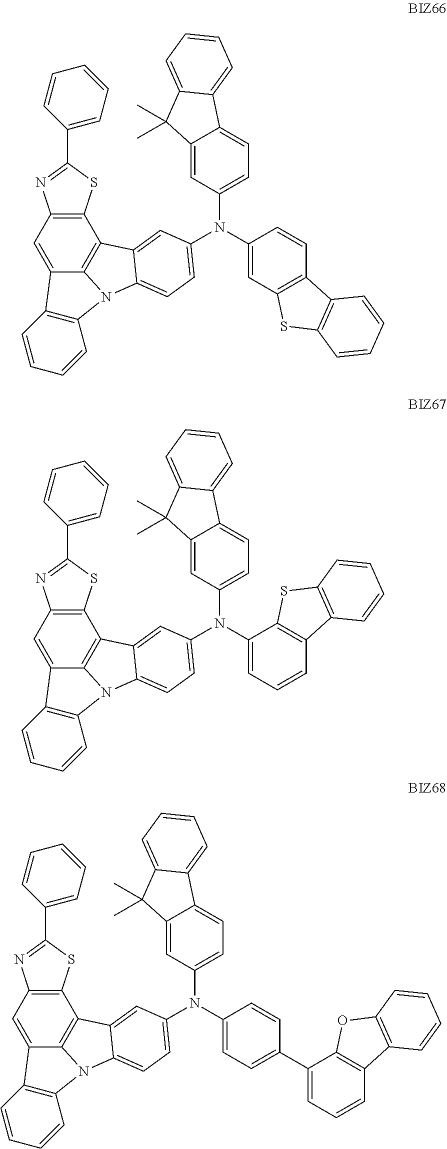 [SMILES: CC1(C)c2ccccc2-c2ccc(N(c3ccc4c(c3)sc3ccccc34)c3ccc4c(c3)c3c5sc(-c6ccccc6)nc5cc5c6ccccc6n4c53)cc21 ; CC1(C)c2ccccc2-c2ccc(N(c3ccc4c(c3)c3c5sc(-c6ccccc6)nc5cc5c6ccccc6n4c53)c3cccc4c3sc3ccccc34)cc21 ; CC1(C)c2ccccc2-c2ccc(N(c3ccc(-c4cccc5c4oc4ccccc45)cc3)c3ccc4c(c3)c3c5sc(-c6ccccc6)nc5cc5c6ccccc6n4c53)cc21]

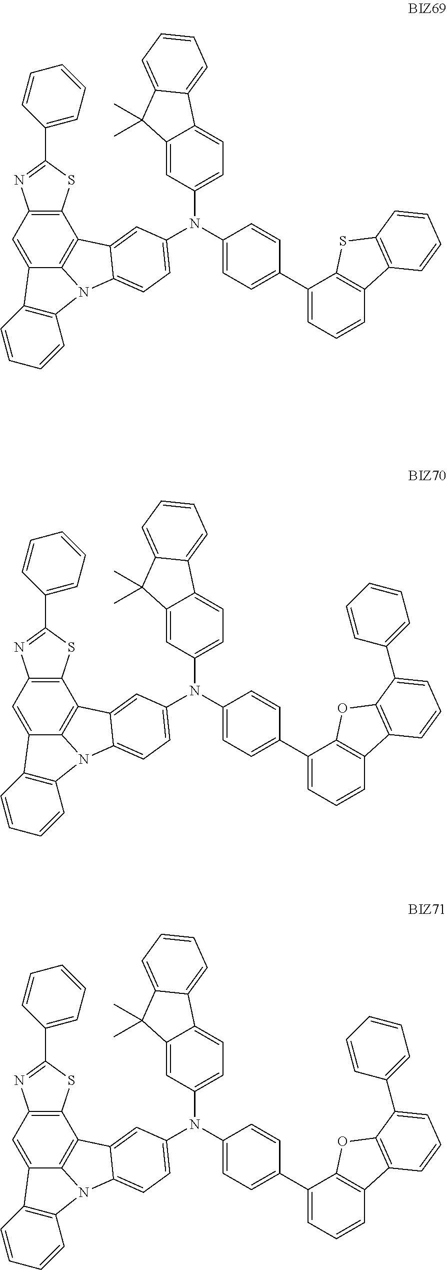 [SMILES: CC1(C)c2ccccc2-c2ccc(N(c3ccc(-c4cccc5c4sc4ccccc45)cc3)c3ccc4c(c3)c3c5sc(-c6ccccc6)nc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5sc(-c6ccccc6)nc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5sc(-c6ccccc6)nc5cc5c6ccccc6n4c53)cc21]

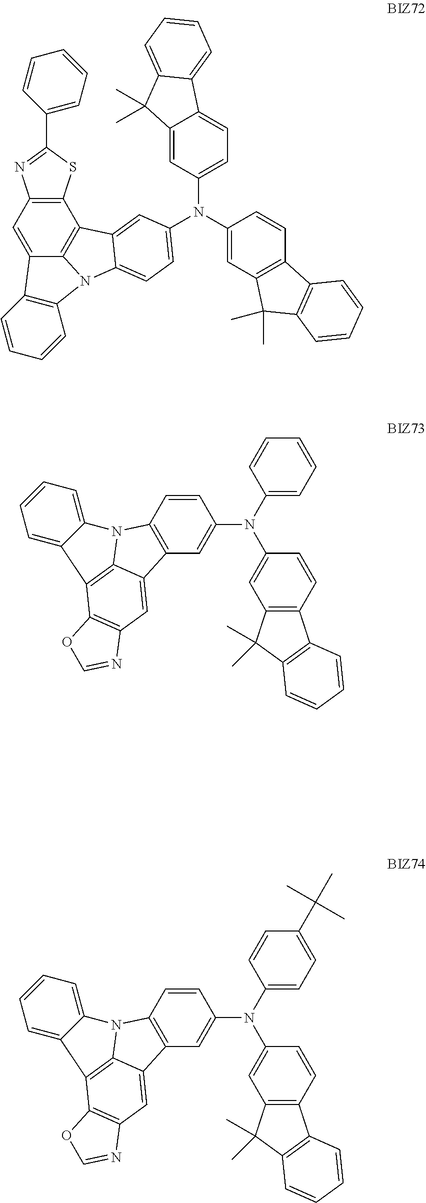 [SMILES: CC1(C)c2ccccc2-c2ccc(N(c3ccc4c(c3)C(C)(C)c3ccccc3-4)c3ccc4c(c3)c3c5sc(-c6ccccc6)nc5cc5c6ccccc6n4c53)cc21 ; CC1(C)c2ccccc2-c2ccc(N(c3ccccc3)c3ccc4c(c3)c3cc5ncoc5c5c6ccccc6n4c35)cc21 ; CC(C)(C)c1ccc(N(c2ccc3c(c2)C(C)(C)c2ccccc2-3)c2ccc3c(c2)c2cc4ncoc4c4c5ccccc5n3c24)cc1]

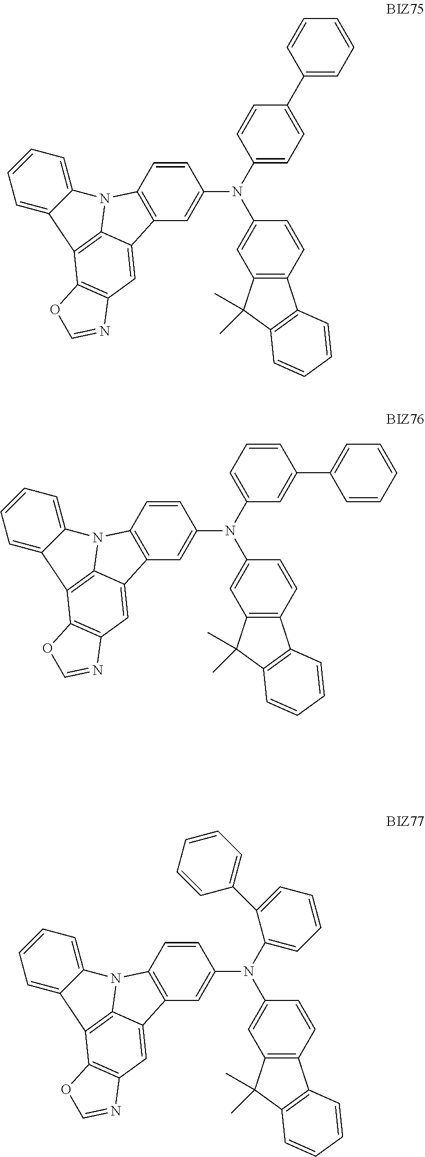 [SMILES: CC1(C)c2ccccc2-c2ccc(N(c3ccc(-c4ccccc4)cc3)c3ccc4c(c3)c3cc5ncoc5c5c6ccccc6n4c35)cc21 ; CC1(C)c2ccccc2-c2ccc(N(c3cccc(-c4ccccc4)c3)c3ccc4c(c3)c3cc5ncoc5c5c6ccccc6n4c35)cc21 ; CC1(C)c2ccccc2-c2ccc(N(c3ccc4c(c3)c3cc5ncoc5c5c6ccccc6n4c35)c3ccccc3-c3ccccc3)cc21]

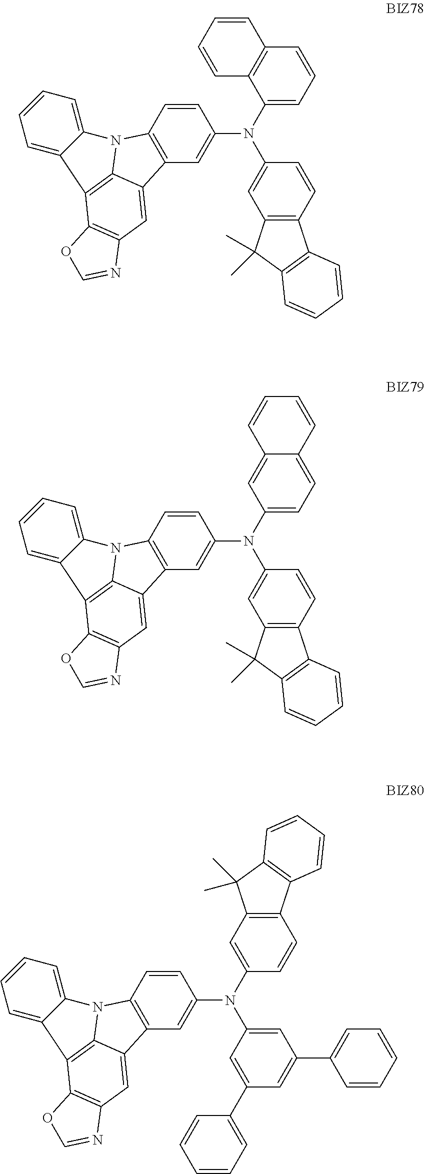 [SMILES: CC1(C)c2ccccc2-c2ccc(N(c3ccc4c(c3)c3cc5ncoc5c5c6ccccc6n4c35)c3cccc4ccccc34)cc21 ; CC1(C)c2ccccc2-c2ccc(N(c3ccc4ccccc4c3)c3ccc4c(c3)c3cc5ncoc5c5c6ccccc6n4c35)cc21 ; CC1(C)c2ccccc2-c2ccc(N(c3cc(-c4ccccc4)cc(-c4ccccc4)c3)c3ccc4c(c3)c3cc5ncoc5c5c6ccccc6n4c35)cc21]

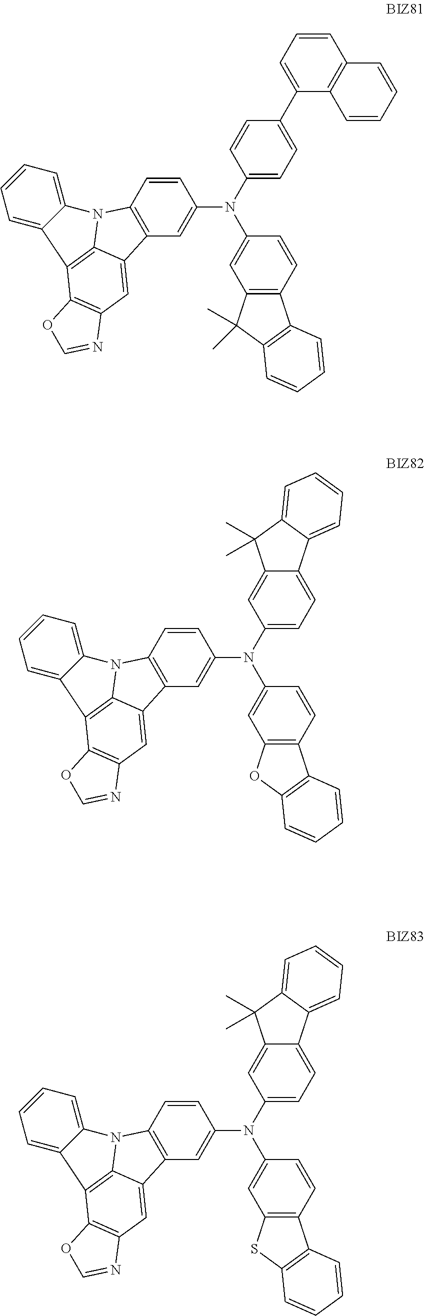 [SMILES: CC1(C)c2ccccc2-c2ccc(N(c3ccc(-c4cccc5ccccc45)cc3)c3ccc4c(c3)c3cc5ncoc5c5c6ccccc6n4c35)cc21 ; CC1(C)c2ccccc2-c2ccc(N(c3ccc4c(c3)oc3ccccc34)c3ccc4c(c3)c3cc5ncoc5c5c6ccccc6n4c35)cc21 ; CC1(C)c2ccccc2-c2ccc(N(c3ccc4c(c3)sc3ccccc34)c3ccc4c(c3)c3cc5ncoc5c5c6ccccc6n4c35)cc21]

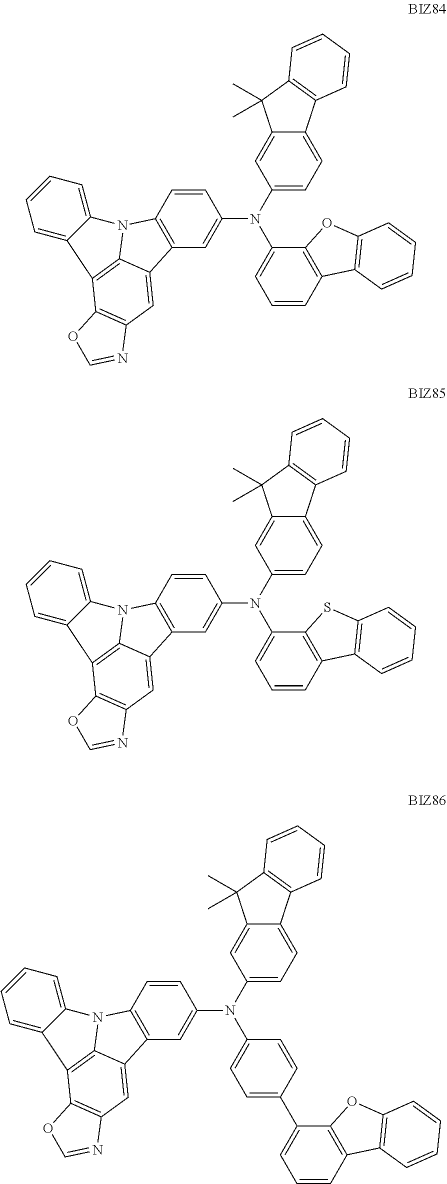 [SMILES: CC1(C)c2ccccc2-c2ccc(N(c3ccc4c(c3)c3cc5ncoc5c5c6ccccc6n4c35)c3cccc4c3oc3ccccc34)cc21 ; CC1(C)c2ccccc2-c2ccc(N(c3ccc4c(c3)c3cc5ncoc5c5c6ccccc6n4c35)c3cccc4c3sc3ccccc34)cc21 ; CC1(C)c2ccccc2-c2ccc(N(c3ccc(-c4cccc5c4oc4ccccc45)cc3)c3ccc4c(c3)c3cc5ncoc5c5c6ccccc6n4c35)cc21]

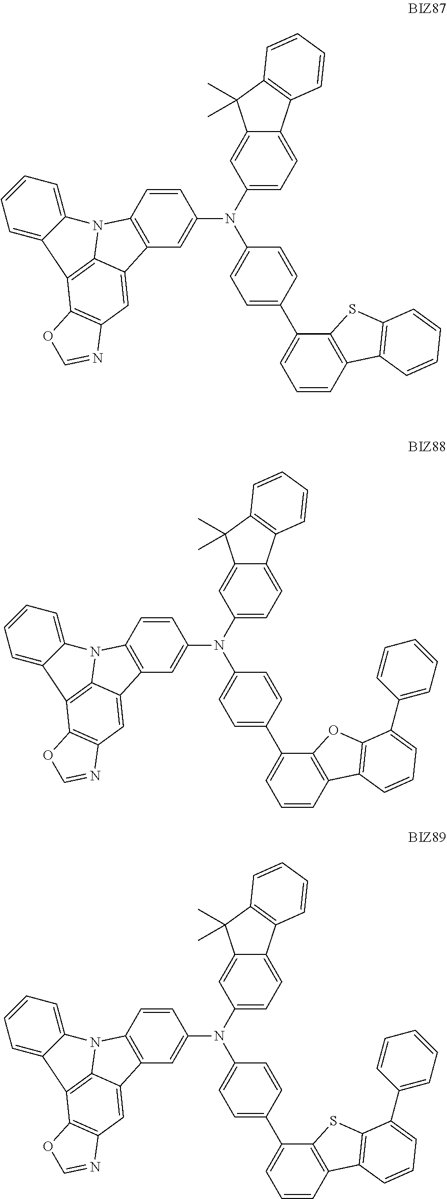 [SMILES: CC1(C)c2ccccc2-c2ccc(N(c3ccc(-c4cccc5c4sc4ccccc45)cc3)c3ccc4c(c3)c3cc5ncoc5c5c6ccccc6n4c35)cc21 ; CC1(C)c2ccccc2-c2ccc(N(c3ccc(-c4cccc5c4oc4c(-c6ccccc6)cccc45)cc3)c3ccc4c(c3)c3cc5ncoc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5ncoc5c5c6ccccc6n4c35)cc21]

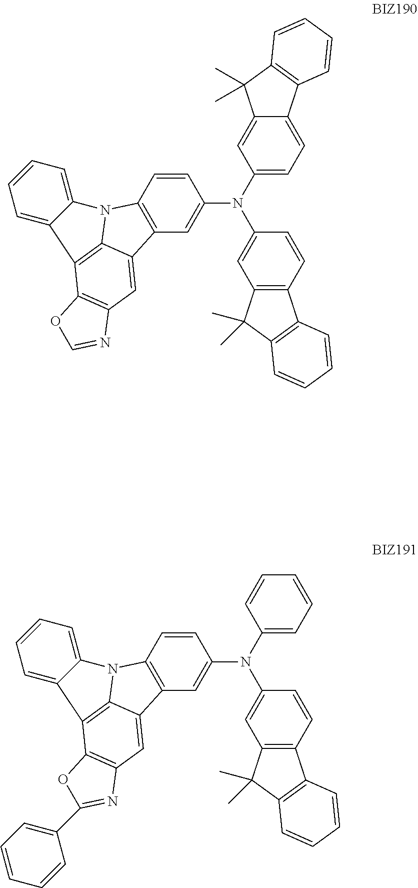 [SMILES: CC1(C)c2ccccc2-c2ccc(N(c3ccc4c(c3)C(C)(C)c3ccccc3-4)c3ccc4c(c3)c3cc5ncoc5c5c6ccccc6n4c35)cc21 ; CC1(C)c2ccccc2-c2ccc(N(c3ccccc3)c3ccc4c(c3)c3cc5nc(-c6ccccc6)oc5c5c6ccccc6n4c35)cc21]

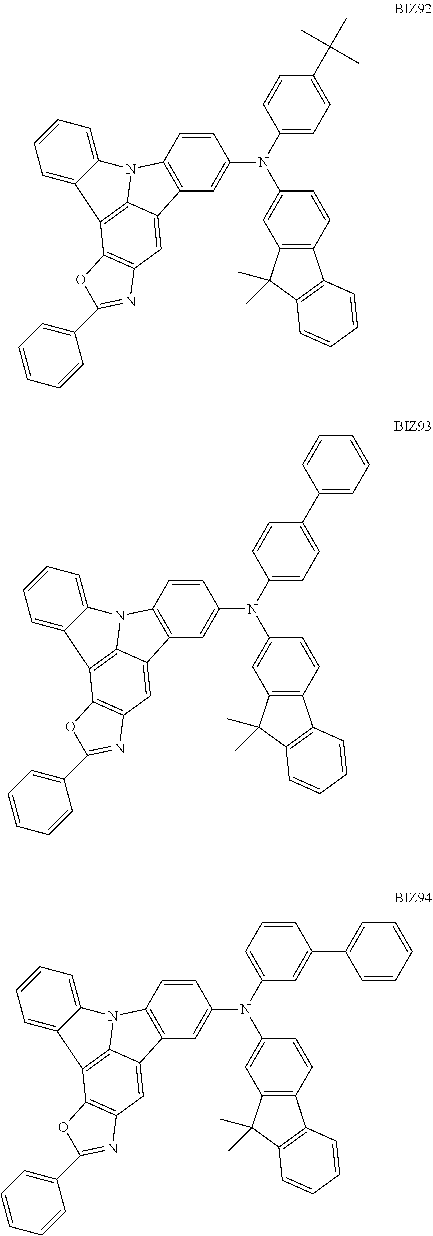 [SMILES: CC(C)(C)c1ccc(N(c2ccc3c(c2)C(C)(C)c2ccccc2-3)c2ccc3c(c2)c2cc4nc(-c5ccccc5)oc4c4c5ccccc5n3c24)cc1 ; CC1(C)c2ccccc2-c2ccc(N(c3ccc(-c4ccccc4)cc3)c3ccc4c(c3)c3cc5nc(-c6ccccc6)oc5c5c6ccccc6n4c35)cc21 ; CC1(C)c2ccccc2-c2ccc(N(c3cccc(-c4ccccc4)c3)c3ccc4c(c3)c3cc5nc(-c6ccccc6)oc5c5c6ccccc6n4c35)cc21]

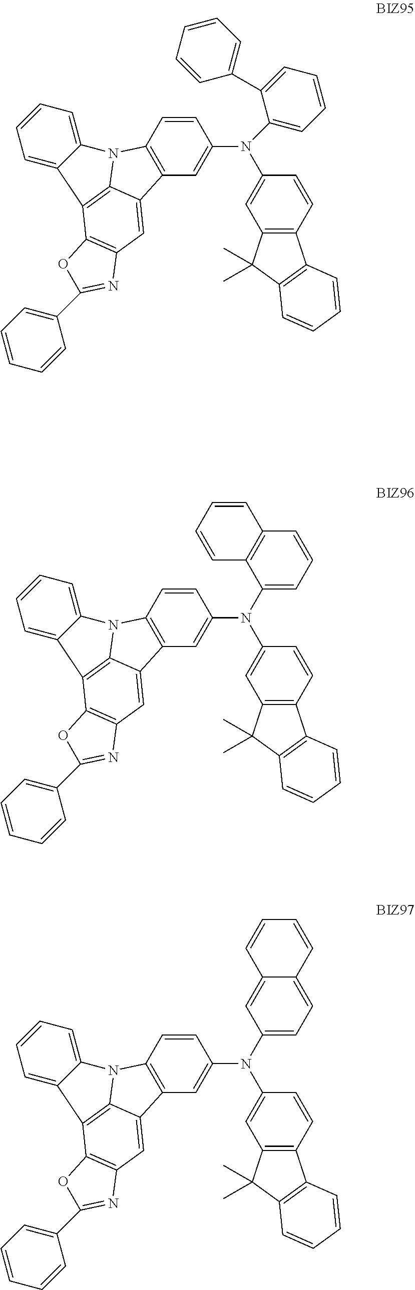 [SMILES: CC1(C)c2ccccc2-c2ccc(N(c3ccc4c(c3)c3cc5nc(-c6ccccc6)oc5c5c6ccccc6n4c35)c3ccccc3-c3ccccc3)cc21 ; CC1(C)c2ccccc2-c2ccc(N(c3ccc4c(c3)c3cc5nc(-c6ccccc6)oc5c5c6ccccc6n4c35)c3cccc4ccccc34)cc21 ; CC1(C)c2ccccc2-c2ccc(N(c3ccc4ccccc4c3)c3ccc4c(c3)c3cc5nc(-c6ccccc6)oc5c5c6ccccc6n4c35)cc21]

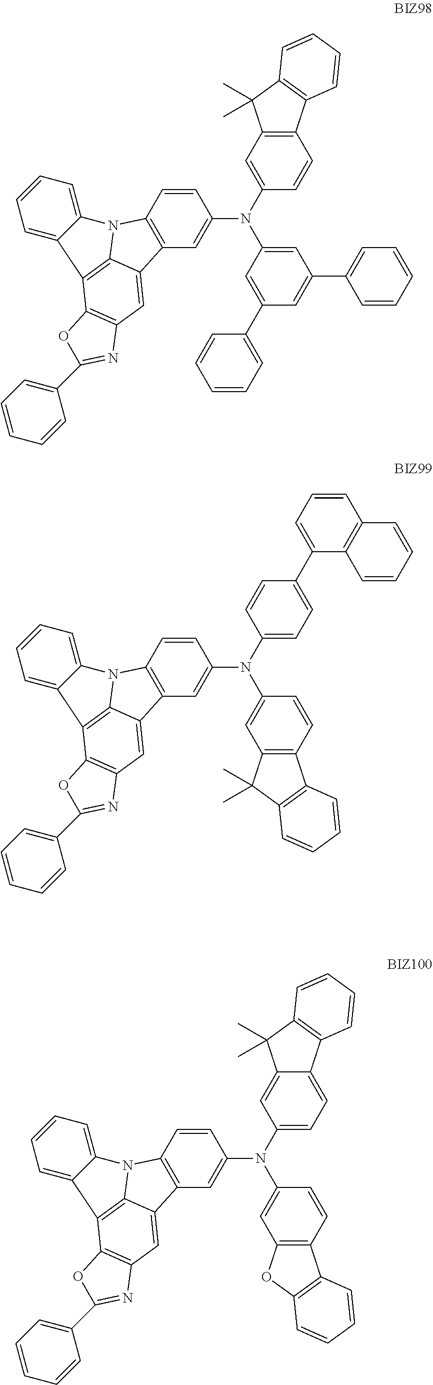 [SMILES: CC1(C)c2ccccc2-c2ccc(N(c3cc(-c4ccccc4)cc(-c4ccccc4)c3)c3ccc4c(c3)c3cc5nc(-c6ccccc6)oc5c5c6ccccc6n4c35)cc21 ; CC1(C)c2ccccc2-c2ccc(N(c3ccc(-c4cccc5ccccc45)cc3)c3ccc4c(c3)c3cc5nc(-c6ccccc6)oc5c5c6ccccc6n4c35)cc21 ; CC1(C)c2ccccc2-c2ccc(N(c3ccc4c(c3)oc3ccccc34)c3ccc4c(c3)c3cc5nc(-c6ccccc6)oc5c5c6ccccc6n4c35)cc21]

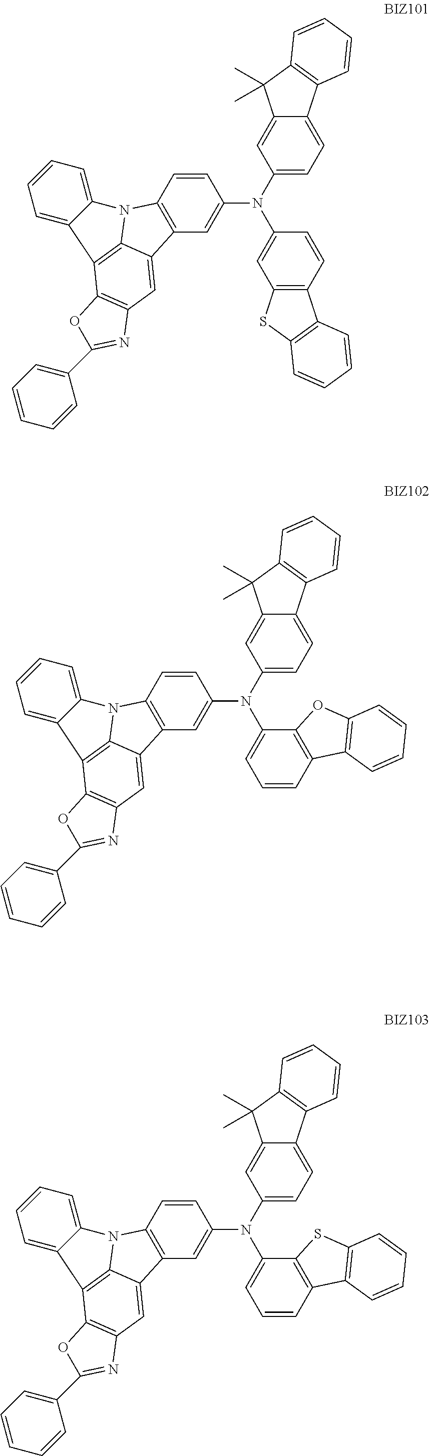 [SMILES: CC1(C)c2ccccc2-c2ccc(N(c3ccc4c(c3)sc3ccccc34)c3ccc4c(c3)c3cc5nc(-c6ccccc6)oc5c5c6ccccc6n4c35)cc21 ; CC1(C)c2ccccc2-c2ccc(N(c3ccc4c(c3)c3cc5nc(-c6ccccc6)oc5c5c6ccccc6n4c35)c3cccc4c3oc3ccccc34)cc21 ; CC1(C)c2ccccc2-c2ccc(N(c3ccc4c(c3)c3cc5nc(-c6ccccc6)oc5c5c6ccccc6n4c35)c3cccc4c3sc3ccccc34)cc21]

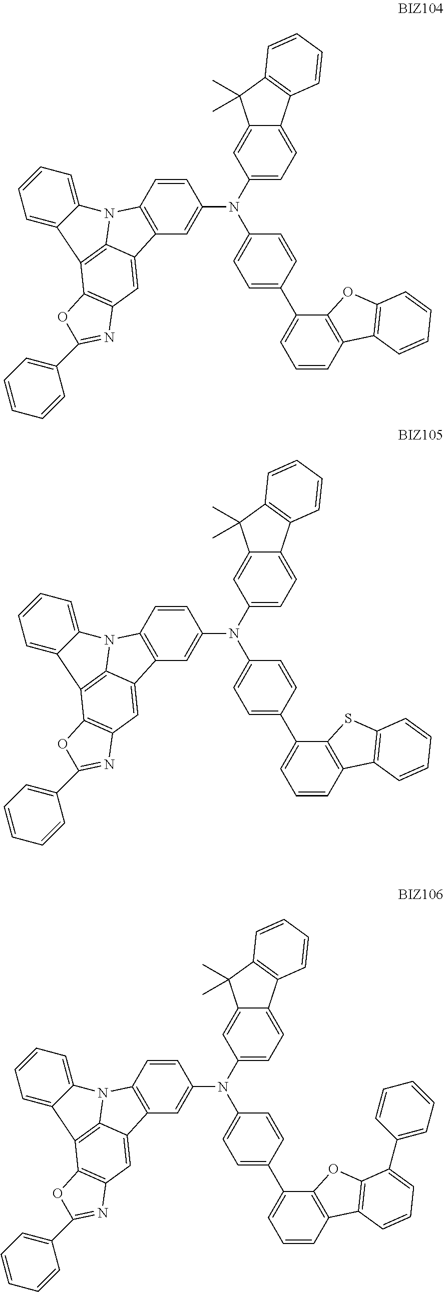 [SMILES: CC1(C)c2ccccc2-c2ccc(N(c3ccc(-c4cccc5c4oc4ccccc45)cc3)c3ccc4c(c3)c3cc5nc(-c6ccccc6)oc5c5c6ccccc6n4c35)cc21 ; CC1(C)c2ccccc2-c2ccc(N(c3ccc(-c4cccc5c4sc4ccccc45)cc3)c3ccc4c(c3)c3cc5nc(-c6ccccc6)oc5c5c6ccccc6n4c35)cc21 ; CC1(C)c2ccccc2-c2ccc(N(c3ccc(-c4cccc5c4oc4c(-c6ccccc6)cccc45)cc3)c3ccc4c(c3)c3cc5nc(-c6ccccc6)oc5c5c6ccccc6n4c35)cc21]

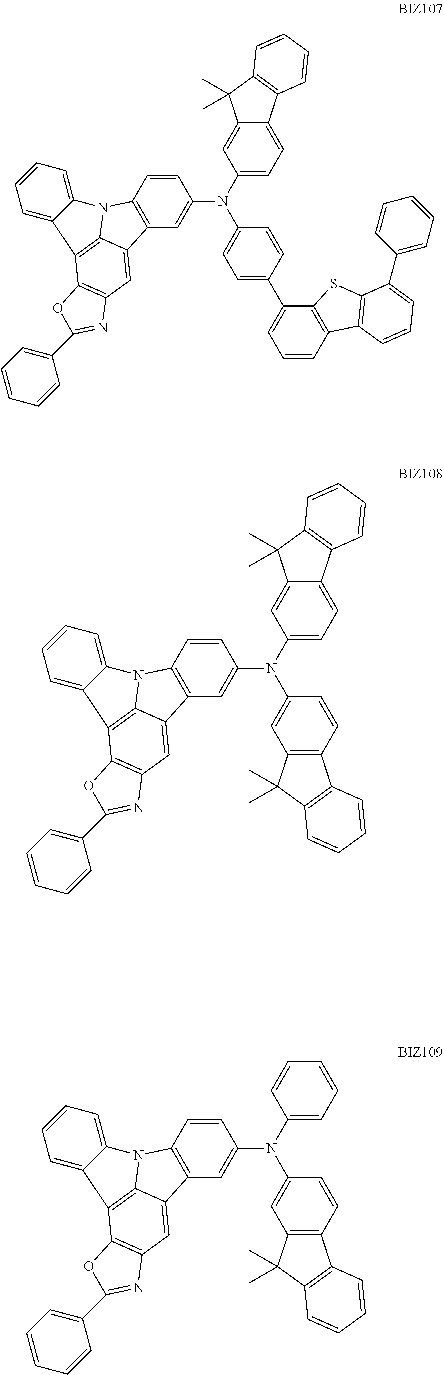 [SMILES: CC1(C)c2ccccc2-c2ccc(N(c3ccc(-c4cccc5c4sc4c(-c6ccccc6)cccc45)cc3)c3ccc4c(c3)c3cc5nc(-c6ccccc6)oc5c5c6ccccc6n4c35)cc21 ; CC1(C)c2ccccc2-c2ccc(N(c3ccc4c(c3)C(C)(C)c3ccccc3-4)c3ccc4c(c3)c3cc5nc(-c6ccccc6)oc5c5c6ccccc6n4c35)cc21 ; CC1(C)c2ccccc2-c2ccc(N(c3ccccc3)c3ccc4c(c3)c3cc5nc(-c6ccccc6)oc5c5c6ccccc6n4c35)cc21]

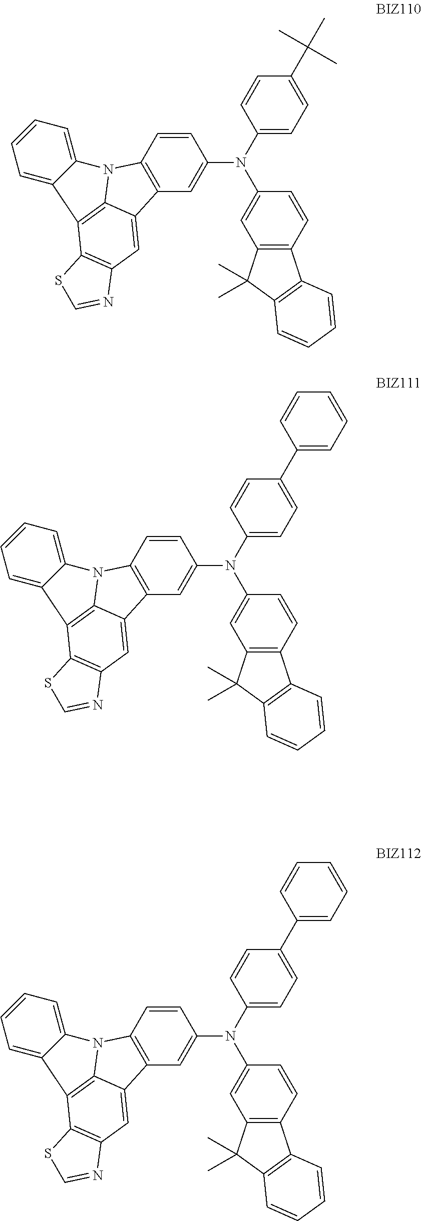

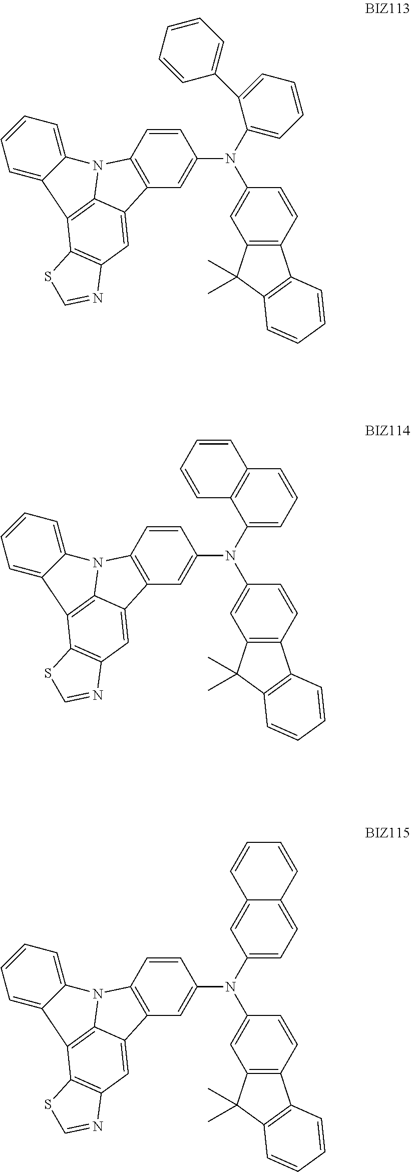 [SMILES: CC1(C)c2ccccc2-c2ccc(N(c3ccc4c(c3)c3cc5ncsc5c5c6ccccc6n4c35)c3ccccc3-c3ccccc3)cc21 ; CC1(C)c2ccccc2-c2ccc(N(c3ccc4c(c3)c3cc5ncsc5c5c6ccccc6n4c35)c3cccc4ccccc34)cc21 ; CC1(C)c2ccccc2-c2ccc(N(c3ccc4ccccc4c3)c3ccc4c(c3)c3cc5ncsc5c5c6ccccc6n4c35)cc21]

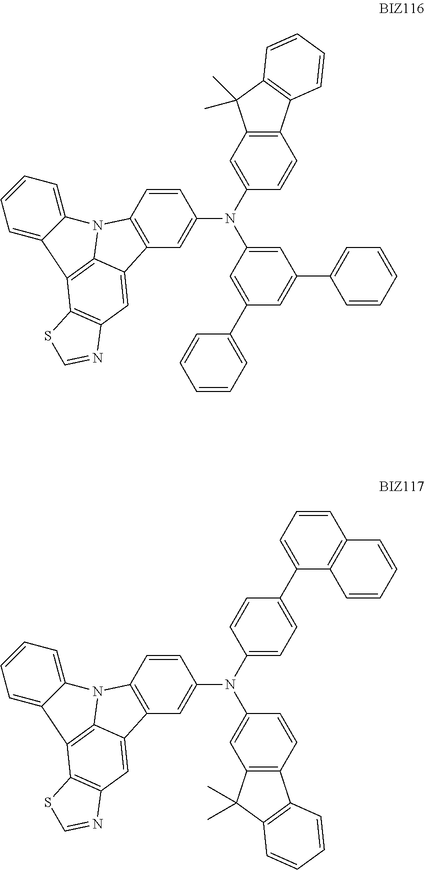 [SMILES: CC1(C)c2ccccc2-c2ccc(N(c3cc(-c4ccccc4)cc(-c4ccccc4)c3)c3ccc4c(c3)c3cc5ncsc5c5c6ccccc6n4c35)cc21 ; CC1(C)c2ccccc2-c2ccc(N(c3ccc(-c4cccc5ccccc45)cc3)c3ccc4c(c3)c3cc5ncsc5c5c6ccccc6n4c35)cc21]

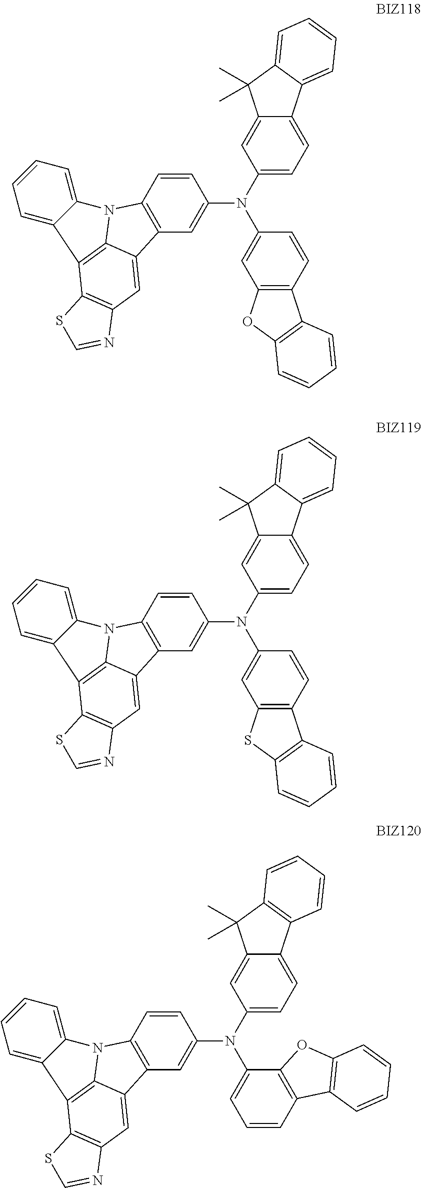 [SMILES: CC1(C)c2ccccc2-c2ccc(N(c3ccc4c(c3)oc3ccccc34)c3ccc4c(c3)c3cc5ncsc5c5c6ccccc6n4c35)cc21 ; CC1(C)c2ccccc2-c2ccc(N(c3ccc4c(c3)sc3ccccc34)c3ccc4c(c3)c3cc5ncsc5c5c6ccccc6n4c35)cc21 ; CC1(C)c2ccccc2-c2ccc(N(c3ccc4c(c3)c3cc5ncsc5c5c6ccccc6n4c35)c3cccc4c3oc3ccccc34)cc21]

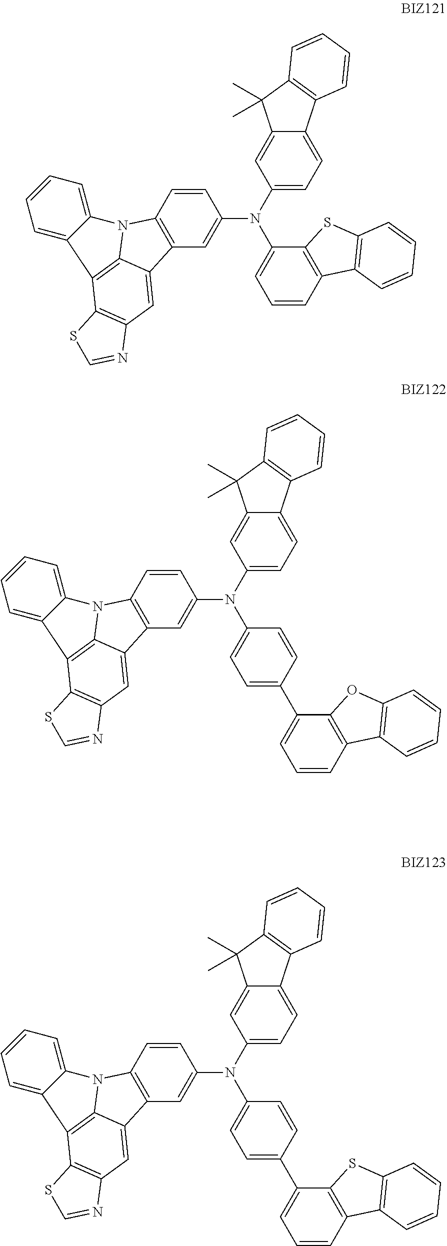 [SMILES: CC1(C)c2ccccc2-c2ccc(N(c3ccc4c(c3)c3cc5ncsc5c5c6ccccc6n4c35)c3cccc4c3sc3ccccc34)cc21 ; CC1(C)c2ccccc2-c2ccc(N(c3ccc(-c4cccc5c4oc4ccccc45)cc3)c3ccc4c(c3)c3cc5ncsc5c5c6ccccc6n4c35)cc21 ; CC1(C)c2ccccc2-c2ccc(N(c3ccc(-c4cccc5c4sc4ccccc45)cc3)c3ccc4c(c3)c3cc5ncsc5c5c6ccccc6n4c35)cc21]

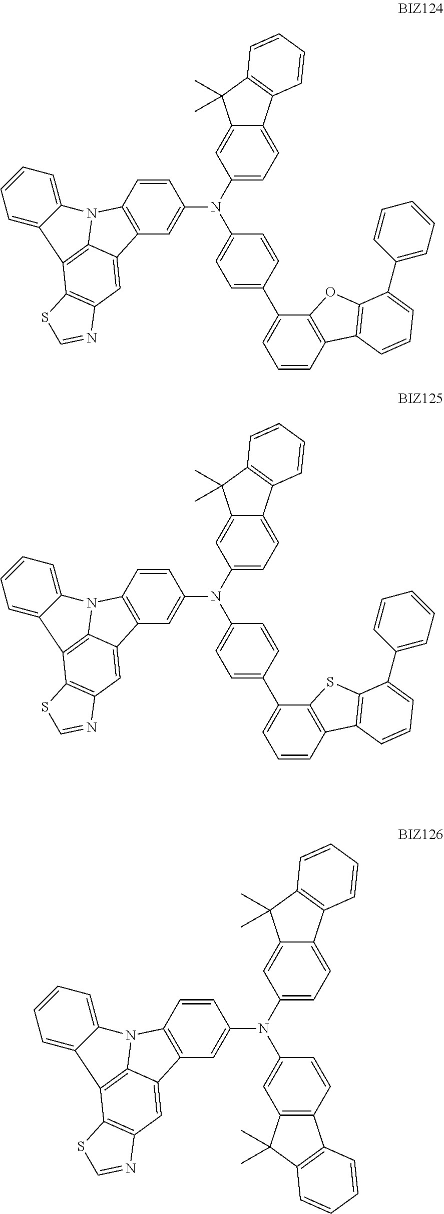 [SMILES: CC1(C)c2ccccc2-c2ccc(N(c3ccc(-c4cccc5c4oc4c(-c6ccccc6)cccc45)cc3)c3ccc4c(c3)c3cc5ncsc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5ncsc5c5c6ccccc6n4c35)cc21 ; CC1(C)c2ccccc2-c2ccc(N(c3ccc4c(c3)C(C)(C)c3ccccc3-4)c3ccc4c(c3)c3cc5ncsc5c5c6ccccc6n4c35)cc21]

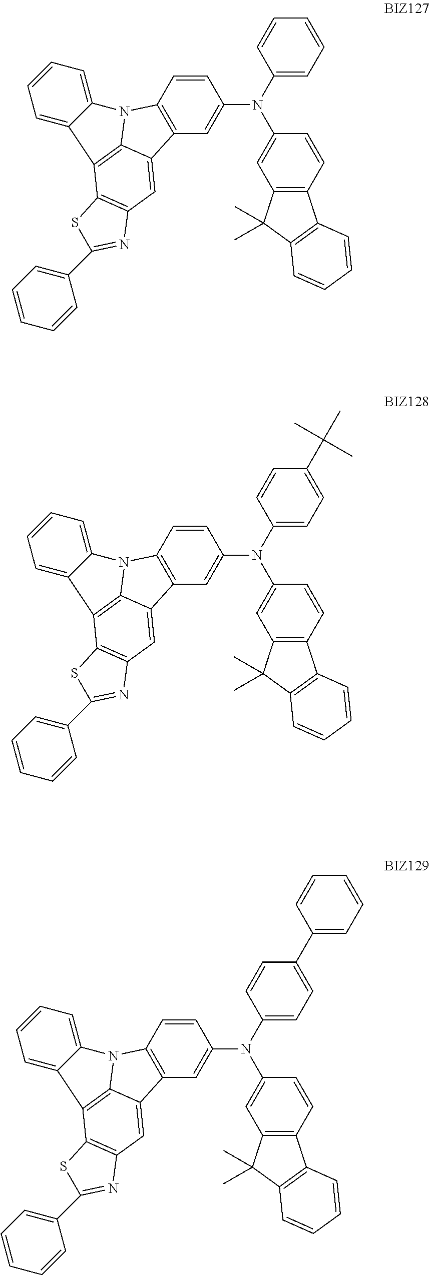 [SMILES: CC1(C)c2ccccc2-c2ccc(N(c3ccccc3)c3ccc4c(c3)c3cc5nc(-c6ccccc6)sc5c5c6ccccc6n4c35)cc21 ; CC(C)(C)c1ccc(N(c2ccc3c(c2)C(C)(C)c2ccccc2-3)c2ccc3c(c2)c2cc4nc(-c5ccccc5)sc4c4c5ccccc5n3c24)cc1 ; CC1(C)c2ccccc2-c2ccc(N(c3ccc(-c4ccccc4)cc3)c3ccc4c(c3)c3cc5nc(-c6ccccc6)sc5c5c6ccccc6n4c35)cc21]

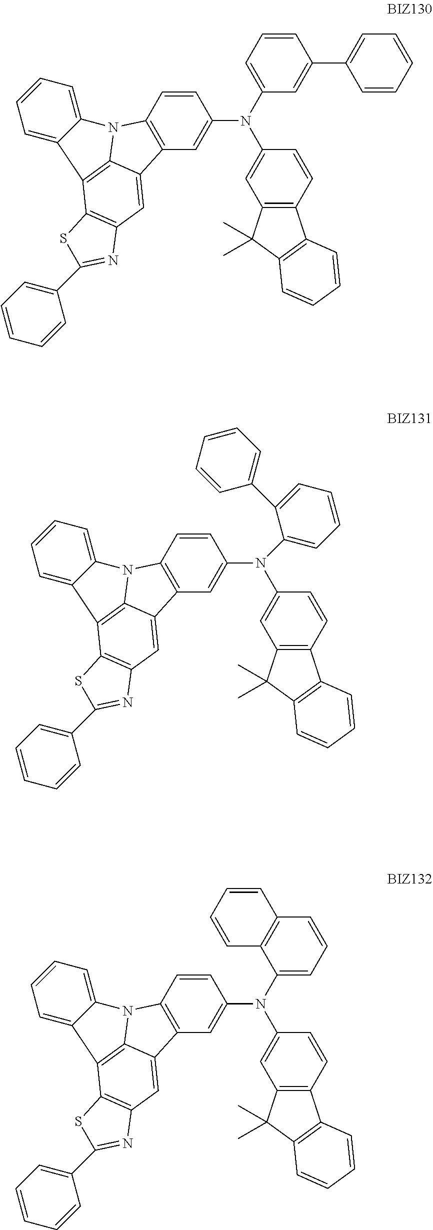 [SMILES: CC1(C)c2ccccc2-c2ccc(N(c3cccc(-c4ccccc4)c3)c3ccc4c(c3)c3cc5nc(-c6ccccc6)sc5c5c6ccccc6n4c35)cc21 ; CC1(C)c2ccccc2-c2ccc(N(c3ccc4c(c3)c3cc5nc(-c6ccccc6)sc5c5c6ccccc6n4c35)c3ccccc3-c3ccccc3)cc21 ; CC1(C)c2ccccc2-c2ccc(N(c3ccc4c(c3)c3cc5nc(-c6ccccc6)sc5c5c6ccccc6n4c35)c3cccc4ccccc34)cc21]

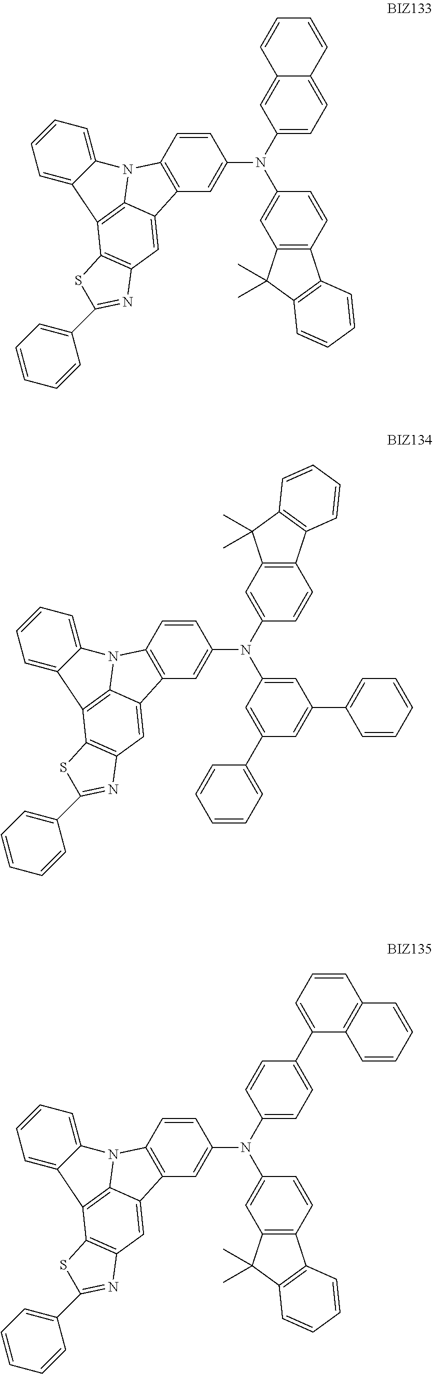 [SMILES: CC1(C)c2ccccc2-c2ccc(N(c3ccc4ccccc4c3)c3ccc4c(c3)c3cc5nc(-c6ccccc6)sc5c5c6ccccc6n4c35)cc21 ; CC1(C)c2ccccc2-c2ccc(N(c3cc(-c4ccccc4)cc(-c4ccccc4)c3)c3ccc4c(c3)c3cc5nc(-c6ccccc6)sc5c5c6ccccc6n4c35)cc21 ; CC1(C)c2ccccc2-c2ccc(N(c3ccc(-c4cccc5ccccc45)cc3)c3ccc4c(c3)c3cc5nc(-c6ccccc6)sc5c5c6ccccc6n4c35)cc21]

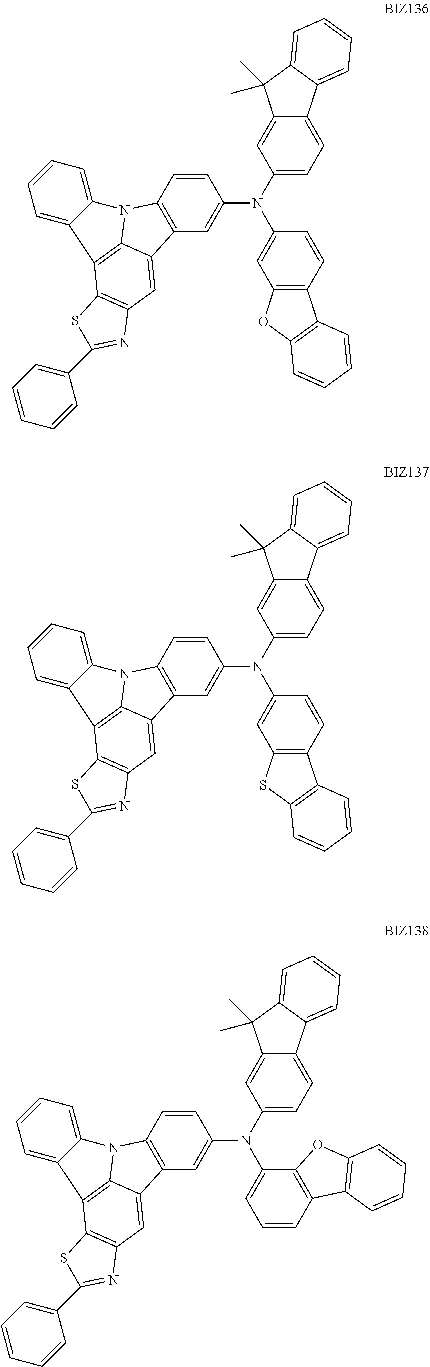 [SMILES: CC1(C)c2ccccc2-c2ccc(N(c3ccc4c(c3)oc3ccccc34)c3ccc4c(c3)c3cc5nc(-c6ccccc6)sc5c5c6ccccc6n4c35)cc21 ; CC1(C)c2ccccc2-c2ccc(N(c3ccc4c(c3)sc3ccccc34)c3ccc4c(c3)c3cc5nc(-c6ccccc6)sc5c5c6ccccc6n4c35)cc21 ; CC1(C)c2ccccc2-c2ccc(N(c3ccc4c(c3)c3cc5nc(-c6ccccc6)sc5c5c6ccccc6n4c35)c3cccc4c3oc3ccccc34)cc21]

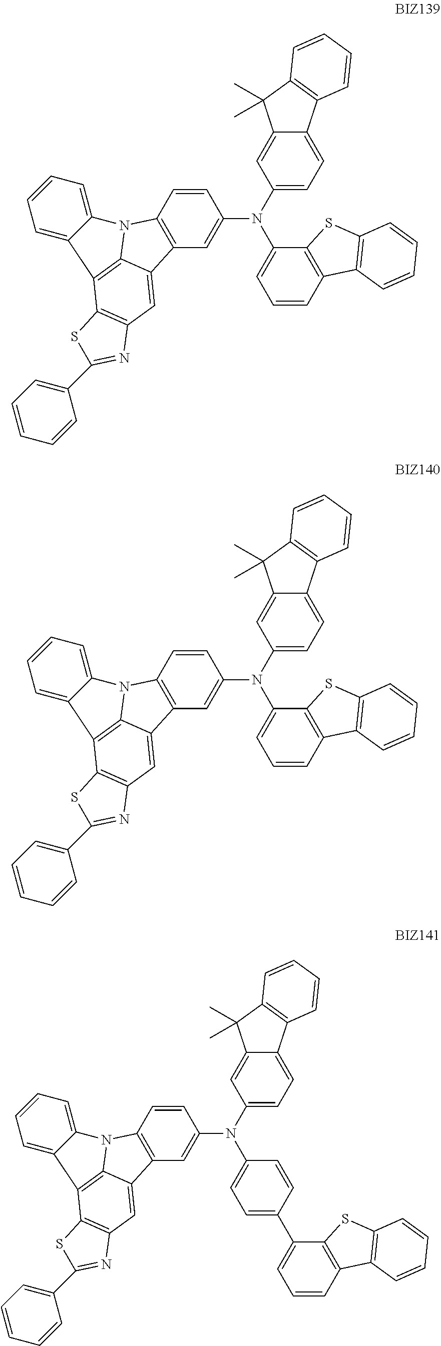 [SMILES: CC1(C)c2ccccc2-c2ccc(N(c3ccc4c(c3)c3cc5nc(-c6ccccc6)sc5c5c6ccccc6n4c35)c3cccc4c3sc3ccccc34)cc21 ; CC1(C)c2ccccc2-c2ccc(N(c3ccc4c(c3)c3cc5nc(-c6ccccc6)sc5c5c6ccccc6n4c35)c3cccc4c3sc3ccccc34)cc21 ; CC1(C)c2ccccc2-c2ccc(N(c3ccc(-c4cccc5c4sc4ccccc45)cc3)c3ccc4c(c3)c3cc5nc(-c6ccccc6)sc5c5c6ccccc6n4c35)cc21]

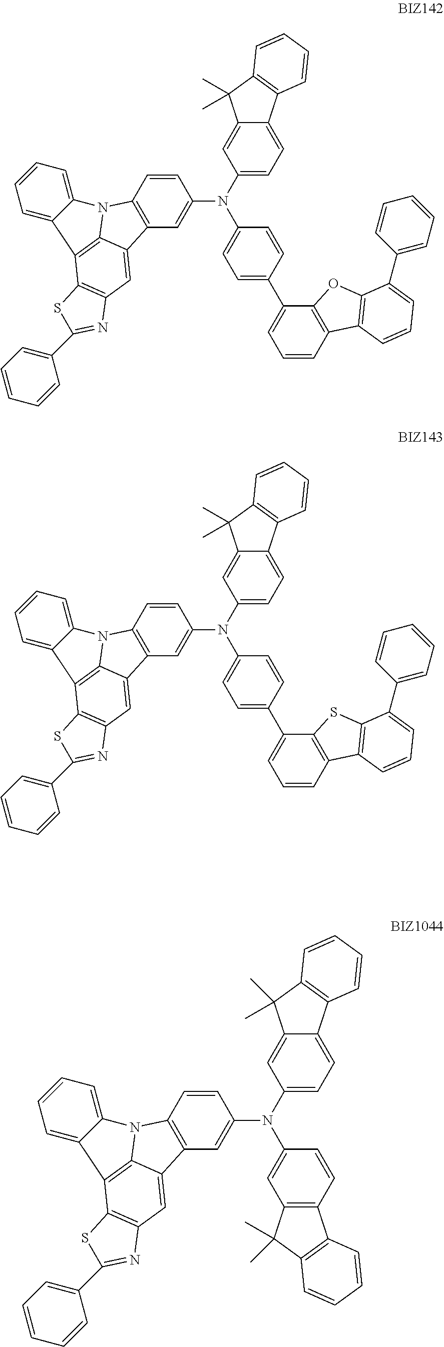 [SMILES: CC1(C)c2ccccc2-c2ccc(N(c3ccc(-c4cccc5c4oc4c(-c6ccccc6)cccc45)cc3)c3ccc4c(c3)c3cc5nc(-c6ccccc6)sc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5nc(-c6ccccc6)sc5c5c6ccccc6n4c35)cc21 ; CC1(C)c2ccccc2-c2ccc(N(c3ccc4c(c3)C(C)(C)c3ccccc3-4)c3ccc4c(c3)c3cc5nc(-c6ccccc6)sc5c5c6ccccc6n4c35)cc21]

### Synthesis

### 1. Synthesis of the Compound BIZ021

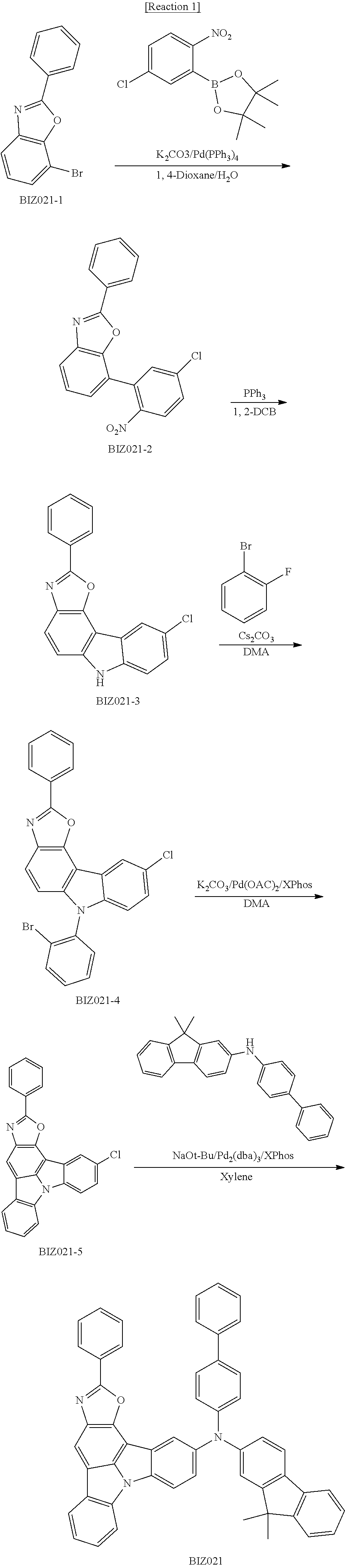

### (1) Compound BIZ021-2

In the one-neck rounded-bottom flask, a mixture of 7-bromo-2-phenylbenzo[d]oxazole (20 g, 0.073 mol), 2-(5-chloro-2-nitrophenyl)-4,4,5,5-tetramethyl-1,3,2-dioxaborolane (22.8 g, 0.080 mol), K₂CO₃ (30.3 g, 0.219 mol), Pd(PPh₃)₄ (palladium-tetrakis(triphenylphosphine), 4.2 g, 0.004 mol), 1,4-dioxane (300 ml) and water (60 ml) was refluxed for 8 hours at 100° C. After the temperature was lowered, the mixture was extracted with dichloromethane, and moisture was removed using MgSO₄. After the solvent was removed, the mixture was dissolved in dichloromethane and is filtered with silica gel. The mixture was concentrated to obtain the compound BIZ021-2 (18 g, 70%).

### (2) Compound BIZ021-3

In the one-neck rounded-bottom flask, a mixture of the compound BIZ021-2 (18 g, 0.051 mol), PPh₃ (33.6 g, 0.128 mol) and 1,2-dichlorobenzene (180 ml) was stirred for 6 hours at 160° C. After the solvent was removed, the mixture was dissolved in dichloromethane and is filtered with silica gel. The mixture was concentrated to obtain the compound BIZ021-3 (11 g, 67%).

### (3) Compound BIZ021-4

In the one-neck rounded-bottom flask, a mixture of the compound BIZ021-3 (11 g, 0.029 mol), 1-bromo-2-fluorobenzene (6.1 g, 0.035 mol), Cs₂CO₃ (22.5 g, 0.069 mol) and dimethylacetamide (110 ml) was stirred for 6 hours at 130° C. After the solvent was removed, the mixture was dissolved in dichloromethane and is filtered with silica gel. The mixture was concentrated to obtain the compound BIZ021-4 (13 g, 80%).

### (4) Compound BIZ021-5

In the one-neck rounded-bottom flask, a mixture of the compound BIZ021-4 (13 g, 0.027 mol), K₂CO₃ (7.6 g, 0.055 mol), Pd(OAC)₂ (0.6 g, 0.003 mol), XPhos (2.6 g, 0.005 mol) and dimethylacetamide (130 ml) was stirred for 6 hours at 130° C. After the solvent was removed, the mixture was dissolved in 1,2-dichlorobenzene and is filtered with silica gel. The mixture was concentrated to obtain the compound BIZ021-5 (6 g, 56%).

### (5) Compound BIZ021

In the one-neck rounded-bottom flask, a mixture of the compound BIZ021-5 (6 g, 0.015 mol), N-([1,1′-biphenyl]-4-yl)-9,9-dimethyl-9H-fluoren-2-amine (6.1 g, 0.017 mol), NaOt-Bu (2.9 g, 0.030 mol), Pd₂(dba)₃ (1.4 g, 0.002 mol), XPhos (1.4 g, 0.003 mol) and xylene (90 ml) was refluxed for 8 hours at 140° C. After the solvent was removed, the mixture was dissolved in 1,2-dichlorobenzene and is filtered with silica gel. The mixture was concentrated to obtain the compound BIZ021 (8 g, 73%).

### 2. Synthesis of the Compound BIZ026

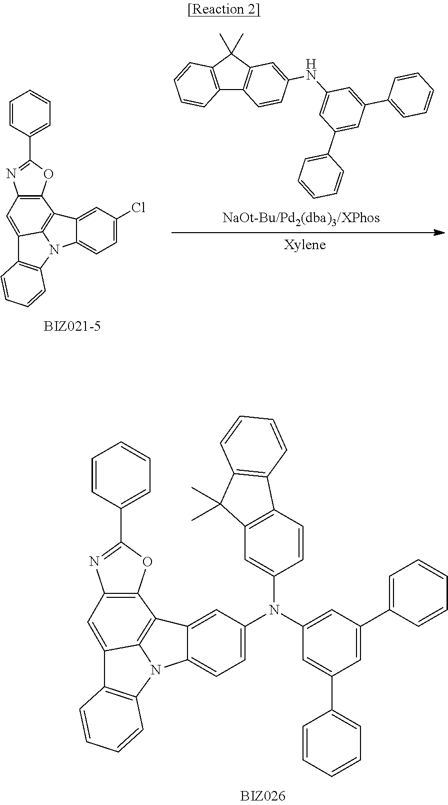 [SMILES: Clc1ccc2c(c1)c1c3oc(-c4ccccc4)nc3cc3c4ccccc4n2c31 ; CC1(C)c2ccccc2-c2ccc(Nc3cc(-c4ccccc4)cc(-c4ccccc4)c3)cc21 ; CC1(C)c2ccccc2-c2ccc(N(c3cc(-c4ccccc4)cc(-c4ccccc4)c3)c3ccc4c(c3)c3c5oc(-c6ccccc6)nc5cc5c6ccccc6n4c53)cc21]

Except using N-([1,1′: 3′, 1″-terphenyl]-5′-yl)-9,9-dimethyl-9H-fluoren-2-amine instead of N-([1,1′-biphenyl]-4-yl)-9,9-dimethyl-9H-fluoren-2-amine in the synthesis process of the compound BIZ021, the same process was performed to obtain the compound BIZ026 (66%).

### 3. Synthesis of the Compound BIZ028

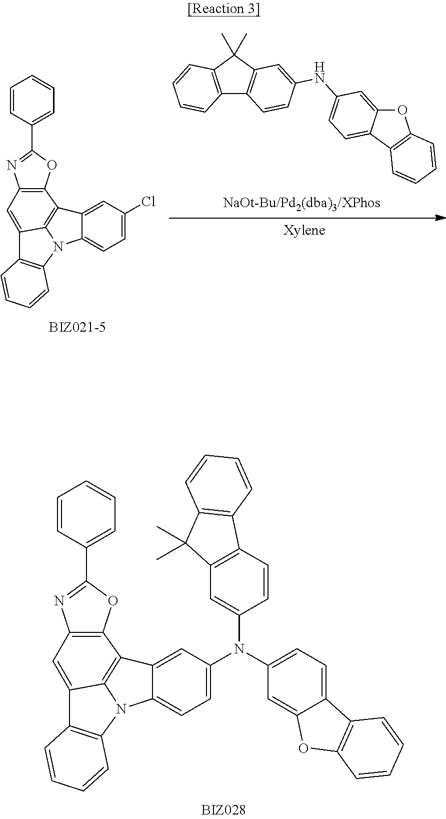 [SMILES: Clc1ccc2c(c1)c1c3oc(-c4ccccc4)nc3cc3c4ccccc4n2c31 ; CC1(C)c2ccccc2-c2ccc(Nc3ccc4c(c3)oc3ccccc34)cc21 ; CC1(C)c2ccccc2-c2ccc(N(c3ccc4c(c3)oc3ccccc34)c3ccc4c(c3)c3c5oc(-c6ccccc6)nc5cc5c6ccccc6n4c53)cc21]

Except using N-(9,9-dimethyl-9H-fluoren-2-yl)dibenzo[b,d]furan-3-amine instead of N-([1,1′-biphenyl]-4-yl)-9,9-dimethyl-9H-fluoren-2-amine in the synthesis process of the compound BIZ021, the same process was performed to obtain the compound BIZ028 (60%).

### 4. Synthesis of the Compound BIZ029

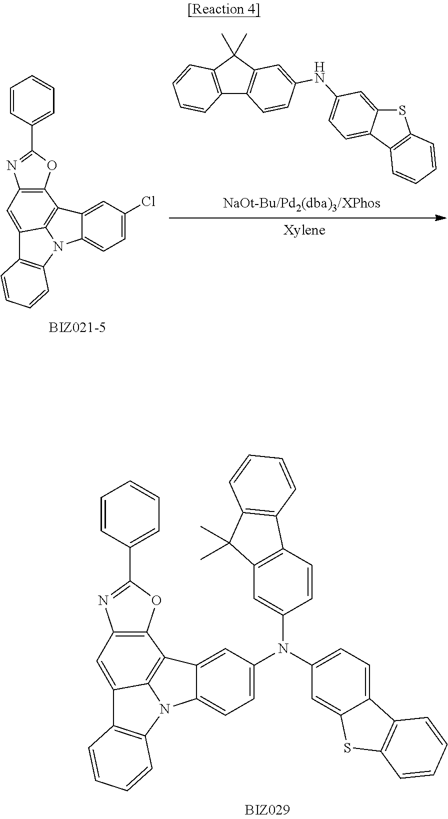 [SMILES: Clc1ccc2c(c1)c1c3oc(-c4ccccc4)nc3cc3c4ccccc4n2c31 ; CC1(C)c2ccccc2-c2ccc(Nc3ccc4c(c3)sc3ccccc34)cc21 ; CC1(C)c2ccccc2-c2ccc(N(c3ccc4c(c3)sc3ccccc34)c3ccc4c(c3)c3c5oc(-c6ccccc6)nc5cc5c6ccccc6n4c53)cc21]

Except using N-(9,9-dimethyl-9H-fluoren-2-yl)dibenzo[b,d]thiophene-3-amine instead of N-([1,1′-biphenyl]-4-yl)-9,9-dimethyl-9H-fluoren-2-amine in the synthesis process of the compound BIZ021, the same process was performed to obtain the compound BIZ029 (58%).

### 5. Synthesis of the Compound BIZ036

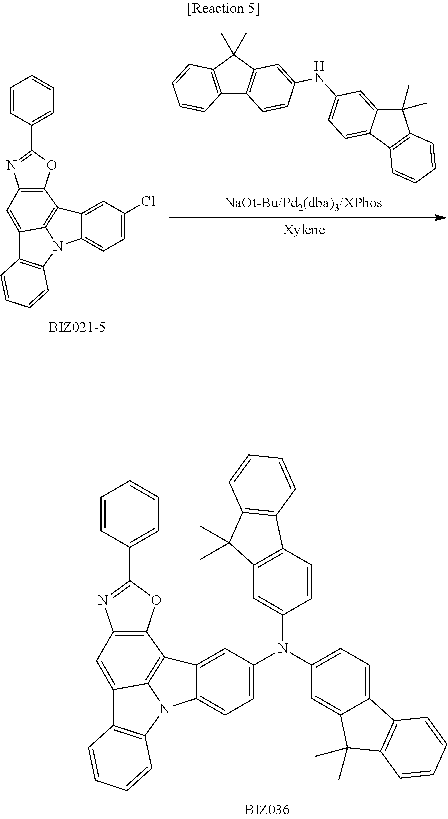 [SMILES: Clc1ccc2c(c1)c1c3oc(-c4ccccc4)nc3cc3c4ccccc4n2c31 ; CC1(C)c2ccccc2-c2ccc(Nc3ccc4c(c3)C(C)(C)c3ccccc3-4)cc21 ; CC1(C)c2ccccc2-c2ccc(N(c3ccc4c(c3)C(C)(C)c3ccccc3-4)c3ccc4c(c3)c3c5oc(-c6ccccc6)nc5cc5c6ccccc6n4c53)cc21]

Except using bis(9,9-dimethyl-9H-fluoren-2-yl)amine instead of N-([1,1′-biphenyl]-4-yl)-9,9-dimethyl-9H-fluoren-2-amine in the synthesis process of the compound BIZ021, the same process was performed to obtain the compound BIZ036 (69%).

### 6. Synthesis of the Compound BIZ057

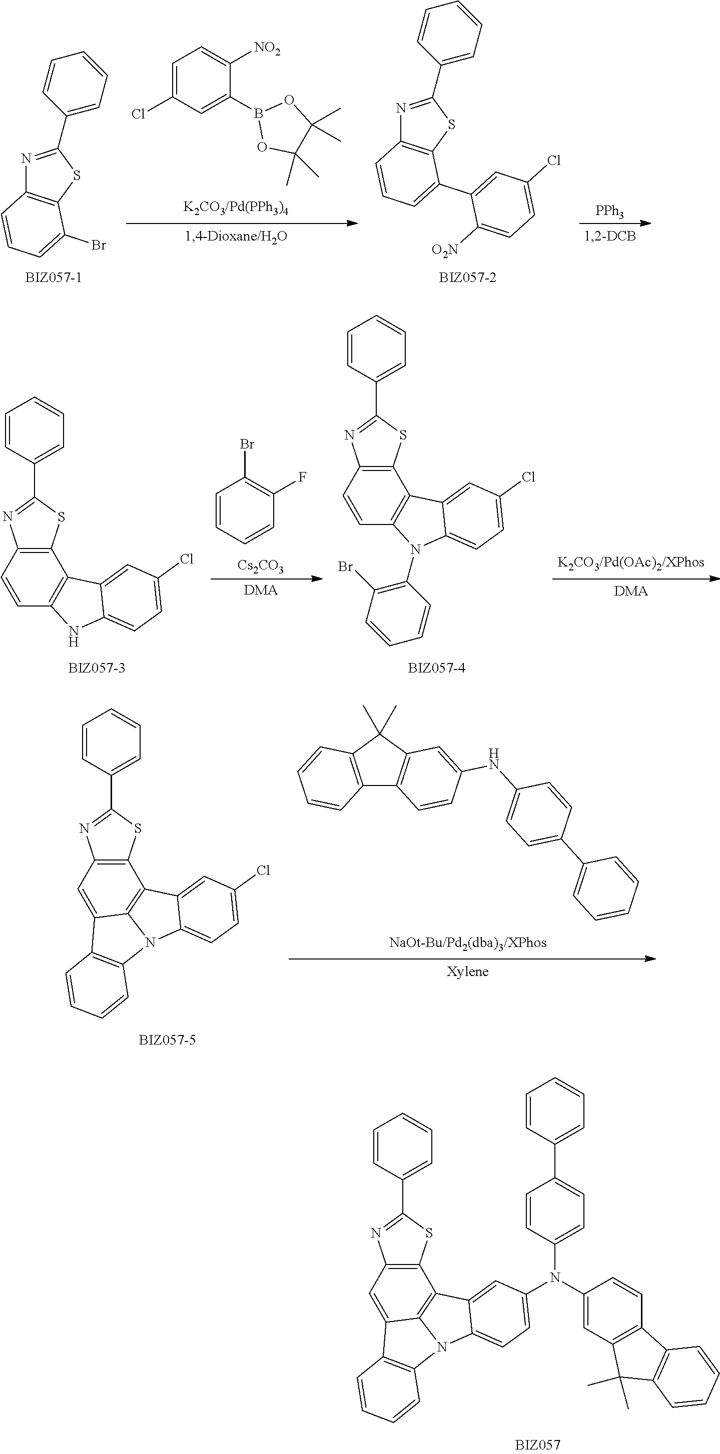

### (1) Compound BIZ057-2

In the one-neck rounded-bottom flask, a mixture of 7-bromo-2-phenylbenzo[d]thiazole (20 g, 0.069 mol), 2-(5-chloro-2-nitrophenyl)-4,4,5,5-tetramethyl-1,3,2-dioxaborolane (23.4 g, 0.083 mol), K₂CO₃ (28.6 g, 0.207 mol), Pd(PPh₃)₄ (4.0 g, 0.003 mol), 1,4-dioxane (300 ml) and water (60 ml) was refluxed for 9 hours at 100° C. After the temperature was lowered, the mixture was extracted with dichloromethane, and moisture was removed using MgSO₄. After the solvent was removed, the mixture was dissolved in dichloromethane and is filtered with silica gel. The mixture was concentrated to obtain the compound BIZ057-2 (17 g, 67%).

### (2) Compound BIZ057-3

In the one-neck rounded-bottom flask, a mixture of the compound BIZ057-2 (17 g, 0.046 mol), PPh₃ (36.4 g, 0.139 mol) and 1,2-dichlorobenzene (170 ml) was stirred for 10 hours at 160° C. After the solvent was removed, the mixture was dissolved in dichloromethane and is filtered with silica gel. The mixture was concentrated to obtain the compound BIZ057-3 (10 g, 64%).

### (3) Compound BIZ057-4

In the one-neck rounded-bottom flask, a mixture of the compound BIZ057-3 (10 g, 0.029 mol), 1-bromo-2-fluorobenzene (6.3 g, 0.036 mol), Cs₂CO₃ (29.2 g, 0.090 mol), and dimethylacetamide (100 ml) was stirred for 7 hours at 130° C. After the solvent was removed, the mixture was dissolved in dichloromethane and is filtered with silica gel. The mixture was concentrated to obtain the compound BIZ057-4 (11 g, 75%).

### (4) Compound BIZ057-5

In the one-neck rounded-bottom flask, a mixture of the compound BIZ057-4 (11 g, 0.022 mol), K₂CO₃ (7.8 g, 0.056 mol), Pd(OAC)₂ (0.5 g, 0.002 mol), XPhos (2.1 g, 0.004 mol), and dimethylacetamide (110 ml) was stirred for 7 hours at 130° C. After the solvent was removed, the mixture was dissolved in 1,2-dichlorobenzene and is filtered with silica gel. The mixture was concentrated to obtain the compound BIZ057-5 (5 g, 54%).

### (5) Compound BIZ057

In the one-neck rounded-bottom flask, a mixture of the compound BIZ057-5 (5 g, 0.012 mol), N-([1,1′-biphenyl]-4-yl)-9,9-dimethyl-9H-fluoren-2-amine (4.9 g, 0.013 mol), NaOt-Bu (2.4 g, 0.024 mol), Pd₂(dba)₃ (1.1 g, 0.001 mol), XPhos (1.1 g, 0.002 mol) and xylene (75 ml) was refluxed for 8 hours at 140° C. After the solvent was removed, the mixture was dissolved in 1,2-dichlorobenzene and is filtered with silica gel. The mixture was concentrated to obtain the compound BIZ057 (7 g, 78%).

### 7. Synthesis of the Compound BIZ062

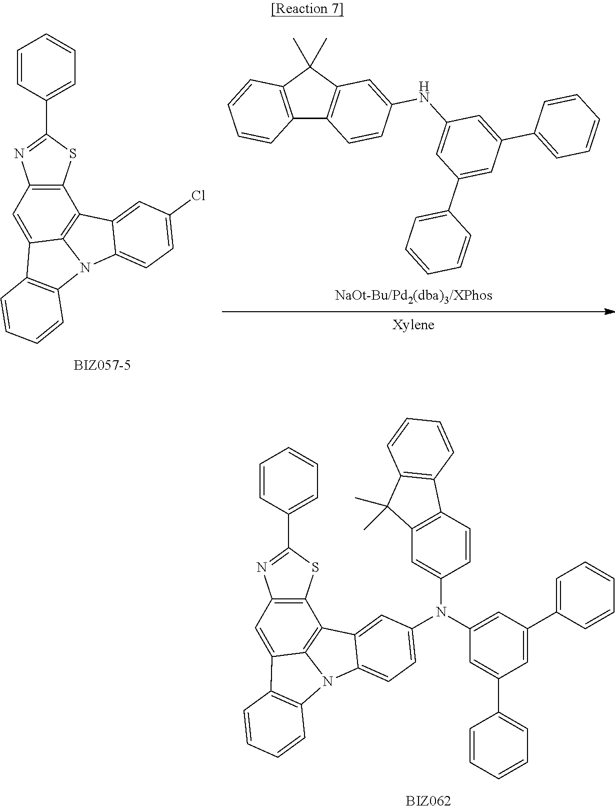 [SMILES: Clc1ccc2c(c1)c1c3sc(-c4ccccc4)nc3cc3c4ccccc4n2c31 ; CC1(C)c2ccccc2-c2ccc(Nc3cc(-c4ccccc4)cc(-c4ccccc4)c3)cc21 ; CC1(C)c2ccccc2-c2ccc(N(c3cc(-c4ccccc4)cc(-c4ccccc4)c3)c3ccc4c(c3)c3c5sc(-c6ccccc6)nc5cc5c6ccccc6n4c53)cc21]

Except using N-([1,1′:3′,1″-terphenyl]-5′-yl)-9,9-dimethyl-9H-fluoren-2-amine instead of N-([1,1′-biphenyl]-4-yl)-9,9-dimethyl-9H-fluoren-2-amine in the synthesis process of the compound BIZ057, the same process was performed to obtain the compound BIZ062 (63%).

### 8. Synthesis of the Compound BIZ064

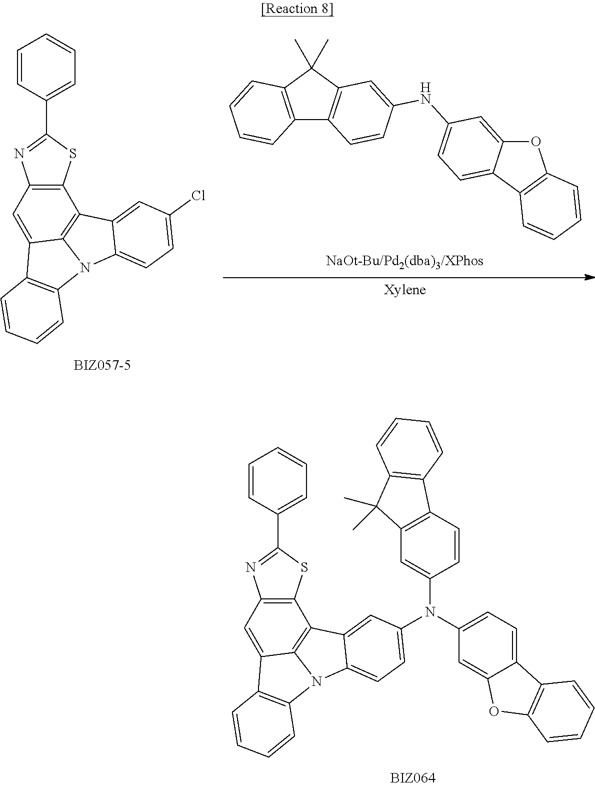 [SMILES: Clc1ccc2c(c1)c1c3sc(-c4ccccc4)nc3cc3c4ccccc4n2c31 ; CC1(C)c2ccccc2-c2ccc(Nc3ccc4c(c3)oc3ccccc34)cc21 ; CC1(C)c2ccccc2-c2ccc(N(c3ccc4c(c3)oc3ccccc34)c3ccc4c(c3)c3c5sc(-c6ccccc6)nc5cc5c6ccccc6n4c53)cc21]

Except using N-(9,9-dimethyl-9H-fluoren-2-yl)dibenzo[b,d]furan-3-amine instead of N-([1,1′-biphenyl]-4-yl)-9,9-dimethyl-9H-fluoren-2-amine in the synthesis process of the compound BIZ057, the same process was performed to obtain the compound BIZ064 (72%).

### 9. Synthesis of the Compound BIZ065

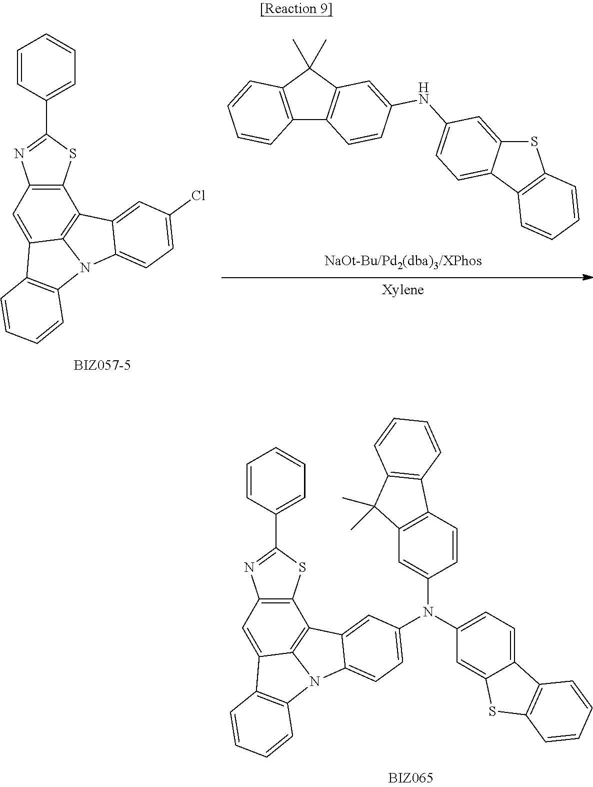 [SMILES: Clc1ccc2c(c1)c1c3sc(-c4ccccc4)nc3cc3c4ccccc4n2c31 ; CC1(C)c2ccccc2-c2ccc(Nc3ccc4c(c3)sc3ccccc34)cc21 ; CC1(C)c2ccccc2-c2ccc(N(c3ccc4c(c3)sc3ccccc34)c3ccc4c(c3)c3c5sc(-c6ccccc6)nc5cc5c6ccccc6n4c53)cc21]

Except using N-(9,9-dimethyl-9H-fluoren-2-yl)dibenzo[b,d]thiophen-3-amine instead of N-([1,1′-biphenyl]-4-yl)-9,9-dimethyl-9H-fluoren-2-amine in the synthesis process of the compound BIZ057, the same process was performed to obtain the compound BIZ065 (64%).

### 10. Synthesis of the Compound BIZ072

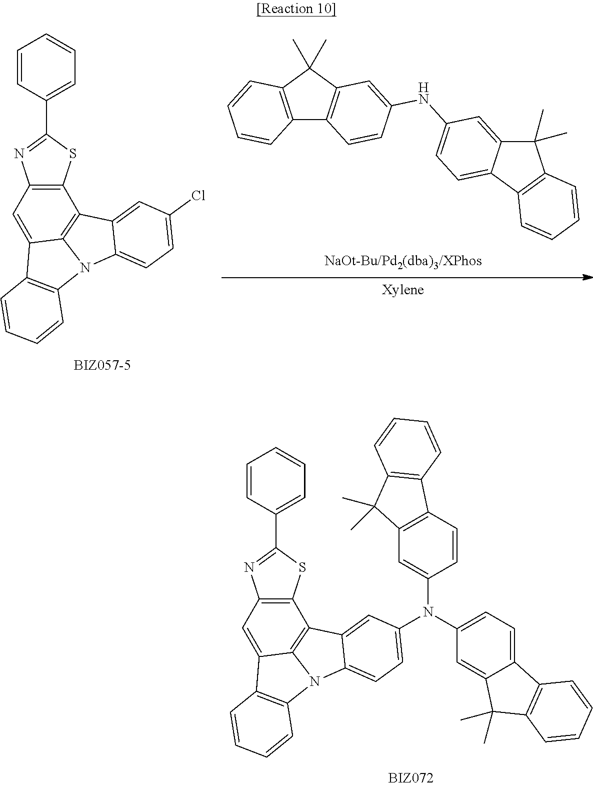 [SMILES: Clc1ccc2c(c1)c1c3sc(-c4ccccc4)nc3cc3c4ccccc4n2c31 ; CC1(C)c2ccccc2-c2ccc(Nc3ccc4c(c3)C(C)(C)c3ccccc3-4)cc21 ; CC1(C)c2ccccc2-c2ccc(N(c3ccc4c(c3)C(C)(C)c3ccccc3-4)c3ccc4c(c3)c3c5sc(-c6ccccc6)nc5cc5c6ccccc6n4c53)cc21]

Except using bis(9,9-dimethyl-9H-fluoren-2-yl)amine instead of N-([1,1′-biphenyl]-4-yl)-9,9-dimethyl-9H-fluoren-2-amine in the synthesis process of the compound BIZ057, the same process was performed to obtain the compound BIZ072 (70%).

### 11. Synthesis of the Compound BIZ093

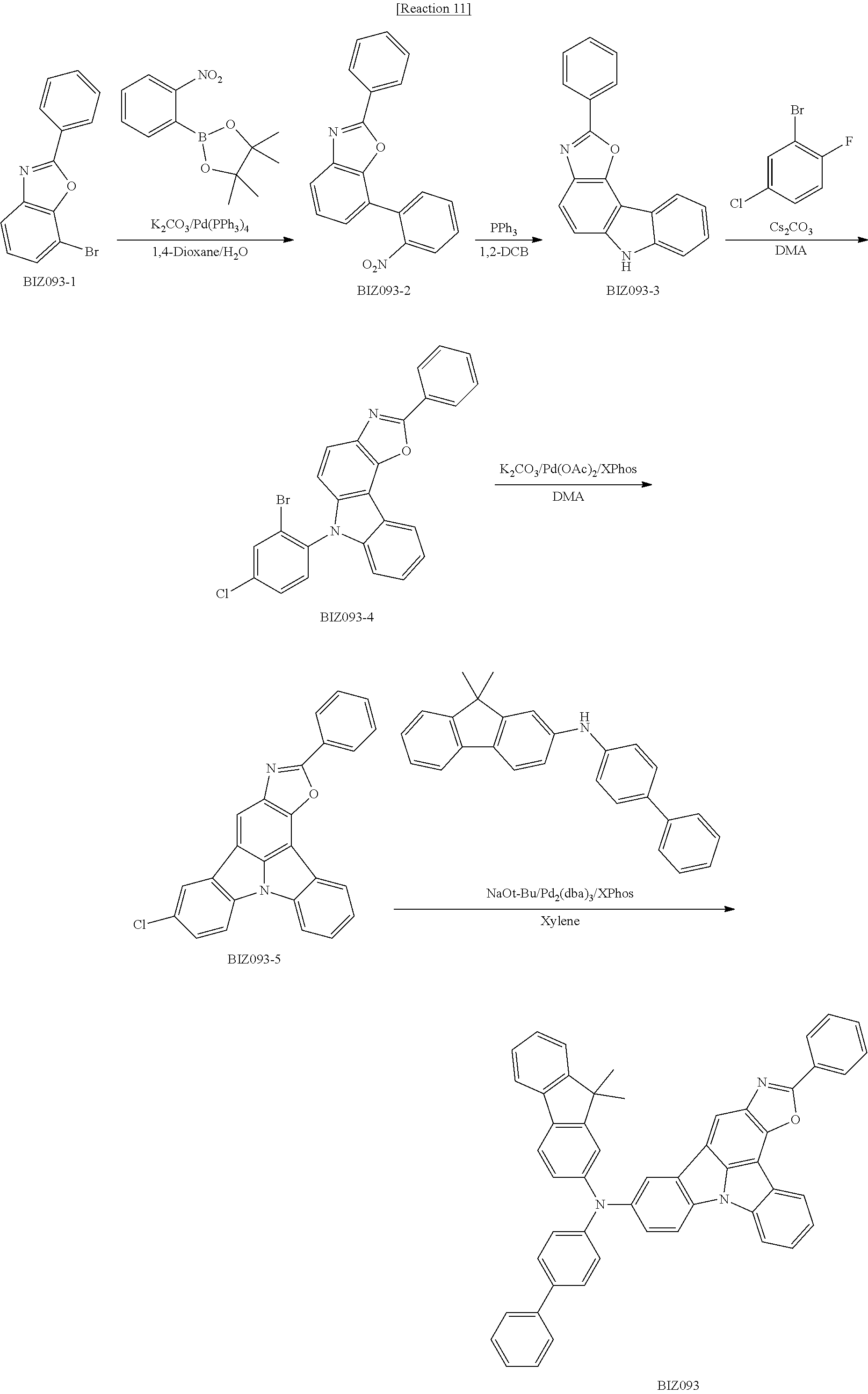

### (1) Compound BIZ093-2

In the one-neck rounded-bottom flask, a mixture of 7-bromo-2-phenylbenzo[d]oxazole (20 g, 0.073 mol), 4,4,5,5-tetramethyl-2-(2-nitrophenyl)-1,3,2-dioxaborolane (20 g, 0.080 mol), K₂CO₃ (30.3 g, 0.219 mol), Pd(PPh₃)₄ (4.2 g, 0.004 mol), 1,4-dioxane (300 ml), and water (60 ml) was refluxed for 8 hours at 100° C. After the temperature was lowered, the mixture was extracted with dichloromethane, and moisture was removed using MgSO₄. After the solvent was removed, the mixture was dissolved in dichloromethane and is filtered with silica gel. The mixture was concentrated to obtain the compound BIZ093-2 (17 g, 74%).

### (2) Compound BIZ093-3

In the one-neck rounded-bottom flask, a mixture of the compound BiZ093-2 (17 g, 0.054 mol), PPh₃ (35.2 g, 0.134 mol) and 1,2-dichlorobenzene (170 ml) was stirred for 8 hours at 160° C. After the solvent was removed, the mixture was dissolved in dichloromethane and is filtered with silica gel. The mixture was concentrated to obtain the compound BIZ093-3 (11 g, 72%).

### (3) Compound BIZ093-4

In the one-neck rounded-bottom flask, a mixture of the compound BIZ093-3 (1 1 g, 0.039 mol), 2-bromo-4-chloro-1-fluorobenzene (8.9 g, 0.043 mol), Cs₂CO₃ (31.5 g, 0.097 mol), dimethylacetamide (110 ml) was stirred for 6 hours at 130° C. After the solvent was removed, the mixture was dissolved in dichloromethane and is filtered with silica gel. The mixture was concentrated to obtain the compound BIZ093-4 (14 g, 76%).

### (4) Compound BIZ093-5

In the one-neck rounded-bottom flask, a mixture of the compound BIZ093-4 (14 g, 0.030 mol), K₂CO₃ (10.2 g, 0.074 mol), Pd(OAC)₂ (0.7 g, 0.003 mol), XPhos (2.7 g, 0.006 mol), and dimethylacetamide (140 ml) was stirred for 7 hours at 130° C. After the solvent was removed, the mixture was dissolved in 1,2-dichlorobenzene and is filtered with silica gel. The mixture was concentrated to obtain the compound BIZ093-5 (7 g, 60%).

### (5) Compound BIZ093

In the one-neck rounded-bottom flask, a mixture of the compound BIZ093-5 (7 g, 0.018 mol), N-([1,1′-biphenyl]-4-yl)-9,9-dimethyl-9H-fluoren-2-amine (7.1 g, 0.020 mol), NaOt-Bu (3.4 g, 0.036 mol), Pd₂(dba)₃ (1.6 g, 0.002 mol), XPhos (1.6 g, 0.004 mol), and xylene (100 ml) was refluxed for 8 hours at 140° C. After the solvent was removed, the mixture was dissolved in 1,2-dichlorobenzene and is filtered with silica gel. The mixture was concentrated to obtain the compound BIZ093 (8 g, 63%).

### 12. Synthesis of the Compound BIZ098

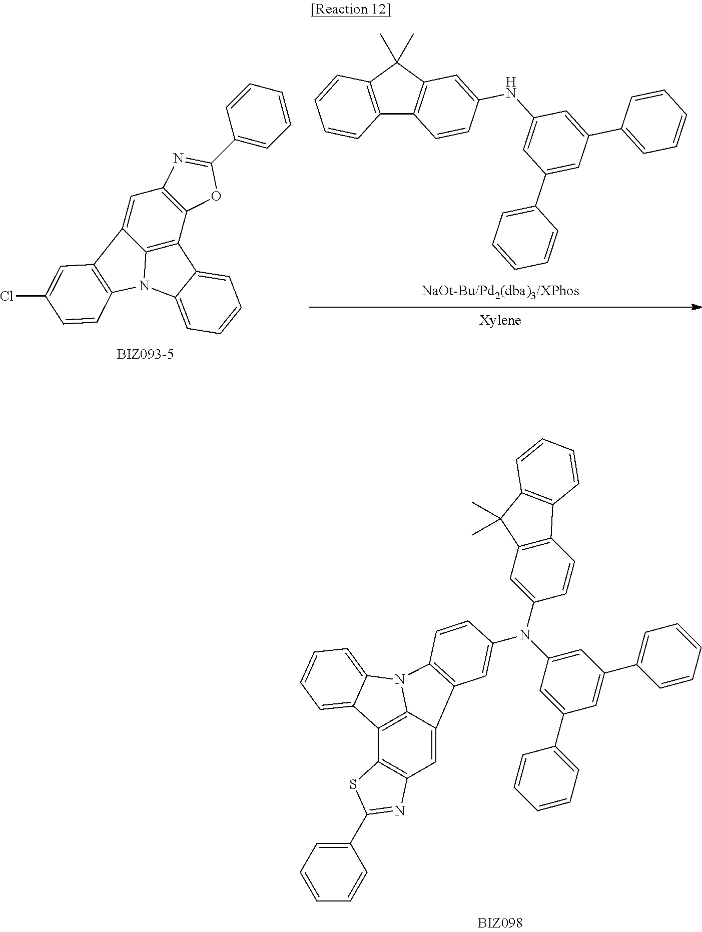

Except using N-([1,1′;3′,1″-terphenyl]-5′-yl)-9,9-dimethyl-9H-fluoren-2-amine instead of N-([1,1′-biphenyl]-4-yl)-9,9-dimethyl-9H-fluoren-2-amine in the synthesis process of the compound BIZ093, the same process was performed to obtain the compound BIZ098 (66%).

### 13. Synthesis of the Compound BIZ100

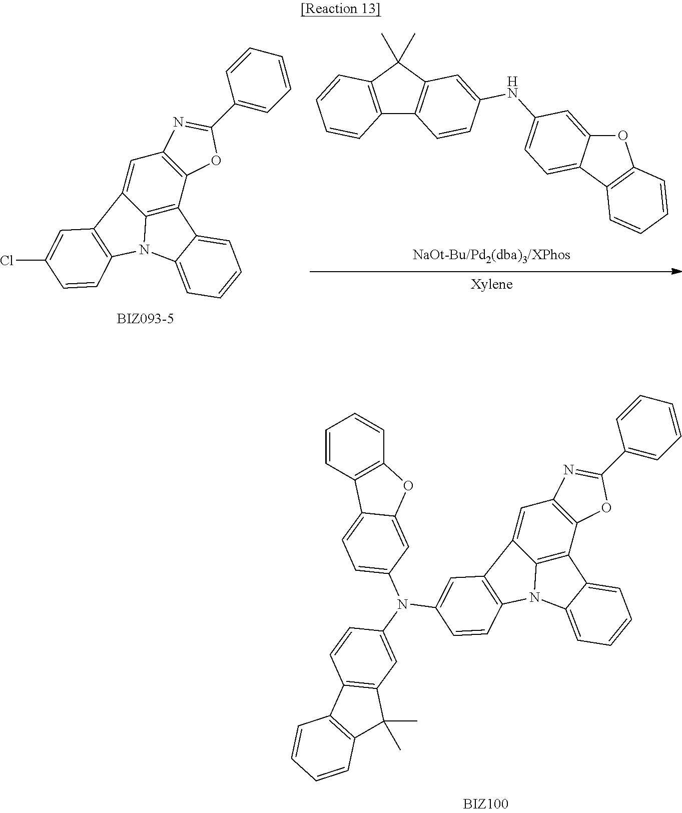

Except using N-(9,9-dimethyl-9H-fluoren-2-yl)dibenzo[b,d]furan-3-amine instead of N-([1,1′-biphenyl]-4-yl)-9,9-dimethyl-9H-fluoren-2-amine in the synthesis process of the compound BIZ093, the same process was performed to obtain the compound BIZ100 (61%).

### 14. Synthesis of the Compound BIZ101

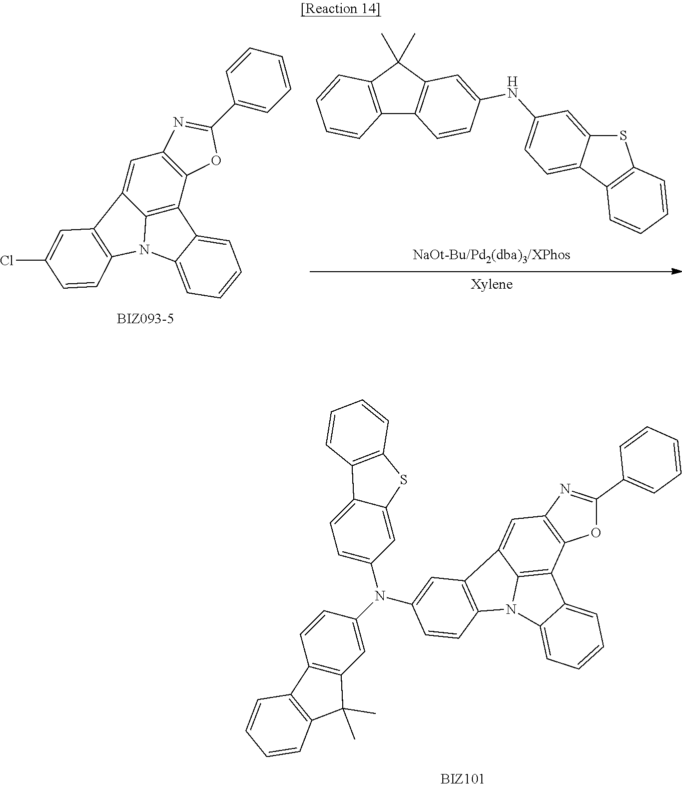

Except using N-(9,9-dimethyl-9H-fluoren-2-yl)dibenzo[b,d]thiophen-3-amine instead of N-([1,1′-biphenyl]-4-yl)-9,9-dimethyl-9H-fluoren-2-amine in the synthesis process of the compound BIZ093, the same process was performed to obtain the compound BIZ101 (71%).

### 15. Synthesis of the Compound BIZ108

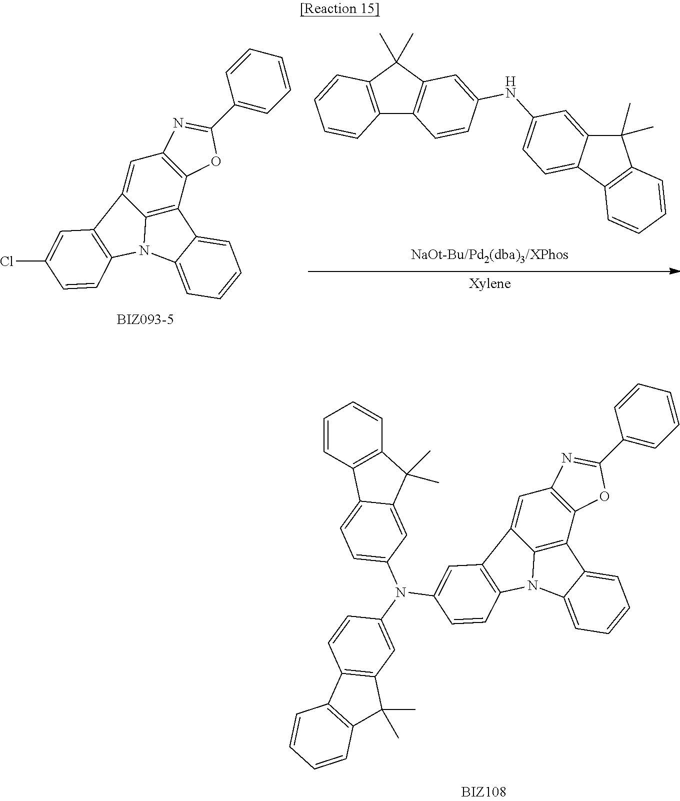

Except using bis(9,9-dimethyl-9H-fluoren-2-yl)amine instead of N-([1,1′-biphenyl]-4-yl)-9,9-dimethyl-9H-fluoren-2-amine in the synthesis process of the compound BIZ093, the same process was performed to obtain the compound BIZ108 (62%).

### 16. Synthesis of the Compound BIZ129

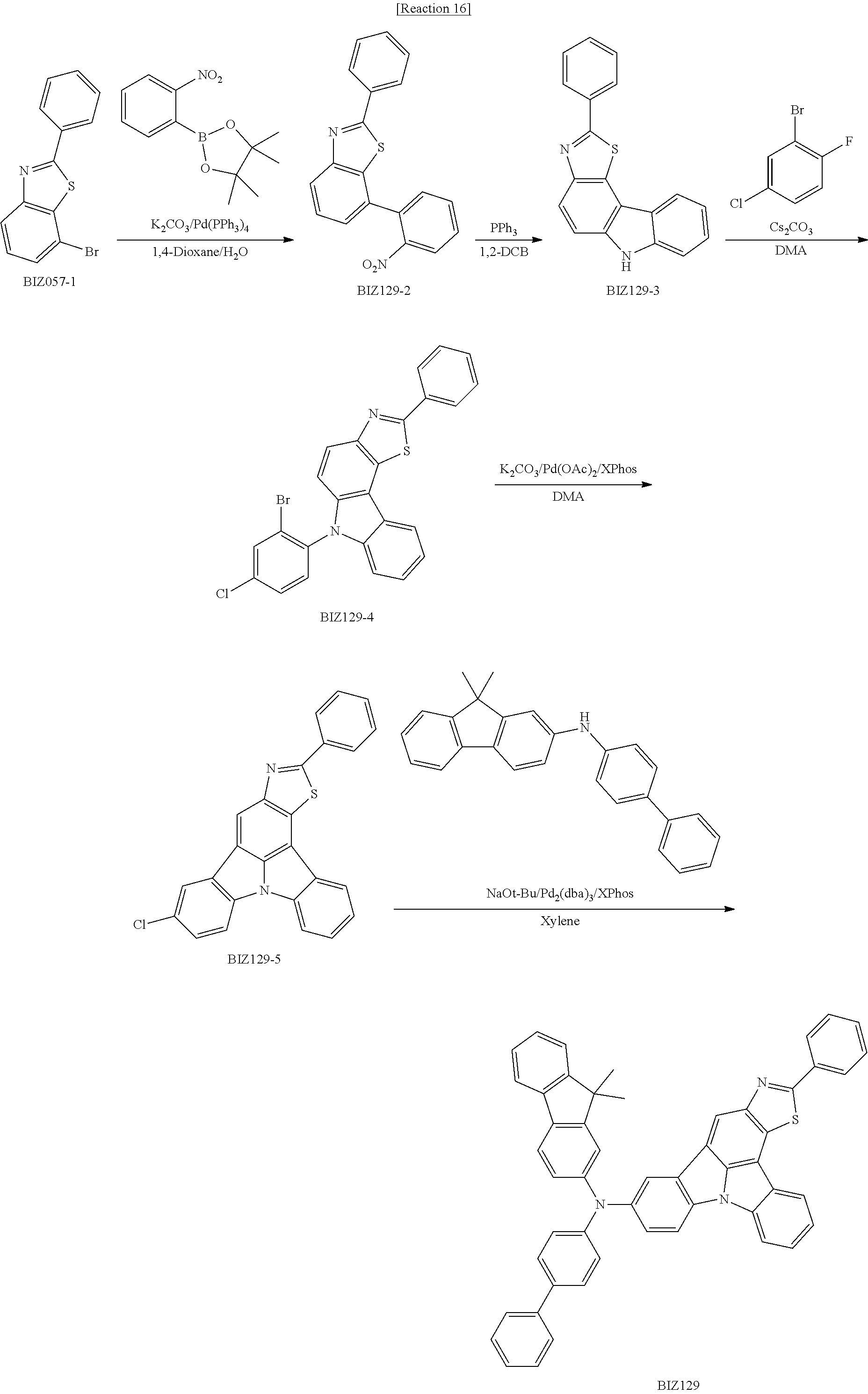

### (1) Compound BIZ129-2

In the one-neck rounded-bottom flask, a mixture of 7-bromo-2-phenylbenzo[d]thiazole (20 g, 0.069 mol), 4,4,5,5-tetramethyl-2-(2-nitrophenyl)-1,3,2-dioxaborolane (20.6 g, 0.083 mol), K₂CO₃ (28.6 g, 0.207 mol), Pd(PPh₃)₄ (4.0 g, 0.003 mol), 1,4-dioxane (300 ml), and water (60 ml) was refluxed for 8 hours at 100° C. After the temperature was lowered, the mixture was extracted with dichloromethane, and moisture was removed using MgSO₄. After the solvent was removed, the mixture was dissolved in dichloromethane and is filtered with silica gel. The mixture was concentrated to obtain the compound BIZ129-2 (16 g, 70%).

### (2) Compound BIZ129-3

In the one-neck rounded-bottom flask, a mixture of the compound BIZ129-2 (16 g, 0.048 mol), PPh₃ (37.9 g, 0.144 mol), and 1,2-dichlorobenzene (160 ml) was stirred for 8 hours at 160° C. After the solvent was removed, the mixture was dissolved in dichloromethane and is filtered with silica gel. The mixture was concentrated to obtain the compound BIZ129-3 (11 g, 76%).

### (3) Compound BIZ129-4

In the one-neck rounded-bottom flask, a mixture of the compound BIZ129-3 (11 g, 0.037 mol), 2-bromo-4-chloro-1-fluorobenzene (9.2 g, 0.044 mol), Cs₂CO₃ (35.7 g, 0.110 mol), and dimethylacetamide (110 ml) was stirred for 8 hours at 130° C. After the solvent was removed, the mixture was dissolved in dichloromethane and is filtered with silica gel. The mixture was concentrated to obtain the compound BIZ129-4 (13 g, 73%).

### (4) Compound BIZ129-5

In the one-neck rounded-bottom flask, a mixture of the compound BIZ129-4 (13 g, 0.027 mol), K₂C03 (11 g, 0.080 mol), Pd(OAC)₂ (0.6 g, 0.003 mol). XPhos (2.5 g, 0.005 mol), and dimethylacetamide (130 ml) was stirred for 8 hours at 130° C. After the solvent was removed, the mixture was dissolved in 1,2-dichlorobenzene and is filtered with silica gel. The mixture was concentrated to obtain the compound BIZ129-5 (6 g, 57%).

### (5) Compound BIZ129

In the one-neck rounded-bottom flask, a mixture of the compound BIZ129-5 (6 g, 0.015 mol), N-([1,1′-biphenyl]-4-yl)-9,9-dimethyl-9H-fluoren-2-amine (5.8 g, 0.016 mol), NaOt-Bu (2.8 g, 0.029 mol), Pd₂(dba)₃ (1.3 g, 0.001 mol), XPhos (1.3 g, 0.003 mol), and xylene (100 ml) was refluxed for 8 hours at 140° C. After the solvent was removed, the mixture was dissolved in 1,2-dichlorobenzene and is filtered with silica gel. The mixture was concentrated to obtain the compound BIZ129 (7 g, 65%).

### 17. Synthesis of the Compound BIZ134

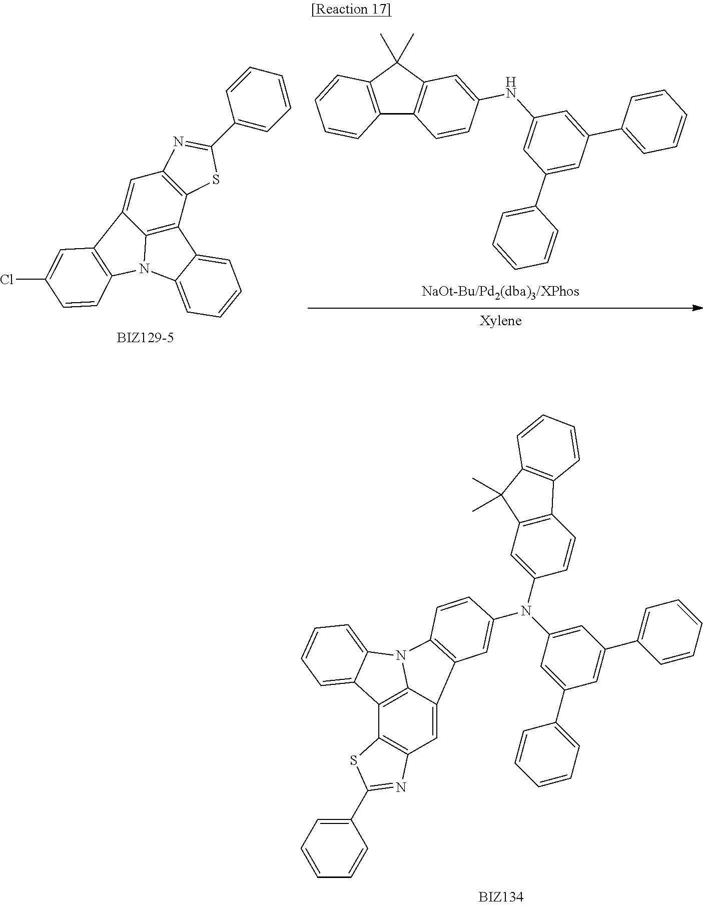

Except using N-([1,1′:3′,1″-terphenyl]-5′-yl)-9,9-dimethyl-9H-fluoren-2-amine instead of N-([1,1′-biphenyl]-4-yl)-9,9-dimethyl-9H-fluoren-2-amine in the synthesis process of the compound BIZ129, the same process was performed to obtain the compound BIZ134 (68%).

### 18. Synthesis of the Compound BIZ136

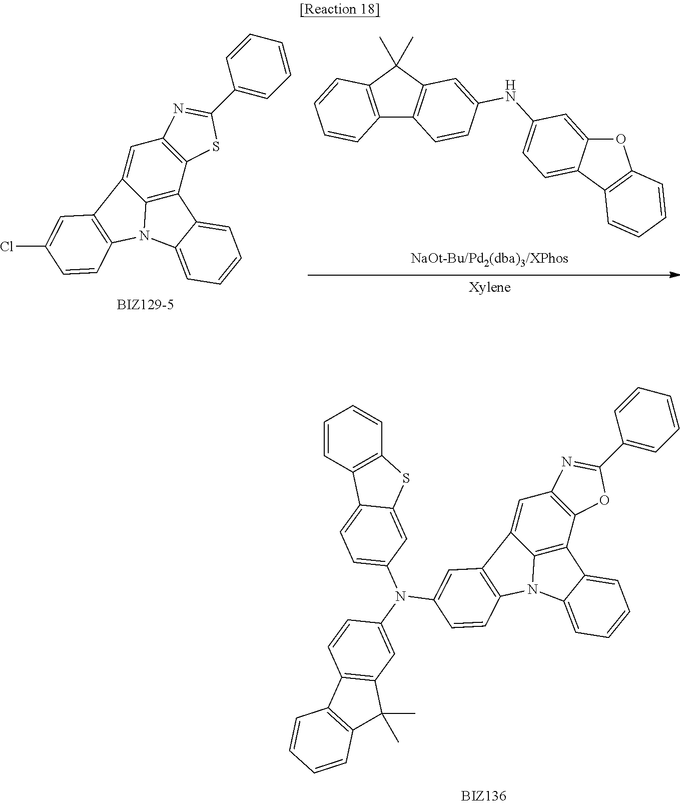

Except using N-(9,9-dimethyl-9H-fluoren-2-yl)dibenzo[b,d]furan-3-amine instead of N-([1,1′-biphenyl]-4-yl)-9,9-dimethyl-9H-fluoren-2-amine in the synthesis process of the compound BIZ129, the same process was performed to obtain the compound BIZ136 (65%).

### 19. Synthesis of the Compound BIZ137

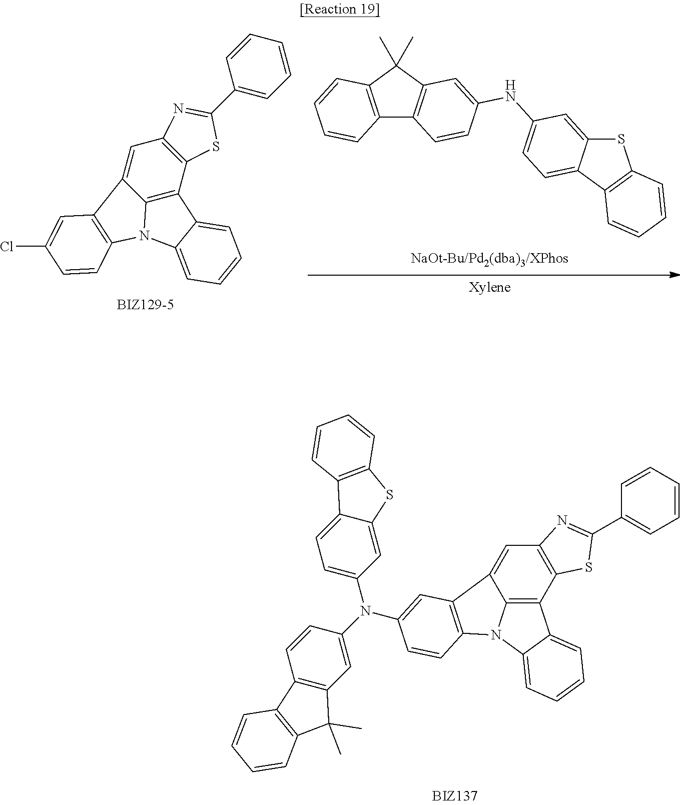

Except using N-(9,9-dimethyl-9H-fluoren-2-yl)dibenzo[b,d]thiophen-3-amine instead of N-([1,1′-biphenyl]-4-yl)-9,9-dimethyl-9H-fluoren-2-amine in the synthesis process of the compound BIZ129, the same process was performed to obtain the compound BIZ137 (61%).

### 20. Synthesis of the Compound BIZ144

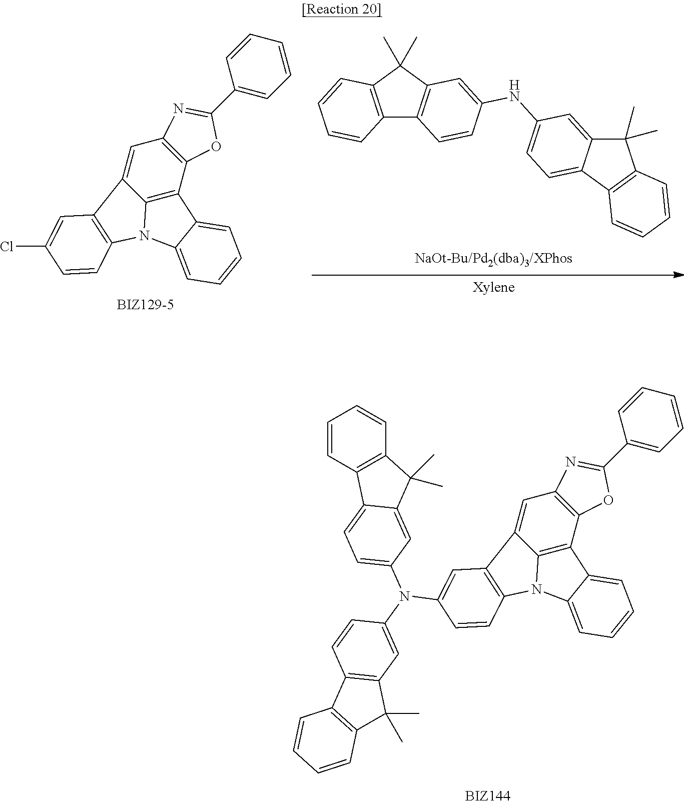

Except using bis(9,9-dimethyl-9H-fluoren-2-yl)amine instead of N-([1,1′-biphenyl]-4-yl)-9,9-dimethyl-9H-fluoren-2-amine in the synthesis process of the compound BIZ129, the same process was performed to obtain the compound BIZ144 (67%).

The present disclosure relates to an OLED, in which the organic compound represented by Formula 1 is included in an emitting material layer, and an organic light emitting device including the OLED. For example, the organic light emitting device may be an organic light emitting display device or an organic lightening device. As an example, an organic light emitting display device, which is a display device including the OLED of the present disclosure, will be mainly described.

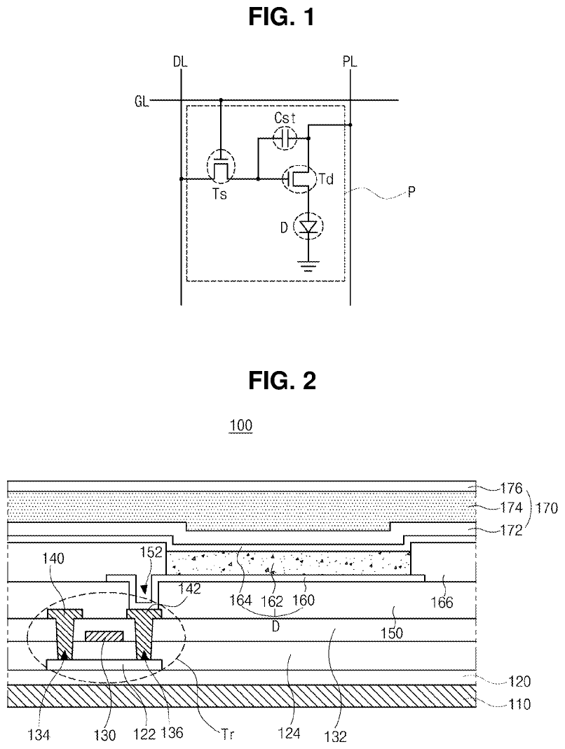

FIG. 1 is a schematic circuit diagram illustrating an organic light emitting display device of the present disclosure.

As illustrated in FIG. 1, a gate line GL and a data line DL, which cross each other to define a pixel region (pixel) P, and a power line PL are formed in an organic light emitting display device. A switching thin film transistor (TFT) Ts, a driving TFT Td, a storage capacitor Cst and an OLED D are formed in the pixel region P. The pixel region P may include a red pixel region, a green pixel region and a blue pixel region.

The switching thin film transistor Ts is connected to the gate line GL and the data line DL, and the driving thin film transistor Td and the storage capacitor Cst are connected between the switching thin film transistor Ts and the power line PL. The OLED D is connected to the driving thin film transistor Td. When the switching thin film transistor Ts is turned on by the gate signal applied through the gate line GL, the data signal applied through the data line DL is applied to a gate electrode of the driving thin film transistor Td and one electrode of the storage capacitor Cst through the switching thin film transistor Ts.

The driving thin film transistor Td is turned on by the data signal applied into the gate electrode so that a current proportional to the data signal is supplied from the power line PL to the OLED D through the driving thin film transistor Td. The OLED D emits light having a luminance proportional to the current flowing through the driving thin film transistor Td. In this case, the storage capacitor Cst is charged with a voltage proportional to the data signal so that the voltage of the gate electrode in the driving thin film transistor Td is kept constant during one frame. Therefore, the organic light emitting display device can display a desired image.

FIG. 2 is a schematic cross-sectional view illustrating an organic light emitting display device according to a first embodiment of the present disclosure.

As illustrated in FIG. 2, the organic light emitting display device 100 includes a substrate 110, a TFT Tr and an OLED D positioned on a planarization layer 150 and connected to the TFT Tr.

The substrate 110 may be a glass substrate or a flexible substrate. For example, the substrate 110 may be a polyimide (PI) substrate, a polyethersulfone (PES) substrate, a polyethylenenaphthalate (PEN) substrate, a polyethylene terephthalate (PET) substrate or a polycarbonate (PC) substrate.

A buffer layer 120 is formed on the substrate, and the TFT Tr is formed on the buffer layer 120. The buffer layer 120 may be omitted.

A semiconductor layer 122 is formed on the buffer layer 120. The semiconductor layer 122 may include an oxide semiconductor material. When the semiconductor layer 122 includes the oxide semiconductor material, a light-shielding pattern (not shown) may be formed under the semiconductor layer 122. The light to the semiconductor layer 122 is shielded or blocked by the light-shielding pattern such that thermal degradation of the semiconductor layer 122 can be prevented. On the other hand, when the semiconductor layer 122 includes polycrystalline silicon, impurities may be doped into both sides of the semiconductor layer 122.

A gate insulating layer 124 is formed on the semiconductor layer 122. The gate insulating layer 124 may be formed of an inorganic insulating material such as silicon oxide or silicon nitride.

A gate electrode 130, which is formed of a conductive material, e.g., metal, is formed on the gate insulating layer 124 to correspond to a center of the semiconductor layer 122. In FIG. 2, the gate insulating layer 124 is formed on an entire surface of the substrate 110. Alternatively, the gate insulating layer 124 may be patterned to have the same shape as the gate electrode 130.

An interlayer insulating layer 132, which is formed of an insulating material, is formed on the gate electrode 130 and over an entire surface of the substrate 110. The interlayer insulating layer 132 may be formed of an inorganic insulating material. e.g., silicon oxide or silicon nitride, or an organic insulating material, e.g., benzocyclobutene or photo-acryl.

The interlayer insulating layer 132 includes first and second semiconductor contact holes 134 and 136 exposing both sides of the semiconductor layer 122. The first and second semiconductor contact holes 134 and 136 are positioned at both sides of the gate electrode 130 to be spaced apart from the gate electrode 130.

In FIG. 2, the first and second semiconductor contact holes 134 and 136 are formed through the gate insulating layer 124. Alternatively, when the gate insulating layer 124 is patterned to have the same shape as the gate electrode 130, the first and second semiconductor contact holes 134 and 136 is formed only through the interlayer insulating layer 132.

A source electrode 140 and a drain electrode 142, which are formed of a conductive material, e.g., metal, are formed on the interlayer insulating layer 132.

The source electrode 140 and the drain electrode 142 are spaced apart from each other with respect to the gate electrode 130 and respectively contact both sides of the semiconductor layer 122 through the first and second semiconductor contact holes 134 and 136.

The semiconductor layer 122, the gate electrode 130, the source electrode 140 and the drain electrode 142 constitute the TFT Tr. The TFT Tr serves as a driving element. Namely, the TFT Tr may correspond to the driving TFT Td (of FIG. 1).

In the TFT Tr in FIG. 2, the gate electrode 130, the source electrode 140, and the drain electrode 142 are positioned over the semiconductor layer 122. Namely, the TFT Tr has a coplanar structure. Alternatively, in the TFT Tr, the gate electrode may be positioned under the semiconductor layer, and the source and drain electrodes may be positioned over the semiconductor layer such that the TFT Tr may have an inverted staggered structure. In this instance, the semiconductor layer may include amorphous silicon.

Although not shown, the gate line and the data line cross each other to define the pixel region, and the switching TFT is formed to be connected to the gate and data lines. The switching TFT is connected to the TFT Tr as the driving element. In addition, the power line, which may be formed to be parallel to and spaced apart from one of the gate and data lines, and the storage capacitor for maintaining the voltage of the gate electrode of the TFT Tr in one frame may be further formed.

A planarization layer 150 is formed on the source and drain electrodes 140 and 142 and over an entire surface of the substrate 110. The planarization layer 150 has a flat top surface and includes a drain contact hole 152 exposing the drain electrode 142 of the TFT Tr.

The OLED D is disposed on the planarization layer 150 and includes a first electrode 160, which is connected to the drain electrode 142 of the TFT Tr, an light emitting layer 162 and a second electrode 164. The light emitting layer 162 and the second electrode 164 are sequentially stacked on the first electrode 160. The OLED D is positioned in each of the red, green and blue pixel regions and respectively emits the red, green and blue light.

The first electrode 160 is separately formed in each pixel region. The first electrode 160 may be an anode and may be formed of a conductive material, e.g., a transparent conductive oxide (TCO), having a relatively high work function. For example, the first electrode 160 may be formed of indium-tin-oxide (ITO), indium-zinc-oxide (IZO), indium-tin-zinc-oxide (ITZO), tin oxide (SnO), zinc oxide (ZnO), indium-copper-oxide (ICO) or aluminum-zinc-oxide (Al:ZnO, AZO).

When the organic light emitting display device 100 of the present disclosure is operated in a bottom-emission type, the first electrode 160 may have a single-layered structure of a transparent conductive oxide layer of the transparent conductive oxide. Alternatively, when the organic light emitting display device 100 of the present disclosure is operated in a top-emission type, the first electrode 160 may further include a reflection layer to have a double-layered structure or a triple-layered structure. For example, the reflection layer may be formed of silver (Ag) or aluminum-palladium-copper (APC) alloy. In the top-emission type OLED, the first electrode 160 may have a double-layered structure of Ag/ITO or APC/ITO or a triple-layered structure of ITO/Ag/ITO or ITO/APC/ITO.

In addition, a bank layer 166 is formed on the planarization layer 150 to cover an edge of the first electrode 160. Namely, the bank layer 166 is positioned at a boundary of the pixel region and exposes a center of the first electrode 160 in the pixel region.

The organic light emitting layer 162 is formed on the first electrode 160. The organic light emitting layer 162 may have a single-layered structure of an emitting material layer (EML) including an emitting material. Alternatively, the organic light emitting layer 162 may further include at least one of a hole injection layer (HIL), a hole transporting layer (HTL), an electron blocking layer (EBL), a hole blocking layer (HBL), an electron transporting layer (ETL) and an electron injection layer (EIL) to have a multi-layered structure. In addition, two or more EMLs may be sequentially stacked. Moreover, two or more EMLs may be disposed to be spaced apart from each other such that the OLED D may have a tandem structure.

In the red pixel region, the organic light emitting layer 162 in the OLED D includes the organic compound of the present disclosure. As a result, the emitting efficiency and the lifespan of the OLED D and the organic light emitting display device 100 are improved.

The second electrode 164 is formed over the substrate 110 where the organic light emitting layer 162 is formed. The second electrode 164 covers an entire surface of the display area and may be formed of a conductive material having a relatively low work function to serve as a cathode. For example, the second electrode 164 may be formed of a high reflective material, e.g., aluminum (Al), magnesium (Mg), calcium (Ca), silver (Ag), their alloy, or their combination. In the top-emission type organic light emitting display device 100, the second electrode 164 may be thin to provide to be transparent (or semi-transparent).

An encapsulation layer (or an encapsulation film) 170 is formed on the second electrode 164 to prevent penetration of moisture into the OLED D. The encapsulation layer 170 includes a first inorganic insulating layer 172, an organic insulating layer 174 and a second inorganic insulating layer 176 sequentially stacked, but it is not limited thereto.

In the bottom-emission type organic light emitting display device 100, a metal plate may be further disposed on the encapsulation layer 170.

The organic light emitting display device 100 may include a color filter layer corresponding to the red, green and blue pixel regions. The color filter layer may include a red color filter, a green color filter and a blue color filter corresponding to the red, green and blue pixel regions, respectively. When the OLED includes the color filter layer, the color purity of the OLED can be further improved.

When the organic light emitting display device 100 is operated in a bottom-emission type, the color filter layer may be disposed between the OLED D and the substrate 110, e.g., between the interlayer insulating layer 132 and the planarization layer 150. Alternatively, the organic light emitting display device 100 is operated in a top-emission type, the color filter layer may be disposed over the OLED D, e.g., over the second electrode 164 or the encapsulation layer 170.

The organic light emitting display device 100 may further include a polarization plate for reducing an ambient light reflection. For example, the polarization plate may be a circular polarization plate. In the bottom-emission type organic light emitting display device 100, the polarization plate may be positioned under the substrate 110. Alternatively, in the top-emission type organic light emitting display device 100, the polarization plate may be positioned on or over the encapsulation layer 170.

In addition, in the top-emission type organic light emitting display device 100, a cover window may be attached to the encapsulation layer 170. In this instance, the substrate 110 and the cover window have a flexible property such that a flexible organic light emitting display device may be provided.

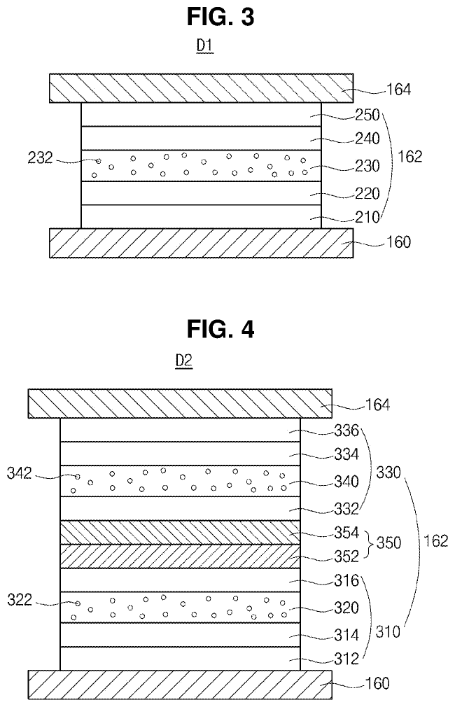

FIG. 3 is a schematic cross-sectional view illustrating an OLED according to a second embodiment.

As shown in FIG. 3, the OLED D1 includes the first and second electrodes 160 and 164 facing each other and the organic emitting layer 162 between the first and second electrodes 160 and 164. The organic emitting layer 162 includes a red EML 230.

The organic light emitting display device 100 (of FIG. 2) may include a red pixel region, a green pixel region and a blue pixel region. The organic light emitting display device 100 may further include a white pixel region. The OLED D1 may be positioned in the red pixel region.

The first electrode 160 is an anode injecting a hole, and the second electrode 164 is a cathode injecting an electron. One of the first and second electrodes 160 and 164 is a reflection electrode, and the other one of the first and second electrodes 160 and 164 is a transparent electrode (or a semi-transparent electrode).

For example, the first electrode 160 may include a transparent conductive material. e.g., ITO or IZO, and the second electrode 164 may be formed of Al, Mg, Ag. AlMg or MgAg.

The organic emitting layer 162 may further include at least one of an HTL 220 under the red EML 230 and an ETL 240 on or over the red EML 230. Namely, the HTL 220 is positioned between the red EML 230 and the first electrode 160, and the ETL 240 is positioned between the red EML 230 and the second electrode 164.

In addition, the organic emitting layer 162 may further include at least one of an HIL 210 under the HTL 220 and an EIL 250 on the ETL 240.

Although not shown, the organic emitting layer 162 may further include at least one of an EBL between the HTL 220 and the red EML 230 and an HBL between the red EML 230 and the ETL 240.

For example, the HIL 210 may include a hole injection material selected from the group consisting of 4,4′,4″-tris(3-methylphenylamino)triphenylamine (MTDATA), 4,4′,4″-tris(N,N-diphenyl-amino)triphenylamine (NATA), 4,4′,4″-tris(N-(naphthalene-1-yl)-N-phenyl-amino)triphenylamine (1T-NATA), 4,4′,4″-tris(N-(naphthalene-2-yl)-N-phenyl-amino)triphenylamine (2T-NATA), copper phthalocyanine (CuPc), tris(4-carbazoyl-9-yl-phenyl)amine (TCTA), N,N′-diphenyl-N,N′-bis(1-naphthyl)-1,1′-biphenyl-4,4″-diamine (NPB or NPD), 1,4,5,8,9,11-hexaazatriphenylenehexacarbonitrile (HAT-CN), 1,3,5-tris[4-(diphenylamino)phenyl]benzene (TDAPB), poly(3,4-ethylenedioxythiphene)polystyrene sulfonate (PEDOT/PSS), N-(biphenyl-4-yl)-9,9-dimethyl-N-(4-(9-phenyl-9H-carbazol-3-yl)phenyl)-9H-fluoren-2-amine), and N,N′-diphenyl-N,N′-di[4-(N,N-diphenyl-amino)phenyl]benzidine (NPNPB), but it is not limited thereto. For example, the hole injection material of the HIL 210 may include a compound in Formula 7. The HIL 210 may have a thickness of 10 to 100 Å.

The HTL 220 may include a hole transporting material selected from the group consisting of N,N′-diphenyl-N,N′-bis(3-methylphenyl)-1,1′-biphenyl-4,4′-diamine (TPD), NPB (NPD), 4,4′-bis(N-carbazolyl)-1,1′-biphenyl (CBP), poly[N,N′-bis(4-butylpnehyl)-N,N′-bis(phenyl)-benzidine] (poly-TPD), (poly[(9,9-dioctylfluorenyl-2,7-diyl)-co-(4,4′-(N-(4-sec-butylphenyl)diphenylamine))] (TFB), di-[4-(N,N-di-p-tolyl-amino)-phenyl]cyclohexane (TAPC), 3,5-di(9H-carbazol-9-yl)-N,N-diphenylaniline (DCDPA), N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine, and N-([1,1′-biphenyl]-4-yl)-9,9-dimethyl-N-(4-(9-phenl-9H-carbazol-3-yl)phenyl)-9H-fluoren-2-amine, but it is not limited thereto. For example, the hole transporting material of the HTL 220 may include a compound in Formula 8. The HTL 220 may have a thickness of 500 to 1500 Å, preferably 700 to 1200 Å.

The ETL 240 may include an electron transporting material selected from the group consisting of tris-(8-hydroxyquinoline aluminum (Alq₃), 2-biphenyl-4-yl-5-(4-t-butylphenyl)-1,3,4-oxadiazole (PBD), spiro-PBD, lithium quinolate (Liq), 1,3,5-tris(N-phenylbenzimidazol-2-yl)benzene (TPBi), bis(2-methyl-8-quinolinolato-N1,O8)-(1,1′-biphenyl-4-olato)aluminum (BAlq), 4,7-diphenyl-1,10-phenanthroline (Bphen), 2,9-bis(naphthalene-2-yl)-4,7-diphenyl-1,10-phenanthroline (NBphen), 2,9-dimethyl-4,7-diphenyl-1,10-phenathroline (BCP), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), 1,3,5-tri(p-pyrid-3-yl-phenyl)benzene (TpPyPB), 2,4,6-tris(3′-(pyridin-3-yl)biphenyl-3-yl)-1,3,5-triazine (TmPPPyTz), poly[9,9-bis(3′-((N,N-dimethyl)-N-ethylammonium)-propyl)-2,7-fluorene]-alt-2,7-(9,9-dioctylfluorene)] (PFNBr), tris(phenylquinoxaline) (TPQ), diphenyl-4-triphenylsilyl-phenylphosphine oxide (TSPO1), and 2-[4-(9,10-Di-2-naphthalen2-yl-2-anthracen-2-yl)phenyl]-1-phenyl-1H-benzimidazole (ZADN), but it is not limited thereto. For example, the electron transporting material of the ETL 240 may include a compound in Formula 9. The ETL 240 may have a thickness of 100 to 500 Å, preferably 200 to 400 Å.

The EIL 250 may include an electron injection material selected from the group consisting of LiF, CsF, NaF, BaF₂, Liq, lithium benzoate, and sodium stearate, but it is not limited thereto. The EIL 250 may have a thickness of 1 to 50 Å, preferably 5 to 30 Å.

The EBL, which is positioned between the HTL 220 and the EML 230 to block the electron transfer from the EML 230 into the HTL 220, may include an electron blocking material selected from the group consisting of TCTA, tris[4-(diethylamino)phenyl]amine, N-(biphenyl-4-yl)-9,9-dimethyl-N-(4-(9-phenyl-9H-carbazol-3-yl)phenyl)-9H-fluoren-2-amine, TAPC, MTDATA, 1,3-bis(carbazol-9-yl)benzene (mCP), 3,3′-bis(N-carbazolyl)-1,1′-biphenyl (mCBP), CuPc, N,N′-bis[4-[bis(3-methylphenyl)amino]phenyl]-N,N′-diphenyl-[1,1′-biphenyl]-4,4′-diamine (DNTPD), TDAPB, DCDPA, and 2,8-bis(9-phenyl-9H-carbazol-3-yl)dibenzo[b,d]thiophene), but it is not limited thereto. The EBL may have a thickness of 10 to 300 Å.

The HBL, which is positioned between the EML 230 and the ETL 240 to block the hole transfer from the EML 230 into the ETL 240, may include the above material of the ETL 240. For example, the HBL may include a hole blocking material selected from the group consisting of BCP, BAlq, Alq₃, PBD, spiro-PBD, Liq, bis-4,6-(3,5-di-3-pyridylphenyl)-2-methylpyrimidine (B3PYMPM), bis[2-(diphenylphosphino)phenyl]ether oxide (DPEPO), 9-(6-9H-carbazol-9-yl)pyridine-3-yl)-9H-3,9′-bicarbazole, and TSPO1, but it is not limited thereto. The HBL may have a thickness of 10 to 300 Å.

In the OLED D1 of the present disclosure, the red EML 230 singly or in combination with at least one of HIL 210, HTL 220, EBL, HBL, ETL 240 and EIL 250 may constitute an emitting part.

The red EML 230 includes a first compound 232 being the organic compound of the present disclosure. Namely, the first compound 232 is represented by one of Formulas 1, 1-1 and 1-2 and is selected from the compounds in Formula 2.

The red EML 230 may further include a second compound being a red dopant, e.g., a red emitter. In this case, the first compound 232 serves as a host. The second compound may include at least one of a fluorescent compound, a delayed fluorescent compound and a phosphorescent compound.

For example, the second compound may include at least one of [bis(2-(4,6-dimethyl)phenylquinoline)](2,2,6,6-tetramethylheptane-3,5-dionate)iridium(III), bis[2-(4-n-hexylphenyl)quinoline](acetylacetonate)iridium(III) (Hex-Ir(phq)₂(acac)), tris[2-(4-n-hexylphenyl)quinoline]iridium(III) (Hex-Ir(phq)₃), tris[2-phenyl-4-methylquinoline]iridium(III) (Ir(Mphq)₃), bis(2-phenylquinoline)(2,2,6,6-tetramethylheptane-3,5-dionate)iridium(III) (Ir(dpm)PQ₂), bis(phenylisoquinoline)(2,2,6,6-tetramethylheptane-3,5-dionate)iridium(III) (Ir(dpm)(piq)₂), bis[(4-n-hexylphenyl)isoquinoline](acetylacetonate)iridium(III) (Hex-Ir(piq)₂(acac)), tris[2-(4-n-hexylphenyl)quinoline]iridium(III) (Hex-Ir(piq)₃), tris(2-(3-methylphenyl)-7-methyl-quinolato)iridium (Ir(dmpq)₃), bis[2-(2-methylphenyl)-7-methyl-quinoline](acetylacetonate)iridium(III) (Ir(dmpq)₂(acac)), bis[2-(3,5-dimethylphenyl)-4-methyl-quinoline](acetylacetonate)iridium(III) (Ir(mphmq)₂(acac)), and tris(dibenzoylmethane)mono(1,10-phenanthroline)europium(III) (Eu(dbm)₃(phen)).

In an aspect of the present disclosure, the second compound may be represented by Formula 3.

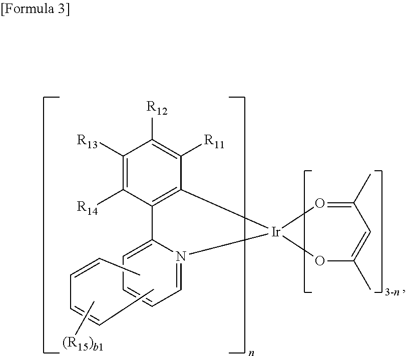

In Formula 3,

n is an integer of 1 to 3, and b1 is an integer of 0 to 4,

each of R₁₁ to R₁₄ is independently selected from the group consisting of hydrogen, a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group, or optionally adjacent two of R₁₁ to R₁₄ is combined to each other to form an aromatic ring or a heteroaromatic ring, and

R₁₅ is independently selected from the group consisting of a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group.

In an aspect of the present disclosure, n may be 2, and b1 may be 0.

In an aspect of the present disclosure, each of R₁₁ to R₁₄ may be hydrogen.

In an aspect of the present disclosure, each of R₁₁ and R₁₄ may be hydrogen, and R₁₂ and R₁₃ may be combined to each other to form an aromatic ring.

In an aspect of the present disclosure, R₁₁, R₁₃ and R₁₄ may be hydrogen, and R₁₂ may be a substituted or unsubstituted C1 to C10 alkyl group, e.g., hexyl (C₆H₁₃).

For example, the second compound may be one of compounds in Formula 4.

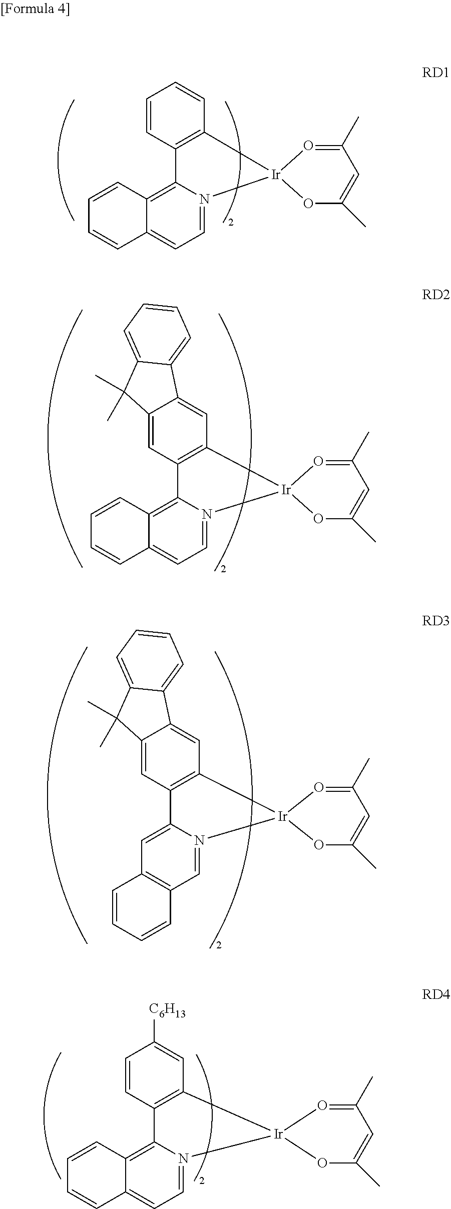

When the red EML 230 includes the first compound 232 being the organic compound, which is represented by Formula 1 and is one of the compounds in Formula 2, and the second compound, which is represented by Formula 3 and is one of the compounds in Formula 4, an energy transfer efficiency from the first compound 232 to the second compound is improved. As a result, in the OLED D1 including the red EML 230 and the organic light emitting display device 100 including the OLED D1, the driving voltage is decreased, and the emitting efficiency and the lifespan are increased.

The red EML 230 may further include a third compound. In this case, the first compound 232 may serve as a p-type host, and the third compound may serve as an n-type host.

The third compound may be represented by Formula 5.

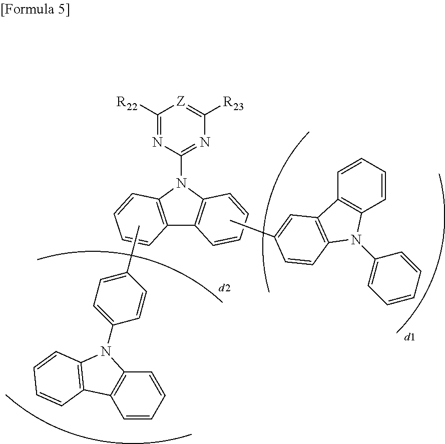

In Formula 5,

each of d1 and d2 is independently 0 or 1,

Z is N or CR₂₁, and each of R₂₁, R₂₂ and R₂₃ is independently selected from the group consisting of hydrogen, a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group,

optionally, when Z is CR₂₁, one of a pair of R₂₁ and R₂₂ or a pair of R₂₁ and R₂₃ is combined to each other to form an aromatic ring or a heteroaromatic ring.

In an aspect of the present disclosure, one of d1 and d2 may be 0, and the other one of d1 and d2 may be 1.

In an aspect of the present disclosure, when Z is N, each of R₂₂ and R₂₃ may be independently a substituted or unsubstituted C6 to C30 aryl group, e.g., phenyl.

For example, the third compound may be one of compounds in Formula 6.

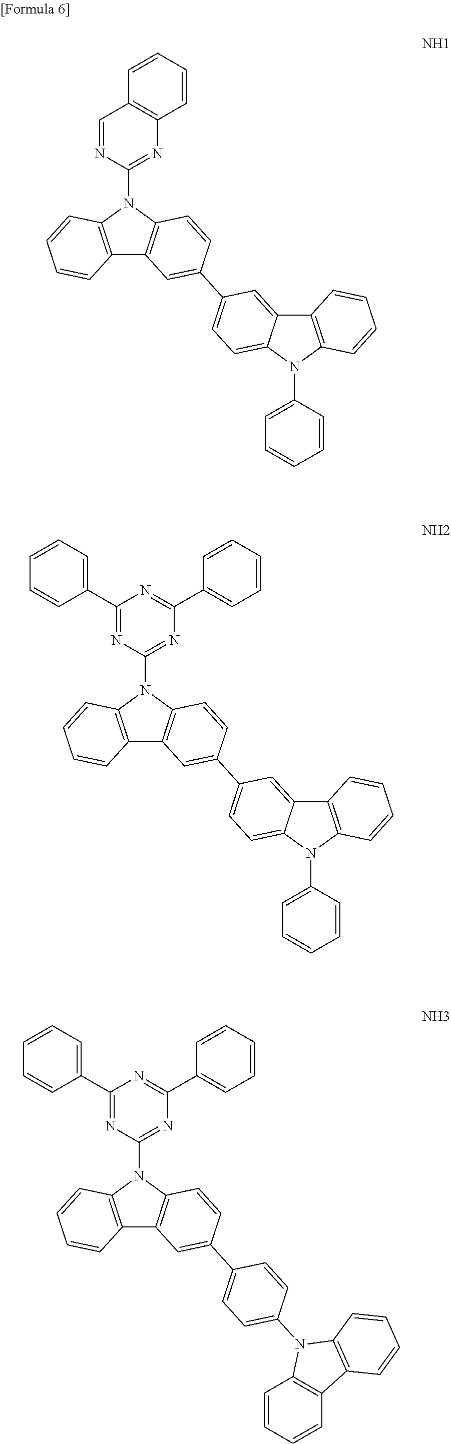 [SMILES: c1ccc(-n2c3ccccc3c3cc(-c4ccc5c(c4)c4ccccc4n5-c4ncc5ccccc5n4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-n3c4ccccc4c4cc(-c5ccc(-n6c7ccccc7c7ccccc76)cc5)ccc43)n2)cc1]

When the red EML 230 includes the first compound 232, which is the organic compound represented by Formula 1 and selected from the compounds in Formula 2, the second compound, which is represented by Formula 3 and selected from the compounds in Formula 4, and the third compound, which is represented by Formula 5 and selected from the compounds in Formula 6, an exciton generation efficiency between the first compound 232 and the third compound is increased, and the generated exciton is efficiently transferred into the second compound. Accordingly, in the OLED D1 including the red EML 230 and the organic light emitting display device 100 including the OLED D1, the driving voltage is further decreased, and the emitting efficiency and the lifespan are further increased.

The red EML 230 may have a thickness of 100 to 400 Å, preferably 200 to 400 Å, but it is not limited thereto.

When the red EML 230 includes the first compound 232 and the second compound, e.g., a dopant, a weight % of the first compound 232 is greater than that of the second compound. For example, in the red EML 230, the first compound 232 may have a weight % of 60 to 99.

When the red EML 230 includes the first compound 232, the second compound and the third compound, a weight % of each of the first compound 232 and the third compound may be greater than that of the second compound, and the weight % of the first compound 232 and the weight % of the third compound may be same or different. For example, the weight % of the first compound 232 and the weight % of the third compound may be same, and the second compound may have a weight % of 1 to 5 in the red EML 230.

As described above, the OLED D1 of the present disclosure is positioned in the red pixel region, and the red EML 230 includes the first compound 232 being the organic compound represented by Formula 1. As a result, in the OLED D1, the driving voltage is decreased, and the emitting efficiency and the lifespan are increased.

FIG. 4 is a schematic cross-sectional view illustrating an OLED according to a third embodiment.

As shown in FIG. 4, the OLED D2 includes the first and second electrodes 16i0 and 164 facing each other and the organic emitting layer 162 between the first and second electrodes 160 and 164. The organic emitting layer 162 includes a first emitting part 310 including a first red EML 320 and a second emitting part 330 including a second red EML 340. The organic emitting layer 162 may further include a charge generation layer (CGL) 350 between the first and second emitting parts 310 and 330.

The organic light emitting display device 100 includes a red pixel region, a green pixel region and a blue pixel region. The organic light emitting display device 100 may further include a white pixel region. The OLED D2 is positioned in the red pixel region.

The first electrode 160 is an anode for injecting a hole, and the second electrode 164 is cathode for injecting an electron. In addition, one of the first and second electrodes 160 and 164 may be a reflection electrode, and the other one of the first and second electrodes 160 and 164 may be a transparent (or a semi-transparent) electrode.

For example, the first electrode 160 may include a transparent conductive material, e.g., ITO or IZO, and the second electrode 164 may be formed of Al, Mg, Ag, AlMg or MgAg.

The CGL 350 is positioned between the first and second emitting parts 310 and 330, and the first emitting part 310, the CGL 350 and the second emitting part 330 are sequentially stacked on the first electrode 160. Namely, the first emitting part 310 is positioned between the first electrode 160 and the CGL 350, and the second emitting part 330 is positioned between the second electrode 164 and the CGL 350.

The first red EML 320 includes a first compound 322 being the organic compound of the present disclosure. Namely, the first compound 322 is represented by one of Formulas 1, 1-1 and 1-2 and is selected from the compounds in Formula 2.

The first red EML 320 may further include a second compound being a red dopant, e.g., a red emitter. In this case, the first compound 322 serves as a host. The second compound may include at least one of a fluorescent compound, a delayed fluorescent compound and a phosphorescent compound. For example, the second compound may be represented by Formula 3 and may be one of the compounds in Formula 4.

When the first red EML 320 includes the first compound 322 being the organic compound, which is represented by Formula 1 and is one of the compounds in Formula 2, and the second compound, which is represented by Formula 3 and is one of the compounds in Formula 4, an energy transfer efficiency from the first compound 322 to the second compound is improved. As a result, in the OLED D2 including the first red EML 320 and the organic light emitting display device 100 including the OLED D2, the driving voltage is decreased, and the emitting efficiency and the lifespan are increased.

The first red EML 320 may further include a third compound. In this case, the first compound 322 may serve as a p-type host, and the third compound may serve as an n-type host. For example, the third compound may be represented by Formula 5 and may be one of the compounds in Formula 6.

When the first red EML 320 includes the first compound 322, which is the organic compound represented by Formula 1 and selected from the compounds in Formula 2, the second compound, which is represented by Formula 3 and selected from the compounds in Formula 4, and the third compound, which is represented by Formula 5 and selected from the compounds in Formula 6, an exciton generation efficiency between the first compound 322 and the third compound is increased, and the generated exciton is efficiently transferred into the second compound. Accordingly, in the OLED D2 including the first red EML 320 and the organic light emitting display device 100 including the OLED D2, the driving voltage is further decreased, and the emitting efficiency and the lifespan are further increased.

The first red EML 320 may have a thickness of 100 to 400 Å, preferably 200 to 400 Å, but it is not limited thereto.

When the first red EML 320 includes the first compound 322 and the second compound, e.g., a dopant, a weight % of the first compound 322 is greater than that of the second compound. For example, in the first red EML 320, the first compound 322 may have a weight % of 60 to 99.

When the first red EML 320 includes the first compound 322, the second compound and the third compound, a weight % of each of the first compound 322 and the third compound may be greater than that of the second compound, and the weight % of the first compound 322 and the weight % of the third compound may be same or different. For example, the weight % of the first compound 322 and the weight % of the third compound may be same, and the second compound may have a weight % of 1 to 5 in the first red EML 320.

The first emitting part 310 may further include at least one of a first HTL 314 under the first red EML 320 and a first ETL 316 on or over the first red EML 320. Namely, the first HTL 314 is positioned between the first red EML 320 and the first electrode 160, and the first ETL 316 is positioned between the first red EML 320 and the CGL 350.

In addition, the first emitting part 310 may further include an HIL 312 between the first electrode 160 and the first HTL 314.

Moreover, the first emitting part 310 may further include at least one of a first EBL between the first HTL 314 and the first red EML 320 and a first HBL between the first red EML 320 and the first ETL 316.

The second red EML 340 includes a fourth compound 342 being the organic compound of the present disclosure. Namely, the fourth compound 342 is represented by one of Formulas 1, 1-1 and 1-2 and is selected from the compounds in Formula 2. The fourth compound 342 and the first compound 322 may be same or different.

The second red EML 340 may further include a fifth compound being a red dopant, e.g., a red emitter. In this case, the fourth compound 342 serves as a host. The fifth compound may include at least one of a fluorescent compound, a delayed fluorescent compound and a phosphorescent compound. For example, the fifth compound may be represented by Formula 3 and may be one of the compounds in Formula 4. The fifth compound and the second compound may be same or different.

When the second red EML 340 includes the fourth compound 342 being the organic compound, which is represented by Formula 1 and is one of the compounds in Formula 2, and the fifth compound, which is represented by Formula 3 and is one of the compounds in Formula 4, an energy transfer efficiency from the fourth compound 342 to the fifth compound is improved. As a result, in the OLED D2 including the second red EML 340 and the organic light emitting display device 100 including the OLED D2, the driving voltage is decreased, and the emitting efficiency and the lifespan are increased.

The second red EML 340 may further include a sixth compound. In this case, the fourth compound 342 may serve as a p-type host, and the sixth compound may serve as an n-type host. For example, the sixth compound may be represented by Formula 5 and may be one of the compounds in Formula 6. The sixth compound and the third compound may be same or different.

When the second red EML 340 includes the fourth compound 342, which is the organic compound represented by Formula 1 and selected from the compounds in Formula 2, the fifth compound, which is represented by Formula 3 and selected from the compounds in Formula 4, and the sixth compound, which is represented by Formula 5 and selected from the compounds in Formula 6, an exciton generation efficiency between the fourth compound 342 and the sixth compound is increased, and the generated exciton is efficiently transferred into the fifth compound. Accordingly, in the OLED D2 including the second red EML 340 and the organic light emitting display device 100 including the OLED D2, the driving voltage is further decreased, and the emitting efficiency and the lifespan are further increased.

The second red EML 340 may have a thickness of 100 to 400 Å, preferably 200 to 400 Å, but it is not limited thereto.

When the second red EML 340 includes fourth compound 342 and the fifth compound, e.g., a dopant, a weight % of the fourth compound 342 is greater than that of the fifth compound. For example, in the second red EML 340, the fourth compound 342 may have a weight % of 60 to 99.

When the second red EML 340 includes the fourth compound 342, the fifth compound and the sixth compound, a weight % of each of the fourth compound 342 and the sixth compound may be greater than that of the fifth compound, and the weight % of the fourth compound 342 and the weight % of the sixth compound may be same or different. For example, the weight % of the fourth compound and the weight % of the sixth compound may be same, and the fifth compound may have a weight % of 1 to 5 in the second red EML 340.

The second emitting part 330 may further include at least one of a second HTL 332 under the second red EML 340 and a second ETL 334 on or over the second red EML 340. Namely, the second HTL 332 is positioned between the second red EML 340 and the CGL 350, and the second ETL 334 is positioned between the second red EML 340 and the second electrode 164.

In addition, the second emitting part 330 may further include an EIL 336 between the second electrode 164 and the second ETL 334.

Moreover, the second emitting part 330 may further include at least one of a second EBL between the second HTL 332 and the second red EML 340 and a second HBL between the second red EML 340 and the second ETL 334.

The HIL 312 may include the above-mention hole injection material and may have a thickness of 10 to 100 Å. Each of the first and second HTLs 314 and 332 may include the above-mention hole transporting material and may have a thickness of 500 to 1500 Å, preferably 700 to 1200 Å.

Each of the first and second ETLs 316 and 334 may include the above-mention electron transporting material and may have a thickness of 100 to 500 Å, preferably 200 to 400 Å. The EIL 336 may include the above-mention electron injection material and may have a thickness of 1 to 50 Å, preferably 5 to 30 Å.

Each of the first and second EBLs may include the above-mention electron blocking material and may have a thickness of 10 to 300 Å. Each of the first and second HBLs may include the above-mention hole blocking material and may have a thickness of 10 to 300 Å.

The CGL 350 is positioned between the first and second emitting parts 310 and 330. Namely, the first and second emitting parts 310 and 330 are connected through the CGL 350. The CGL 350 may be a P-N junction CGL of an N-type CGL 352 and a P-type CGL 354.

The N-type CGL 352 is positioned between the first HTL 316 and the second HTL 332, and the P-type CGL 354 is positioned between the N-type CGL 352 and the second HTL 332.

The N-type CGL 352 may be an organic layer doped with an alkali metal, e.g., Li, Na, K and Cs, and/or an alkali earth metal, e.g., Mg, Sr, Ba and Ra. For example, the N-type CGL 352 may be formed of an N-type charge generation material including a host being the organic material. e.g., 4,7-dipheny-1,10-phenanthroline (Bphen) and MTDATA, a dopant being an alkali metal and/or an alkali earth metal, and the dopant may be doped with a weight % of 0.01 to 30.

The P-type CGL 354 may be formed of a P-type charge generation material including an inorganic material, e.g., tungsten oxide (WOx), molybdenum oxide (MoOx), beryllium oxide (Be2O3) and vanadium oxide (V2O5), an organic material, e.g., NPD, HAT-CN, F4TCNQ, TPD, TNB, TCTA and N,N′-dioctyl-3,4,9,10-perylenedicarboximide (PTCDI-C8).

In FIG. 4, each of the first and second red EMLs 320 and 340 includes the organic compound of the present disclosure. Alternatively, one of the first and second red EMLs 320 and 340 may include the organic compound of the present disclosure, and the other one of the first and second red EMLs 320 and 340 may include an organic compound, which is other than the organic compound of the present disclosure, as a host.

As described above, the OLED D2 of the present disclosure is positioned in the red pixel region, and at least one of the first and second red EMLs 320 and 340 includes the organic compound represented by Formula 1. As a result, in the OLED D2, the driving voltage is decreased, and the emitting efficiency and the lifespan are increased.

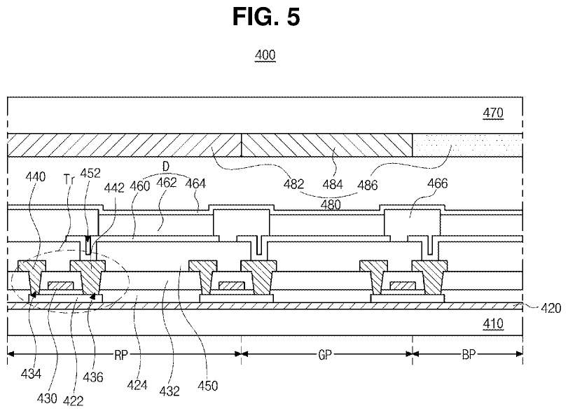

FIG. 5 is a schematic cross-sectional view illustrating an organic light emitting display device according to a fourth embodiment of the present disclosure.

As shown in FIG. 5, the organic light emitting display device 400 includes a first substrate 410, where a red pixel region RP, a green pixel region GP and a blue pixel region BP are defined, a second substrate 470 facing the first substrate 410, an OLED D, which is positioned between the first and second substrates 410 and 470 and providing white emission, and a color filter layer 480 between the OLED D and the second substrate 470.

Each of the first and second substrates 410 and 470 may be a glass substrate or a flexible substrate. For example, the flexible substrate may be a polyimide (PI) substrate, a polyethersulfone (PES) substrate, a polyethylenenaphthalate (PEN) substrate, a polyethylene terephthalate (PET) substrate or a polycarbonate (PC) substrate.

A buffer layer 420 is formed on the first substrate 410, and the TFT Tr corresponding to each of the red, green and blue pixel regions RP. GP and BP is formed on the buffer layer 420. The buffer layer 420 may be omitted.

A semiconductor layer 422 is formed on the buffer layer 420. The semiconductor layer 422 may include an oxide semiconductor material or polycrystalline silicon.

A gate insulating layer 424 is formed on the semiconductor layer 422. The gate insulating layer 424 may be formed of an inorganic insulating material such as silicon oxide or silicon nitride.

A gate electrode 430, which is formed of a conductive material, e.g., metal, is formed on the gate insulating layer 424 to correspond to a center of the semiconductor layer 422.

An interlayer insulating layer 432, which is formed of an insulating material, is formed on the gate electrode 430. The interlayer insulating layer 432 may be formed of an inorganic insulating material, e.g., silicon oxide or silicon nitride, or an organic insulating material, e.g., benzocyclobutene or photo-acryl.

The interlayer insulating layer 432 includes first and second contact holes 434 and 436 exposing both sides of the semiconductor layer 422. The first and second contact holes 434 and 436 are positioned at both sides of the gate electrode 430 to be spaced apart from the gate electrode 430.

A source electrode 440 and a drain electrode 442, which are formed of a conductive material, e.g., metal, are formed on the interlayer insulating layer 432.

The source electrode 440 and the drain electrode 442 are spaced apart from each other with respect to the gate electrode 430 and respectively contact both sides of the semiconductor layer 422 through the first and second contact holes 434 and 436.

The semiconductor layer 422, the gate electrode 430, the source electrode 440 and the drain electrode 442 constitute the TFT Tr. The TFT Tr serves as a driving element. Namely, the TFT Tr may correspond to the driving TFT Td (of FIG. 1).

Although not shown, the gate line and the data line cross each other to define the pixel region, and the switching TFT is formed to be connected to the gate and data lines. The switching TFT is connected to the TFT Tr as the driving element.

In addition, the power line, which may be formed to be parallel to and spaced apart from one of the gate and data lines, and the storage capacitor for maintaining the voltage of the gate electrode of the TFT Tr in one frame may be further formed.

A planarization layer 450, which includes a drain contact hole 452 exposing the drain electrode 442 of the TFT Tr, is formed to cover the TFT Tr.

A first electrode 460, which is connected to the drain electrode 442 of the TFT Tr through the drain contact hole 452, is separately formed in each pixel region and on a planarization layer 450. The first electrode 460 may be an anode and may include a transparent conductive layer being formed of a conductive material having a relatively high work function, e.g., a transparent conductive oxide (TCO). The first electrode 460 may further include a reflection electrode or a reflection layer. For example, the reflection electrode or the reflection layer may be formed of silver (Ag) or aluminum-palladium-copper (APC) alloy. In the top-emission organic light emitting display device 400, the first electrode 460 may have a double-layered structure of Ag/ITO or APC/ITO or a triple-layered structure of ITO/Ag/ITO or ITO/APC/ITO.

A bank layer 466 covering an edge of the first electrode 460 is formed on the planarization layer 450. The bank layer 466 is positioned at a boundary of the red, green and blue pixel regions RP. GP and BP and exposes a center of the first electrode 460 in the red, green and blue pixel regions RP, GP and BP. Since the OLED D emits the white light in the red, green and blue pixel regions RP. GP and BP, the organic emitting layer 462 may be formed as a common layer in the red, green and blue pixel regions RP, GP and BP without separation in the red, green and blue pixel regions RP, GP and BP. The bank layer 466 may be formed to prevent the current leakage at an edge of the first electrode 460 and may be omitted.

An organic light emitting layer 462 is formed on the first electrode 460. As described below, the organic light emitting layer 462 includes at least two emitting parts, and each emitting part includes at least one EMLs. As a result, the OLED D provide white emission.

In this case, at least one of a plurality of EMLs includes the organic compound represented by Formula 1 and provide red emission.

A second electrode 464 is formed over the first substrate 410 where the organic emitting layer 462 is formed. In the organic light emitting display device 400, since the light emitted from the organic emitting layer 462 is incident to the color filter layer 480 through the second electrode 464, the second electrode 464 has a thin profile for transmitting the light.

The first electrode 460, the organic emitting layer 462 and the second electrode 464 constitute the OLED D.

The color filter layer 480 is positioned over the OLED D and includes a red color filter 482, a green color filter 484 and a blue color filter 486 respectively corresponding to the red, green and blue pixel regions RP, GP and BP. The red color filter 482 may include at least one a red dye and a red pigment, the green color filter 484 may include at least one of a green dye and a green pigment, and the blue color filter 486 may include at least one of a blue dye and a blue pigment.

An encapsulation layer (or an encapsulation film) may be formed to prevent penetration of moisture into the OLED D. For example, the encapsulation layer (or the encapsulation film) may include a first inorganic insulating layer, an organic insulating layer and a second inorganic insulating layer sequentially stacked, but it is not limited thereto.

Although not shown, the color filter layer 480 may be attached to the OLED D by using an adhesive layer. Alternatively, the color filter layer 480 may be formed directly on the OLED D or the encapsulation layer.

A polarization plate for reducing an ambient light reflection may be disposed over the top-emission type OLED D. For example, the polarization plate may be a circular polarization plate.

In the organic light emitting display device 400 of FIG. 5, the first electrode 460 and the second electrode 464 are a reflective electrode and a transparent (semitransparent) electrode, respectively, and the color filter layer 480 is disposed over the OLED D. Alternatively, the first electrode 460 and the second electrode 464 are a transparent (semitransparent) electrode and a reflective electrode, respectively, and the color filter layer 480 may be disposed between the OLED D and the first substrate 410.

A color conversion layer (not shown) may be formed between the OLED D and the color filter layer 480. The color conversion layer may include a red color conversion layer, a green color conversion layer and a blue color conversion layer respectively corresponding to the red, green and blue pixel regions RP, GP and BP. The white light from the OLED D is converted into the red light, the green light and the blue light by the red, green and blue color conversion layer, respectively. For example, the color conversion layer may include a quantum dot. The color purity of the organic light emitting display device 400 may be further improved due to the color conversion layer.

The color conversion layer may be included instead of the color filter layer 480.

As described above, in the organic light emitting display device 400, the OLED D in the red, green and blue pixel regions RP. GP and BP provides white emission, and the white light from the OLED D passes through the red color filter 482, the green color filter 484 and the blue color filter 486 in the red pixel region RP, the green pixel region GP and the blue pixel region BP such that the red light, the green light and the blue light are provided from the red pixel region RP, the green pixel region GP and the blue pixel region BP, respectively.

In FIG. 5, the OLED D emitting the w % bite light is used for a display device. Alternatively, the OLED D may be formed on an entire surface of a substrate without at least one of the driving element and the color filter layer to be used for a lightening device. The display device and the lightening device each including the OLED D of the present disclosure may be referred to as an organic light emitting device.

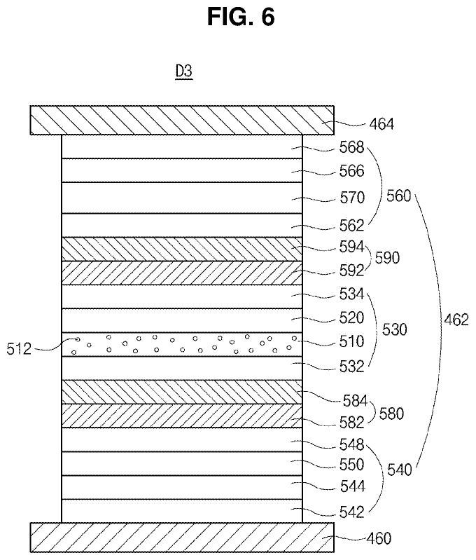

FIG. 6 is a schematic cross-sectional view illustrating an OLED according to a fifth embodiment of the present disclosure.

Referring to FIG. 6, the organic emitting layer 462 includes a first emitting part 530 including a red EML 510, a second emitting part 540 including a first blue EML 550 and a third emitting part 560 including a second blue EML 570. In addition, the organic emitting layer 462 may further include a first charge generation layer (CGL) 580 between the first and second emitting parts 530 and 540 and a second CGL 590 between the first and third emitting parts 530 and 560. Moreover, the first emitting part 530 may further include a green EML 520.

The organic light emitting display device 400 includes a red pixel region, a green pixel region and a blue pixel region. The OLED D3 is positioned in the red, green and blue pixel regions.

The first electrode 460 is an anode for injecting a hole, and the second electrode 464 is cathode for injecting an electron. In addition, one of the first and second electrodes 460 and 464 may be a reflection electrode, and the other one of the first and second electrodes 460 and 464 may be a transparent (or a semi-transparent) electrode.

For example, the first electrode 460 may include a transparent conductive material. e.g., ITO or IZO, and the second electrode 464 may be formed of Al, Mg, Ag, AlMg or MgAg.

The second emitting part 540 is positioned between the first electrode 460 and the first emitting part 530, and the third emitting part 560 is positioned between the first emitting part 530 and the second electrode 464. In addition, the second emitting part 540 is positioned between the first electrode 460 and the first CGL 580, and the third emitting part 560 is positioned between the second CGL 590 and the second electrode 464. Namely, the second emitting part 540, the first CGL 580, the first emitting part 530, the second CGL 590 and the third emitting part 560 are sequentially stacked on the first electrode 460.

In the first emitting part 530, the green EML 520 is positioned on the red EML 510.

The red EML 510 includes a first compound 512 being the organic compound of the present disclosure. Namely, the first compound 512 is represented by one of Formulas 1, 1-1 and 1-2 and is selected from the compounds in Formula 2.

The red EML 510 may further include a second compound being a red dopant, e.g., a red emitter. In this case, the first compound 512 serves as a host. The second compound may include at least one of a fluorescent compound, a delayed fluorescent compound and a phosphorescent compound. For example, the second compound may be represented by Formula 3 and may be one of the compounds in Formula 4.

When the red EML 510 includes the first compound 512 being the organic compound, which is represented by Formula 1 and is one of the compounds in Formula 2, and the second compound, which is represented by Formula 3 and is one of the compounds in Formula 4, an energy transfer efficiency from the first compound 512 to the second compound is improved. As a result, in the OLED D3 including the red EML 510 and the organic light emitting display device 400 including the OLED D3, the driving voltage is decreased, and the emitting efficiency and the lifespan are increased.

The red EML 510 may further include a third compound. In this case, the first compound 512 may serve as a p-type host, and the third compound may serve as an n-type host. For example, the third compound may be represented by Formula 5 and may be one of the compounds in Formula 6.

When the red EML 510 includes the first compound 512, which is the organic compound represented by Formula 1 and selected from the compounds in Formula 2, the second compound, which is represented by Formula 3 and selected from the compounds in Formula 4, and the third compound, which is represented by Formula 5 and selected from the compounds in Formula 6, an exciton generation efficiency between the first compound 512 and the third compound is increased, and the generated exciton is efficiently transferred into the second compound. Accordingly, in the OLED D3 including the red EML 510 and the organic light emitting display device 400 including the OLED D3, the driving voltage is further decreased, and the emitting efficiency and the lifespan are further increased.

The red EML 510 may have a thickness of 100 to 400 Å, preferably 200 to 400 Å, but it is not limited thereto.

When the red EML 510 includes the first compound 512 and the second compound, e.g., a dopant, a weight % of the first compound 512 is greater than that of the second compound. For example, in the red EML 510, the first compound 512 may have a weight % of 60 to 99.

When the red EML 510 includes the first compound 512, the second compound and the third compound, a weight % of each of the first compound 512 and the third compound may be greater than that of the second compound, and the weight % of the first compound 512 and the weight % of the third compound may be same or different. For example, the weight % of the first compound 512 and the weight % of the third compound may be same, and the second compound may have a weight % of 1 to 5 in the red EML 510.

The green EML 520 may include a green host and a green dopant. The green host may include a p-type host and an n-type host. The green EML 520 may have a thickness of 100 to 400 Å, preferably 200 to 400 Å.

The green host may be a green host material being one of mCP-CN, CBP, mCBP, mCP, DPEPO, 2,8-bis(diphenylphosphoryl)dibenzothiophene (PPT), TmPyPB, PYD-2Cz, 2,8-di(9H-carbazol-9-yl)dibenzothiophene (DCzDBT), 3′, 5′-di(carbazol-9-yl)-[1,1′-bipheyl]-3,5-dicarbonitrile (DCzTPA), 4′-(9H-carbazol-9-yl)biphenyl-3,5-dicarbonitrile (pCzB-2CN), 3′-(9H-carbazol-9-yl)biphenyl-3,5-dicarbonitrile (mCzB-2CN), TSPO1, and 9-(9-phenyl-9H-carbazol-6-yl)-9H-carbazole (CCP), but it is not limited thereto.

The green dopant may be a green dopant material being one of [bis(2-phenylpyridine)](pyridyl-2-benzofuro[2,3-b]pyridine)iridium), tris[2-phenylpyridine]iridium(III) (Ir(ppy)3), fac-tris(2-phenylpyridine)iridium(II) (fac-Ir(ppy)3), bis(2-phenylpyridine)(acetylacetonate)iridium(II) (Ir(ppy)2(acac)), tris[2-(p-tolyl)pyridine]iridium(III) (Ir(mppy)3), bis(2-(naphthalene-2-yl)pyrdine)(acetylacetonate)iridium(III) (Ir(npy)2acac), tris(2-phenyl-3-methyl-pyridine)iridium (Ir(3mppy)3), and fac-tris(2-(3-p-xylyl)phenyl)pyridine iridium(III) (TEG), but it is not limited thereto.

The first emitting part 530 may further include at least one of a first HTL 532 under the red EML 510 and a first ETL 534 over the red EML 510. When the first emitting part 530 includes the green EML 520, the first ETL 534 is disposed on the green EML 520.

The first emitting part 530 may further include at least one of a first EBL between the red EML 510 and the first HTL 532 and a first HBL between the green EML 520 and the first ETL 534.

The second emitting part 540 may further include at least one of a second HTL 544 under the first blue EML 550 and a second ETL 548 over the first blue EML 550. The second emitting part 540 may further include an HIL 542 between the first electrode 460 and the second HTL 544.

The second emitting part 540 may further include at least one of a second EBL between the first blue EML 550 and the second HTL 544 and a second HBL between the first blue EML 550 and the second ETL 548.

The third emitting part 560 may further include at least one of a third HTL 562 under the second blue EML 570 and a third ETL 566 over the second blue EML 570. The third emitting part 560 may further include an EIL 568 between the second electrode 464 and the third ETL 566.

The third emitting part 560 may further include at least one of a third EBL between the second blue EML 570 and the third HTL 562 and a third HBL between the second blue EML 570 and the third ETL 566.

The first blue EML 550 in the second emitting part 540 may include a first blue host and a first blue dopant, and the second blue EML 570 in the second emitting part 560 may include a second blue host and a second blue dopant.

For example, each of the first and second blue hosts may include a blue host material being one of mCP, mCP-CN, mCBP, CBP-CN, CBP, 9-(3-(9H-Carbazol-9-yl)phenyl)-3-(diphenylphosphoryl)-9H-carbazole (mCPPO1), 3,5-di(9H-carbazol-9-yl)biphenyl (Ph-mCP), TSPO1, 9-(3′-(9H-carbazol-9-yl)-[1,1′-biphenyl]-3-yl)-9H-pyrido[2,3-b]indole (CzBPCb), bis(2-methylphenyl)diphenylsilane (UGH-1), 1,4-bis(triphenylsilyl)benzene (UGH-2), 1,3-bis(triphenylsilyl)benzene (UGH-3), 9,9-spiorobifluoren-2-yl-diphenyl-phosphine oxide (SPPO1), and 9,9′-(5-(triphenylsilyl)-1,3-phenylene)bis(9H-carbazole) (SimCP), but it is not limited thereto.

Each of the first and second blue dopants may include a blue dopant material being one of perylene, 4,4′-bis[4-(di-p-tolylamino)styryl]biphenyl (DPAVBi), 4-(di-p-tolylamino)-4-4′-[(di-p-tolylamino)styryl]stilbene (DPAVB), 4,4′-bis[4-(diphenylamino)styryl]biphenyl (BDAVBi), 2,5,8,11-Tetra-tetr-butylperylene (TBPe), Bepp2, 9-(9-phenylcarbazole-3-yl)-10-(naphthalene-1-yl)anthracene (PCAN), mer-tris(1-phenyl-3-methylimidazolin-2-ylidene-C,C(2)′ iridium(III) (mer-Ir(pmi)₃), fac-tris(1,3-diphenyl-benzimidazolin-2-ylidene-C,C(2)′ iridium(III) (fac-Ir(dpbic)₃), bis(3,4,5-trifluoro-2-(2-pyridyl)phenyl-(2-carboxypyridyl)iridium(III) (Ir(tfpd)₂pic), tris(2-(4,6-difluorophenyl)pyridine))iridium(III) (Ir(Fppy)₃), and bis[2-(4,6-difluorophenyl)pyridinato-C²,N](picolinato)iridium(III); FIrpic), but it is not limited thereto.

In an aspect to the present disclosure, each of the first and second blue EMLs 550 and 570 may include an anthracene derivative as a blue host and a boron derivative as a blue dopant.

The HIL 542 may include the above-mention hole injection material and may have a thickness of 10 to 100 Å. Each of the first to third HTLs 532, 544 and 562 may include the above-mention hole transporting material and may have a thickness of 500 to 1500 Å, preferably 700 to 1200 Å.

Each of the first to third ETLs 534, 548 and 566 may include the above-mention electron transporting material and may have a thickness of 100 to 500 Å, preferably 200 to 400 Å. The EIL 568 may include the above-mention electron injection material and may have a thickness of 1 to 50 Å, preferably 5 to 30 Å.

Each of the first to third EBLs may include the above-mention electron blocking material and may have a thickness of 10 to 300 Å. Each of the first to third HBLs may include the above-mention hole blocking material and may have a thickness of 10 to 300 Å.

The first CGL 580 is positioned between the first emitting part 530 and the second emitting part 540, and the second CGL 590 is positioned between the first emitting part 530 and the third emitting part 560. Namely, the first and second emitting parts 530 and 540 are connected through the first CGL 580, and the first and third emitting parts 530 and 560 are connected through the second CGL 590.

The first CGL 580 may be a P-N junction CGL of an N-type CGL 582 and a P-type CGL 584, and the second CGL 590 may be a P-N junction CGL of an N-type CGL 592 and a P-type CGL 594.

In the first CGL 580, the N-type CGL 582 is positioned between the first HTL 532 and the second ETL 548, and the P-type CGL 584 is positioned between the N-type CGL 582 and the first HTL 532.

In the second CGL 590, the N-type CGL 592 is positioned between the first ETL 534 and the third HTL 562, and the P-type CGL 594 is positioned between the N-type CGL 592 and the third HTL 562.

Each of the N-type CGL 582 in the first CGL 580 and the N-type CGL 592 in the second CGL 590 may include the above-mentioned N-type charge generation material, and each of the P-type CGL 584 in the first CGL 580 and the P-type CGL 594 in the second CGL 590 may include the above-mentioned P-type charge generation material.

As described above, the OLED D3 includes the first emitting part 530, which includes the red and green EMLs 510 and 520, the second emitting part 540, which includes the first blue EML 550, and the third emitting part 560, which includes the second blue EML 570, so that the white emission is provided from the OLED D3.

In this case, the red EML 510 includes the organic compound represented by Formula 1. As a result, in the OLED D3, the driving voltage is decreased, and the emitting efficiency and the lifespan are increased.

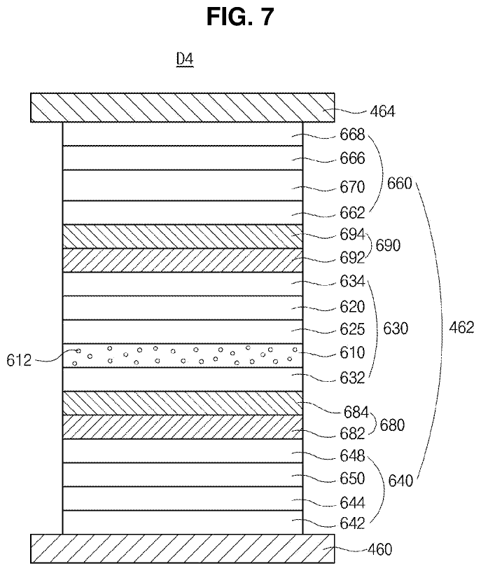

FIG. 7 is a schematic cross-sectional view illustrating an OLED according to a sixth embodiment.

Referring to FIG. 7, the organic emitting layer 462 includes a first emitting part 630 including a red EML 610, a green EML 620 and a yellow-green EML 625, a second emitting part 640 including a first blue EML 650 and a third emitting part 660 including a second blue EML 670. In addition, the organic emitting layer 462 may further include a first charge generation layer (CGL) 680 between the first and second emitting parts 630 and 640 and a second CGL 690 between the first and third emitting parts 630 and 660.

The organic light emitting display device 400 includes a red pixel region, a green pixel region and a blue pixel region. The OLED D4 is positioned in the red, green and blue pixel regions.

The first electrode 460 is an anode for injecting a hole, and the second electrode 464 is cathode for injecting an electron. In addition, one of the first and second electrodes 460 and 464 may be a reflection electrode, and the other one of the first and second electrodes 460 and 464 may be a transparent (or a semi-transparent) electrode.

For example, the first electrode 460 may include a transparent conductive material, e.g., ITO or IZO, and the second electrode 464 may be formed of Al, Mg, Ag, AlMg or MgAg.

The second emitting part 640 is positioned between the first electrode 460 and the first emitting part 630, and the third emitting part 660 is positioned between the first emitting part 630 and the second electrode 464. In addition, the second emitting part 640 is positioned between the first electrode 460 and the first CGL 680, and the third emitting part 660 is positioned between the second CGL 690 and the second electrode 464. Namely, the second emitting part 640, the first CGL 680, the first emitting part 630, the second CGL 690 and the third emitting part 660 are sequentially stacked on the first electrode 460.

In the first emitting part 630, the yellow-green EML 625 is positioned between the red EML 610 and the green EML 620. Namely, the red EML 610, the yellow-green EML 625 and the green EML 620 are sequentially stacked so that the EML in the first emitting part 630 has a triple-layered structure.

The red EML 610 includes a first compound 612 being the organic compound of the present disclosure. Namely, the first compound 612 is represented by one of Formulas 1, 1-1 and 1-2 and is selected from the compounds in Formula 2.

The red EML 610 may further include a second compound being a red dopant, e.g., a red emitter. In this case, the first compound 612 serves as a host. The second compound may include at least one of a fluorescent compound, a delayed fluorescent compound and a phosphorescent compound. For example, the second compound may be represented by Formula 3 and may be one of the compounds in Formula 4.

When the red EML 610 includes the first compound 612 being the organic compound, which is represented by Formula 1 and is one of the compounds in Formula 2, and the second compound, which is represented by Formula 3 and is one of the compounds in Formula 4, an energy transfer efficiency from the first compound 612 to the second compound is improved. As a result, in the OLED D4 including the red EML 610 and the organic light emitting display device 400 including the OLED D4, the driving voltage is decreased, and the emitting efficiency and the lifespan are increased.

The red EML 610 may further include a third compound. In this case, the first compound 612 may serve as a p-type host, and the third compound may serve as an n-type host. For example, the third compound may be represented by Formula 5 and may be one of the compounds in Formula 6.

When the red EML 610 includes the first compound 612, which is the organic compound represented by Formula 1 and selected from the compounds in Formula 2, the second compound, which is represented by Formula 3 and selected from the compounds in Formula 4, and the third compound, which is represented by Formula 5 and selected from the compounds in Formula 6, an exciton generation efficiency between the first compound 612 and the third compound is increased, and the generated exciton is efficiently transferred into the second compound. Accordingly, in the OLED D4 including the red EML 610 and the organic light emitting display device 400 including the OLED D4, the driving voltage is further decreased, and the emitting efficiency and the lifespan are further increased.

The red EML 610 may have a thickness of 100 to 400 Å, preferably 200 to 400 Å, but it is not limited thereto.

When the red EML 610 includes the first compound 612 and the second compound, e.g., a dopant, a weight % of the first compound 612 is greater than that of the second compound. For example, in the red EML 610, the first compound 612 may have a weight % of 60 to 99.

When the red EML 610 includes the first compound 612, the second compound and the third compound, a weight % of each of the first compound 612 and the third compound may be greater than that of the second compound, and the weight % of the first compound 612 and the weight % of the third compound may be same or different. For example, the weight % of the first compound 612 and the weight % of the third compound may be same, and the second compound may have a weight % of 1 to 5 in the red EML 610.

The green EML 620 may include a green host and a green dopant. The green host may include a p-type host and an n-type host. The green EML 620 may have a thickness of 100 to 400 Å, preferably 200 to 400 Å.

The green host may be the above-mentioned green host material, and he green dopant may be the above-mentioned green dopant material.

The yellow-green EML 625 may include a yellow-green host and a yellow-green dopant. The yellow-green host may include a p-type host and an n-type host. The yellow-green EML 625 may have a thickness of 100 to 400 Å, preferably 200 to 400 Å.

The yellow-green host may be same as the green host.

The yellow-green dopant may be a yellow-green dopant material being one of 5,6,11,12-tetraphenylnaphthalene (Rubrene), 2,8-di-tert-butyl-5,11-bis(4-tert-butylphenyl)-6,12-diphenyltetracene (TBRb), bis(2-phenylbenzothiazolato)(acetylacetonate)irdium(III) (Ir(BT)₂(acac)), bis(2-(9,9-diethytl-fluoren-2-yl)-1-phenyl-1H-benzo[d]imdiazolato)(acetylacetonate)iridium(III) (Ir(fbi)₂(acac)), bis(2-phenylpyridine)(3-(pyridine-2-yl)-2H-chromen-2-onate)iridium(III) (fac-Ir(ppy)₂Pc), bis(2-(2,4-difluorophenyl)quinoline)(picolinate)iridium(III) (FPQIrpic), and bis(4-phenylthieno[3,2-c]pyridinato-N,C2′) (acetylacetonate) iridium(III) (PO-01).

The first emitting part 630 may further include at least one of a first HTL 632 under the red EML 610 and a first ETL 634 over the green EML 620.

The first emitting part 630 may further include at least one of a first EBL between the red EML 610 and the first HTL 632 and a first HBL between the green EML 620 and the first ETL 634.

The second emitting part 640 may further include at least one of a second HTL 644 under the first blue EML 650 and a second ETL 648 over the first blue EML 650. The second emitting part 640 may further include an HIL 642 between the first electrode 460 and the second HTL 644.

The second emitting part 640 may further include at least one of a second EBL between the first blue EML 650 and the second HTL 644 and a second HBL between the first blue EML 650 and the second ETL 648.

The third emitting part 660 may further include at least one of a third HTL 662 under the second blue EML 670 and a third ETL 666 over the second blue EML 670. The third emitting part 660 may further include an EIL 668 between the second electrode 464 and the third ETL 666.

The third emitting part 660 may further include at least one of a third EBL between the second blue EML 670 and the third HTL 662 and a third HBL between the second blue EML 670 and the third ETL 666.

The first blue EML 650 in the second emitting part 640 may include a first blue host and a first blue dopant, and the second blue EML 670 in the second emitting part 660 may include a second blue host and a second blue dopant.

The HIL 642 may include the above-mention hole injection material and may have a thickness of 10 to 100 Å. Each of the first to third HTLs 632, 644 and 662 may include the above-mention hole transporting material and may have a thickness of 500 to 1500 Å, preferably 700 to 1200 Å.

Each of the first to third ETLs 634, 648 and 666 may include the above-mention electron transporting material and may have a thickness of 100 to 500 Å, preferably 200 to 400 Å. The EIL 668 may include the above-mention electron injection material and may have a thickness of 1 to 50 Å, preferably 5 to 30 Å.

Each of the first to third EBLs may include the above-mention electron blocking material and may have a thickness of 10 to 300 Å. Each of the first to third HBLs may include the above-mention hole blocking material and may have a thickness of 10 to 300 Å.

The first CGL 680 is positioned between the first emitting part 630 and the second emitting part 640, and the second CGL 690 is positioned between the first emitting part 630 and the third emitting part 660. Namely, the first and second emitting parts 630 and 640 are connected through the first CGL 680, and the first and third emitting parts 630 and 660 are connected through the second CGL 690.

The first CGL 680 may be a P-N junction CGL of an N-type CGL 682 and a P-type CGL 684, and the second CGL 690 may be a P-N junction CGL of an N-type CGL 692 and a P-type CGL 694.

In the first CGL 680, the N-type CGL 682 is positioned between the first HTL 632 and the second ETL 648, and the P-type CGL 684 is positioned between the N-type CGL 682 and the first HTL 632.

In the second CGL 690, the N-type CGL 692 is positioned between the first ETL 634 and the third HTL 662, and the P-type CGL 694 is positioned between the N-type CGL 692 and the third HTL 662.

Each of the N-type CGL 682 in the first CGL 680 and the N-type CGL 692 in the second CGL 690 may include the above-mentioned N-type charge generation material, and each of the P-type CGL 684 in the first CGL 680 and the P-type CGL 694 in the second CGL 690 may include the above-mentioned P-type charge generation material.

As described above, the OLED D4 includes the first emitting part 630, which includes the red, yellow-green and green EMLs 610, 625 and 620, the second emitting part 640, which includes the first blue EML 650, and the third emitting part 660, which includes the second blue EML 670, so that the white emission is provided from the OLED D4.

In this case, the red EML 610 includes the organic compound represented by Formula 1. As a result, in the OLED D4, the driving voltage is decreased, and the emitting efficiency and the lifespan are increased.

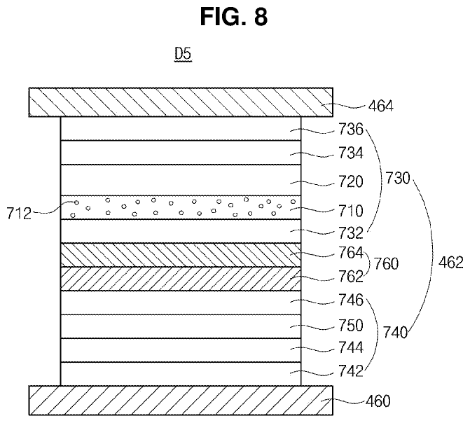

FIG. 8 is a schematic cross-sectional view illustrating an OLED according to a seventh embodiment.

Referring to FIG. 8, the organic emitting layer 462 includes a first emitting part 730 including a red EML 710 and a green EML 720 and a second emitting part 740 including a blue EML 750. In addition, the organic emitting layer 462 may further include a charge generation layer (CGL) 760 between the first and second emitting parts 730 and 740.

The organic light emitting display device 400 includes a red pixel region, a green pixel region and a blue pixel region. The OLED D5 is positioned in the red, green and blue pixel regions.

The first electrode 460 is an anode for injecting a hole, and the second electrode 464 is cathode for injecting an electron. In addition, one of the first and second electrodes 460 and 464 may be a reflection electrode, and the other one of the first and second electrodes 460 and 464 may be a transparent (or a semi-transparent) electrode.

For example, the first electrode 460 may include a transparent conductive material. e.g., ITO or IZO, and the second electrode 464 may be formed of Al, Mg, Ag, AlMg or MgAg.

The first emitting part 730 is positioned between the CGL 760 and the second electrode 464, and the second emitting part 740 is positioned between the first electrode 460 and the CGL 760. Alternatively, the first emitting part 730 may be positioned between the first electrode 460 and the CGL 760, and the second emitting part 740 may be positioned between the CGL 760 and the second electrode 464.

In the first emitting part 730, the green EML 720 is positioned on the red EML 710.

The red EML 710 includes a first compound 712 being the organic compound of the present disclosure. Namely, the first compound 712 is represented by one of Formulas 1, 1-1 and 1-2 and is selected from the compounds in Formula 2.

The red EML 710 may further include a second compound being a red dopant, e.g., a red emitter. In this case, the first compound 712 serves as a host. The second compound may include at least one of a fluorescent compound, a delayed fluorescent compound and a phosphorescent compound. For example, the second compound may be represented by Formula 3 and may be one of the compounds in Formula 4.

When the red EML 710 includes the first compound 712 being the organic compound, which is represented by Formula 1 and is one of the compounds in Formula 2, and the second compound, which is represented by Formula 3 and is one of the compounds in Formula 4, an energy transfer efficiency from the first compound 712 to the second compound is improved. As a result, in the OLED D5 including the red EML 710 and the organic light emitting display device 400 including the OLED D5, the driving voltage is decreased, and the emitting efficiency and the lifespan are increased.

The red EML 710 may further include a third compound. In this case, the first compound 712 may serve as a p-type host, and the third compound may serve as an n-type host. For example, the third compound may be represented by Formula 5 and may be one of the compounds in Formula 6.

When the red EML 710 includes the first compound 712, which is the organic compound represented by Formula 1 and selected from the compounds in Formula 2, the second compound, which is represented by Formula 3 and selected from the compounds in Formula 4, and the third compound, which is represented by Formula 5 and selected from the compounds in Formula 6, an exciton generation efficiency between the first compound 712 and the third compound is increased, and the generated exciton is efficiently transferred into the second compound. Accordingly, in the OLED D5 including the red EML 710 and the organic light emitting display device 400 including the OLED D5, the driving voltage is further decreased, and the emitting efficiency and the lifespan are further increased.

The red EML 710 may have a thickness of 100 to 400 Å, preferably 200 to 400 Å, but it is not limited thereto.

When the red EML 710 includes the first compound 712 and the second compound, e.g., a dopant, a weight % of the first compound 712 is greater than that of the second compound. For example, in the red EML 710, the first compound 712 may have a weight % of 60 to 99.

When the red EML 710 includes the first compound 712, the second compound and the third compound, a weight % of each of the first compound 712 and the third compound may be greater than that of the second compound, and the weight % of the first compound 712 and the weight % of the third compound may be same or different. For example, the weight % of the first compound 712 and the weight % of the third compound may be same, and the second compound may have a weight % of 1 to 5 in the red EML 710.

The green EML 720 may include a green host and a green dopant. The green host may include a p-type host and an n-type host. The green EML 720 may have a thickness of 100 to 400 Å, preferably 200 to 400 Å.

The green host may be the above-mentioned green host material, and the green dopant may be the above-mentioned green dopant material.

The first emitting part 730 may further include at least one of a first HTL 732 under the red EML 710 and a first ETL 734 over the red EML 710. When the first emitting part 730 includes the green EML 720, the first ETL 734 is disposed on the green EML 720. The first emitting part 730 may further include an EIL 736 between the first ETL 734 and the second electrode 464.

The first emitting part 730 may further include at least one of a first EBL between the red EML 710 and the first HTL 732 and a first HBL between the green EML 720 and the first ETL 734.

The second emitting part 740 may further include at least one of a second HTL 744 under the blue EML 750 and a second ETL 746 over the blue EML 750. The second emitting part 740 may further include an HIL 742 between the first electrode 460 and the second HTL 744.

The second emitting part 740 may further include at least one of a second EBL between the blue EML 750 and the second HTL 744 and a second HBL between the blue EML 750 and the second ETL 746.

The blue EML 750 in the second emitting part 740 may include a blue host and a blue dopant.

The HIL 742 may include the above-mention hole injection material and may have a thickness of 10 to 100 Å. Each of the first and second HTLs 732 and 744 may include the above-mention hole transporting material and may have a thickness of 500 to 1500 Å, preferably 700 to 1200 Å.

Each of the first and second ETLs 734 and 746 may include the above-mention electron transporting material and may have a thickness of 100 to 500 Å, preferably 200 to 400 Å. The EIL 736 may include the above-mention electron injection material and may have a thickness of 1 to 50 Å, preferably 5 to 30 Å.

Each of the first and second EBLs may include the above-mention electron blocking material and may have a thickness of 10 to 300 Å. Each of the first and second HBLs may include the above-mention hole blocking material and may have a thickness of 10 to 300 Å.

The CGL 760 is positioned between the first emitting part 730 and the second emitting part 740. Namely, the first and second emitting parts 730 and 740 are connected through the CGL 760. The CGL 760 may be a P-N junction CGL of an N-type CGL 762 and a P-type CGL 764.

In the CGL 760, the N-type CGL 762 is positioned between the first HTL 732 and the second ETL 746, and the P-type CGL 764 is positioned between the N-type CGL 762 and the first HTL 732.

The N-type CGL 762 may include the above-mentioned N-type charge generation material, and the P-type CGL 764 may include the above-mentioned P-type charge generation material.

As described above, the OLED D5 includes the first emitting part 730, which includes the red, and green EMLs 710 and 720, and the second emitting part 740, which includes the blue EML 750, so that the white emission is provided from the OLED D5.

In this case, the red EML 710 includes the organic compound represented by Formula 1. As a result, in the OLED D5, the driving voltage is decreased, and the emitting efficiency and the lifespan are increased.

### [OLED]

An anode (ITO), an HIL (the compound in Formula 7, 50 Å), an HTL (the compound in Formula 8, 1000 Å), an EML (a p-type host, an n-type host and a dopant (3 wt %), 250 Å), an ETL (the compound in Formula 9, 300 Å), an EIL (LiF, 20 Å) and a cathode (Al, 1000 Å) was sequentially deposited to form an OLED. The p-type host and the n-type host were included with the same weight %.

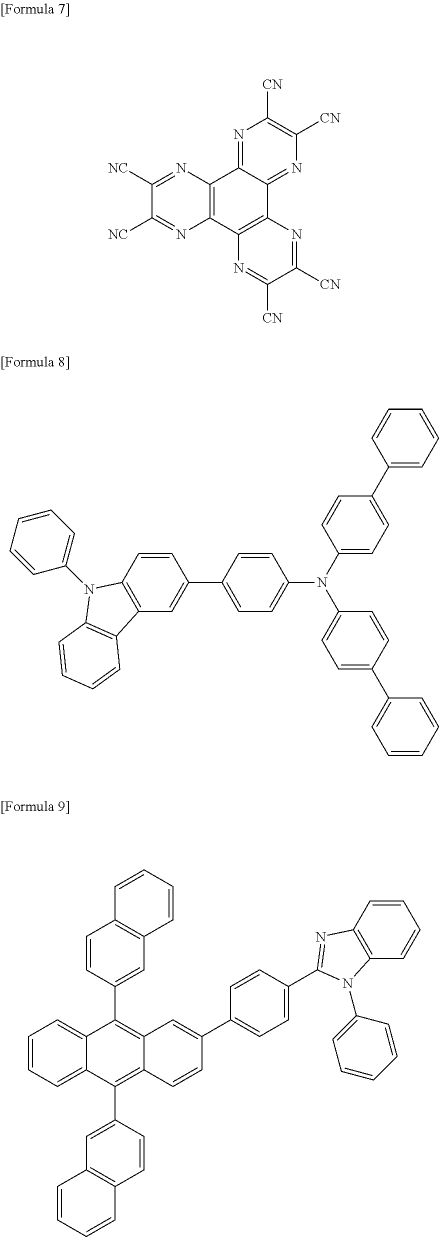 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

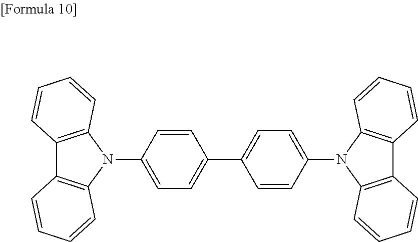 [SMILES: c1ccc2c(c1)c1ccccc1n2-c1ccc(-c2ccc(-n3c4ccccc4c4ccccc43)cc2)cc1]

### 1. Comparative Example (Ref)

The compound in Formula 10, the compound NH1 in Formula 6 and the compound RD1 in Formula 4 were used as the p-type host, the n-type host and the dopant, respectively. ((p-type host):(n-type host)=1:1 (wt %))

### 2. Examples

### (1) Example 1 (Ex1)

The compound BIZ21 in Formula 2, the compound NH1 in Formula 6 and the compound RD1 in Formula 4 were used as the p-type host, the n-type host and the dopant, respectively. ((p-type host):(n-type host)=1:1 (wt %))

### (2) Example 2 (Ex2)

The compound BIZ26 in Formula 2, the compound NH in Formula 6 and the compound RD1 in Formula 4 were used as the p-type host, the n-type host and the dopant, respectively. ((p-type host):(n-type host)=1:1 (wt %))

### (3) Example 3 (Ex3)

The compound BIZ28 in Formula 2, the compound NH in Formula 6 and the compound RD1 in Formula 4 were used as the p-type host, the n-type host and the dopant, respectively. ((p-type host):(n-type host)=1:1 (wt %))

### (4) Example 4 (Ex4)

The compound BIZ29 in Formula 2, the compound NH1 in Formula 6 and the compound RD1 in Formula 4 were used as the p-type host, the n-type host and the dopant, respectively. ((p-type host):(n-type host)=1:1 (wt %))

### (5) Example 5 (Ex5)

The compound BIZ36 in Formula 2, the compound NH1 in Formula 6 and the compound RD1 in Formula 4 were used as the p-type host, the n-type host and the dopant, respectively. ((p-type host):(n-type host)=1:1 (wt %))

### (6) Example 6 (Ex6)

The compound BIZ57 in Formula 2, the compound NH1 in Formula 6 and the compound RD1 in Formula 4 were used as the p-type host, the n-type host and the dopant, respectively. ((p-type host):(n-type host)=1:1 (wt %))

### (7) Example 7 (Ex7)

The compound BIZ62 in Formula 2, the compound NH1 in Formula 6 and the compound RD1 in Formula 4 were used as the p-type host, the n-type host and the dopant, respectively. ((p-type host):(n-type host)=1:1 (wt %))

### (8) Example 8 (Ex8)

The compound BIZ64 in Formula 2, the compound NH1 in Formula 6 and the compound RD1 in Formula 4 were used as the p-type host, the n-type host and the dopant, respectively. ((p-type host):(n-type host)=1:1 (wt %))

### (9) Example 9 (Ex9)

The compound BIZ65 in Formula 2, the compound NH in Formula 6 and the compound RD1 in Formula 4 were used as the p-type host, the n-type host and the dopant, respectively. ((p-type host):(n-type host)=1:1 (wt %))

### (10) Example 10 (Ex10)

The compound BIZ72 in Formula 2, the compound NH1 in Formula 6 and the compound RD1 in Formula 4 were used as the p-type host, the n-type host and the dopant, respectively. ((p-type host):(n-type host)=1:1 (wt %))

### (11) Example 11 (Ex11)

The compound BIZ93 in Formula 2, the compound NH1 in Formula 6 and the compound RD1 in Formula 4 were used as the p-type host, the n-type host and the dopant, respectively. ((p-type host):(n-type host)=1:1 (wt %))

### (12) Example 12 (Ex12)

The compound BIZ98 in Formula 2, the compound NH1 in Formula 6 and the compound RD1 in Formula 4 were used as the p-type host, the n-type host and the dopant, respectively. ((p-type host):(n-type host)=1:1 (wt %))

### (13) Example 13 (Ex13)

The compound BIZ100 in Formula 2, the compound NH1 in Formula 6 and the compound RD1 in Formula 4 were used as the p-type host, the n-type host and the dopant, respectively. ((p-type host):(n-type host)=1:1 (wt %))

### (14) Example 14 (Ex14)

The compound BIZ101 in Formula 2, the compound NH1 in Formula 6 and the compound RD1 in Formula 4 were used as the p-type host, the n-type host and the dopant, respectively. ((p-type host):(n-type host)=1:1 (wt %))

### (15) Example 15 (Ex15)

The compound BIZ108 in Formula 2, the compound NH1 in Formula 6 and the compound RD1 in Formula 4 were used as the p-type host, the n-type host and the dopant, respectively. ((p-type host):(n-type host)=1:1 (wt %))

### (16) Example 16 (Ex16)

The compound BIZ129 in Formula 2, the compound NH1 in Formula 6 and the compound RD1 in Formula 4 were used as the p-type host, the n-type host and the dopant, respectively. ((p-type host):(n-type host)=1:1 (wt %))

### (17) Example 17 (Ex17)

The compound BIZ134 in Formula 2, the compound NH1 in Formula 6 and the compound RD1 in Formula 4 were used as the p-type host, the n-type host and the dopant, respectively. ((p-type host):(n-type host)=1:1 (wt %))

### (18) Example 18 (Ex18)

The compound BIZ136 in Formula 2, the compound NH1 in Formula 6 and the compound RD1 in Formula 4 were used as the p-type host, the n-type host and the dopant, respectively. ((p-type host):(n-type host)=1:1 (wt %))

### (19) Example 19 (Ex19)

The compound BZ137 in Formula 2 the compound NH1 in Formula 6 and the compound RD1 in Formula 4 were used as the p-type host, the n-type host and the dopant, respectively. ((p-type host):(n-type host)=1:1 (%% t %))

### (20) Example 20 (Ex21)

The compound BIZ144 in Formula 2, the compound NH1 in Formula 6 and the compound RD1 in Formula 4 were used as the p-type host, the n-type host and the dopant, respectively. ((p-type host):(n-type host)=1:1 (1%%))

The emitting properties, i.e., a driving voltage (ΔV), an emitting efficiency and a lifespan, of the OLED in Comparative Example and Examples 1 to 20 were measured and listed in Table 1. The emitting properties of the OLED were measured at the room temperature using a current source (KEITHLEY) and a photometer (PR 650).

TABLE 1

| | p-host | ΔV | efficiency | lifespan |
|---|---|---|---|---|
| Ref | Formula 10 | 0.00 | 100% | 100% |
| Ex1 | BIZ21 | −0.13 | 114% | 105% |
| Ex2 | BIZ26 | −0.16 | 117% | 105% |
| Ex3 | BIZ28 | −0.13 | 114% | 105% |
| Ex4 | BIZ29 | −0.11 | 112% | 106% |
| Ex5 | BIZ36 | 0.00 | 101% | 106% |
| Ex6 | BIZ57 | −0.07 | 107% | 106% |
| Ex7 | BIZ62 | −0.10 | 111% | 106% |
| Ex8 | BIZ64 | −0.07 | 108% | 106% |
| Ex9 | BIZ65 | −0.05 | 106% | 106% |
| Ex10 | BIZ72 | −0.09 | 110% | 105% |
| Ex11 | BIZ93 | −0.22 | 123% | 104% |
| Ex12 | BIZ98 | −0.18 | 118% | 105% |
| Ex13 | BIZ100 | −0.14 | 115% | 105% |
| Ex14 | BIZ101 | −0.13 | 114% | 105% |
| Ex15 | BIZ108 | −0.23 | 123% | 104% |
| Ex16 | BIZ129 | −0.13 | 114% | 105% |
| Ex17 | BIZ134 | −0.09 | 109% | 106% |
| Ex18 | BIZ136 | −0.05 | 106% | 106% |
| Ex19 | BIZ137 | −0.04 | 105% | 106% |
| Ex20 | BIZ144 | −0.11 | 111% | 105% |

As shown in Table 1, in comparison to the OLED of Comparative Example, in the OLED of Examples 1 to 20, which uses the organic compound of the present disclosure, the driving voltage is decreased, and the emitting efficiency and the lifespan are increased.

For example, as the compounds BIZ21, BIZ26, BIZ28, BIZ29, BIZ36, BIZ57, BIZ62, BIZ64, BIZ65, BIZ72, BIZ93, BIZ98, BIZ101, BIZ108, BIZ129, BIZ134, BIZ136, BIZ137 and BIZ144, when in Formula 1, R₃ is phenyl and at least one of Ar₁ and Ar₂ or at least one of Ar₃ and Ar₄ is a C6 to C30 aryl group substituted with a C1 to C10 alkyl group, e.g., dimethylfluorenyl, the OLED has a significant advantage in at least one of the driving voltage, the emitting efficiency and the lifespan.

It will be apparent to those skilled in the art that various modifications and variations can be made in the embodiments of the present disclosure without departing from the spirit or scope of the present disclosure. Thus, it is intended that the modifications and variations cover this disclosure provided they come within the scope of the appended claims and their equivalents.

What is claimed is:

1. An organic compound represented by Formula 1: 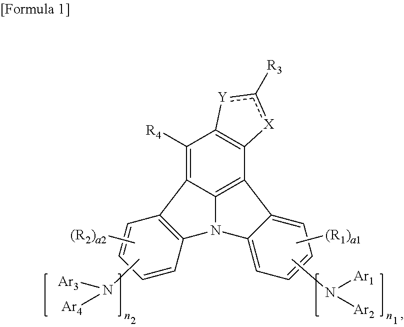
wherein one of X and Y is nitrogen (N), and the other one of X and Y is oxygen (O) or sulfur (S),
each of R₁ and R₂ is independently selected from the group consisting of hydrogen, deuterium, a substituted or unsubstituted C1 to C10 alkyl group and a substituted or unsubstituted C6 to C30 aryl group,
each of a1 and a2 is independently an integer of 0 to 4,
each of R₃ and R₄ is independently selected from the group consisting of hydrogen, deuterium, a substituted or unsubstituted C1 to C10 alkyl group and a substituted or unsubstituted C6 to C30 aryl group,
each of Ar₁ to Ar₄ is independently selected from the group consisting of a substituted or unsubstituted C6 to C30 aryl group, a substituted or unsubstituted C3 to C30 cycloalkyl group, a substituted or unsubstituted C6 to C30 aryl oxy group, a substituted or unsubstituted C6 to C30 arylthioxy group and a substituted or unsubstituted C5 to C30 heteroaryl group,
each of n1 and n2 is independently 0 or 1, n1 and n2 are not 0 simultaneously,
the sum of a1 and n1 is less than or equal to 4, and
the sum of a2 and n2 is less than or equal to 4.

2. The organic compound according to claim 1, wherein the Formula 1 is represented by Formula 1-1 or Formula 1-2: 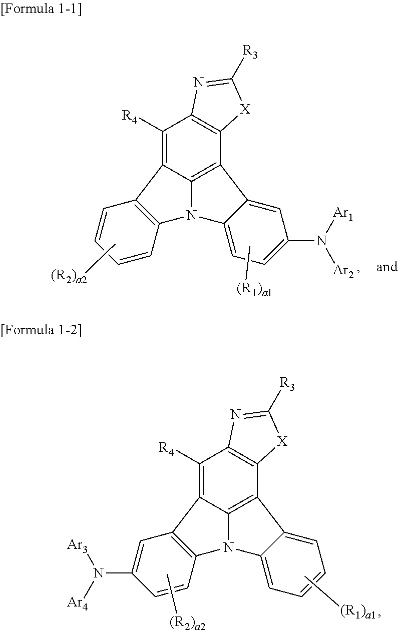
wherein in the Formula 1-1, the definitions of X, R₁ to R₄, a1, a2, Ar₁ and Ar₂ are same as those in Formula 1, and
wherein in the Formula 1-2, the definitions of X, R₁ to R₄, a1, a2, Ar₃ and Ar₄ are same as those in Formula 1.

3. The organic compound according to claim 1, wherein the organic compound is one of compounds in Formula 2:

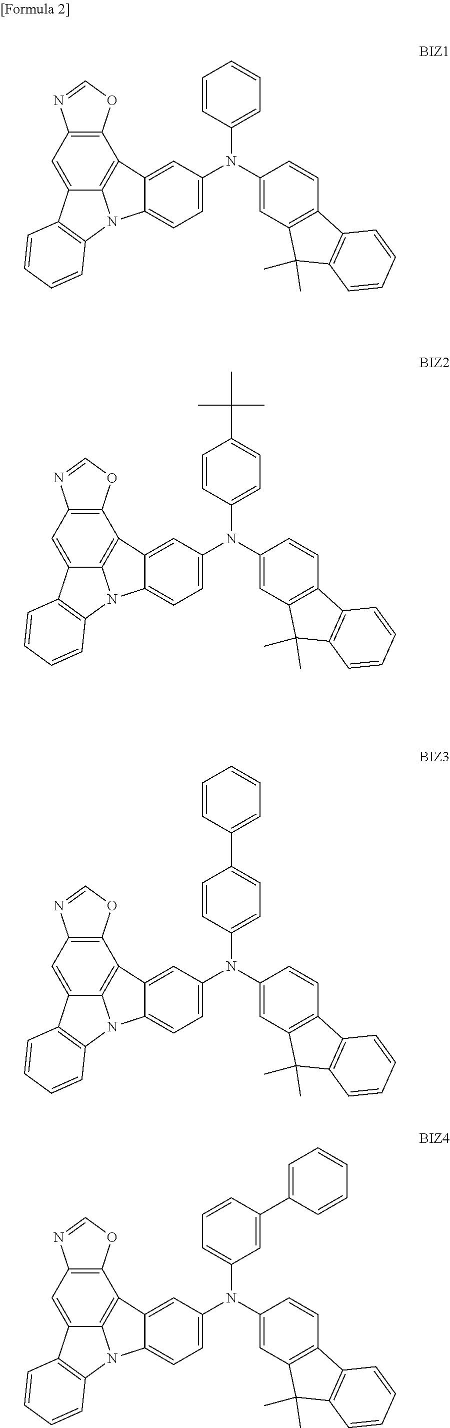 [SMILES: CC1(C)c2ccccc2-c2ccc(N(c3ccccc3)c3ccc4c(c3)c3c5ocnc5cc5c6ccccc6n4c53)cc21 ; CC(C)(C)c1ccc(N(c2ccc3c(c2)C(C)(C)c2ccccc2-3)c2ccc3c(c2)c2c4ocnc4cc4c5ccccc5n3c42)cc1 ; CC1(C)c2ccccc2-c2ccc(N(c3ccc(-c4ccccc4)cc3)c3ccc4c(c3)c3c5ocnc5cc5c6ccccc6n4c53)cc21 ; CC1(C)c2ccccc2-c2ccc(N(c3cccc(-c4ccccc4)c3)c3ccc4c(c3)c3c5ocnc5cc5c6ccccc6n4c53)cc21]

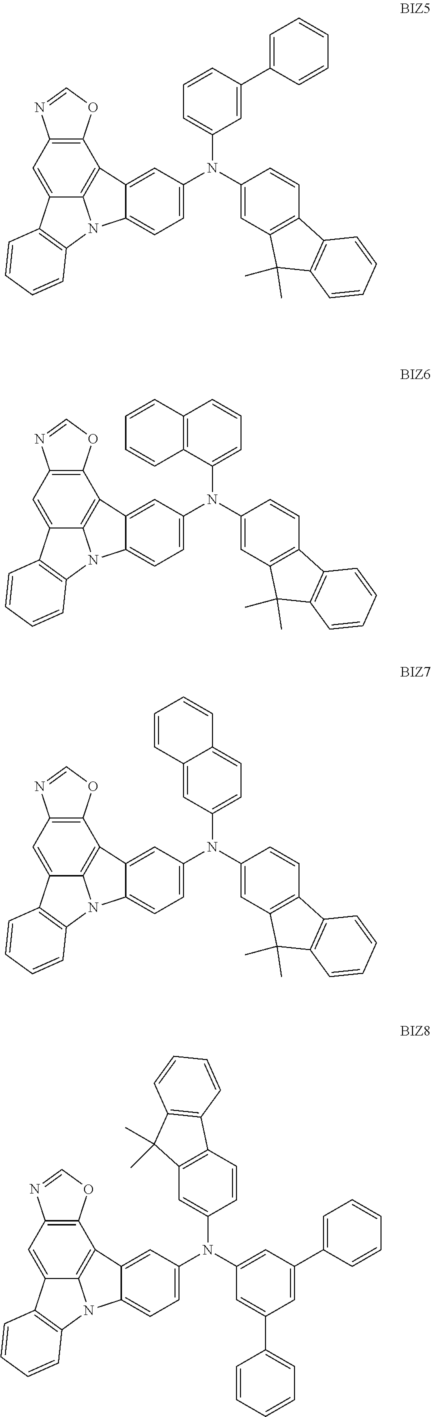 [SMILES: CC1(C)c2ccccc2-c2ccc(N(c3cccc(-c4ccccc4)c3)c3ccc4c(c3)c3c5ocnc5cc5c6ccccc6n4c53)cc21 ; CC1(C)c2ccccc2-c2ccc(N(c3ccc4c(c3)c3c5ocnc5cc5c6ccccc6n4c53)c3cccc4ccccc34)cc21 ; CC1(C)c2ccccc2-c2ccc(N(c3ccc4ccccc4c3)c3ccc4c(c3)c3c5ocnc5cc5c6ccccc6n4c53)cc21 ; CC1(C)c2ccccc2-c2ccc(N(c3cc(-c4ccccc4)cc(-c4ccccc4)c3)c3ccc4c(c3)c3c5ocnc5cc5c6ccccc6n4c53)cc21]

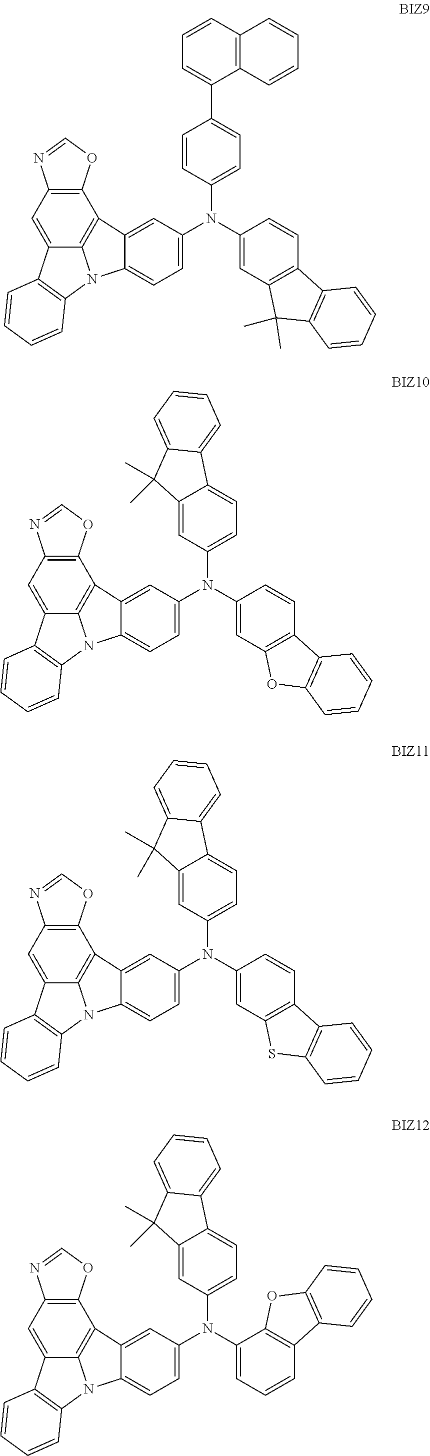 [SMILES: CC1(C)c2ccccc2-c2ccc(N(c3ccc(-c4cccc5ccccc45)cc3)c3ccc4c(c3)c3c5ocnc5cc5c6ccccc6n4c53)cc21 ; CC1(C)c2ccccc2-c2ccc(N(c3ccc4c(c3)oc3ccccc34)c3ccc4c(c3)c3c5ocnc5cc5c6ccccc6n4c53)cc21 ; CC1(C)c2ccccc2-c2ccc(N(c3ccc4c(c3)sc3ccccc34)c3ccc4c(c3)c3c5ocnc5cc5c6ccccc6n4c53)cc21 ; CC1(C)c2ccccc2-c2ccc(N(c3ccc4c(c3)c3c5ocnc5cc5c6ccccc6n4c53)c3cccc4c3oc3ccccc34)cc21]

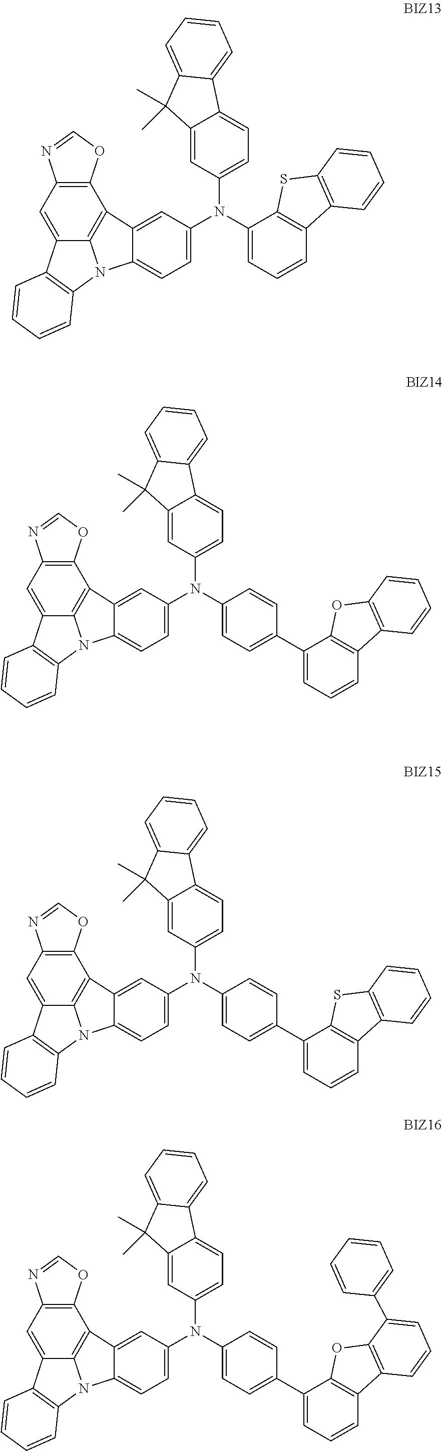 [SMILES: CC1(C)c2ccccc2-c2ccc(N(c3ccc4c(c3)c3c5ocnc5cc5c6ccccc6n4c53)c3cccc4c3sc3ccccc34)cc21 ; CC1(C)c2ccccc2-c2ccc(N(c3ccc(-c4cccc5c4oc4ccccc45)cc3)c3ccc4c(c3)c3c5ocnc5cc5c6ccccc6n4c53)cc21 ; CC1(C)c2ccccc2-c2ccc(N(c3ccc(-c4cccc5c4sc4ccccc45)cc3)c3ccc4c(c3)c3c5ocnc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5ocnc5cc5c6ccccc6n4c53)cc21]

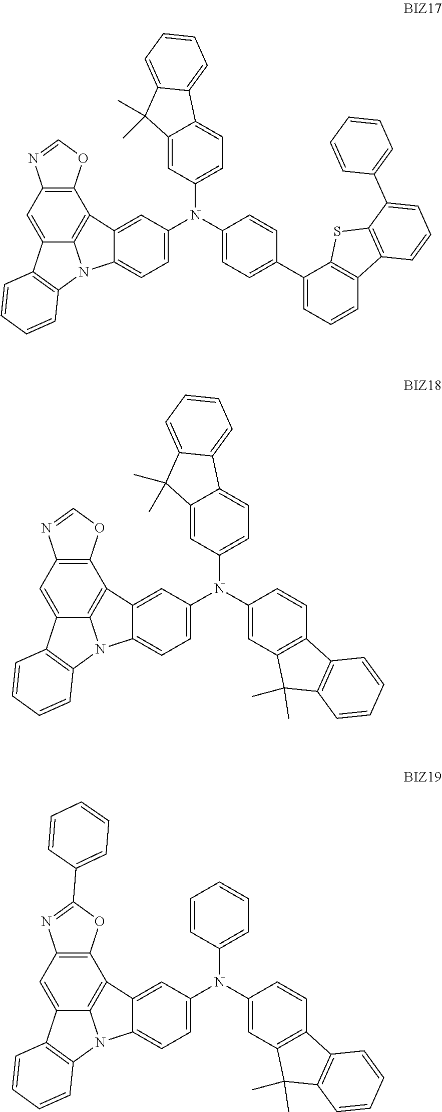 [SMILES: CC1(C)c2ccccc2-c2ccc(N(c3ccc(-c4cccc5c4sc4c(-c6ccccc6)cccc45)cc3)c3ccc4c(c3)c3c5ocnc5cc5c6ccccc6n4c53)cc21 ; CC1(C)c2ccccc2-c2ccc(N(c3ccc4c(c3)C(C)(C)c3ccccc3-4)c3ccc4c(c3)c3c5ocnc5cc5c6ccccc6n4c53)cc21 ; CC1(C)c2ccccc2-c2ccc(N(c3ccccc3)c3ccc4c(c3)c3c5oc(-c6ccccc6)nc5cc5c6ccccc6n4c53)cc21]

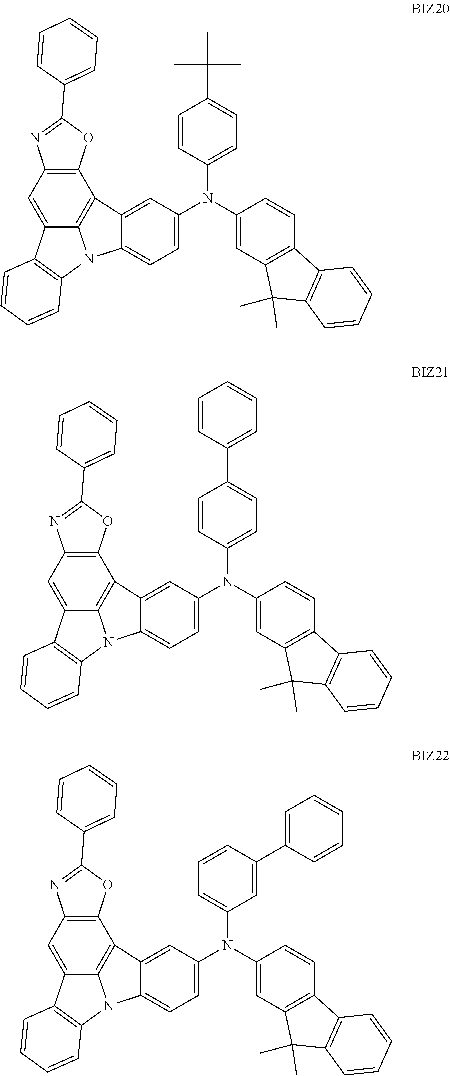 [SMILES: CC(C)(C)c1ccc(N(c2ccc3c(c2)C(C)(C)c2ccccc2-3)c2ccc3c(c2)c2c4oc(-c5ccccc5)nc4cc4c5ccccc5n3c42)cc1 ; CC1(C)c2ccccc2-c2ccc(N(c3ccc(-c4ccccc4)cc3)c3ccc4c(c3)c3c5oc(-c6ccccc6)nc5cc5c6ccccc6n4c53)cc21 ; CC1(C)c2ccccc2-c2ccc(N(c3cccc(-c4ccccc4)c3)c3ccc4c(c3)c3c5oc(-c6ccccc6)nc5cc5c6ccccc6n4c53)cc21]

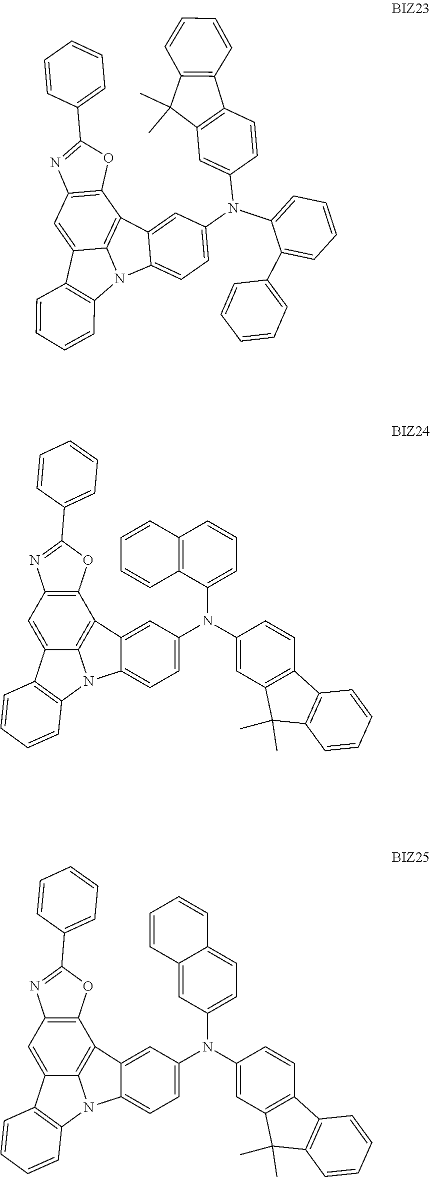 [SMILES: CC1(C)c2ccccc2-c2ccc(N(c3ccc4c(c3)c3c5oc(-c6ccccc6)nc5cc5c6ccccc6n4c53)c3ccccc3-c3ccccc3)cc21 ; CC1(C)c2ccccc2-c2ccc(N(c3ccc4c(c3)c3c5oc(-c6ccccc6)nc5cc5c6ccccc6n4c53)c3cccc4ccccc34)cc21 ; CC1(C)c2ccccc2-c2ccc(N(c3ccc4ccccc4c3)c3ccc4c(c3)c3c5oc(-c6ccccc6)nc5cc5c6ccccc6n4c53)cc21]

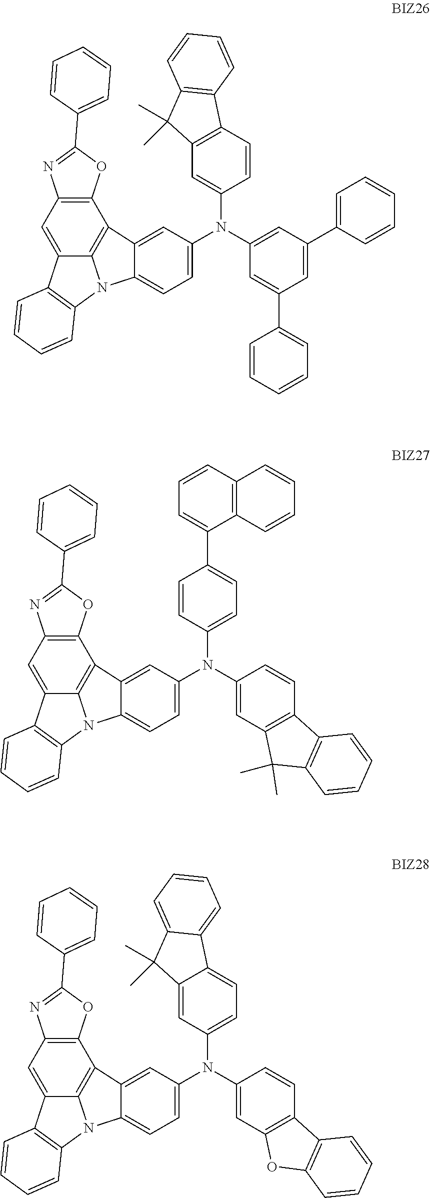 [SMILES: CC1(C)c2ccccc2-c2ccc(N(c3cc(-c4ccccc4)cc(-c4ccccc4)c3)c3ccc4c(c3)c3c5oc(-c6ccccc6)nc5cc5c6ccccc6n4c53)cc21 ; CC1(C)c2ccccc2-c2ccc(N(c3ccc(-c4cccc5ccccc45)cc3)c3ccc4c(c3)c3c5oc(-c6ccccc6)nc5cc5c6ccccc6n4c53)cc21 ; CC1(C)c2ccccc2-c2ccc(N(c3ccc4c(c3)oc3ccccc34)c3ccc4c(c3)c3c5oc(-c6ccccc6)nc5cc5c6ccccc6n4c53)cc21]

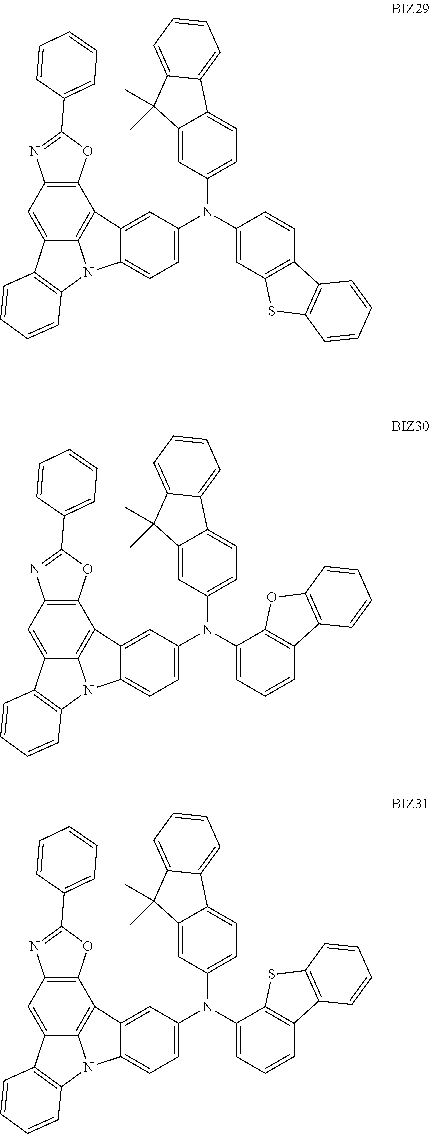 [SMILES: CC1(C)c2ccccc2-c2ccc(N(c3ccc4c(c3)sc3ccccc34)c3ccc4c(c3)c3c5oc(-c6ccccc6)nc5cc5c6ccccc6n4c53)cc21 ; CC1(C)c2ccccc2-c2ccc(N(c3ccc4c(c3)c3c5oc(-c6ccccc6)nc5cc5c6ccccc6n4c53)c3cccc4c3oc3ccccc34)cc21 ; CC1(C)c2ccccc2-c2ccc(N(c3ccc4c(c3)c3c5oc(-c6ccccc6)nc5cc5c6ccccc6n4c53)c3cccc4c3sc3ccccc34)cc21]

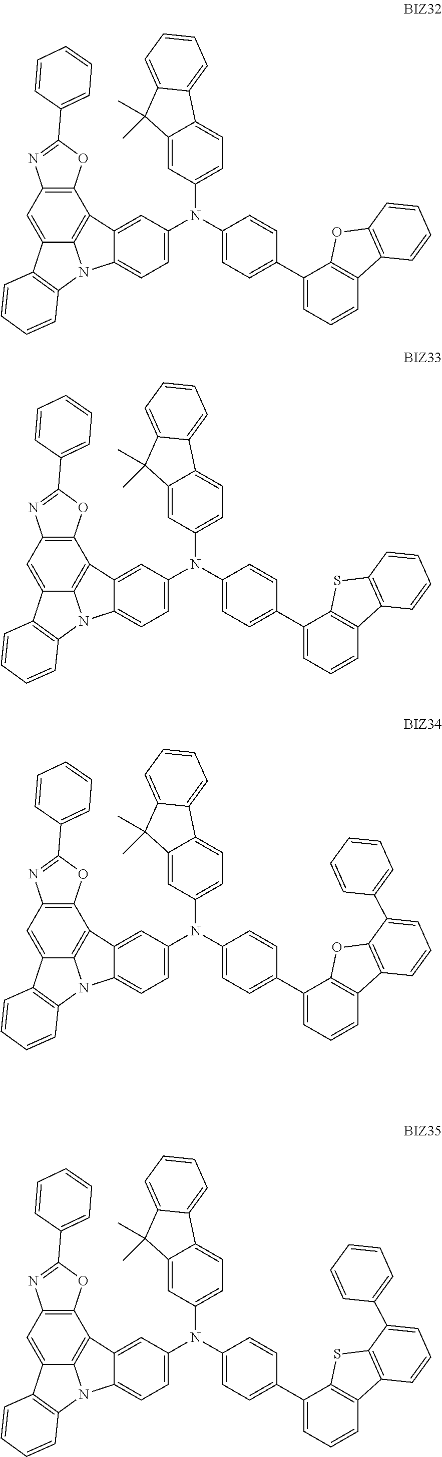 [SMILES: CC1(C)c2ccccc2-c2ccc(N(c3ccc(-c4cccc5c4oc4ccccc45)cc3)c3ccc4c(c3)c3c5oc(-c6ccccc6)nc5cc5c6ccccc6n4c53)cc21 ; CC1(C)c2ccccc2-c2ccc(N(c3ccc(-c4cccc5c4sc4ccccc45)cc3)c3ccc4c(c3)c3c5oc(-c6ccccc6)nc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5oc(-c6ccccc6)nc5cc5c6ccccc6n4c53)cc21 ; CC1(C)c2ccccc2-c2ccc(N(c3ccc(-c4cccc5c4sc4c(-c6ccccc6)cccc45)cc3)c3ccc4c(c3)c3c5oc(-c6ccccc6)nc5cc5c6ccccc6n4c53)cc21]

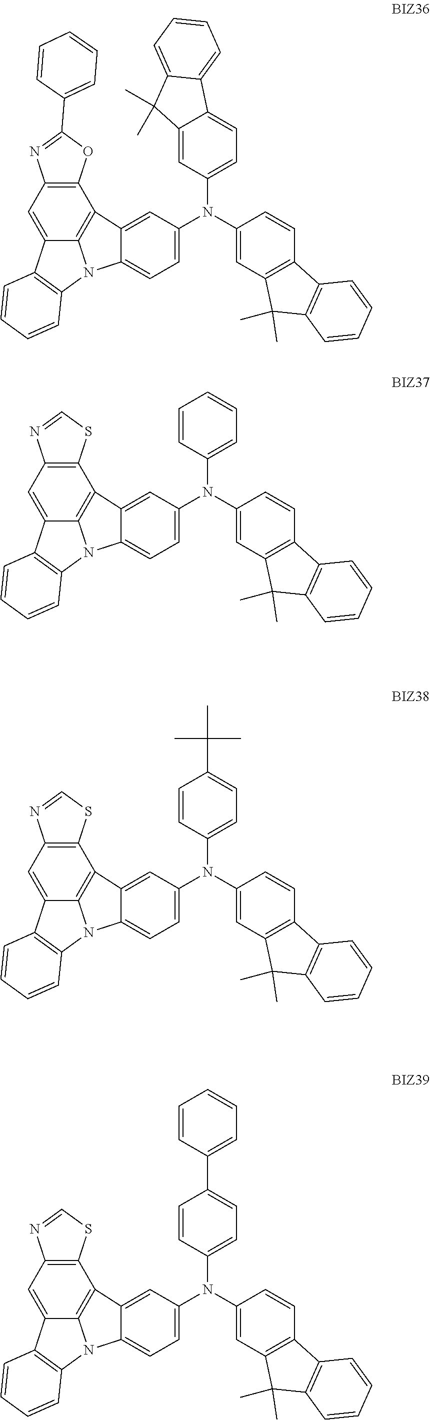 [SMILES: CC1(C)c2ccccc2-c2ccc(N(c3ccc4c(c3)C(C)(C)c3ccccc3-4)c3ccc4c(c3)c3c5oc(-c6ccccc6)nc5cc5c6ccccc6n4c53)cc21 ; CC1(C)c2ccccc2-c2ccc(N(c3ccccc3)c3ccc4c(c3)c3c5scnc5cc5c6ccccc6n4c53)cc21 ; CC(C)(C)c1ccc(N(c2ccc3c(c2)C(C)(C)c2ccccc2-3)c2ccc3c(c2)c2c4scnc4cc4c5ccccc5n3c42)cc1 ; CC1(C)c2ccccc2-c2ccc(N(c3ccc(-c4ccccc4)cc3)c3ccc4c(c3)c3c5scnc5cc5c6ccccc6n4c53)cc21]

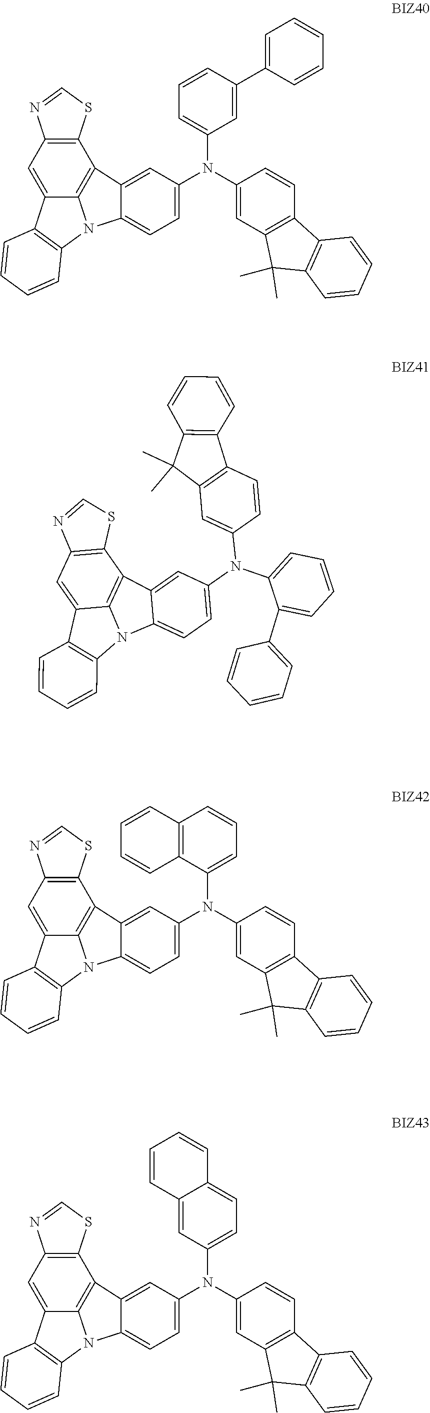 [SMILES: CC1(C)c2ccccc2-c2ccc(N(c3cccc(-c4ccccc4)c3)c3ccc4c(c3)c3c5scnc5cc5c6ccccc6n4c53)cc21 ; CC1(C)c2ccccc2-c2ccc(N(c3ccc4c(c3)c3c5scnc5cc5c6ccccc6n4c53)c3ccccc3-c3ccccc3)cc21 ; CC1(C)c2ccccc2-c2ccc(N(c3ccc4c(c3)c3c5scnc5cc5c6ccccc6n4c53)c3cccc4ccccc34)cc21 ; CC1(C)c2ccccc2-c2ccc(N(c3ccc4ccccc4c3)c3ccc4c(c3)c3c5scnc5cc5c6ccccc6n4c53)cc21]

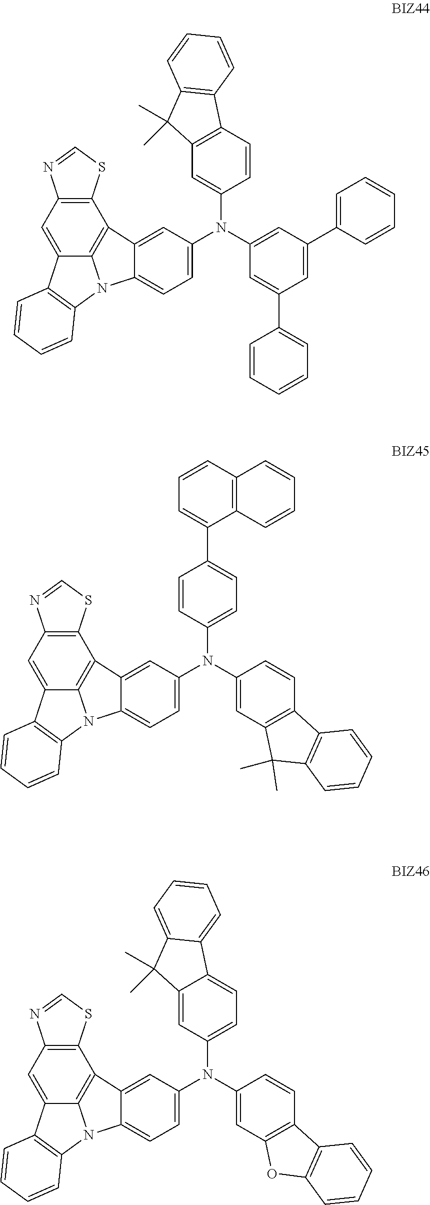 [SMILES: CC1(C)c2ccccc2-c2ccc(N(c3cc(-c4ccccc4)cc(-c4ccccc4)c3)c3ccc4c(c3)c3c5scnc5cc5c6ccccc6n4c53)cc21 ; CC1(C)c2ccccc2-c2ccc(N(c3ccc(-c4cccc5ccccc45)cc3)c3ccc4c(c3)c3c5scnc5cc5c6ccccc6n4c53)cc21 ; CC1(C)c2ccccc2-c2ccc(N(c3ccc4c(c3)oc3ccccc34)c3ccc4c(c3)c3c5scnc5cc5c6ccccc6n4c53)cc21]

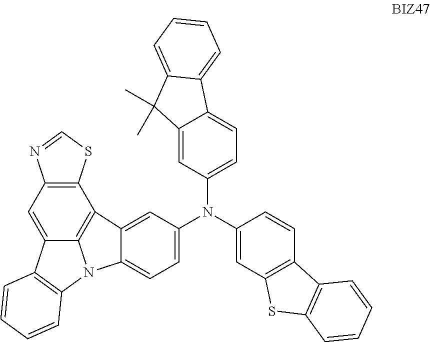 [SMILES: CC1(C)c2ccccc2-c2ccc(N(c3ccc4c(c3)sc3ccccc34)c3ccc4c(c3)c3c5scnc5cc5c6ccccc6n4c53)cc21]

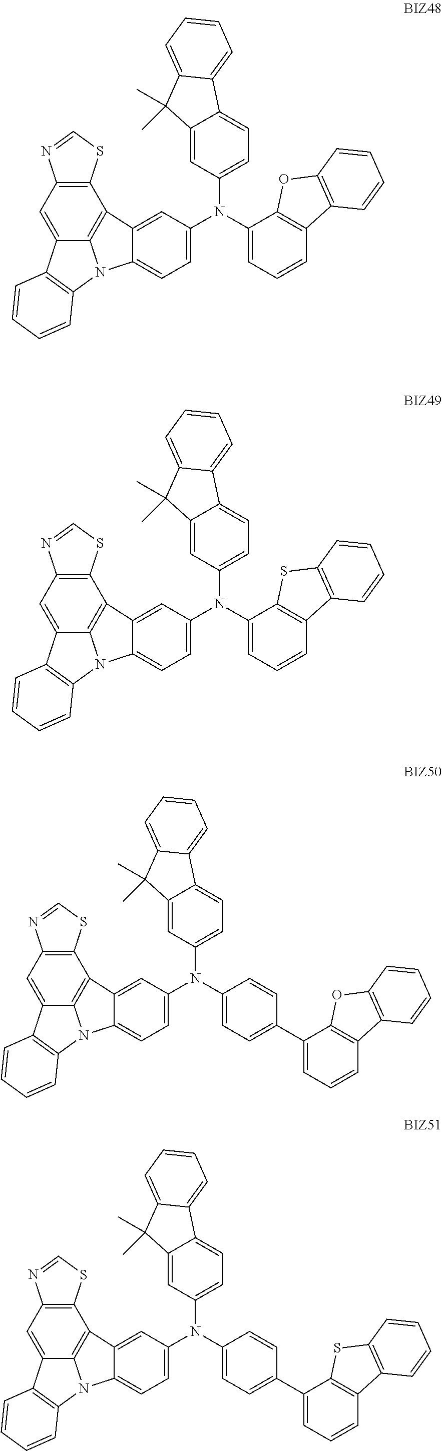 [SMILES: CC1(C)c2ccccc2-c2ccc(N(c3ccc4c(c3)c3c5scnc5cc5c6ccccc6n4c53)c3cccc4c3oc3ccccc34)cc21 ; CC1(C)c2ccccc2-c2ccc(N(c3ccc4c(c3)c3c5scnc5cc5c6ccccc6n4c53)c3cccc4c3sc3ccccc34)cc21 ; CC1(C)c2ccccc2-c2ccc(N(c3ccc(-c4cccc5c4oc4ccccc45)cc3)c3ccc4c(c3)c3c5scnc5cc5c6ccccc6n4c53)cc21 ; CC1(C)c2ccccc2-c2ccc(N(c3ccc(-c4cccc5c4sc4ccccc45)cc3)c3ccc4c(c3)c3c5scnc5cc5c6ccccc6n4c53)cc21]

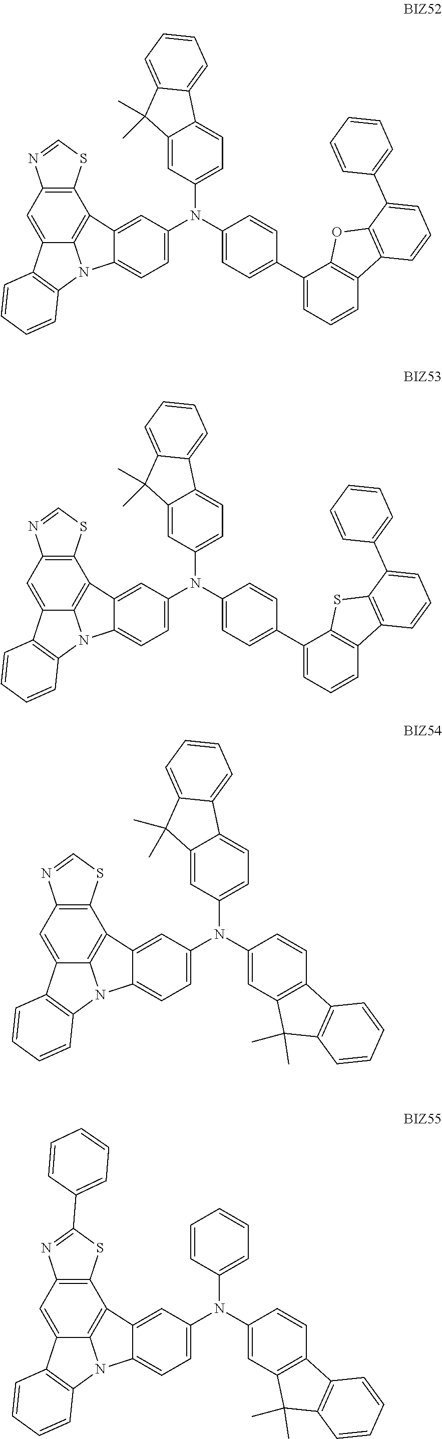 [SMILES: CC1(C)c2ccccc2-c2ccc(N(c3ccc(-c4cccc5c4oc4c(-c6ccccc6)cccc45)cc3)c3ccc4c(c3)c3c5scnc5cc5c6ccccc6n4c53)cc21 ; CC1(C)c2ccccc2-c2ccc(N(c3ccc(-c4cccc5c4sc4c(-c6ccccc6)cccc45)cc3)c3ccc4c(c3)c3c5scnc5cc5c6ccccc6n4c53)cc21 ; CC1(C)c2ccccc2-c2ccc(N(c3ccc4c(c3)C(C)(C)c3ccccc3-4)c3ccc4c(c3)c3c5scnc5cc5c6ccccc6n4c53)cc21 ; CC1(C)c2ccccc2-c2ccc(N(c3ccccc3)c3ccc4c(c3)c3c5sc(-c6ccccc6)nc5cc5c6ccccc6n4c53)cc21]

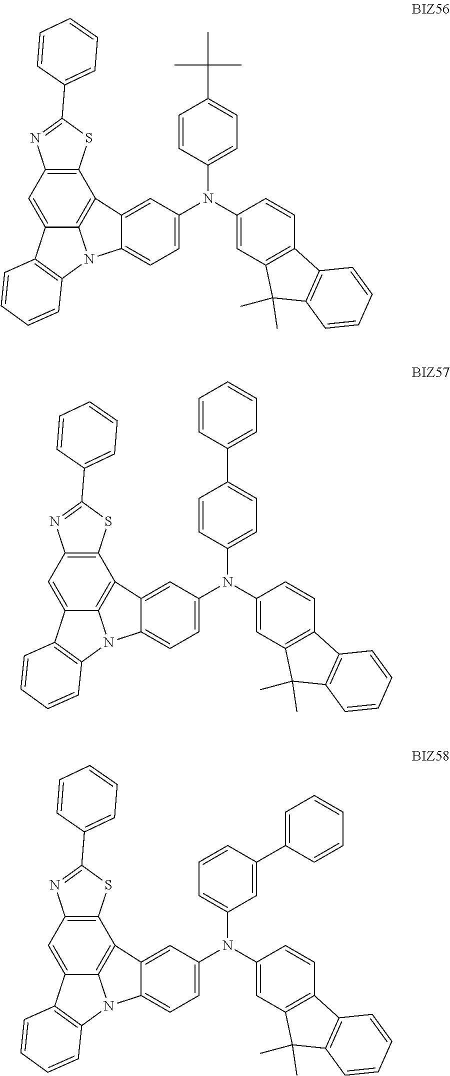 [SMILES: CC(C)(C)c1ccc(N(c2ccc3c(c2)C(C)(C)c2ccccc2-3)c2ccc3c(c2)c2c4sc(-c5ccccc5)nc4cc4c5ccccc5n3c42)cc1 ; CC1(C)c2ccccc2-c2ccc(N(c3ccc(-c4ccccc4)cc3)c3ccc4c(c3)c3c5sc(-c6ccccc6)nc5cc5c6ccccc6n4c53)cc21 ; CC1(C)c2ccccc2-c2ccc(N(c3cccc(-c4ccccc4)c3)c3ccc4c(c3)c3c5sc(-c6ccccc6)nc5cc5c6ccccc6n4c53)cc21]

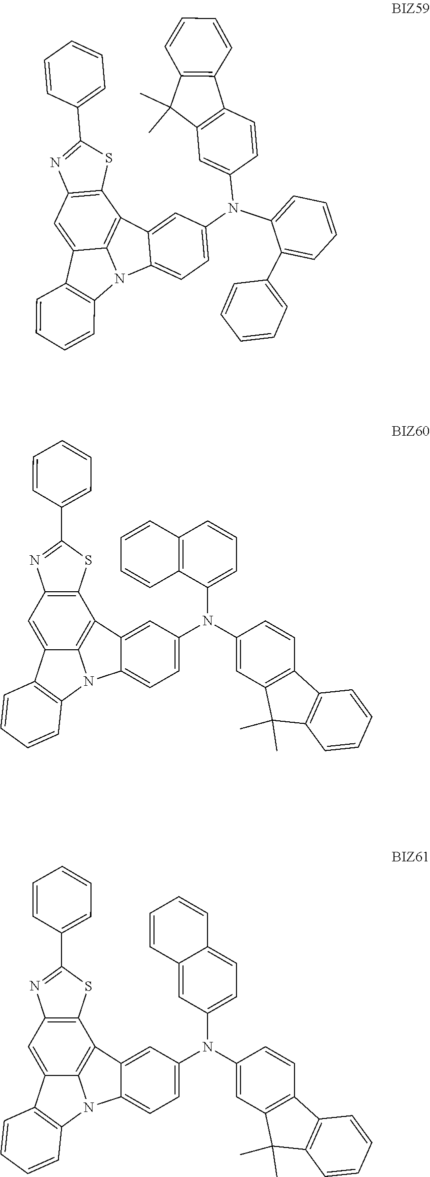 [SMILES: CC1(C)c2ccccc2-c2ccc(N(c3ccc4c(c3)c3c5sc(-c6ccccc6)nc5cc5c6ccccc6n4c53)c3ccccc3-c3ccccc3)cc21 ; CC1(C)c2ccccc2-c2ccc(N(c3ccc4c(c3)c3c5sc(-c6ccccc6)nc5cc5c6ccccc6n4c53)c3cccc4ccccc34)cc21 ; CC1(C)c2ccccc2-c2ccc(N(c3ccc4ccccc4c3)c3ccc4c(c3)c3c5sc(-c6ccccc6)nc5cc5c6ccccc6n4c53)cc21]

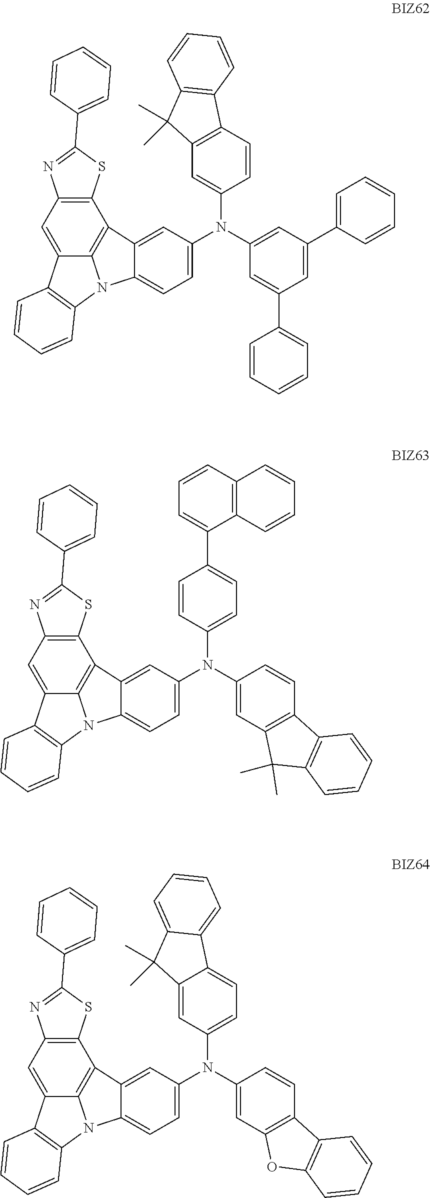 [SMILES: CC1(C)c2ccccc2-c2ccc(N(c3cc(-c4ccccc4)cc(-c4ccccc4)c3)c3ccc4c(c3)c3c5sc(-c6ccccc6)nc5cc5c6ccccc6n4c53)cc21 ; CC1(C)c2ccccc2-c2ccc(N(c3ccc(-c4cccc5ccccc45)cc3)c3ccc4c(c3)c3c5sc(-c6ccccc6)nc5cc5c6ccccc6n4c53)cc21 ; CC1(C)c2ccccc2-c2ccc(N(c3ccc4c(c3)oc3ccccc34)c3ccc4c(c3)c3c5sc(-c6ccccc6)nc5cc5c6ccccc6n4c53)cc21]

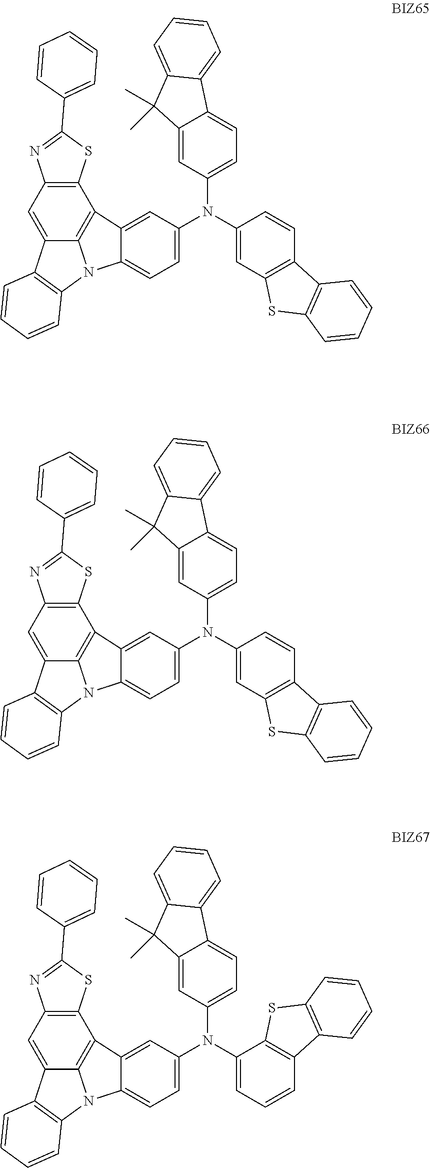 [SMILES: CC1(C)c2ccccc2-c2ccc(N(c3ccc4c(c3)sc3ccccc34)c3ccc4c(c3)c3c5sc(-c6ccccc6)nc5cc5c6ccccc6n4c53)cc21 ; CC1(C)c2ccccc2-c2ccc(N(c3ccc4c(c3)sc3ccccc34)c3ccc4c(c3)c3c5sc(-c6ccccc6)nc5cc5c6ccccc6n4c53)cc21 ; CC1(C)c2ccccc2-c2ccc(N(c3ccc4c(c3)c3c5sc(-c6ccccc6)nc5cc5c6ccccc6n4c53)c3cccc4c3sc3ccccc34)cc21]

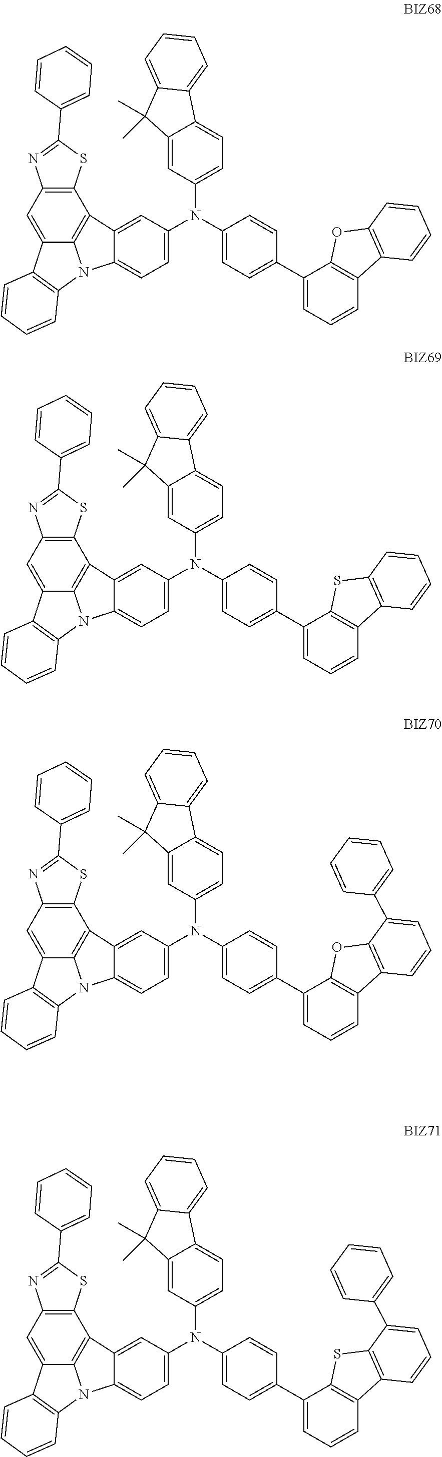 [SMILES: CC1(C)c2ccccc2-c2ccc(N(c3ccc(-c4cccc5c4oc4ccccc45)cc3)c3ccc4c(c3)c3c5sc(-c6ccccc6)nc5cc5c6ccccc6n4c53)cc21 ; CC1(C)c2ccccc2-c2ccc(N(c3ccc(-c4cccc5c4sc4ccccc45)cc3)c3ccc4c(c3)c3c5sc(-c6ccccc6)nc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5sc(-c6ccccc6)nc5cc5c6ccccc6n4c53)cc21 ; CC1(C)c2ccccc2-c2ccc(N(c3ccc(-c4cccc5c4sc4c(-c6ccccc6)cccc45)cc3)c3ccc4c(c3)c3c5sc(-c6ccccc6)nc5cc5c6ccccc6n4c53)cc21]

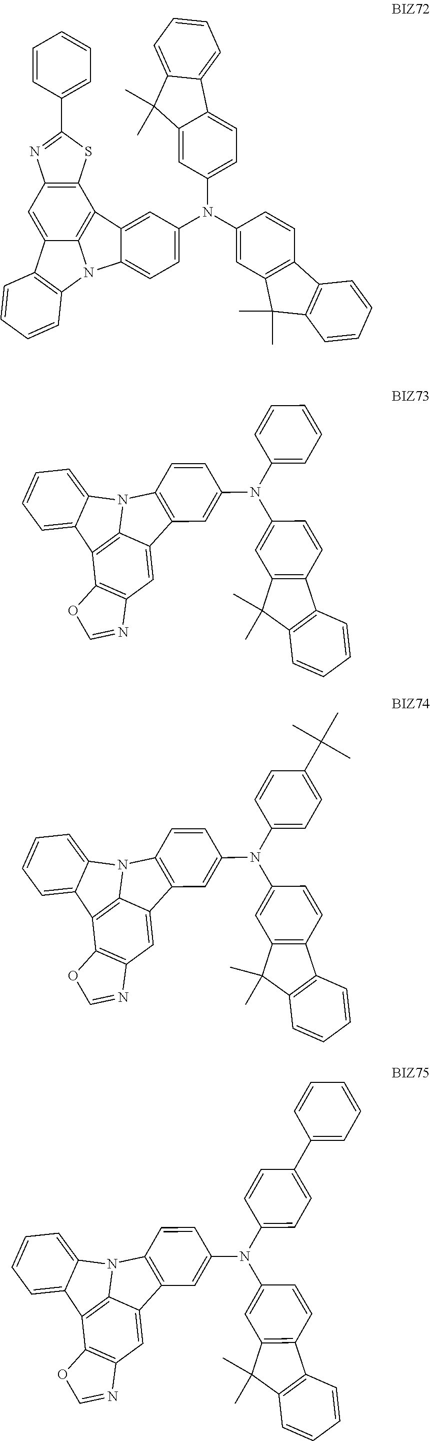

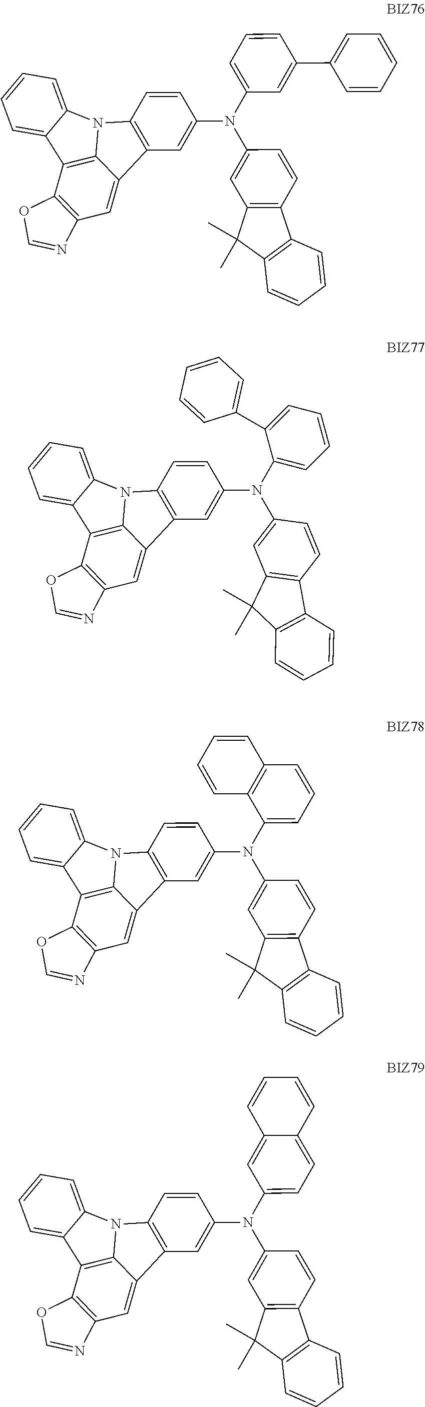

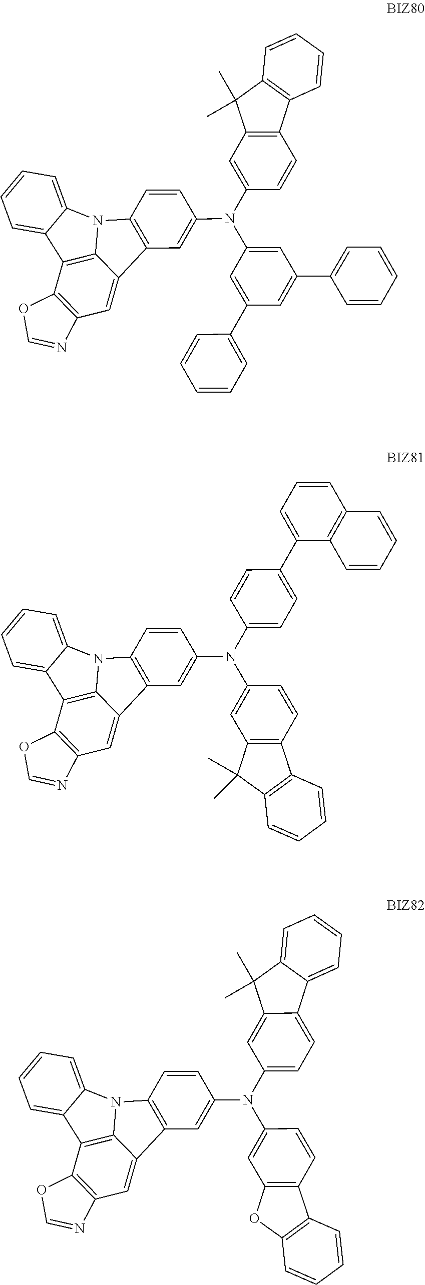 [SMILES: CC1(C)c2ccccc2-c2ccc(N(c3cc(-c4ccccc4)cc(-c4ccccc4)c3)c3ccc4c(c3)c3cc5ncoc5c5c6ccccc6n4c35)cc21 ; CC1(C)c2ccccc2-c2ccc(N(c3ccc(-c4cccc5ccccc45)cc3)c3ccc4c(c3)c3cc5ncoc5c5c6ccccc6n4c35)cc21 ; CC1(C)c2ccccc2-c2ccc(N(c3ccc4c(c3)oc3ccccc34)c3ccc4c(c3)c3cc5ncoc5c5c6ccccc6n4c35)cc21]

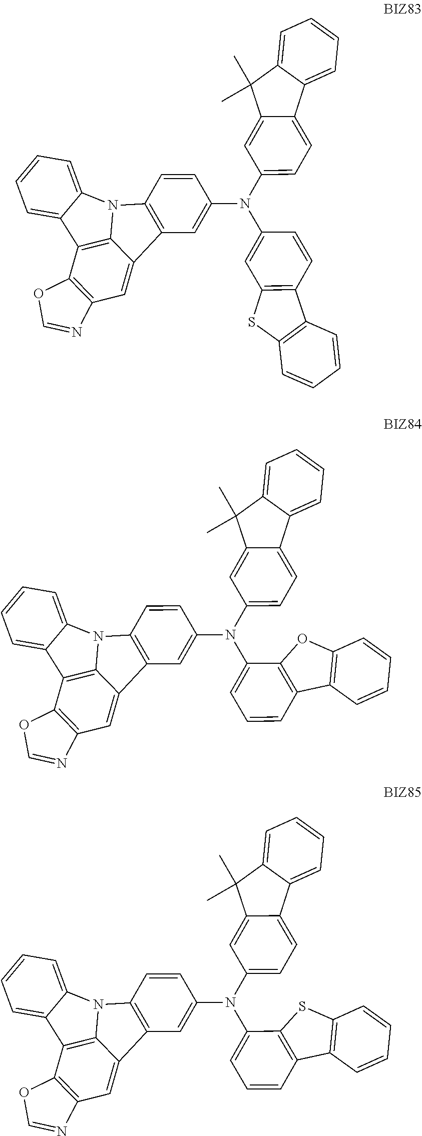 [SMILES: CC1(C)c2ccccc2-c2ccc(N(c3ccc4c(c3)sc3ccccc34)c3ccc4c(c3)c3cc5ncoc5c5c6ccccc6n4c35)cc21 ; CC1(C)c2ccccc2-c2ccc(N(c3ccc4c(c3)c3cc5ncoc5c5c6ccccc6n4c35)c3cccc4c3oc3ccccc34)cc21 ; CC1(C)c2ccccc2-c2ccc(N(c3ccc4c(c3)c3cc5ncoc5c5c6ccccc6n4c35)c3cccc4c3sc3ccccc34)cc21]

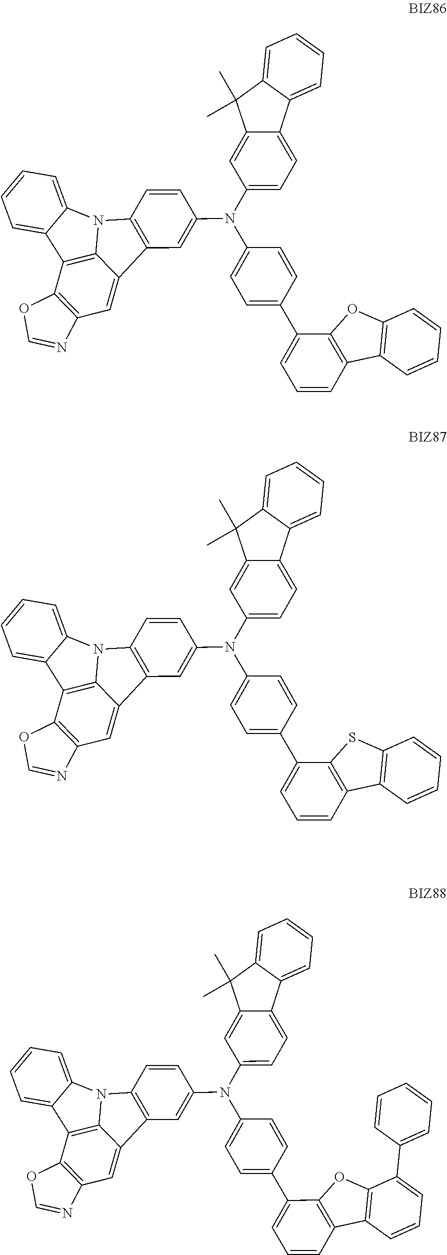 [SMILES: CC1(C)c2ccccc2-c2ccc(N(c3ccc(-c4cccc5c4oc4ccccc45)cc3)c3ccc4c(c3)c3cc5ncoc5c5c6ccccc6n4c35)cc21 ; CC1(C)c2ccccc2-c2ccc(N(c3ccc(-c4cccc5c4sc4ccccc45)cc3)c3ccc4c(c3)c3cc5ncoc5c5c6ccccc6n4c35)cc21 ; CC1(C)c2ccccc2-c2ccc(N(c3ccc(-c4cccc5c4oc4c(-c6ccccc6)cccc45)cc3)c3ccc4c(c3)c3cc5ncoc5c5c6ccccc6n4c35)cc21]

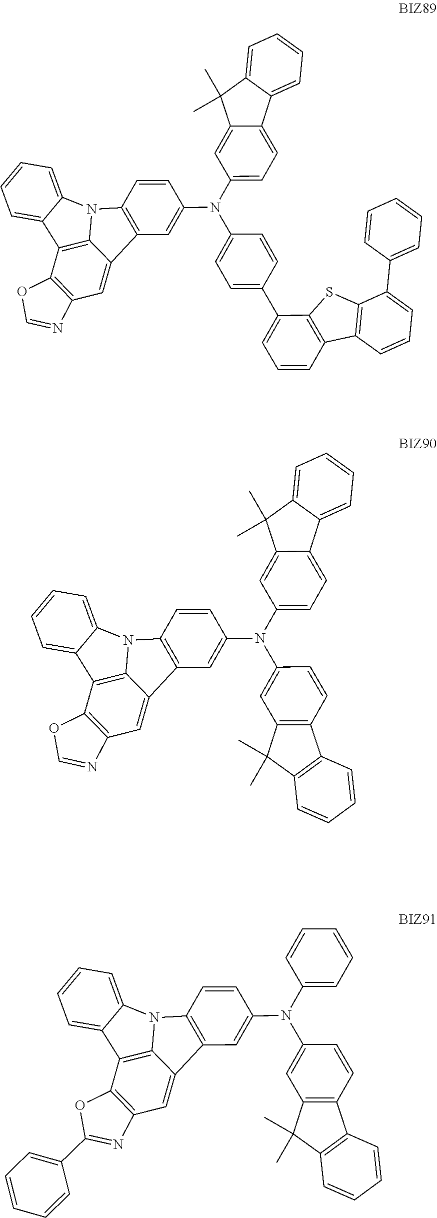 [SMILES: CC1(C)c2ccccc2-c2ccc(N(c3ccc(-c4cccc5c4sc4c(-c6ccccc6)cccc45)cc3)c3ccc4c(c3)c3cc5ncoc5c5c6ccccc6n4c35)cc21 ; CC1(C)c2ccccc2-c2ccc(N(c3ccc4c(c3)C(C)(C)c3ccccc3-4)c3ccc4c(c3)c3cc5ncoc5c5c6ccccc6n4c35)cc21 ; CC1(C)c2ccccc2-c2ccc(N(c3ccccc3)c3ccc4c(c3)c3cc5nc(-c6ccccc6)oc5c5c6ccccc6n4c35)cc21]

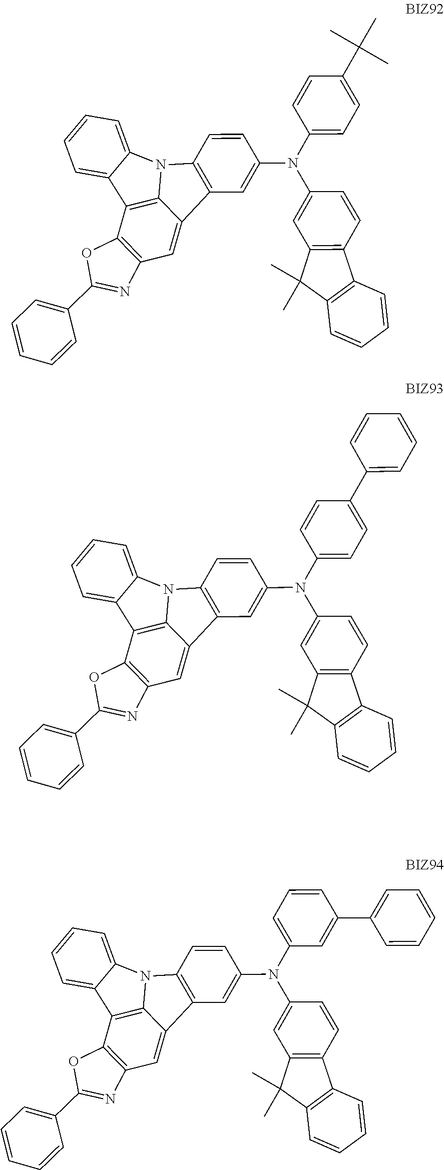 [SMILES: CC(C)(C)c1ccc(N(c2ccc3c(c2)C(C)(C)c2ccccc2-3)c2ccc3c(c2)c2cc4nc(-c5ccccc5)oc4c4c5ccccc5n3c24)cc1 ; CC1(C)c2ccccc2-c2ccc(N(c3ccc(-c4ccccc4)cc3)c3ccc4c(c3)c3cc5nc(-c6ccccc6)oc5c5c6ccccc6n4c35)cc21 ; CC1(C)c2ccccc2-c2ccc(N(c3cccc(-c4ccccc4)c3)c3ccc4c(c3)c3cc5nc(-c6ccccc6)oc5c5c6ccccc6n4c35)cc21]

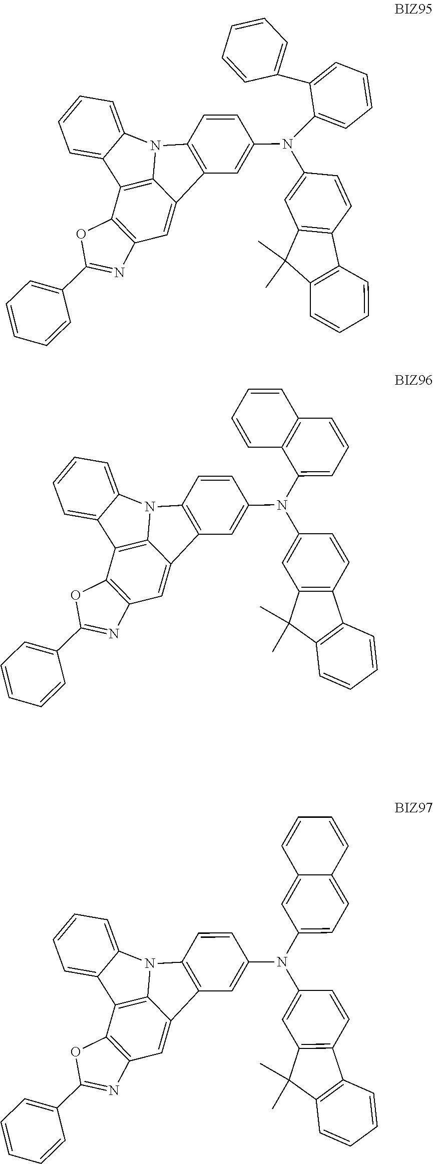 [SMILES: CC1(C)c2ccccc2-c2ccc(N(c3ccc4c(c3)c3cc5nc(-c6ccccc6)oc5c5c6ccccc6n4c35)c3ccccc3-c3ccccc3)cc21 ; CC1(C)c2ccccc2-c2ccc(N(c3ccc4c(c3)c3cc5nc(-c6ccccc6)oc5c5c6ccccc6n4c35)c3cccc4ccccc34)cc21 ; CC1(C)c2ccccc2-c2ccc(N(c3ccc4ccccc4c3)c3ccc4c(c3)c3cc5nc(-c6ccccc6)oc5c5c6ccccc6n4c35)cc21]

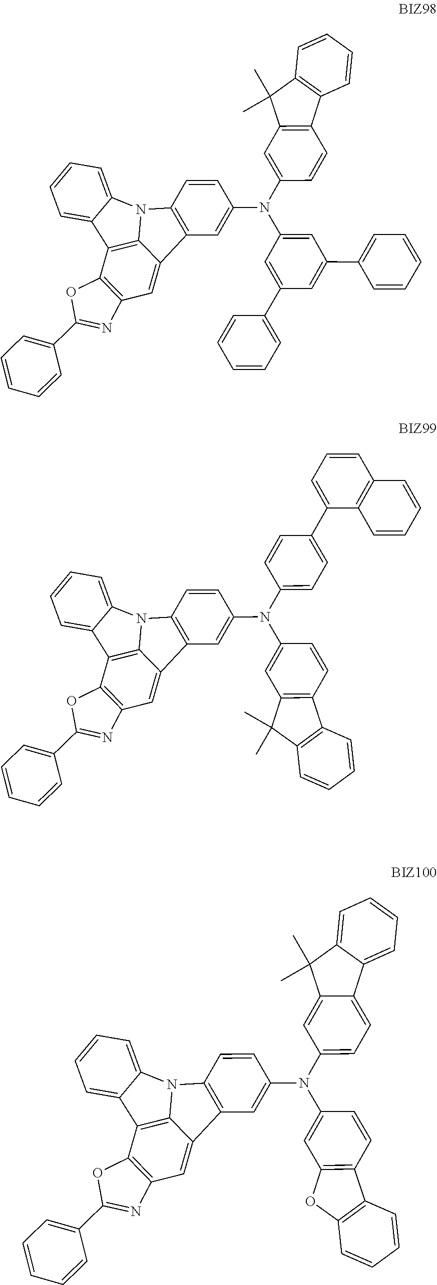 [SMILES: CC1(C)c2ccccc2-c2ccc(N(c3cc(-c4ccccc4)cc(-c4ccccc4)c3)c3ccc4c(c3)c3cc5nc(-c6ccccc6)oc5c5c6ccccc6n4c35)cc21 ; CC1(C)c2ccccc2-c2ccc(N(c3ccc(-c4cccc5ccccc45)cc3)c3ccc4c(c3)c3cc5nc(-c6ccccc6)oc5c5c6ccccc6n4c35)cc21 ; CC1(C)c2ccccc2-c2ccc(N(c3ccc4c(c3)oc3ccccc34)c3ccc4c(c3)c3cc5nc(-c6ccccc6)oc5c5c6ccccc6n4c35)cc21]

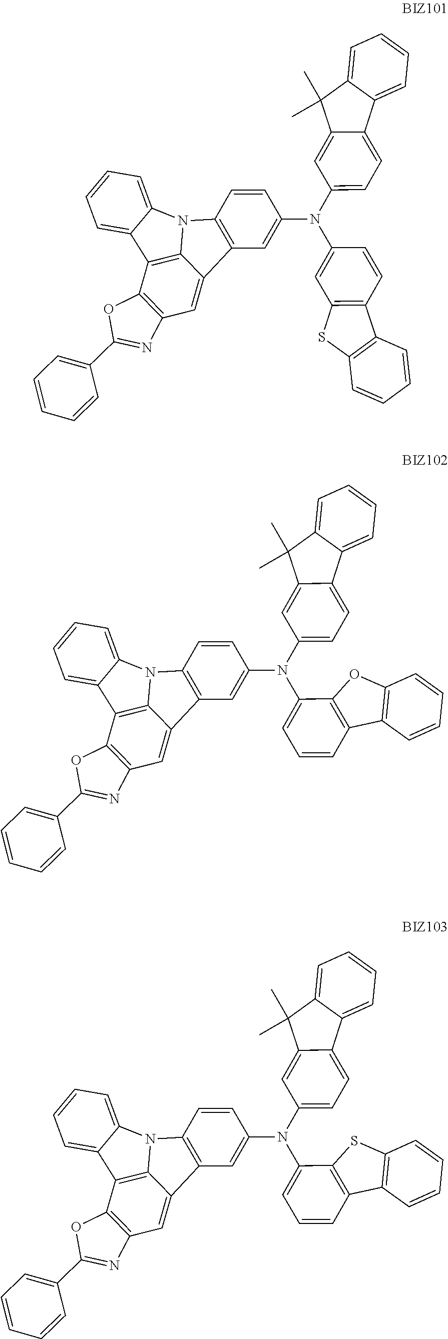 [SMILES: CC1(C)c2ccccc2-c2ccc(N(c3ccc4c(c3)sc3ccccc34)c3ccc4c(c3)c3cc5nc(-c6ccccc6)oc5c5c6ccccc6n4c35)cc21 ; CC1(C)c2ccccc2-c2ccc(N(c3ccc4c(c3)c3cc5nc(-c6ccccc6)oc5c5c6ccccc6n4c35)c3cccc4c3oc3ccccc34)cc21 ; CC1(C)c2ccccc2-c2ccc(N(c3ccc4c(c3)c3cc5nc(-c6ccccc6)oc5c5c6ccccc6n4c35)c3cccc4c3sc3ccccc34)cc21]

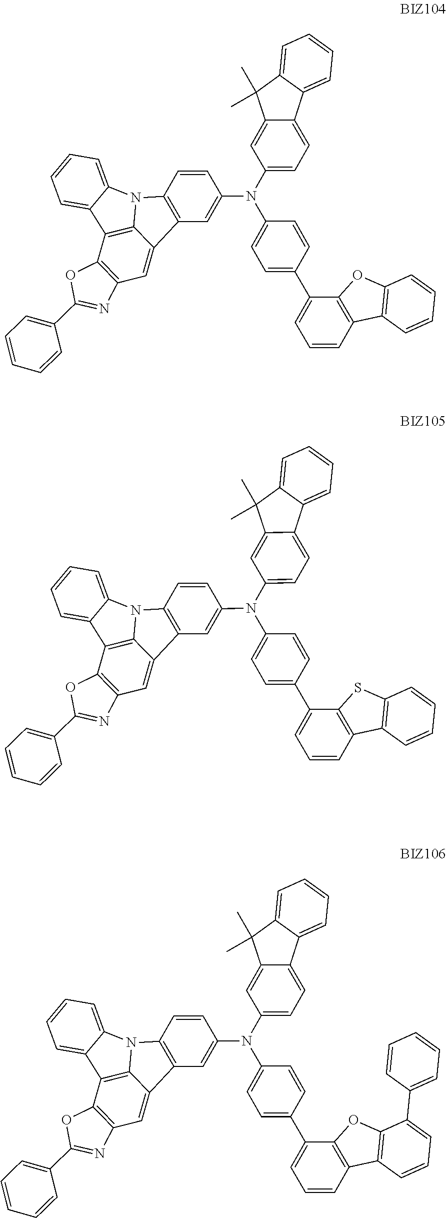 [SMILES: CC1(C)c2ccccc2-c2ccc(N(c3ccc(-c4cccc5c4oc4ccccc45)cc3)c3ccc4c(c3)c3cc5nc(-c6ccccc6)oc5c5c6ccccc6n4c35)cc21 ; CC1(C)c2ccccc2-c2ccc(N(c3ccc(-c4cccc5c4sc4ccccc45)cc3)c3ccc4c(c3)c3cc5nc(-c6ccccc6)oc5c5c6ccccc6n4c35)cc21 ; CC1(C)c2ccccc2-c2ccc(N(c3ccc(-c4cccc5c4oc4c(-c6ccccc6)cccc45)cc3)c3ccc4c(c3)c3cc5nc(-c6ccccc6)oc5c5c6ccccc6n4c35)cc21]

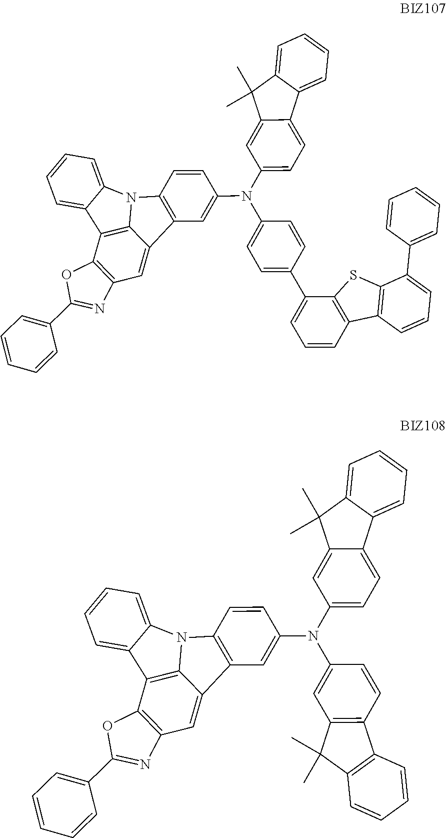 [SMILES: CC1(C)c2ccccc2-c2ccc(N(c3ccc(-c4cccc5c4sc4c(-c6ccccc6)cccc45)cc3)c3ccc4c(c3)c3cc5nc(-c6ccccc6)oc5c5c6ccccc6n4c35)cc21 ; CC1(C)c2ccccc2-c2ccc(N(c3ccc4c(c3)C(C)(C)c3ccccc3-4)c3ccc4c(c3)c3cc5nc(-c6ccccc6)oc5c5c6ccccc6n4c35)cc21]

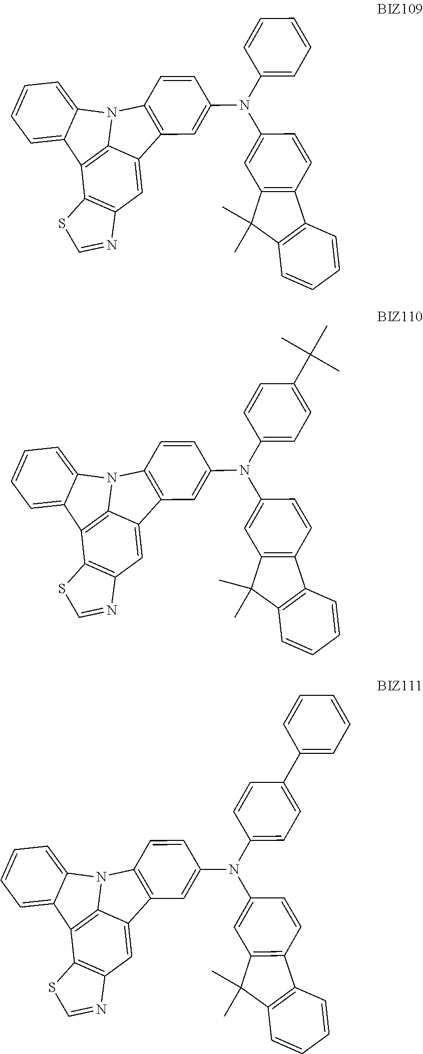

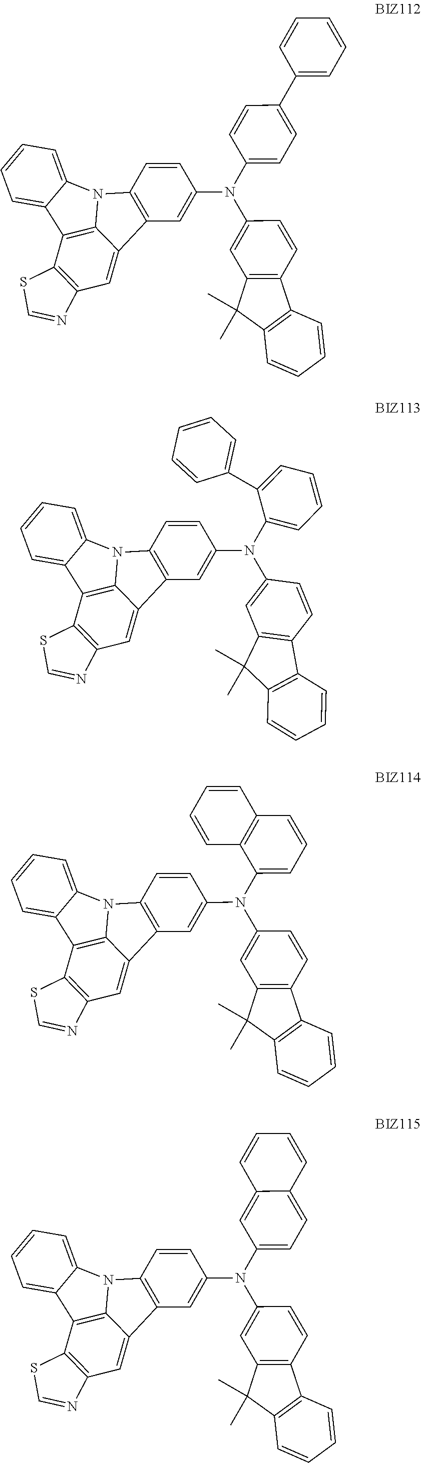 [SMILES: CC1(C)c2ccccc2-c2ccc(N(c3ccc(-c4ccccc4)cc3)c3ccc4c(c3)c3cc5ncsc5c5c6ccccc6n4c35)cc21 ; CC1(C)c2ccccc2-c2ccc(N(c3ccc4c(c3)c3cc5ncsc5c5c6ccccc6n4c35)c3ccccc3-c3ccccc3)cc21 ; CC1(C)c2ccccc2-c2ccc(N(c3ccc4c(c3)c3cc5ncsc5c5c6ccccc6n4c35)c3cccc4ccccc34)cc21 ; CC1(C)c2ccccc2-c2ccc(N(c3ccc4ccccc4c3)c3ccc4c(c3)c3cc5ncsc5c5c6ccccc6n4c35)cc21]

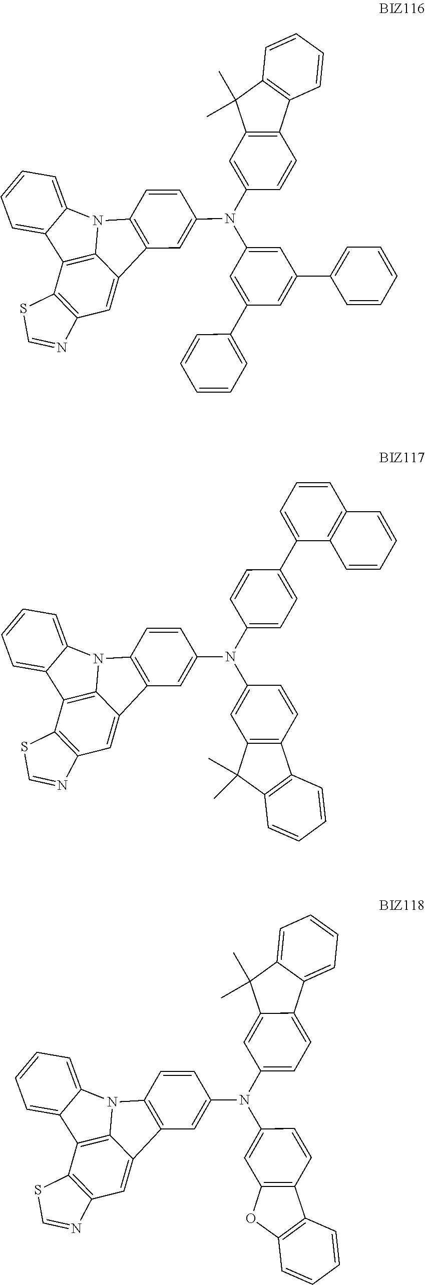 [SMILES: CC1(C)c2ccccc2-c2ccc(N(c3cc(-c4ccccc4)cc(-c4ccccc4)c3)c3ccc4c(c3)c3cc5ncsc5c5c6ccccc6n4c35)cc21 ; CC1(C)c2ccccc2-c2ccc(N(c3ccc(-c4cccc5ccccc45)cc3)c3ccc4c(c3)c3cc5ncsc5c5c6ccccc6n4c35)cc21 ; CC1(C)c2ccccc2-c2ccc(N(c3ccc4c(c3)oc3ccccc34)c3ccc4c(c3)c3cc5ncsc5c5c6ccccc6n4c35)cc21]

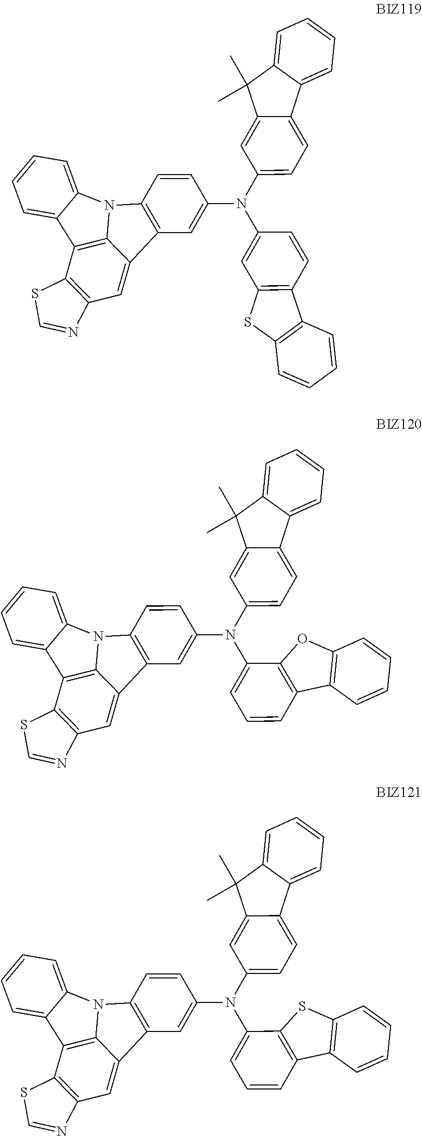 [SMILES: CC1(C)c2ccccc2-c2ccc(N(c3ccc4c(c3)sc3ccccc34)c3ccc4c(c3)c3cc5ncsc5c5c6ccccc6n4c35)cc21 ; CC1(C)c2ccccc2-c2ccc(N(c3ccc4c(c3)c3cc5ncsc5c5c6ccccc6n4c35)c3cccc4c3oc3ccccc34)cc21 ; CC1(C)c2ccccc2-c2ccc(N(c3ccc4c(c3)c3cc5ncsc5c5c6ccccc6n4c35)c3cccc4c3sc3ccccc34)cc21]

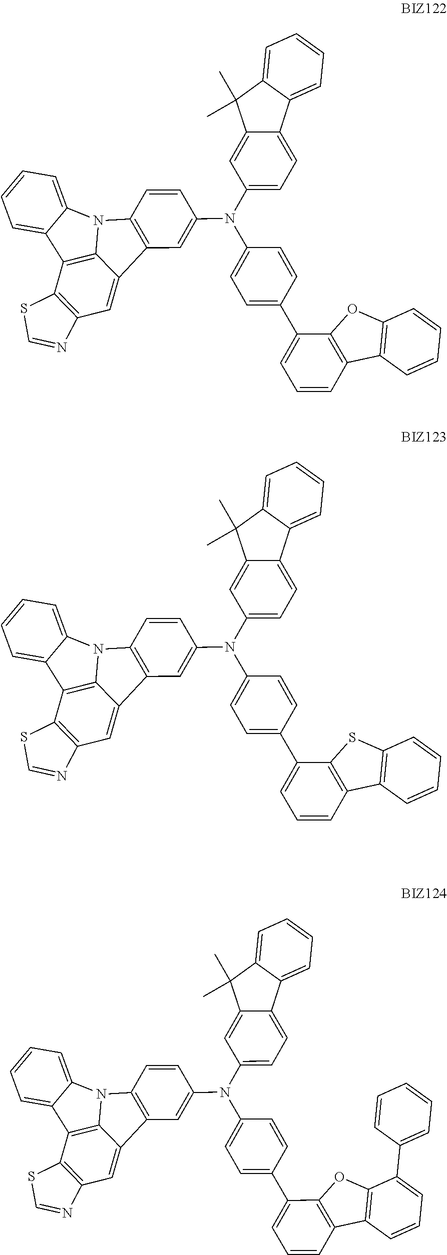 [SMILES: CC1(C)c2ccccc2-c2ccc(N(c3ccc(-c4cccc5c4oc4ccccc45)cc3)c3ccc4c(c3)c3cc5ncsc5c5c6ccccc6n4c35)cc21 ; CC1(C)c2ccccc2-c2ccc(N(c3ccc(-c4cccc5c4sc4ccccc45)cc3)c3ccc4c(c3)c3cc5ncsc5c5c6ccccc6n4c35)cc21 ; CC1(C)c2ccccc2-c2ccc(N(c3ccc(-c4cccc5c4oc4c(-c6ccccc6)cccc45)cc3)c3ccc4c(c3)c3cc5ncsc5c5c6ccccc6n4c35)cc21]

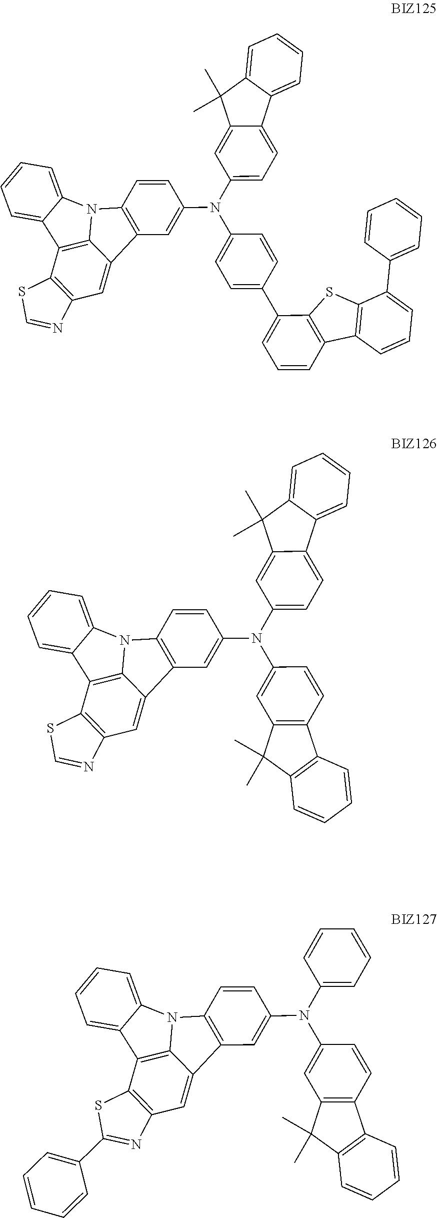 [SMILES: CC1(C)c2ccccc2-c2ccc(N(c3ccc(-c4cccc5c4sc4c(-c6ccccc6)cccc45)cc3)c3ccc4c(c3)c3cc5ncsc5c5c6ccccc6n4c35)cc21 ; CC1(C)c2ccccc2-c2ccc(N(c3ccc4c(c3)C(C)(C)c3ccccc3-4)c3ccc4c(c3)c3cc5ncsc5c5c6ccccc6n4c35)cc21 ; CC1(C)c2ccccc2-c2ccc(N(c3ccccc3)c3ccc4c(c3)c3cc5nc(-c6ccccc6)sc5c5c6ccccc6n4c35)cc21]

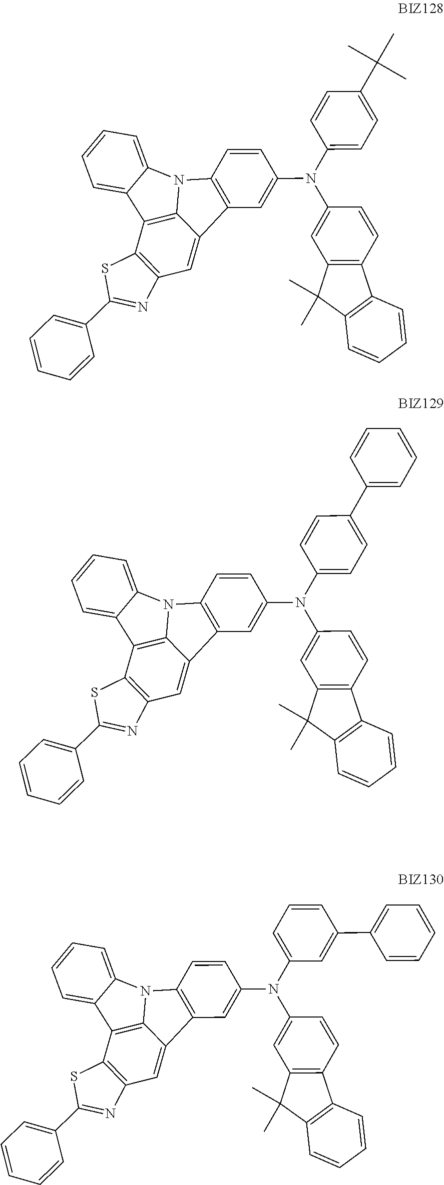 [SMILES: CC(C)(C)c1ccc(N(c2ccc3c(c2)C(C)(C)c2ccccc2-3)c2ccc3c(c2)c2cc4nc(-c5ccccc5)sc4c4c5ccccc5n3c24)cc1 ; CC1(C)c2ccccc2-c2ccc(N(c3ccc(-c4ccccc4)cc3)c3ccc4c(c3)c3cc5nc(-c6ccccc6)sc5c5c6ccccc6n4c35)cc21 ; CC1(C)c2ccccc2-c2ccc(N(c3cccc(-c4ccccc4)c3)c3ccc4c(c3)c3cc5nc(-c6ccccc6)sc5c5c6ccccc6n4c35)cc21]

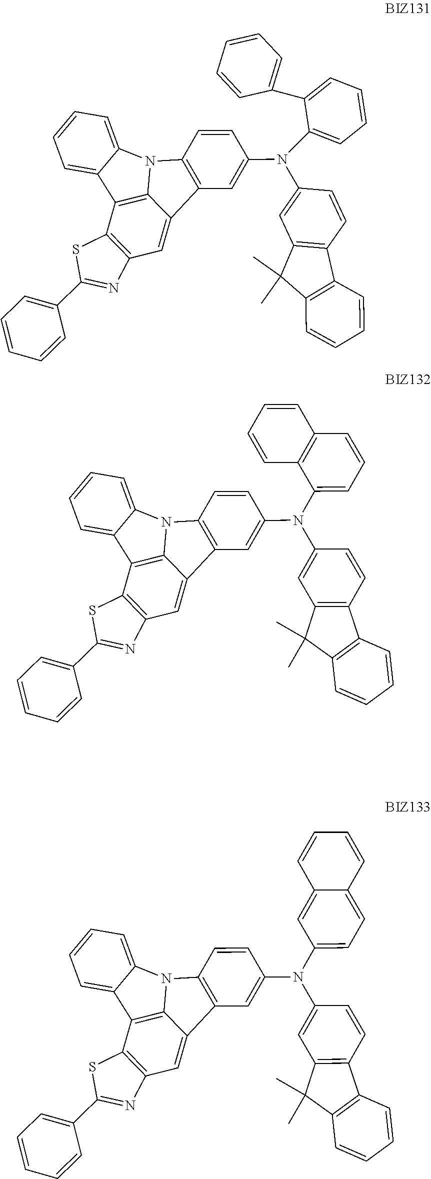 [SMILES: CC1(C)c2ccccc2-c2ccc(N(c3ccc4c(c3)c3cc5nc(-c6ccccc6)sc5c5c6ccccc6n4c35)c3ccccc3-c3ccccc3)cc21 ; CC1(C)c2ccccc2-c2ccc(N(c3ccc4c(c3)c3cc5nc(-c6ccccc6)sc5c5c6ccccc6n4c35)c3cccc4ccccc34)cc21 ; CC1(C)c2ccccc2-c2ccc(N(c3ccc4ccccc4c3)c3ccc4c(c3)c3cc5nc(-c6ccccc6)sc5c5c6ccccc6n4c35)cc21]

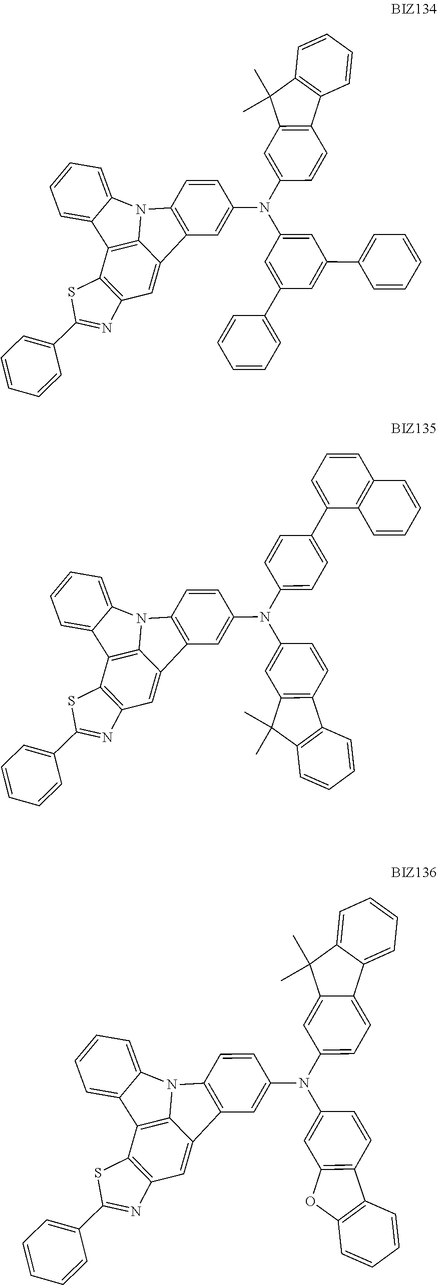 [SMILES: CC1(C)c2ccccc2-c2ccc(N(c3cc(-c4ccccc4)cc(-c4ccccc4)c3)c3ccc4c(c3)c3cc5nc(-c6ccccc6)sc5c5c6ccccc6n4c35)cc21 ; CC1(C)c2ccccc2-c2ccc(N(c3ccc(-c4cccc5ccccc45)cc3)c3ccc4c(c3)c3cc5nc(-c6ccccc6)sc5c5c6ccccc6n4c35)cc21 ; CC1(C)c2ccccc2-c2ccc(N(c3ccc4c(c3)oc3ccccc34)c3ccc4c(c3)c3cc5nc(-c6ccccc6)sc5c5c6ccccc6n4c35)cc21]

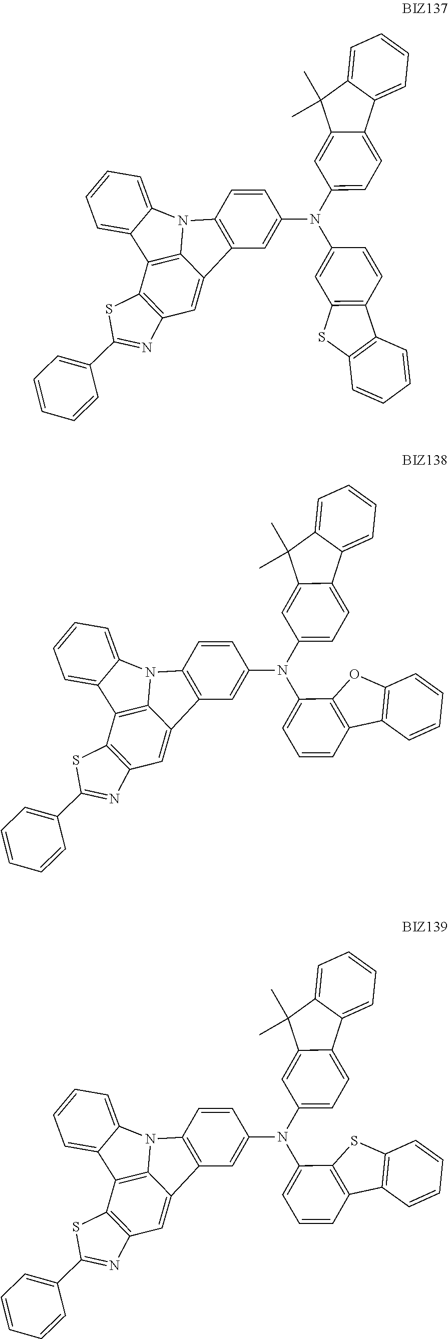 [SMILES: CC1(C)c2ccccc2-c2ccc(N(c3ccc4c(c3)sc3ccccc34)c3ccc4c(c3)c3cc5nc(-c6ccccc6)sc5c5c6ccccc6n4c35)cc21 ; CC1(C)c2ccccc2-c2ccc(N(c3ccc4c(c3)c3cc5nc(-c6ccccc6)sc5c5c6ccccc6n4c35)c3cccc4c3oc3ccccc34)cc21 ; CC1(C)c2ccccc2-c2ccc(N(c3ccc4c(c3)c3cc5nc(-c6ccccc6)sc5c5c6ccccc6n4c35)c3cccc4c3sc3ccccc34)cc21]

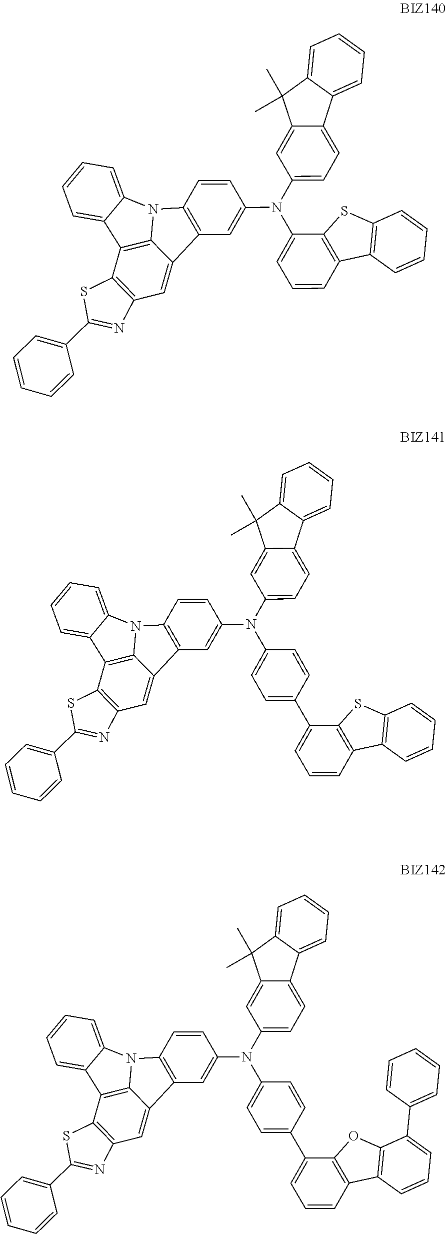 [SMILES: CC1(C)c2ccccc2-c2ccc(N(c3ccc4c(c3)c3cc5nc(-c6ccccc6)sc5c5c6ccccc6n4c35)c3cccc4c3sc3ccccc34)cc21 ; CC1(C)c2ccccc2-c2ccc(N(c3ccc(-c4cccc5c4sc4ccccc45)cc3)c3ccc4c(c3)c3cc5nc(-c6ccccc6)sc5c5c6ccccc6n4c35)cc21 ; CC1(C)c2ccccc2-c2ccc(N(c3ccc(-c4cccc5c4oc4c(-c6ccccc6)cccc45)cc3)c3ccc4c(c3)c3cc5nc(-c6ccccc6)sc5c5c6ccccc6n4c35)cc21]

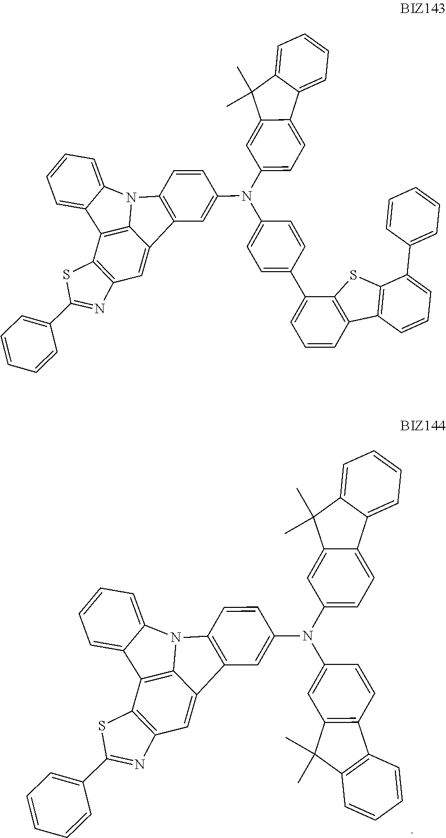 [SMILES: CC1(C)c2ccccc2-c2ccc(N(c3ccc(-c4cccc5c4sc4c(-c6ccccc6)cccc45)cc3)c3ccc4c(c3)c3cc5nc(-c6ccccc6)sc5c5c6ccccc6n4c35)cc21 ; CC1(C)c2ccccc2-c2ccc(N(c3ccc4c(c3)C(C)(C)c3ccccc3-4)c3ccc4c(c3)c3cc5nc(-c6ccccc6)sc5c5c6ccccc6n4c35)cc21]

4. An organic light emitting diode, comprising:
a first electrode;
a second electrode facing the first electrode; and
a first emitting part including a first red emitting material layer, the first emitting part positioned between the first electrode and the second electrode,
wherein the first red emitting material layer includes a first compound represented by Formula 1.

5. The organic light emitting diode according to claim 4, wherein the first red emitting material layer further includes a second compound represented by Formula 3: 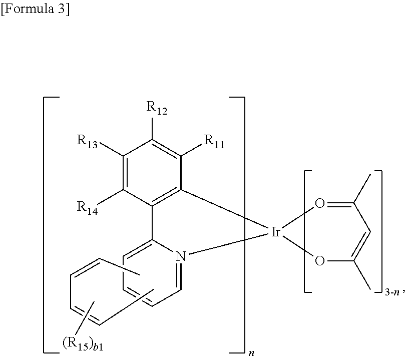
wherein in the Formula 3,
n is an integer of 1 to 3, and b1 is an integer of 0 to 4,
each of R₁₁ to R₁₄ is independently selected from the group consisting of hydrogen, a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group, or adjacent two of R₁₁ to R₁₄ is combined to each other to form an aromatic ring or a heteroaromatic ring, and
R₁₅ is independently selected from the group consisting of a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group.

6. The organic light emitting diode according to claim 5, wherein the second compound is one of compounds in Formula 4:

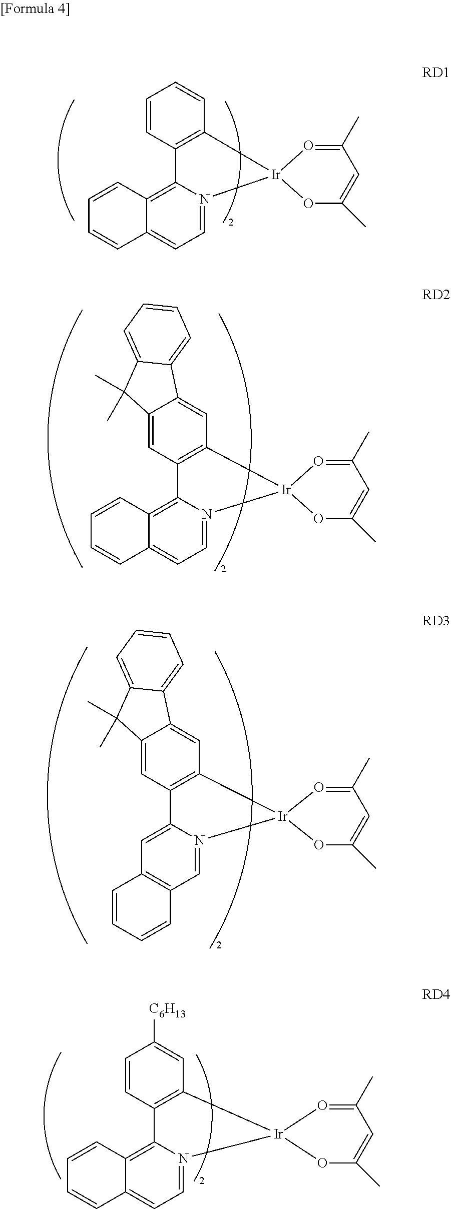

7. The organic light emitting diode according to claim 5, wherein the first red emitting material layer further includes a third compound represented by Formula 5: 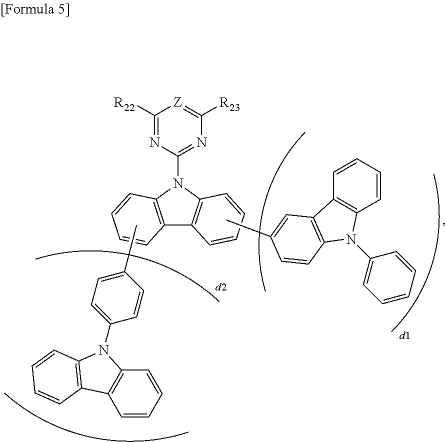
wherein in the Formula 5,
each of d1 and d2 is independently 0 or 1,
Z is N or CR₂₁, and each of R₂₁, R₂₂ and R₃ is independently selected from the group consisting of hydrogen, a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group, and
optionally, when Z is CR₂₁, one of a pair of R₂₁ and R₂₂ or a pair of R₂₁ and R₂₃ is combined to each other to form an aromatic ring or a heteroaromatic ring.

8. The organic light emitting diode according to claim 7, wherein the third compound is one of compounds in Formula 6:

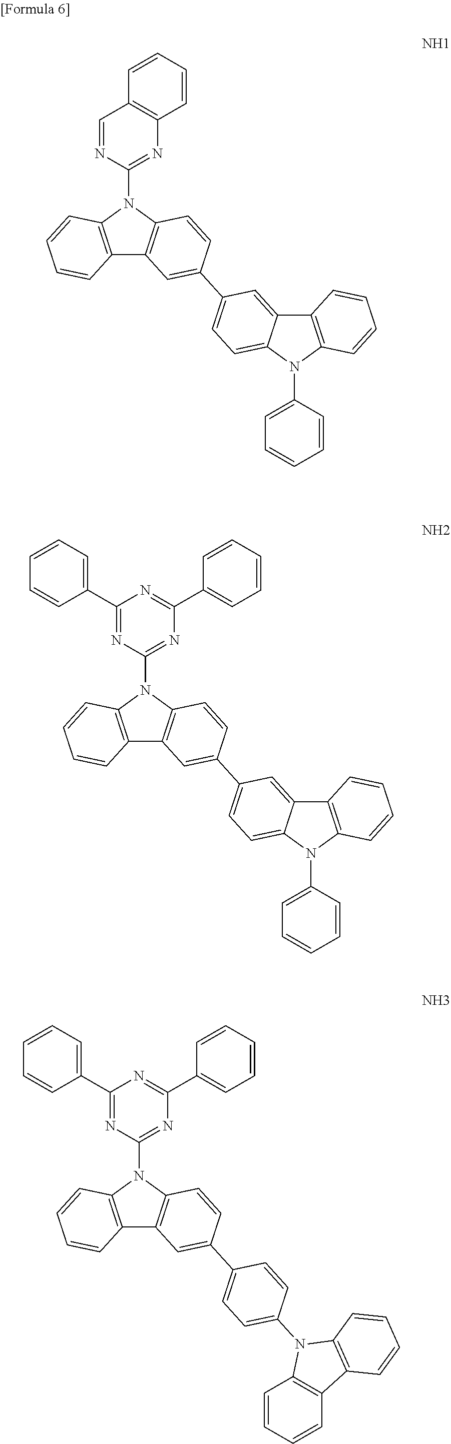 [SMILES: c1ccc(-n2c3ccccc3c3cc(-c4ccc5c(c4)c4ccccc4n5-c4ncc5ccccc5n4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-n3c4ccccc4c4cc(-c5ccc(-n6c7ccccc7c7ccccc76)cc5)ccc43)n2)cc1]

9. The organic light emitting diode according to claim 7, wherein a weight % of each of the first and third compound is greater than a weight % of the second compound.

10. The organic light emitting diode according to claim 4, wherein the first emitting part further includes at least one of a hole injection layer, a hole transporting layer, an electron blocking layer, a hole blocking layer, an electron transporting layer, and an electron injection layer.

11. The organic light emitting diode according to claim 4, further comprising:
a second emitting part including a second red emitting material layer and positioned between the first emitting part and the second electrode, and
a charge generation layer positioned between the first emitting part and the second emitting part.

12. The organic light emitting diode according to claim 11, wherein the second red emitting material layer further includes a fourth compound represented by Formula 1.

13. The organic light emitting diode according to claim 11, wherein the second emitting part further includes at least one of a hole injection layer, a hole transporting layer, an electron blocking layer, a hole blocking layer, an electron transporting layer, and an electron injection layer.

14. The organic light emitting diode according to claim 4, further comprising:
a second emitting part including a first blue emitting material layer and positioned between the first electrode and the first emitting part;
a first charge generation layer between the first emitting part and the second emitting part;
a third emitting part including a second blue emitting material layer and positioned between the first emitting part and the second electrode; and
a second charge generation layer between the first emitting part and the third emitting part.

15. The organic light emitting diode according to claim 14, wherein the first emitting material part further includes a green emitting material layer between the first red emitting material layer and the second charge generation layer.

16. The organic light emitting diode according to claim 15, wherein the first emitting material part further includes a yellow-green emitting material layer between the first red emitting material layer and the green emitting material layer.

17. The organic light emitting diode according to claim 14, wherein each of the second emitting part and the third emitting part independently further includes at least one of a hole injection layer, a hole transporting layer, an electron blocking layer, a hole blocking layer, an electron transporting layer and an electron injection layer.

18. The organic light emitting diode according to claim 4, wherein the first compound is one of compounds in Formula 2:

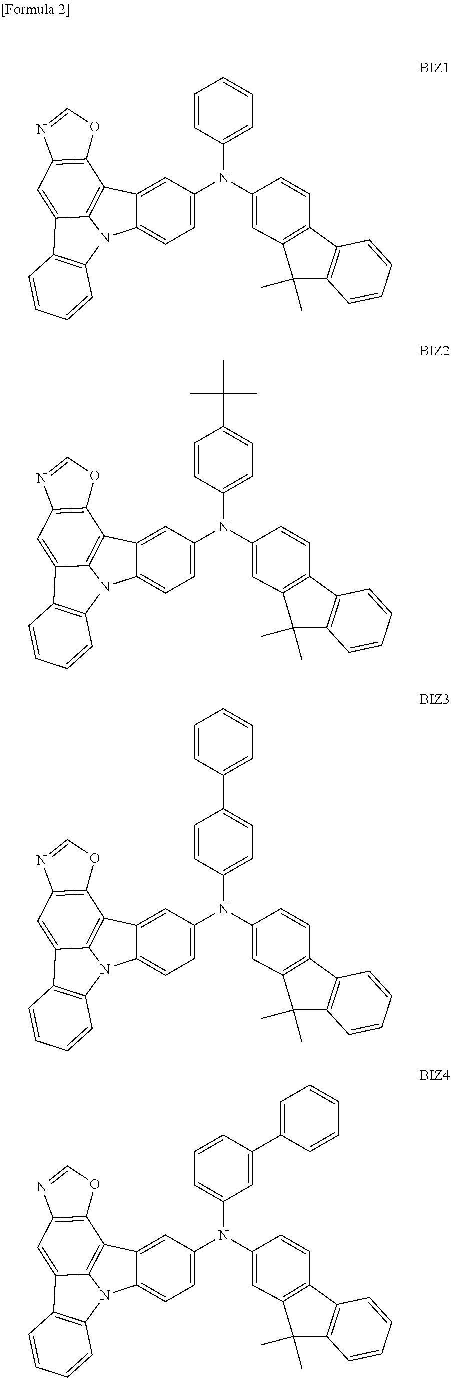

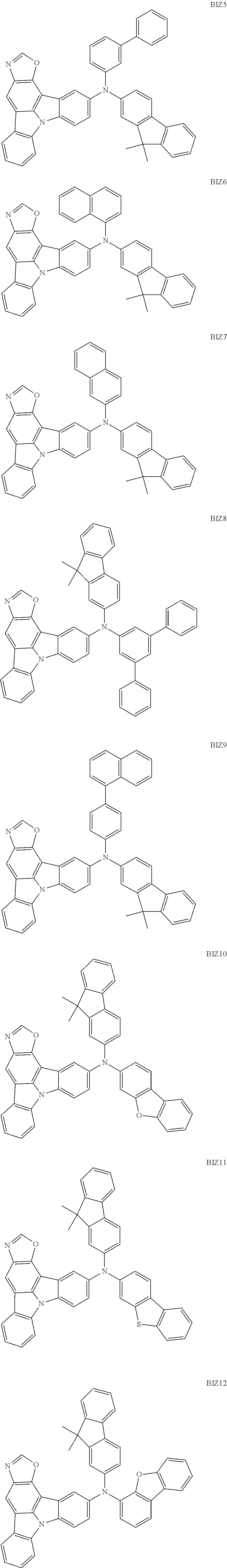

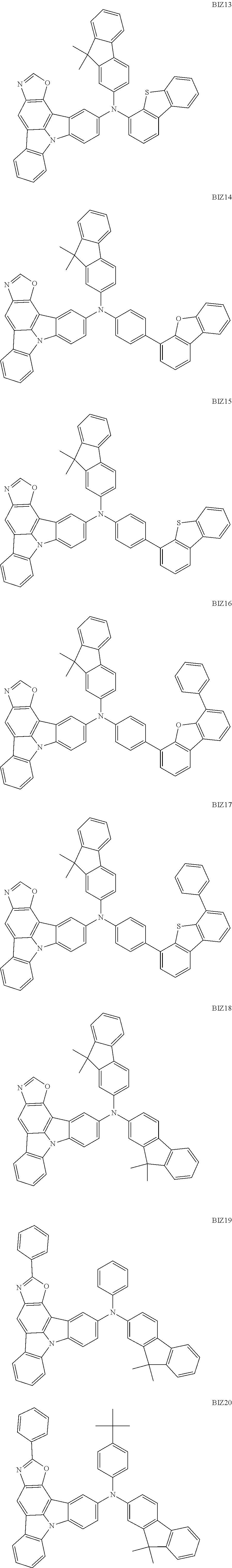

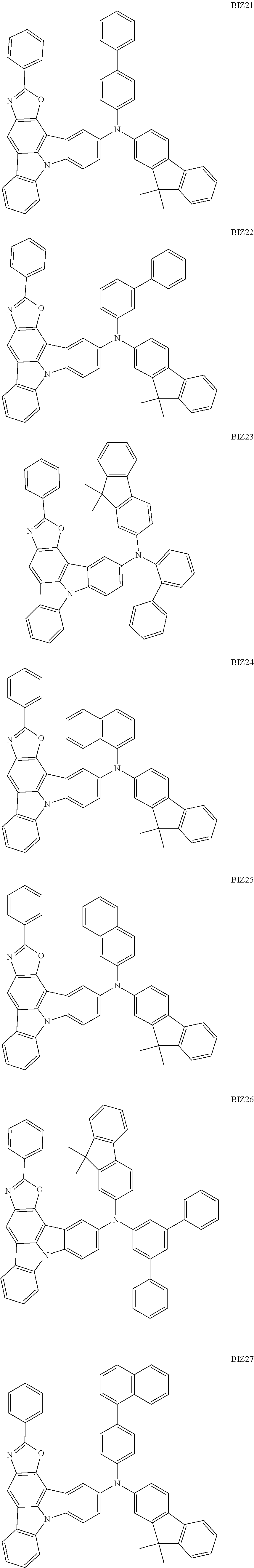

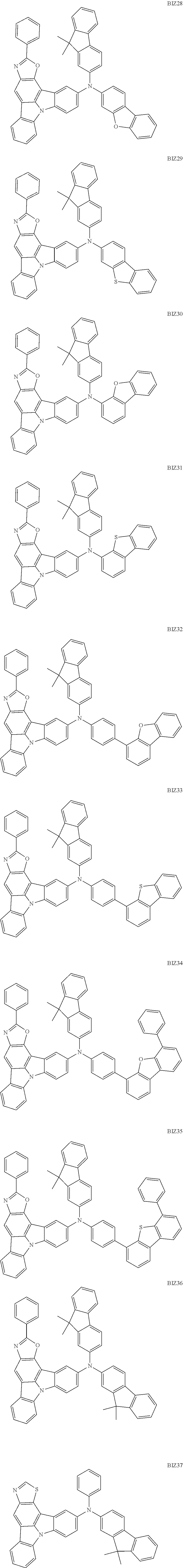

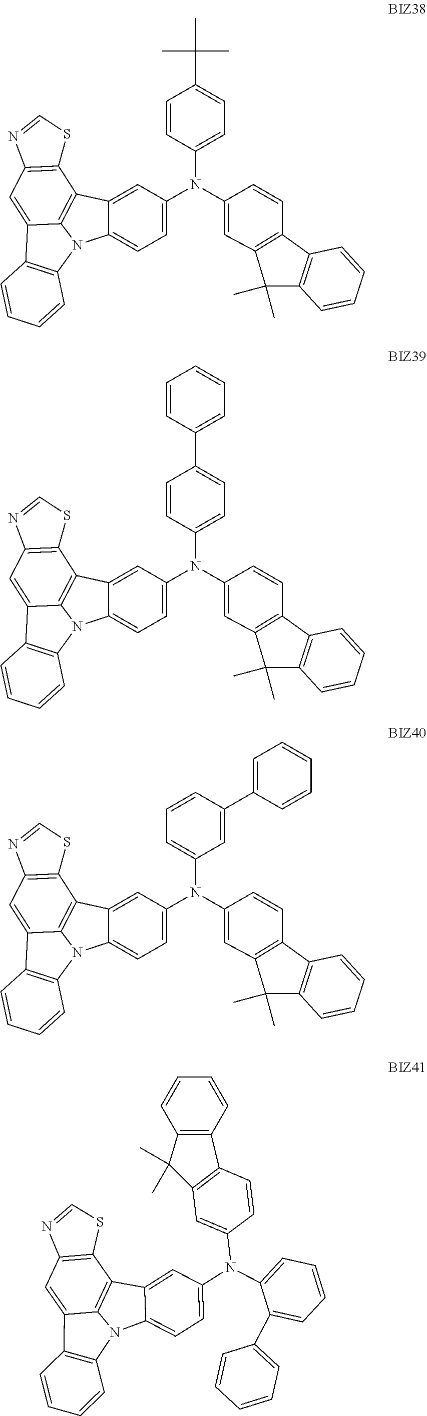

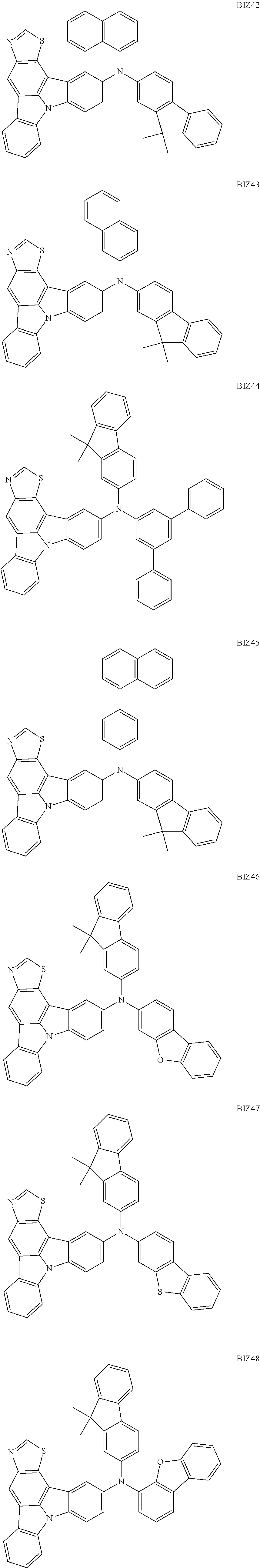

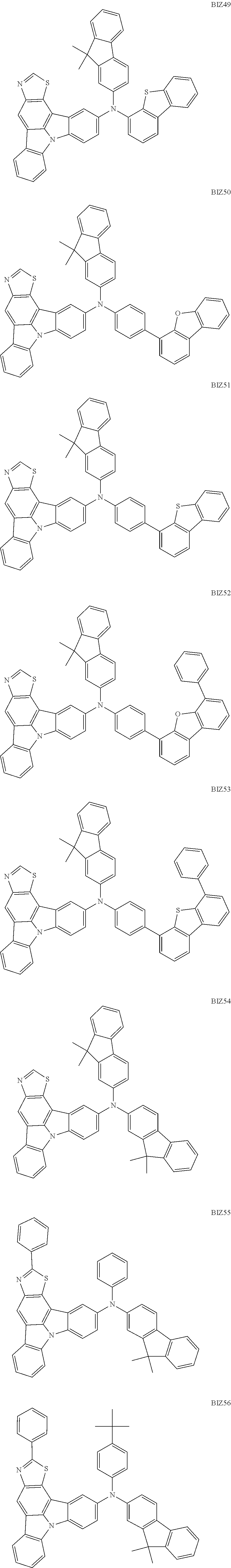

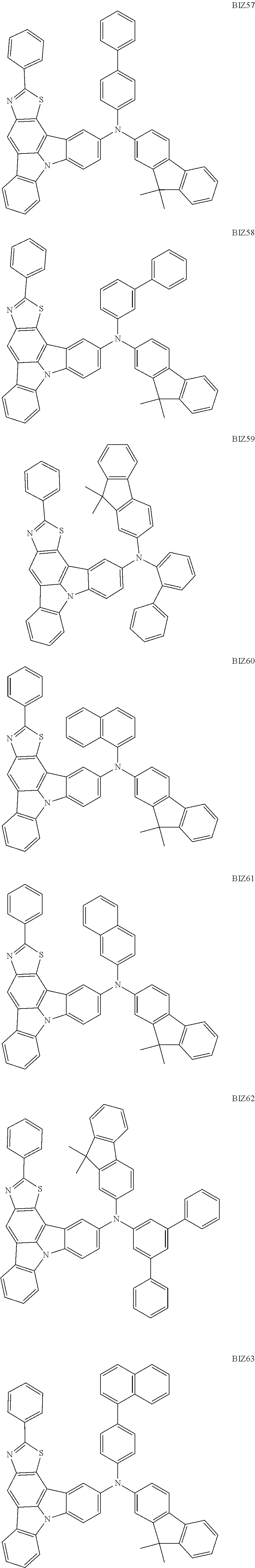

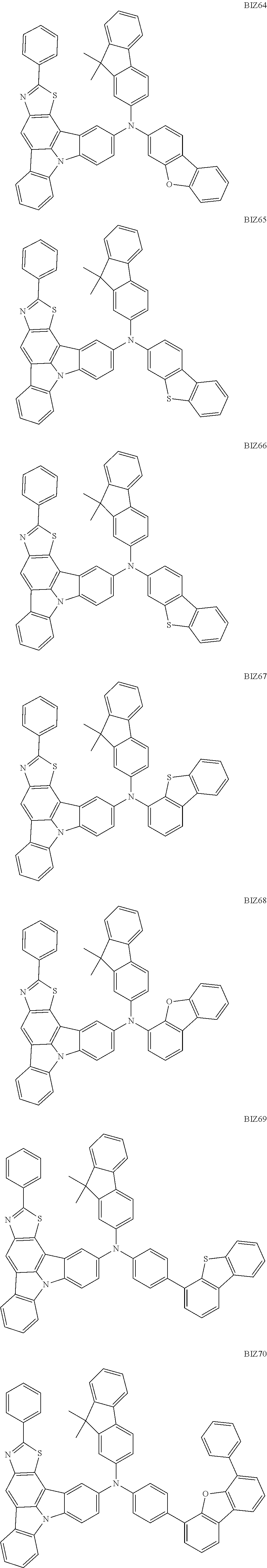

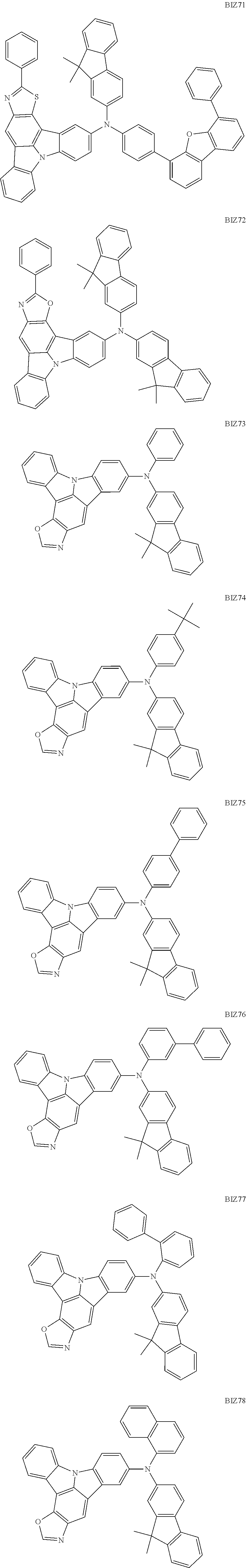

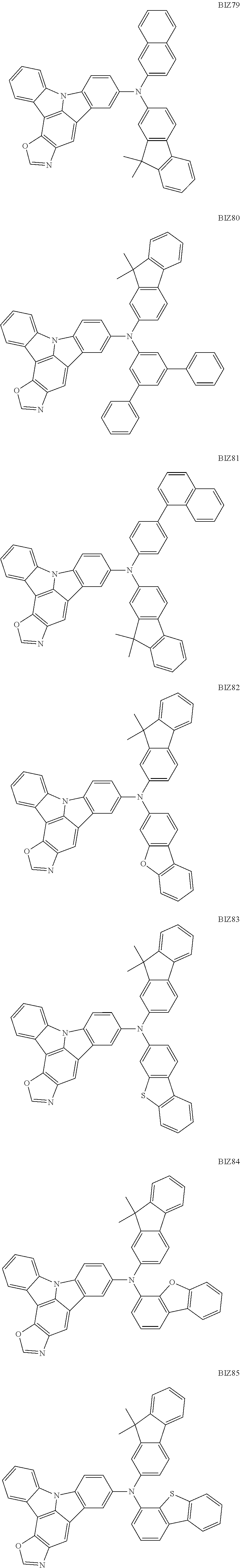

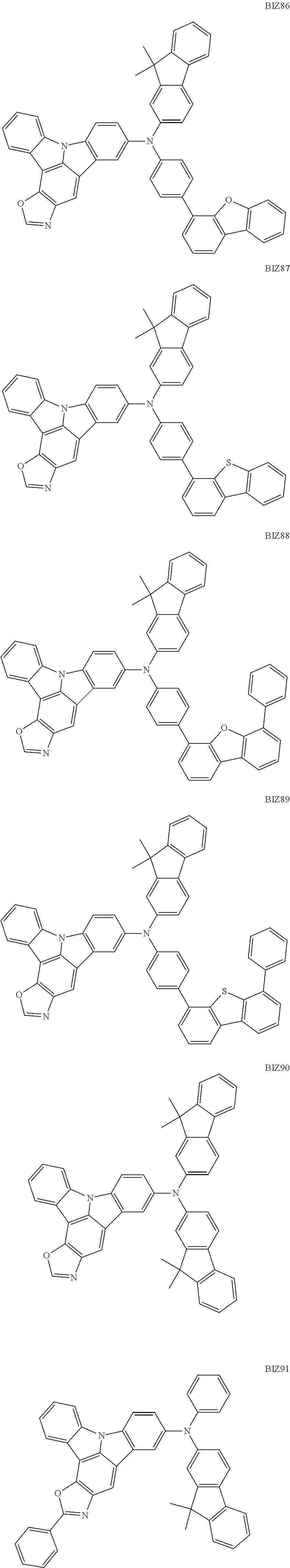

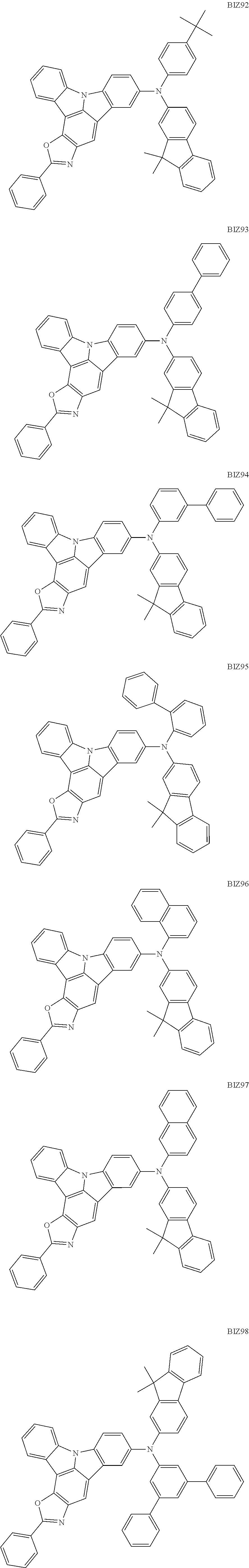

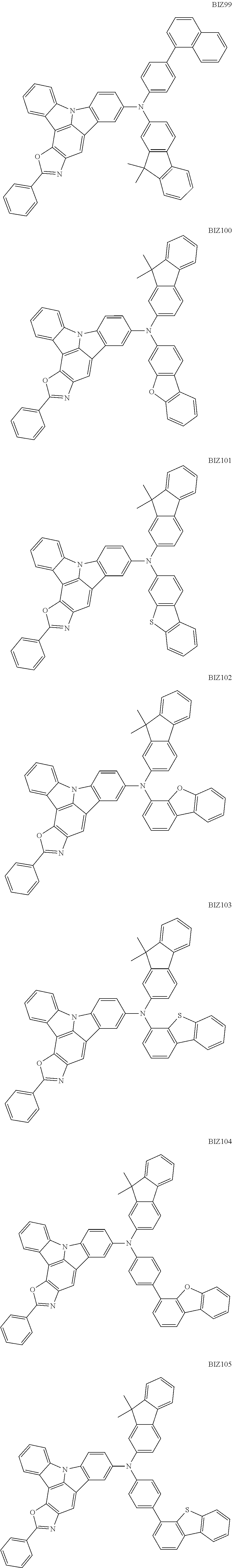

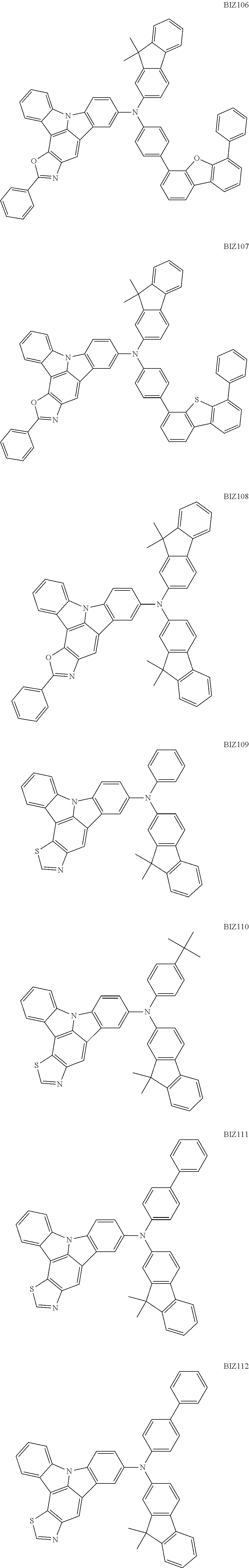

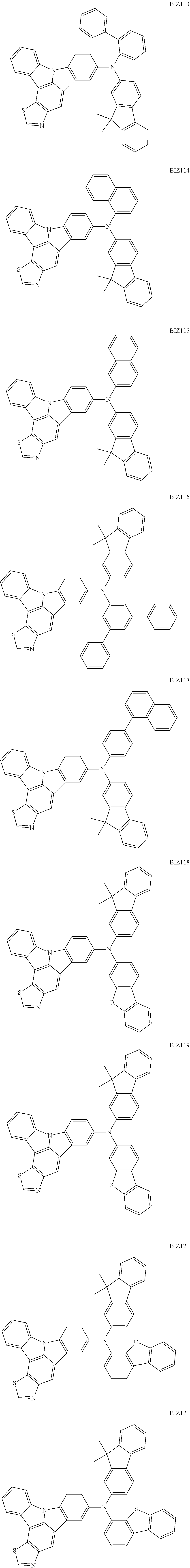

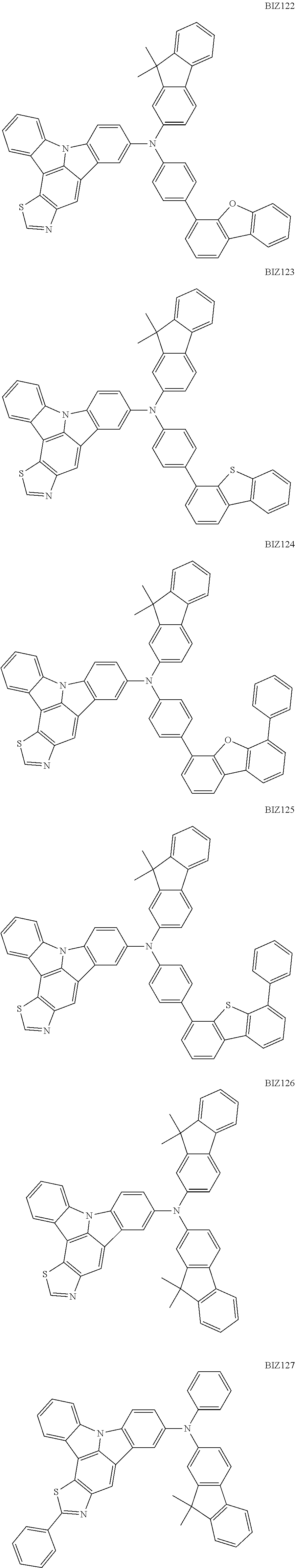

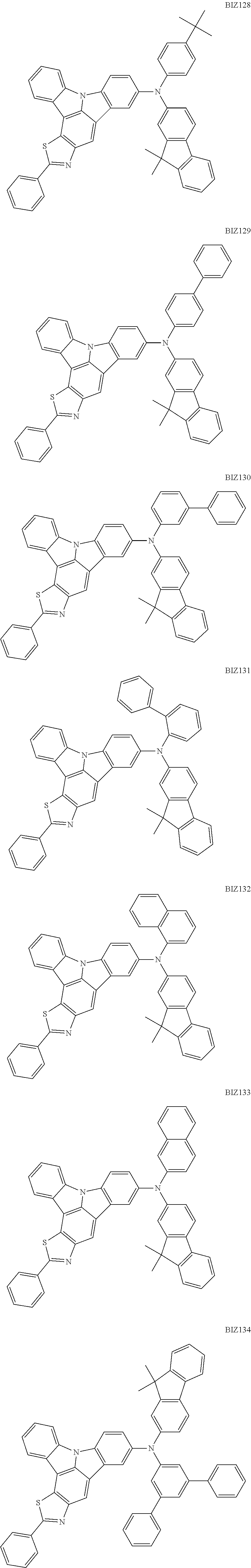

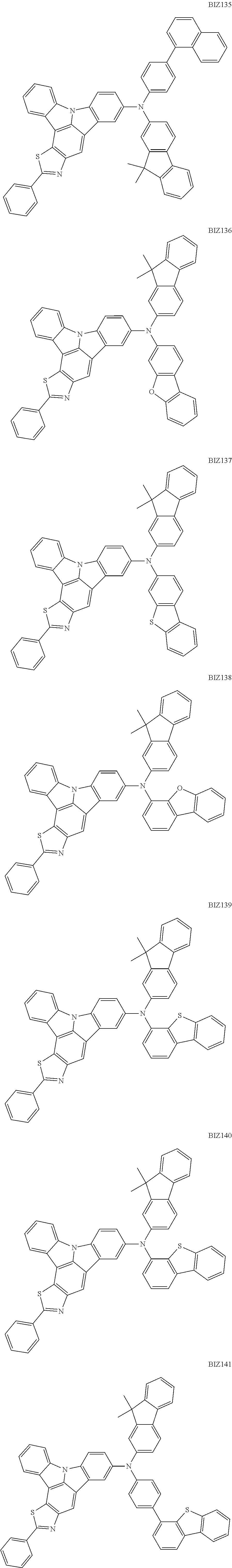

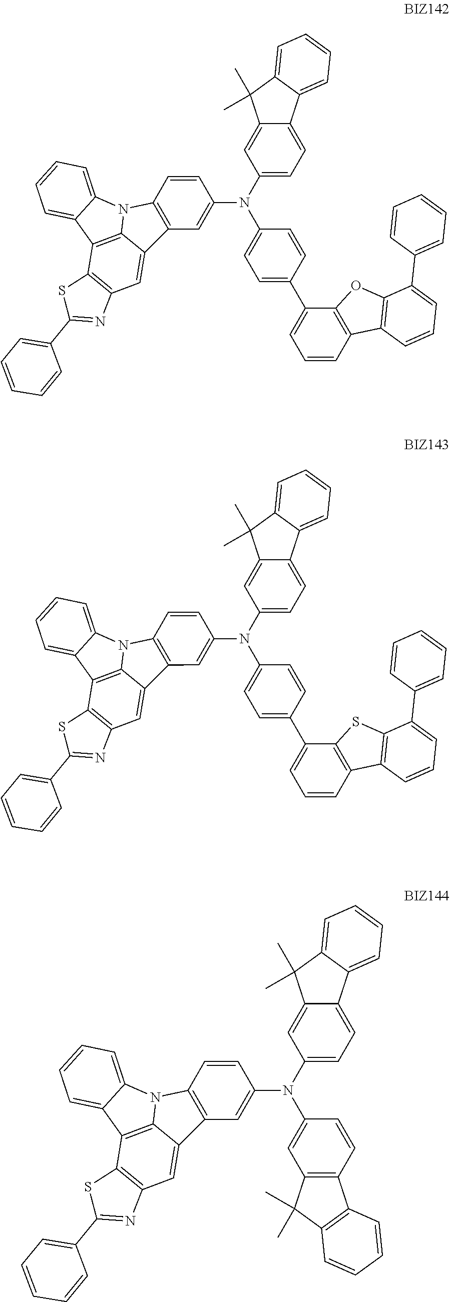 [SMILES: CC1(C)c2ccccc2-c2ccc(N(c3ccc(-c4cccc5c4oc4c(-c6ccccc6)cccc45)cc3)c3ccc4c(c3)c3cc5nc(-c6ccccc6)sc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5nc(-c6ccccc6)sc5c5c6ccccc6n4c35)cc21 ; CC1(C)c2ccccc2-c2ccc(N(c3ccc4c(c3)C(C)(C)c3ccccc3-4)c3ccc4c(c3)c3cc5nc(-c6ccccc6)sc5c5c6ccccc6n4c35)cc21]

19. An organic light emitting device, comprising:
a substrate;
an organic light emitting diode disposed on the substrate and including a first electrode, a second electrode facing the first electrode, a first emitting part positioned between the first electrode and the second electrode and including a first red emitting material layer, and
an encapsulation covering the organic light emitting diode,
wherein the first red emitting material layer includes the organic compound of claim 1.

20. The organic light emitting device according to claim 19, wherein the first red emitting material layer further includes a second compound represented by Formula 3: 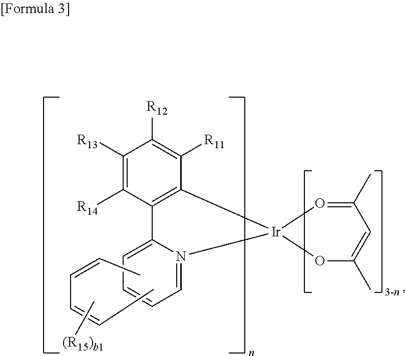
wherein in the Formula 3,
n is an integer of 1 to 3, and b1 is an integer of 0 to 4,
each of R₁₁ to R₁₄ is independently selected from the group consisting of hydrogen, a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group, or adjacent two of R₁₁ to R₁₄ is combined to each other to form an aromatic ring or a heteroaromatic ring, and
R₁₅ is independently selected from the group consisting of a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group.

21. The organic light emitting device according to claim 20, wherein the first red emitting material layer further includes a third compound represented by Formula 5: 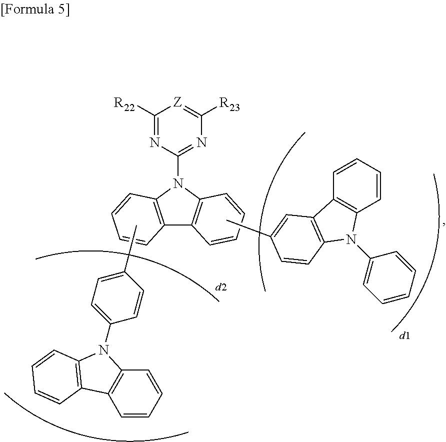
wherein in the Formula 5,
each of d1 and d2 is independently 0 or 1,
Z is N or CR₂₁, and each of R₂₁, R₂₂ and R₂₃ is independently selected from the group consisting of hydrogen, a substituted or unsubstituted C1 to C10 alkyl group, a substituted or unsubstituted C6 to C30 aryl group, a substituted or unsubstituted C3 to C30 cycloalkyl group and a substituted or unsubstituted C5 to C30 heteroaryl group, and
optionally, when Z is CR₂₁, one of a pair of R₂₁ and R₂₂ or a pair of R₂₁ and R₂₃ is combined to each other to form an aromatic ring or a heteroaromatic ring.

22. The organic light emitting device according to claim 19, wherein the organic light emitting diode further includes:
a second emitting part including a first blue emitting material layer and positioned between the first electrode and the first emitting part;
a first charge generation layer between the first emitting part and the second emitting part;
a third emitting part including a second blue emitting material layer and positioned between the first emitting part and the second electrode; and
a second charge generation layer between the first emitting part and the third emitting part.

23. The organic light emitting device according to claim 19, wherein the organic compound is one of compounds in Formula 2: 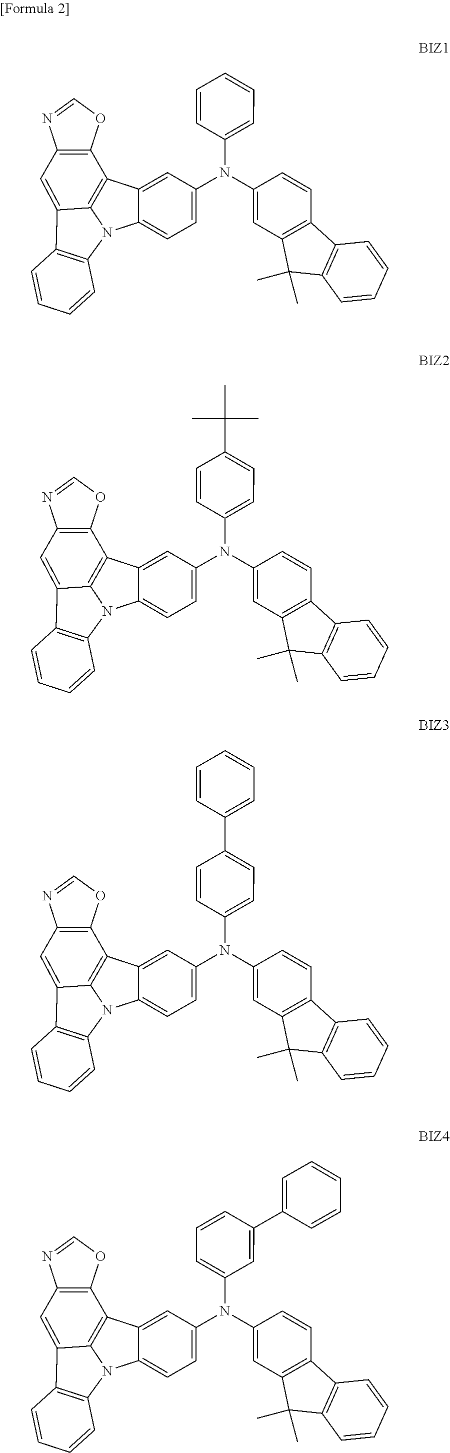 [SMILES: CC1(C)c2ccccc2-c2ccc(N(c3ccccc3)c3ccc4c(c3)c3c5ocnc5cc5c6ccccc6n4c53)cc21 ; CC(C)(C)c1ccc(N(c2ccc3c(c2)C(C)(C)c2ccccc2-3)c2ccc3c(c2)c2c4ocnc4cc4c5ccccc5n3c42)cc1 ; CC1(C)c2ccccc2-c2ccc(N(c3ccc(-c4ccccc4)cc3)c3ccc4c(c3)c3c5ocnc5cc5c6ccccc6n4c53)cc21 ; CC1(C)c2ccccc2-c2ccc(N(c3cccc(-c4ccccc4)c3)c3ccc4c(c3)c3c5ocnc5cc5c6ccccc6n4c53)cc21] 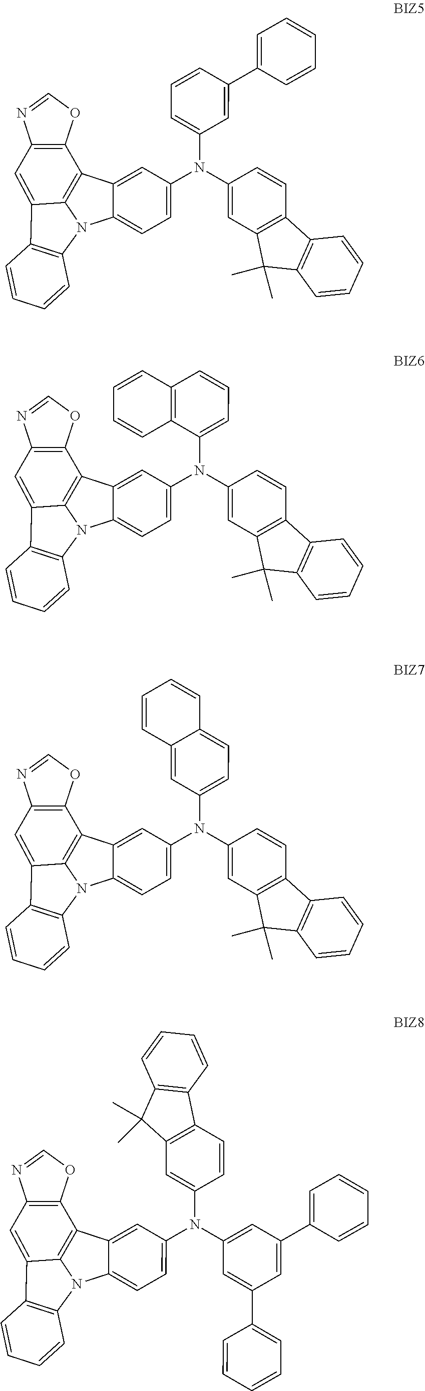 [SMILES: CC1(C)c2ccccc2-c2ccc(N(c3cccc(-c4ccccc4)c3)c3ccc4c(c3)c3c5ocnc5cc5c6ccccc6n4c53)cc21 ; CC1(C)c2ccccc2-c2ccc(N(c3ccc4c(c3)c3c5ocnc5cc5c6ccccc6n4c53)c3cccc4ccccc34)cc21 ; CC1(C)c2ccccc2-c2ccc(N(c3ccc4ccccc4c3)c3ccc4c(c3)c3c5ocnc5cc5c6ccccc6n4c53)cc21 ; CC1(C)c2ccccc2-c2ccc(N(c3cc(-c4ccccc4)cc(-c4ccccc4)c3)c3ccc4c(c3)c3c5ocnc5cc5c6ccccc6n4c53)cc21] 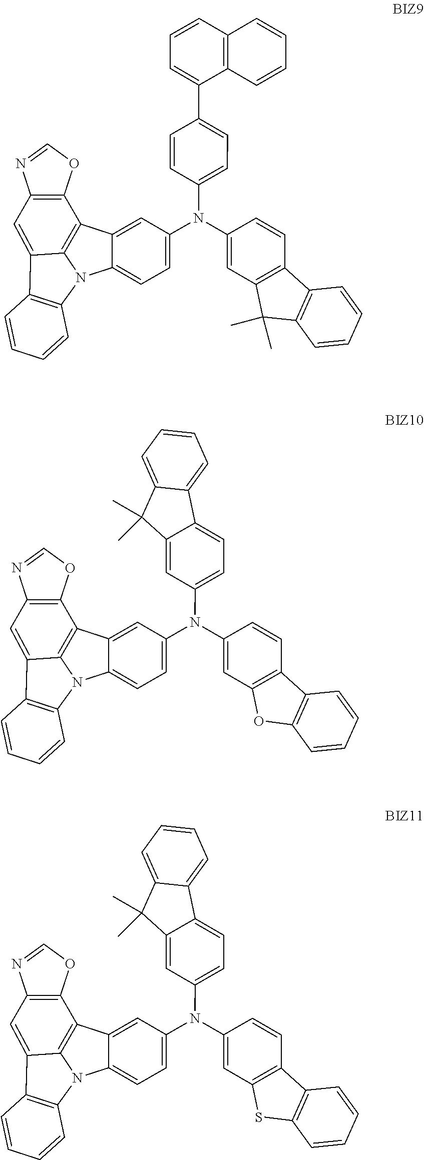 [SMILES: CC1(C)c2ccccc2-c2ccc(N(c3ccc(-c4cccc5ccccc45)cc3)c3ccc4c(c3)c3c5ocnc5cc5c6ccccc6n4c53)cc21 ; CC1(C)c2ccccc2-c2ccc(N(c3ccc4c(c3)oc3ccccc34)c3ccc4c(c3)c3c5ocnc5cc5c6ccccc6n4c53)cc21 ; CC1(C)c2ccccc2-c2ccc(N(c3ccc4c(c3)sc3ccccc34)c3ccc4c(c3)c3c5ocnc5cc5c6ccccc6n4c53)cc21] 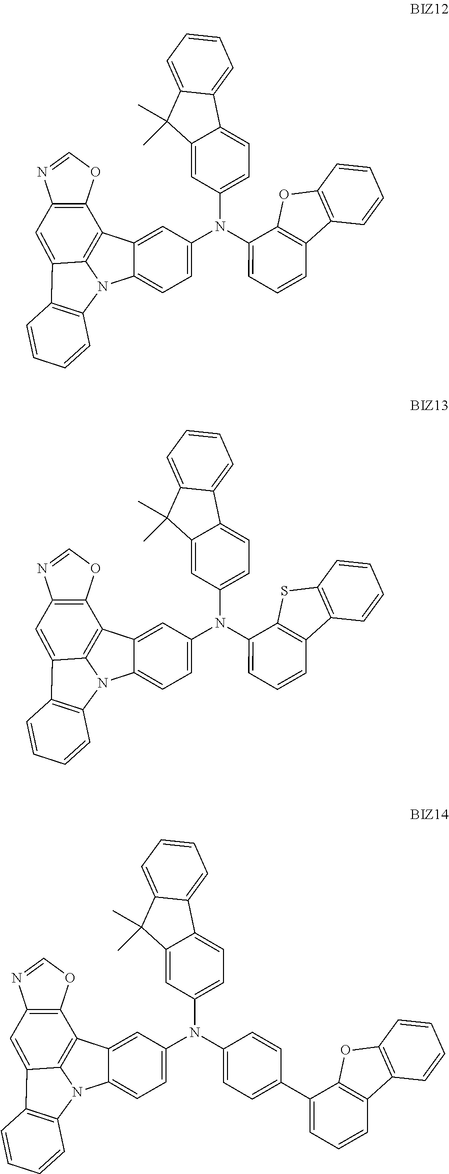 [SMILES: CC1(C)c2ccccc2-c2ccc(N(c3ccc4c(c3)c3c5ocnc5cc5c6ccccc6n4c53)c3cccc4c3oc3ccccc34)cc21 ; CC1(C)c2ccccc2-c2ccc(N(c3ccc4c(c3)c3c5ocnc5cc5c6ccccc6n4c53)c3cccc4c3sc3ccccc34)cc21 ; CC1(C)c2ccccc2-c2ccc(N(c3ccc(-c4cccc5c4oc4ccccc45)cc3)c3ccc4c(c3)c3c5ocnc5cc5c6ccccc6n4c53)cc21] 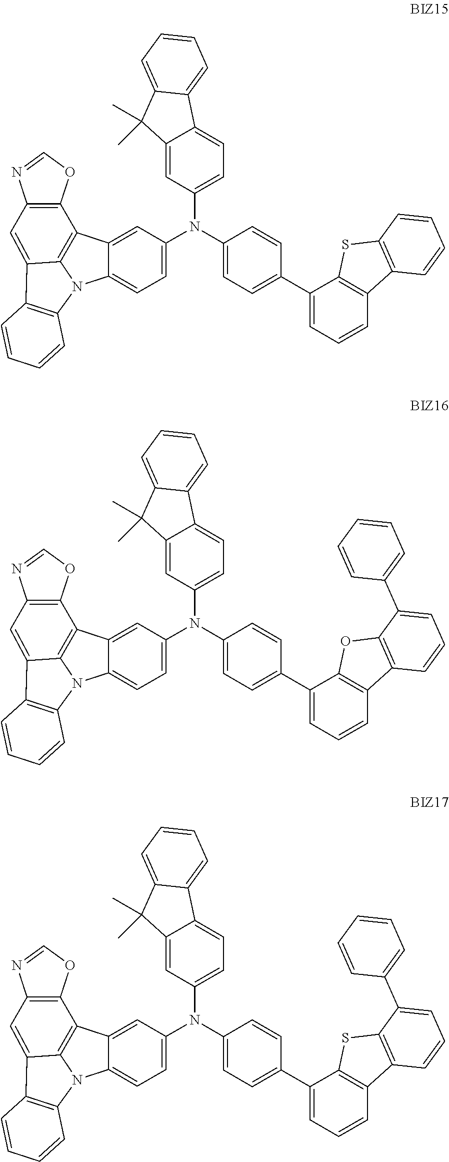 [SMILES: CC1(C)c2ccccc2-c2ccc(N(c3ccc(-c4cccc5c4sc4ccccc45)cc3)c3ccc4c(c3)c3c5ocnc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5ocnc5cc5c6ccccc6n4c53)cc21 ; CC1(C)c2ccccc2-c2ccc(N(c3ccc(-c4cccc5c4sc4c(-c6ccccc6)cccc45)cc3)c3ccc4c(c3)c3c5ocnc5cc5c6ccccc6n4c53)cc21] 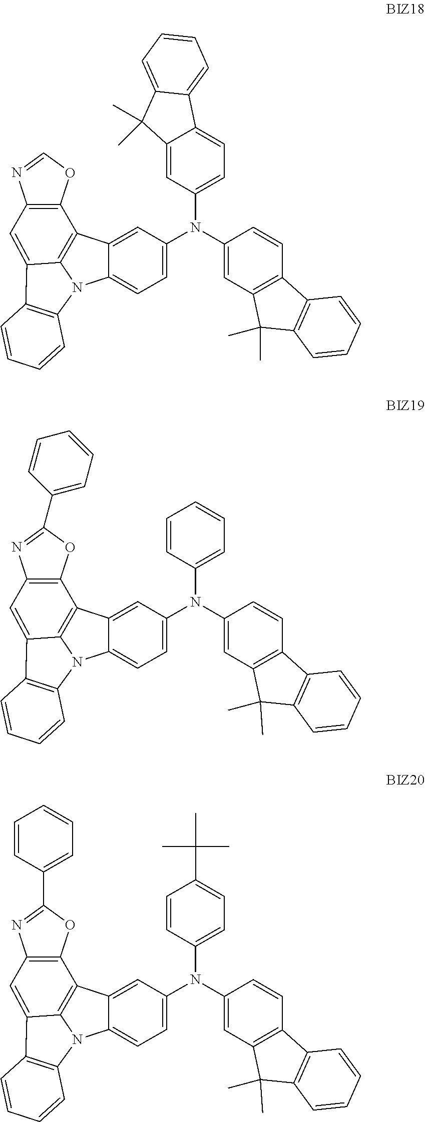 [SMILES: CC1(C)c2ccccc2-c2ccc(N(c3ccc4c(c3)C(C)(C)c3ccccc3-4)c3ccc4c(c3)c3c5ocnc5cc5c6ccccc6n4c53)cc21 ; CC1(C)c2ccccc2-c2ccc(N(c3ccccc3)c3ccc4c(c3)c3c5oc(-c6ccccc6)nc5cc5c6ccccc6n4c53)cc21 ; CC(C)(C)c1ccc(N(c2ccc3c(c2)C(C)(C)c2ccccc2-3)c2ccc3c(c2)c2c4oc(-c5ccccc5)nc4cc4c5ccccc5n3c42)cc1] 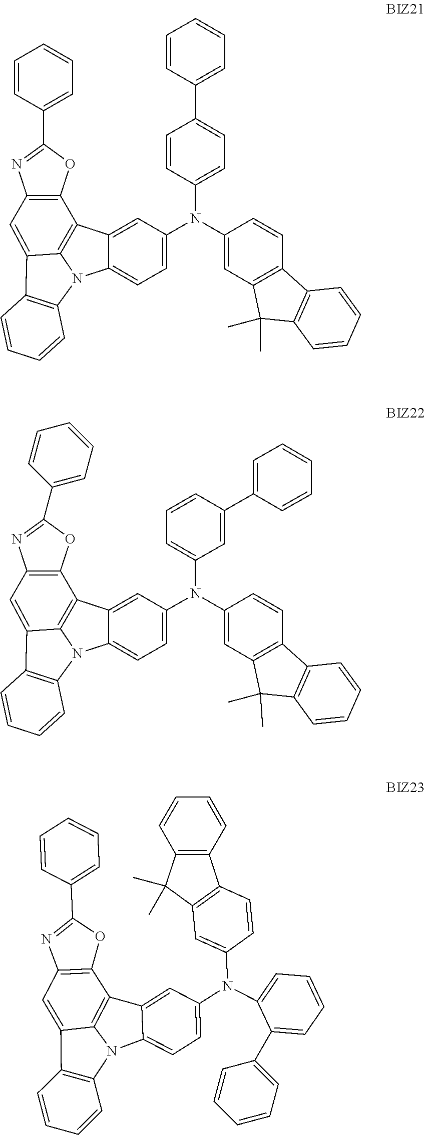 [SMILES: CC1(C)c2ccccc2-c2ccc(N(c3ccc(-c4ccccc4)cc3)c3ccc4c(c3)c3c5oc(-c6ccccc6)nc5cc5c6ccccc6n4c53)cc21 ; CC1(C)c2ccccc2-c2ccc(N(c3cccc(-c4ccccc4)c3)c3ccc4c(c3)c3c5oc(-c6ccccc6)nc5cc5c6ccccc6n4c53)cc21 ; CC1(C)c2ccccc2-c2ccc(N(c3ccc4c(c3)c3c5oc(-c6ccccc6)nc5cc5c6ccccc6n4c53)c3ccccc3-c3ccccc3)cc21] 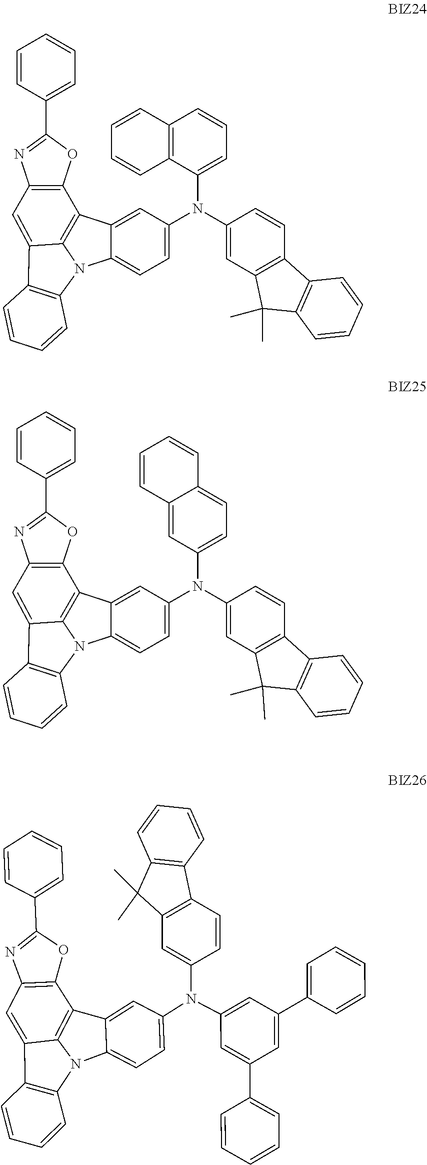 [SMILES: CC1(C)c2ccccc2-c2ccc(N(c3ccc4c(c3)c3c5oc(-c6ccccc6)nc5cc5c6ccccc6n4c53)c3cccc4ccccc34)cc21 ; CC1(C)c2ccccc2-c2ccc(N(c3ccc4ccccc4c3)c3ccc4c(c3)c3c5oc(-c6ccccc6)nc5cc5c6ccccc6n4c53)cc21 ; CC1(C)c2ccccc2-c2ccc(N(c3cc(-c4ccccc4)cc(-c4ccccc4)c3)c3ccc4c(c3)c3c5oc(-c6ccccc6)nc5cc5c6ccccc6n4c53)cc21] 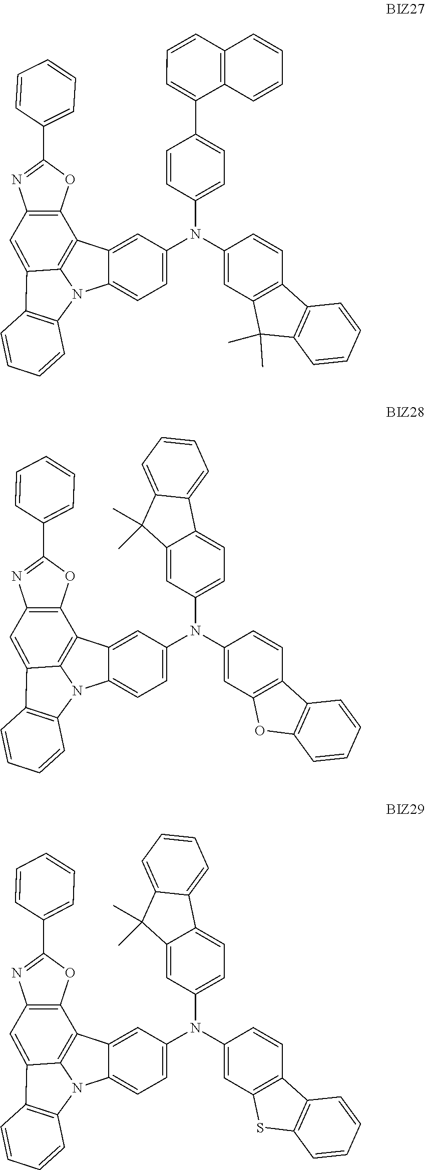 [SMILES: CC1(C)c2ccccc2-c2ccc(N(c3ccc(-c4cccc5ccccc45)cc3)c3ccc4c(c3)c3c5oc(-c6ccccc6)nc5cc5c6ccccc6n4c53)cc21 ; CC1(C)c2ccccc2-c2ccc(N(c3ccc4c(c3)oc3ccccc34)c3ccc4c(c3)c3c5oc(-c6ccccc6)nc5cc5c6ccccc6n4c53)cc21 ; CC1(C)c2ccccc2-c2ccc(N(c3ccc4c(c3)sc3ccccc34)c3ccc4c(c3)c3c5oc(-c6ccccc6)nc5cc5c6ccccc6n4c53)cc21] 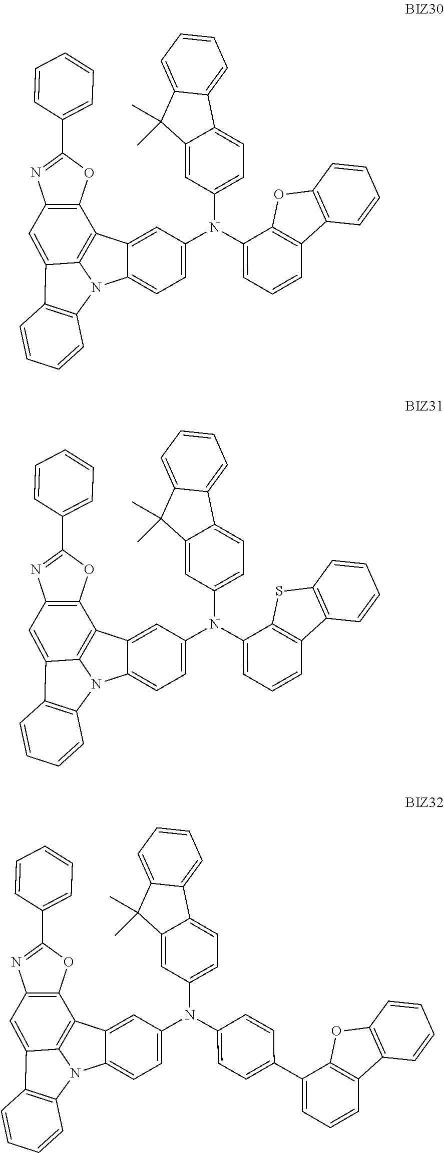 [SMILES: CC1(C)c2ccccc2-c2ccc(N(c3ccc4c(c3)c3c5oc(-c6ccccc6)nc5cc5c6ccccc6n4c53)c3cccc4c3oc3ccccc34)cc21 ; CC1(C)c2ccccc2-c2ccc(N(c3ccc4c(c3)c3c5oc(-c6ccccc6)nc5cc5c6ccccc6n4c53)c3cccc4c3sc3ccccc34)cc21 ; CC1(C)c2ccccc2-c2ccc(N(c3ccc(-c4cccc5c4oc4ccccc45)cc3)c3ccc4c(c3)c3c5oc(-c6ccccc6)nc5cc5c6ccccc6n4c53)cc21] 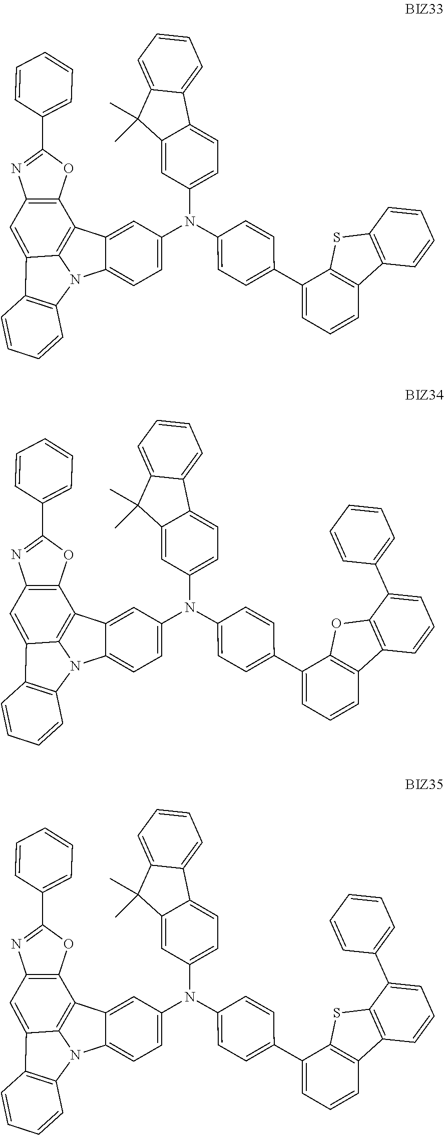 [SMILES: CC1(C)c2ccccc2-c2ccc(N(c3ccc(-c4cccc5c4sc4ccccc45)cc3)c3ccc4c(c3)c3c5oc(-c6ccccc6)nc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5oc(-c6ccccc6)nc5cc5c6ccccc6n4c53)cc21 ; CC1(C)c2ccccc2-c2ccc(N(c3ccc(-c4cccc5c4sc4c(-c6ccccc6)cccc45)cc3)c3ccc4c(c3)c3c5oc(-c6ccccc6)nc5cc5c6ccccc6n4c53)cc21] 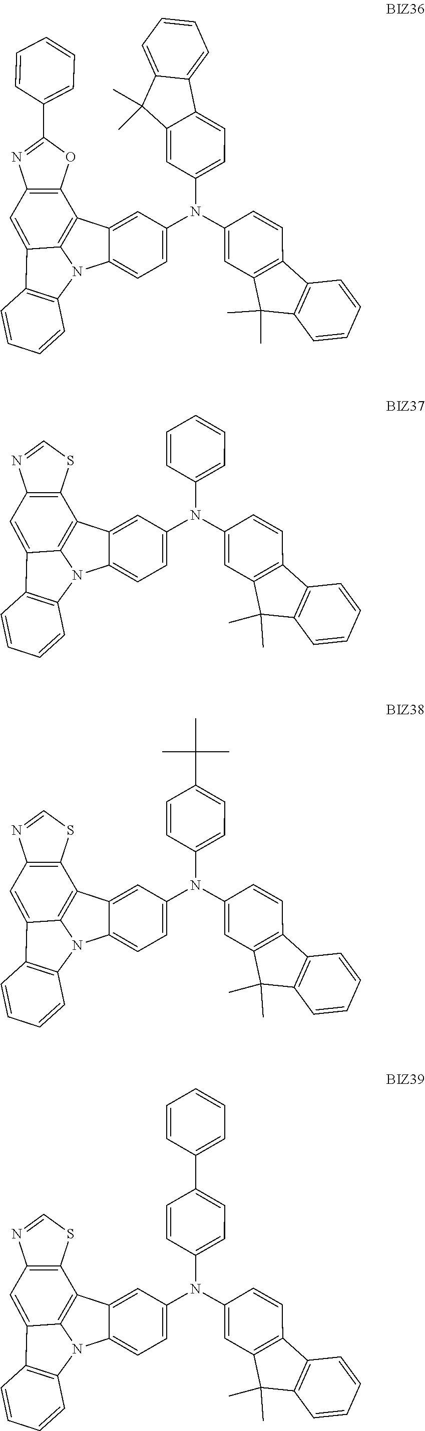 [SMILES: CC1(C)c2ccccc2-c2ccc(N(c3ccc4c(c3)C(C)(C)c3ccccc3-4)c3ccc4c(c3)c3c5oc(-c6ccccc6)nc5cc5c6ccccc6n4c53)cc21 ; CC1(C)c2ccccc2-c2ccc(N(c3ccccc3)c3ccc4c(c3)c3c5scnc5cc5c6ccccc6n4c53)cc21 ; CC(C)(C)c1ccc(N(c2ccc3c(c2)C(C)(C)c2ccccc2-3)c2ccc3c(c2)c2c4scnc4cc4c5ccccc5n3c42)cc1 ; CC1(C)c2ccccc2-c2ccc(N(c3ccc(-c4ccccc4)cc3)c3ccc4c(c3)c3c5scnc5cc5c6ccccc6n4c53)cc21] 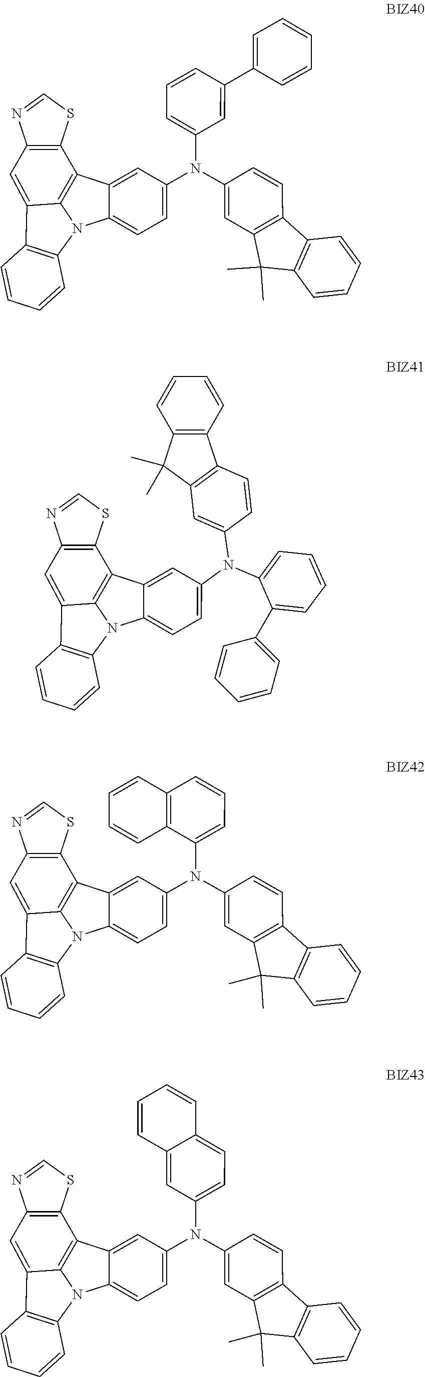 [SMILES: CC1(C)c2ccccc2-c2ccc(N(c3cccc(-c4ccccc4)c3)c3ccc4c(c3)c3c5scnc5cc5c6ccccc6n4c53)cc21 ; CC1(C)c2ccccc2-c2ccc(N(c3ccc4c(c3)c3c5scnc5cc5c6ccccc6n4c53)c3ccccc3-c3ccccc3)cc21 ; CC1(C)c2ccccc2-c2ccc(N(c3ccc4c(c3)c3c5scnc5cc5c6ccccc6n4c53)c3cccc4ccccc34)cc21 ; CC1(C)c2ccccc2-c2ccc(N(c3ccc4ccccc4c3)c3ccc4c(c3)c3c5scnc5cc5c6ccccc6n4c53)cc21] 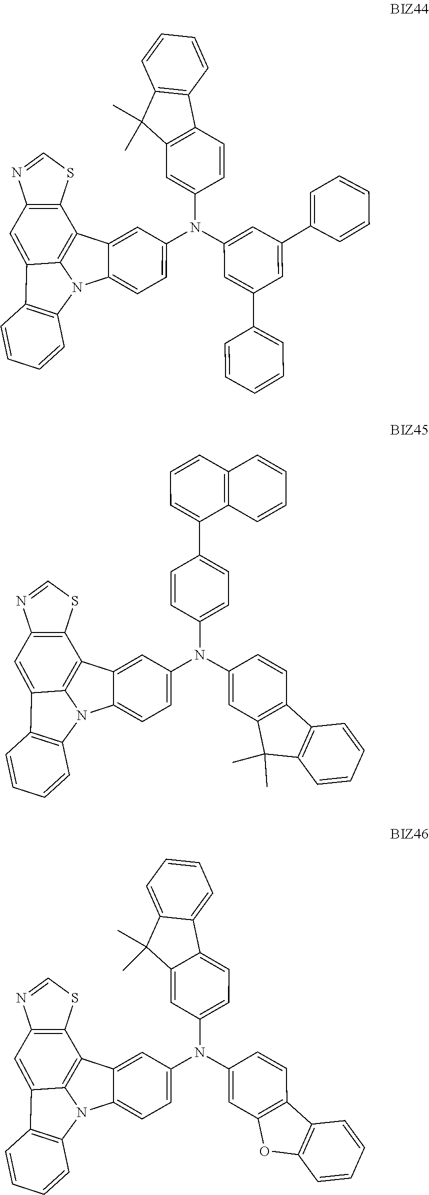 [SMILES: CC1(C)c2ccccc2-c2ccc(N(c3cc(-c4ccccc4)cc(-c4ccccc4)c3)c3ccc4c(c3)c3c5scnc5cc5c6ccccc6n4c53)cc21 ; CC1(C)c2ccccc2-c2ccc(N(c3ccc(-c4cccc5ccccc45)cc3)c3ccc4c(c3)c3c5scnc5cc5c6ccccc6n4c53)cc21 ; CC1(C)c2ccccc2-c2ccc(N(c3ccc4c(c3)oc3ccccc34)c3ccc4c(c3)c3c5scnc5cc5c6ccccc6n4c53)cc21] 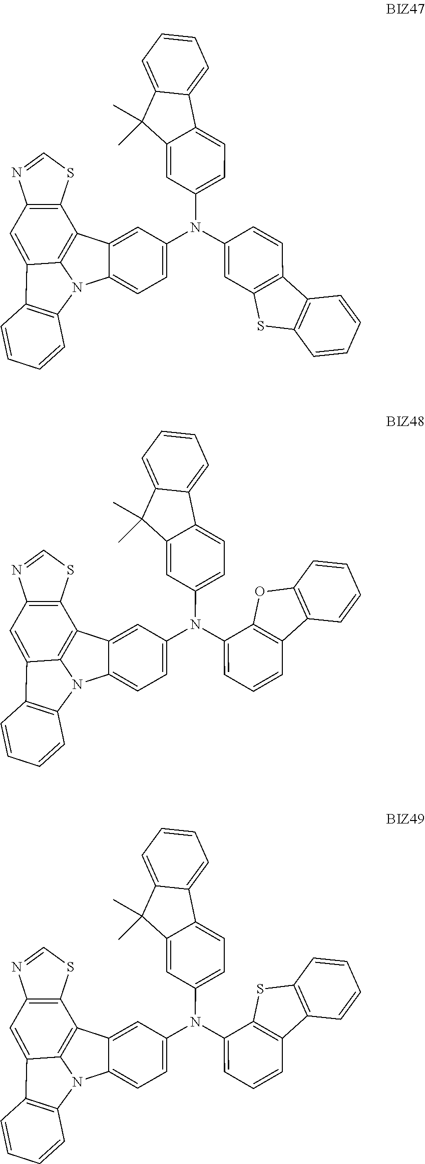 [SMILES: CC1(C)c2ccccc2-c2ccc(N(c3ccc4c(c3)sc3ccccc34)c3ccc4c(c3)c3c5scnc5cc5c6ccccc6n4c53)cc21 ; CC1(C)c2ccccc2-c2ccc(N(c3ccc4c(c3)c3c5scnc5cc5c6ccccc6n4c53)c3cccc4c3oc3ccccc34)cc21 ; CC1(C)c2ccccc2-c2ccc(N(c3ccc4c(c3)c3c5scnc5cc5c6ccccc6n4c53)c3cccc4c3sc3ccccc34)cc21] 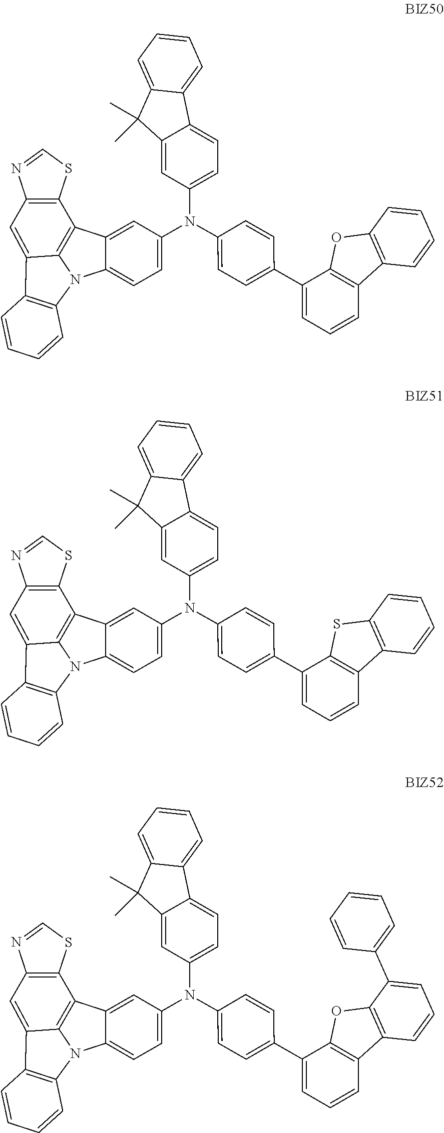 [SMILES: CC1(C)c2ccccc2-c2ccc(N(c3ccc(-c4cccc5c4oc4ccccc45)cc3)c3ccc4c(c3)c3c5scnc5cc5c6ccccc6n4c53)cc21 ; CC1(C)c2ccccc2-c2ccc(N(c3ccc(-c4cccc5c4sc4ccccc45)cc3)c3ccc4c(c3)c3c5scnc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5scnc5cc5c6ccccc6n4c53)cc21] 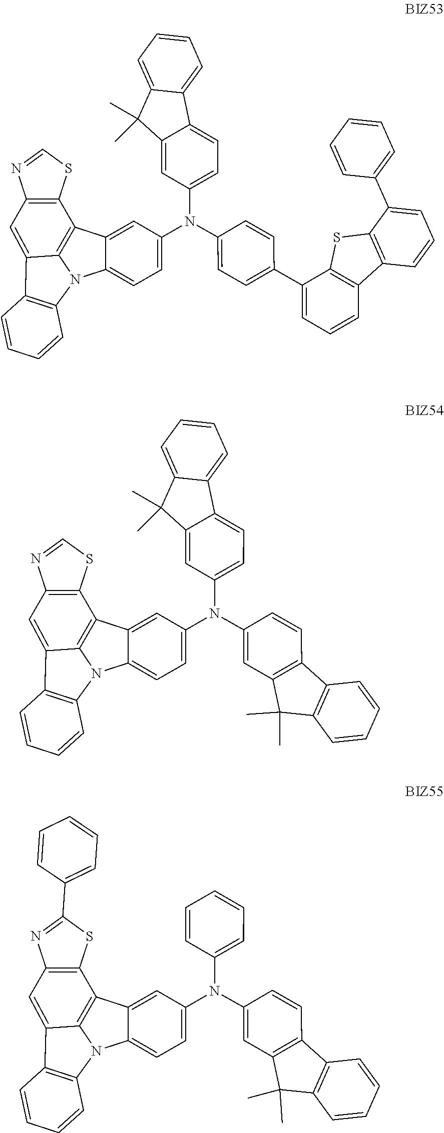 [SMILES: CC1(C)c2ccccc2-c2ccc(N(c3ccc(-c4cccc5c4sc4c(-c6ccccc6)cccc45)cc3)c3ccc4c(c3)c3c5scnc5cc5c6ccccc6n4c53)cc21 ; CC1(C)c2ccccc2-c2ccc(N(c3ccc4c(c3)C(C)(C)c3ccccc3-4)c3ccc4c(c3)c3c5scnc5cc5c6ccccc6n4c53)cc21 ; CC1(C)c2ccccc2-c2ccc(N(c3ccccc3)c3ccc4c(c3)c3c5sc(-c6ccccc6)nc5cc5c6ccccc6n4c53)cc21] 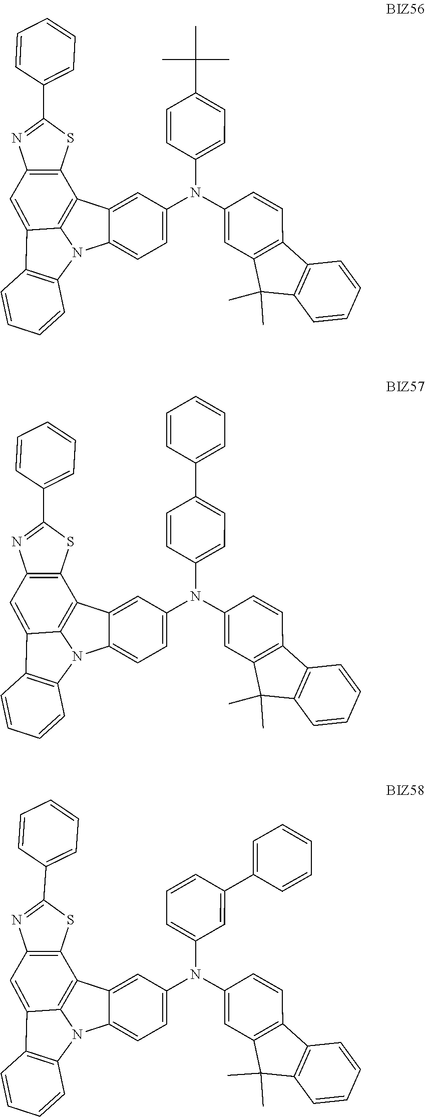 [SMILES: CC(C)(C)c1ccc(N(c2ccc3c(c2)C(C)(C)c2ccccc2-3)c2ccc3c(c2)c2c4sc(-c5ccccc5)nc4cc4c5ccccc5n3c42)cc1 ; CC1(C)c2ccccc2-c2ccc(N(c3ccc(-c4ccccc4)cc3)c3ccc4c(c3)c3c5sc(-c6ccccc6)nc5cc5c6ccccc6n4c53)cc21 ; CC1(C)c2ccccc2-c2ccc(N(c3cccc(-c4ccccc4)c3)c3ccc4c(c3)c3c5sc(-c6ccccc6)nc5cc5c6ccccc6n4c53)cc21] 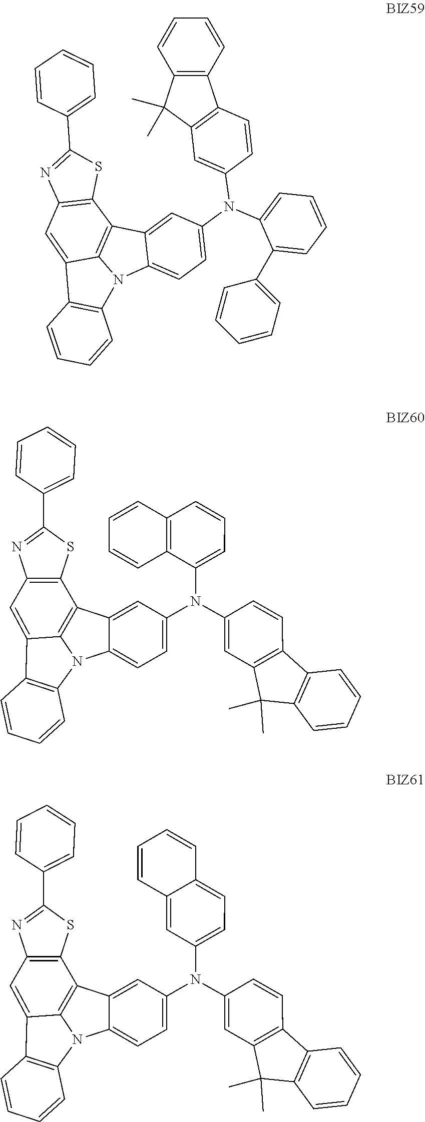 [SMILES: CC1(C)c2ccccc2-c2ccc(N(c3ccc4c(c3)c3c5sc(-c6ccccc6)nc5cc5c6ccccc6n4c53)c3ccccc3-c3ccccc3)cc21 ; CC1(C)c2ccccc2-c2ccc(N(c3ccc4c(c3)c3c5sc(-c6ccccc6)nc5cc5c6ccccc6n4c53)c3cccc4ccccc34)cc21 ; CC1(C)c2ccccc2-c2ccc(N(c3ccc4ccccc4c3)c3ccc4c(c3)c3c5sc(-c6ccccc6)nc5cc5c6ccccc6n4c53)cc21] 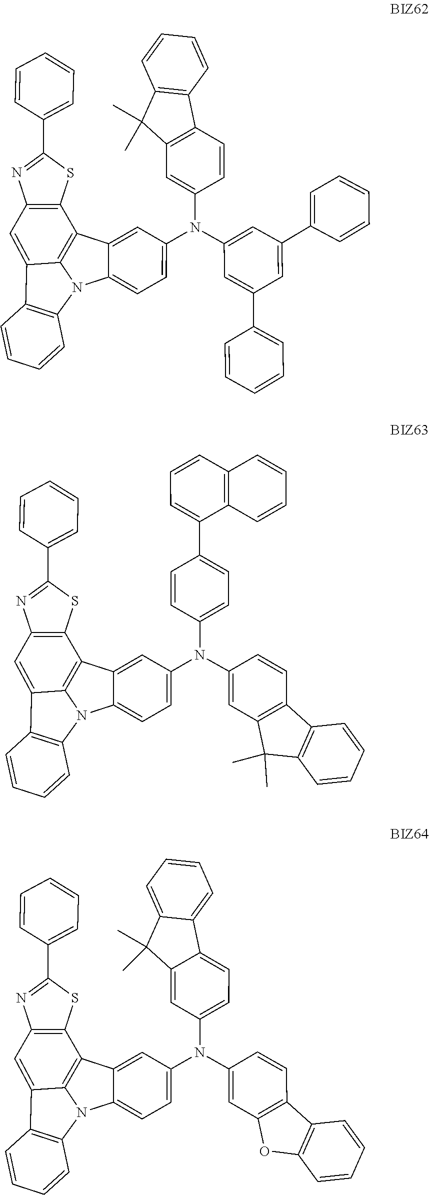 [SMILES: CC1(C)c2ccccc2-c2ccc(N(c3cc(-c4ccccc4)cc(-c4ccccc4)c3)c3ccc4c(c3)c3c5sc(-c6ccccc6)nc5cc5c6ccccc6n4c53)cc21 ; CC1(C)c2ccccc2-c2ccc(N(c3ccc(-c4cccc5ccccc45)cc3)c3ccc4c(c3)c3c5sc(-c6ccccc6)nc5cc5c6ccccc6n4c53)cc21 ; CC1(C)c2ccccc2-c2ccc(N(c3ccc4c(c3)oc3ccccc34)c3ccc4c(c3)c3c5sc(-c6ccccc6)nc5cc5c6ccccc6n4c53)cc21] 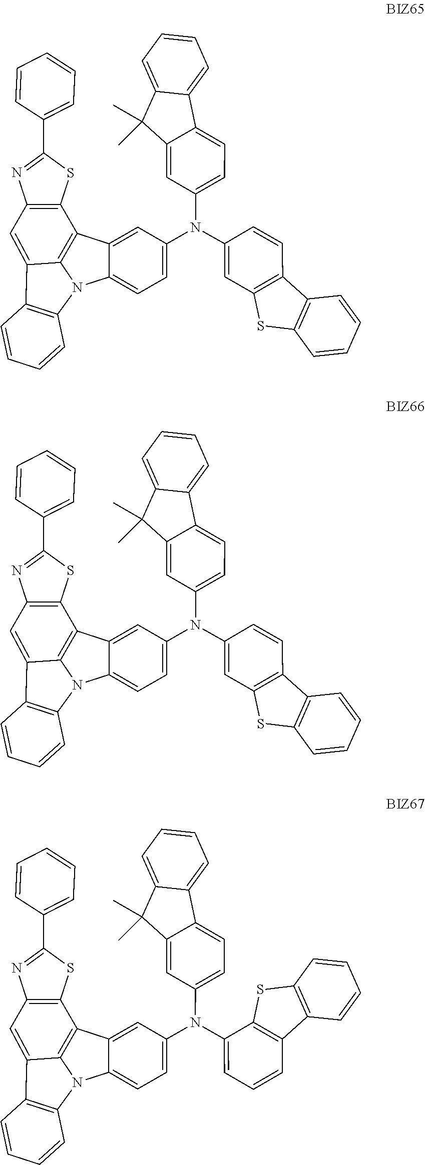 [SMILES: CC1(C)c2ccccc2-c2ccc(N(c3ccc4c(c3)sc3ccccc34)c3ccc4c(c3)c3c5sc(-c6ccccc6)nc5cc5c6ccccc6n4c53)cc21 ; CC1(C)c2ccccc2-c2ccc(N(c3ccc4c(c3)sc3ccccc34)c3ccc4c(c3)c3c5sc(-c6ccccc6)nc5cc5c6ccccc6n4c53)cc21 ; CC1(C)c2ccccc2-c2ccc(N(c3ccc4c(c3)c3c5sc(-c6ccccc6)nc5cc5c6ccccc6n4c53)c3cccc4c3sc3ccccc34)cc21] 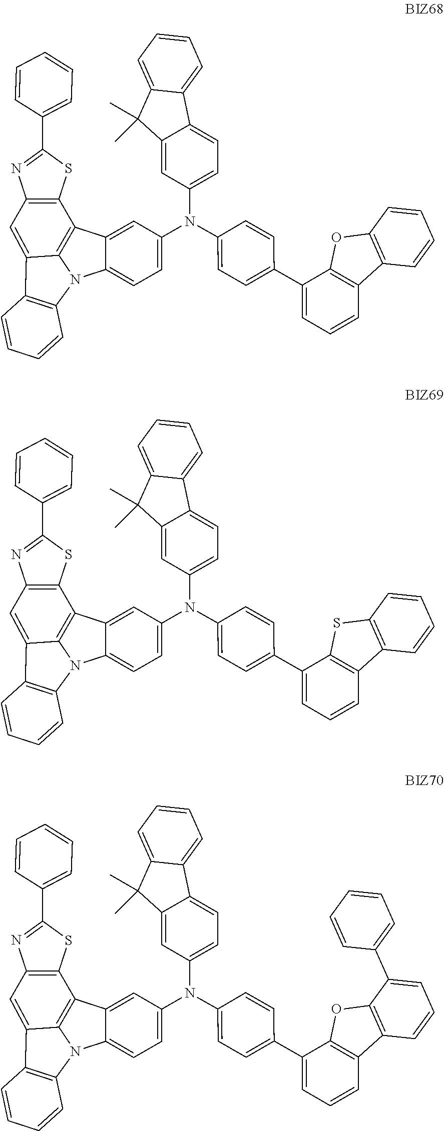 [SMILES: CC1(C)c2ccccc2-c2ccc(N(c3ccc(-c4cccc5c4oc4ccccc45)cc3)c3ccc4c(c3)c3c5sc(-c6ccccc6)nc5cc5c6ccccc6n4c53)cc21 ; CC1(C)c2ccccc2-c2ccc(N(c3ccc(-c4cccc5c4sc4ccccc45)cc3)c3ccc4c(c3)c3c5sc(-c6ccccc6)nc5cc5c6ccccc6n4c53)cc21 ; CC1(C)c2ccccc2-c2ccc(N(c3ccc(-c4cccc5c4oc4c(-c6ccccc6)cccc45)cc3)c3ccc4c(c3)c3c5sc(-c6ccccc6)nc5cc5c6ccccc6n4c53)cc21] 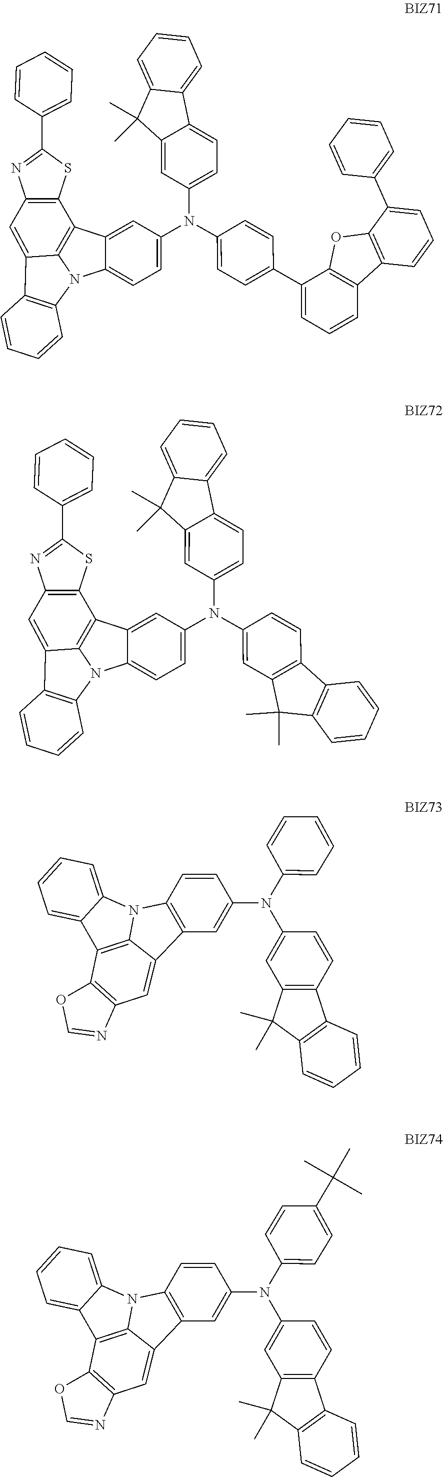 [SMILES: CC1(C)c2ccccc2-c2ccc(N(c3ccc(-c4cccc5c4oc4c(-c6ccccc6)cccc45)cc3)c3ccc4c(c3)c3c5sc(-c6ccccc6)nc5cc5c6ccccc6n4c53)cc21 ; CC1(C)c2ccccc2-c2ccc(N(c3ccc4c(c3)C(C)(C)c3ccccc3-4)c3ccc4c(c3)c3c5sc(-c6ccccc6)nc5cc5c6ccccc6n4c53)cc21 ; CC1(C)c2ccccc2-c2ccc(N(c3ccccc3)c3ccc4c(c3)c3cc5ncoc5c5c6ccccc6n4c35)cc21 ; CC(C)(C)c1ccc(N(c2ccc3c(c2)C(C)(C)c2ccccc2-3)c2ccc3c(c2)c2cc4ncoc4c4c5ccccc5n3c24)cc1] 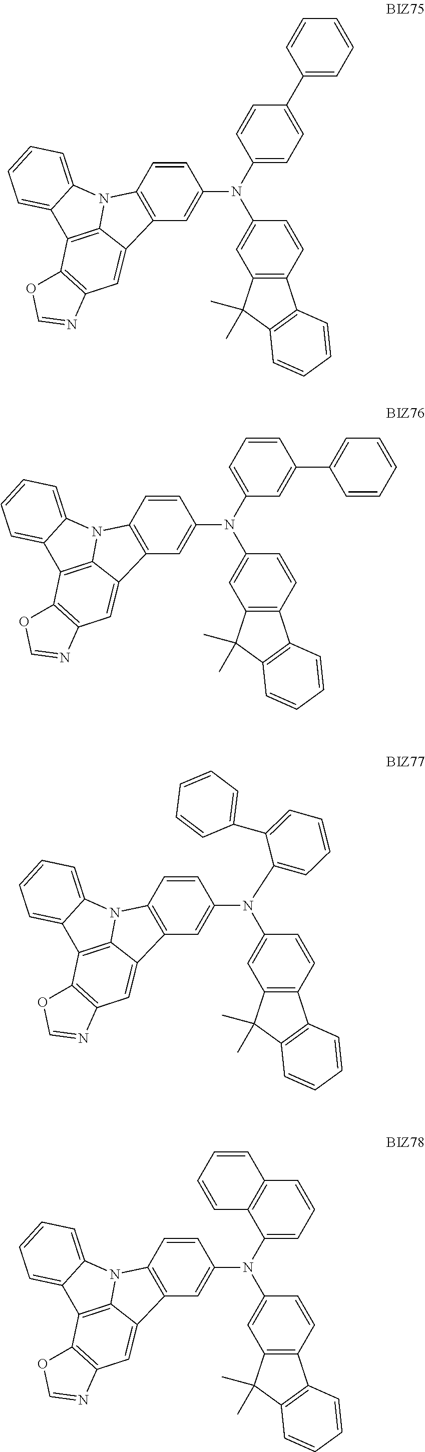 [SMILES: CC1(C)c2ccccc2-c2ccc(N(c3ccc(-c4ccccc4)cc3)c3ccc4c(c3)c3cc5ncoc5c5c6ccccc6n4c35)cc21 ; CC1(C)c2ccccc2-c2ccc(N(c3cccc(-c4ccccc4)c3)c3ccc4c(c3)c3cc5ncoc5c5c6ccccc6n4c35)cc21 ; CC1(C)c2ccccc2-c2ccc(N(c3ccc4c(c3)c3cc5ncoc5c5c6ccccc6n4c35)c3ccccc3-c3ccccc3)cc21 ; CC1(C)c2ccccc2-c2ccc(N(c3ccc4c(c3)c3cc5ncoc5c5c6ccccc6n4c35)c3cccc4ccccc34)cc21] 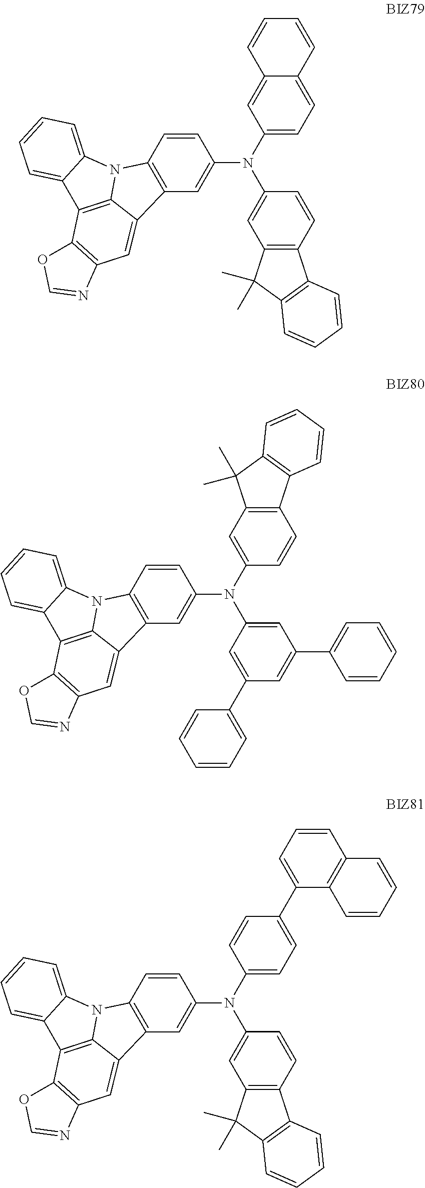 [SMILES: CC1(C)c2ccccc2-c2ccc(N(c3ccc4ccccc4c3)c3ccc4c(c3)c3cc5ncoc5c5c6ccccc6n4c35)cc21 ; CC1(C)c2ccccc2-c2ccc(N(c3cc(-c4ccccc4)cc(-c4ccccc4)c3)c3ccc4c(c3)c3cc5ncoc5c5c6ccccc6n4c35)cc21 ; CC1(C)c2ccccc2-c2ccc(N(c3ccc(-c4cccc5ccccc45)cc3)c3ccc4c(c3)c3cc5ncoc5c5c6ccccc6n4c35)cc21] 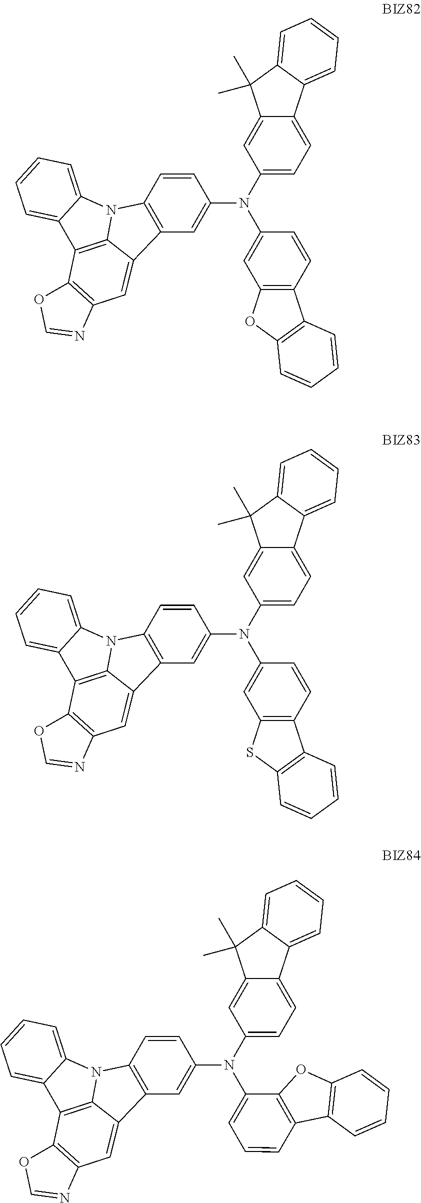 [SMILES: CC1(C)c2ccccc2-c2ccc(N(c3ccc4c(c3)oc3ccccc34)c3ccc4c(c3)c3cc5ncoc5c5c6ccccc6n4c35)cc21 ; CC1(C)c2ccccc2-c2ccc(N(c3ccc4c(c3)sc3ccccc34)c3ccc4c(c3)c3cc5ncoc5c5c6ccccc6n4c35)cc21 ; CC1(C)c2ccccc2-c2ccc(N(c3ccc4c(c3)c3cc5ncoc5c5c6ccccc6n4c35)c3cccc4c3oc3ccccc34)cc21] 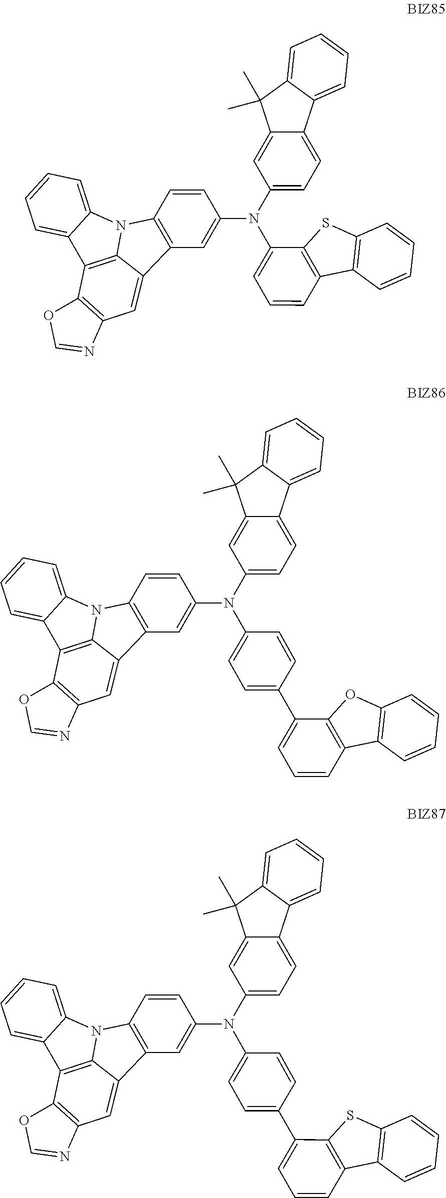 [SMILES: CC1(C)c2ccccc2-c2ccc(N(c3ccc4c(c3)c3cc5ncoc5c5c6ccccc6n4c35)c3cccc4c3sc3ccccc34)cc21 ; CC1(C)c2ccccc2-c2ccc(N(c3ccc(-c4cccc5c4oc4ccccc45)cc3)c3ccc4c(c3)c3cc5ncoc5c5c6ccccc6n4c35)cc21 ; CC1(C)c2ccccc2-c2ccc(N(c3ccc(-c4cccc5c4sc4ccccc45)cc3)c3ccc4c(c3)c3cc5ncoc5c5c6ccccc6n4c35)cc21] 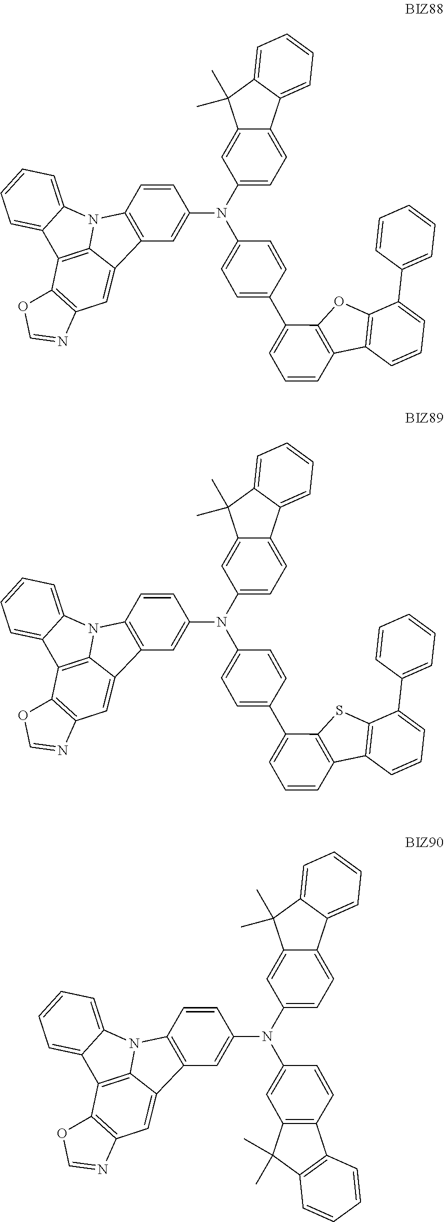 [SMILES: CC1(C)c2ccccc2-c2ccc(N(c3ccc(-c4cccc5c4oc4c(-c6ccccc6)cccc45)cc3)c3ccc4c(c3)c3cc5ncoc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5ncoc5c5c6ccccc6n4c35)cc21 ; CC1(C)c2ccccc2-c2ccc(N(c3ccc4c(c3)C(C)(C)c3ccccc3-4)c3ccc4c(c3)c3cc5ncoc5c5c6ccccc6n4c35)cc21] 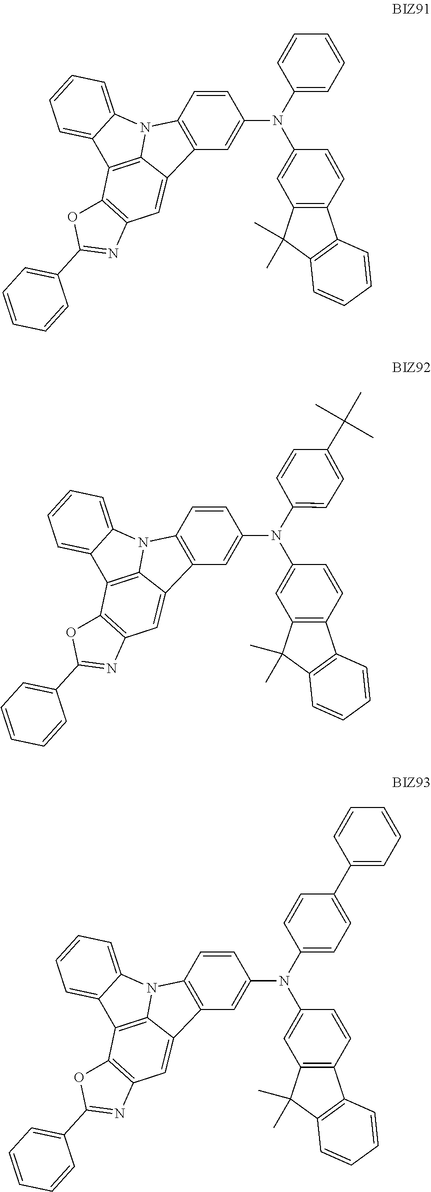 [SMILES: CC1(C)c2ccccc2-c2ccc(N(c3ccccc3)c3ccc4c(c3)c3cc5nc(-c6ccccc6)oc5c5c6ccccc6n4c35)cc21 ; CC(C)(C)c1ccc(N(c2ccc3c(c2)C(C)(C)c2ccccc2-3)c2ccc3c(c2)c2cc4nc(-c5ccccc5)oc4c4c5ccccc5n3c24)cc1 ; CC1(C)c2ccccc2-c2ccc(N(c3ccc(-c4ccccc4)cc3)c3ccc4c(c3)c3cc5nc(-c6ccccc6)oc5c5c6ccccc6n4c35)cc21] 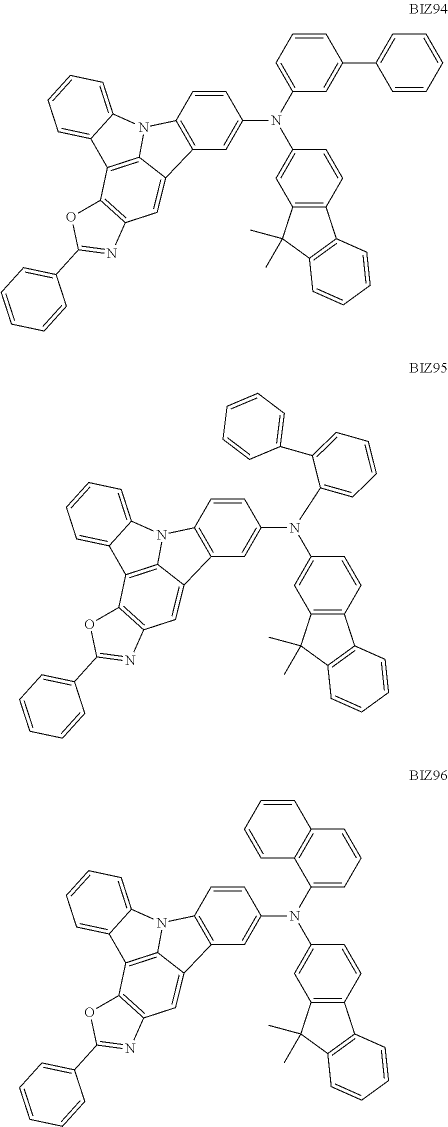 [SMILES: CC1(C)c2ccccc2-c2ccc(N(c3cccc(-c4ccccc4)c3)c3ccc4c(c3)c3cc5nc(-c6ccccc6)oc5c5c6ccccc6n4c35)cc21 ; CC1(C)c2ccccc2-c2ccc(N(c3ccc4c(c3)c3cc5nc(-c6ccccc6)oc5c5c6ccccc6n4c35)c3ccccc3-c3ccccc3)cc21 ; CC1(C)c2ccccc2-c2ccc(N(c3ccc4c(c3)c3cc5nc(-c6ccccc6)oc5c5c6ccccc6n4c35)c3cccc4ccccc34)cc21] 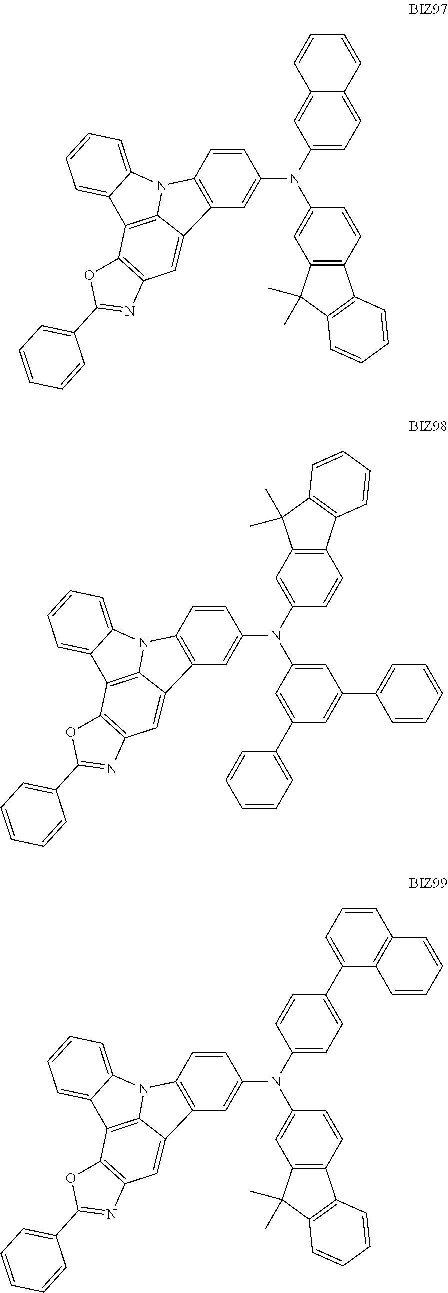 [SMILES: CC1(C)c2ccccc2-c2ccc(N(c3ccc4ccccc4c3)c3ccc4c(c3)c3cc5nc(-c6ccccc6)oc5c5c6ccccc6n4c35)cc21 ; CC1(C)c2ccccc2-c2ccc(N(c3cc(-c4ccccc4)cc(-c4ccccc4)c3)c3ccc4c(c3)c3cc5nc(-c6ccccc6)oc5c5c6ccccc6n4c35)cc21 ; CC1(C)c2ccccc2-c2ccc(N(c3ccc(-c4cccc5ccccc45)cc3)c3ccc4c(c3)c3cc5nc(-c6ccccc6)oc5c5c6ccccc6n4c35)cc21] 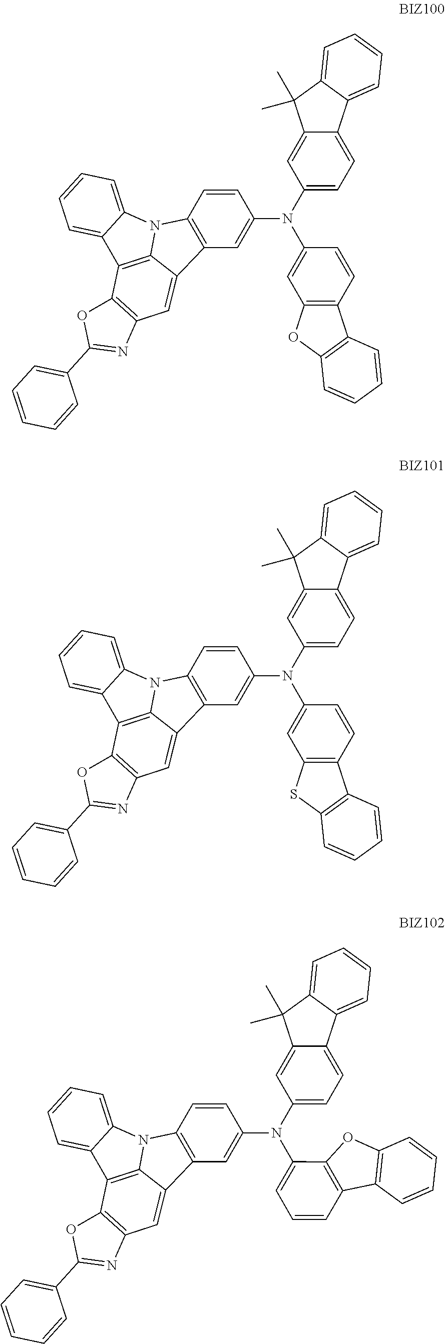 [SMILES: CC1(C)c2ccccc2-c2ccc(N(c3ccc4c(c3)oc3ccccc34)c3ccc4c(c3)c3cc5nc(-c6ccccc6)oc5c5c6ccccc6n4c35)cc21 ; CC1(C)c2ccccc2-c2ccc(N(c3ccc4c(c3)sc3ccccc34)c3ccc4c(c3)c3cc5nc(-c6ccccc6)oc5c5c6ccccc6n4c35)cc21 ; CC1(C)c2ccccc2-c2ccc(N(c3ccc4c(c3)c3cc5nc(-c6ccccc6)oc5c5c6ccccc6n4c35)c3cccc4c3oc3ccccc34)cc21] 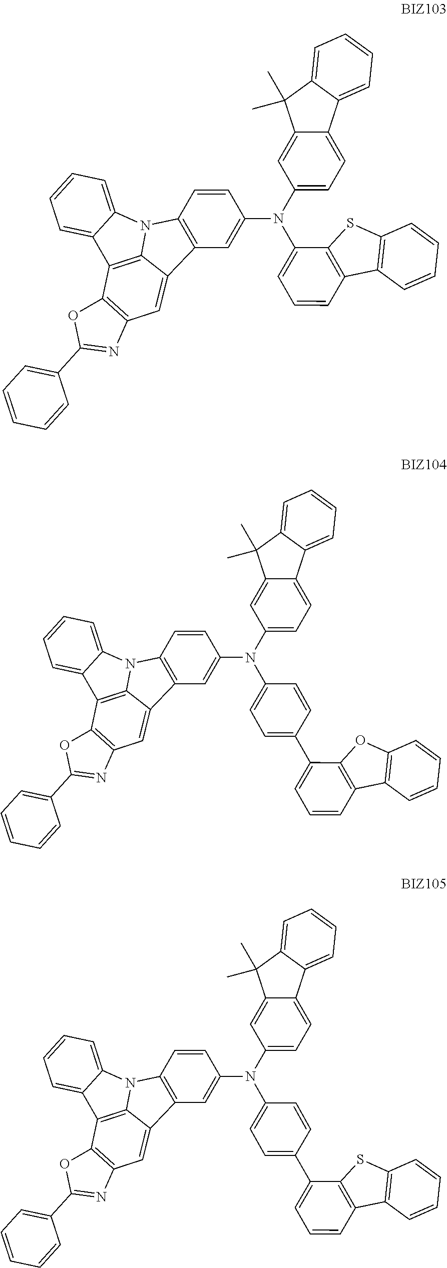 [SMILES: CC1(C)c2ccccc2-c2ccc(N(c3ccc4c(c3)c3cc5nc(-c6ccccc6)oc5c5c6ccccc6n4c35)c3cccc4c3sc3ccccc34)cc21 ; CC1(C)c2ccccc2-c2ccc(N(c3ccc(-c4cccc5c4oc4ccccc45)cc3)c3ccc4c(c3)c3cc5nc(-c6ccccc6)oc5c5c6ccccc6n4c35)cc21 ; CC1(C)c2ccccc2-c2ccc(N(c3ccc(-c4cccc5c4sc4ccccc45)cc3)c3ccc4c(c3)c3cc5nc(-c6ccccc6)oc5c5c6ccccc6n4c35)cc21] 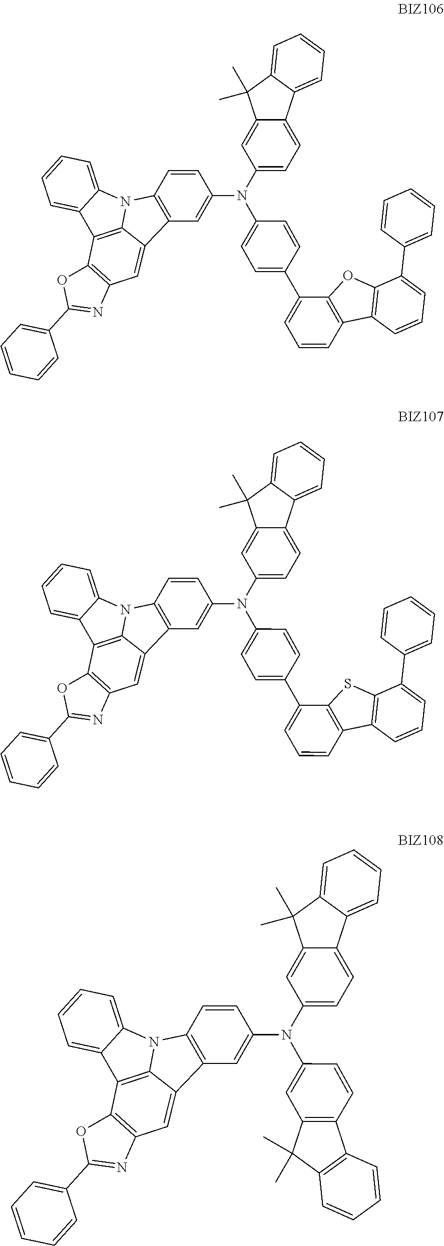 [SMILES: CC1(C)c2ccccc2-c2ccc(N(c3ccc(-c4cccc5c4oc4c(-c6ccccc6)cccc45)cc3)c3ccc4c(c3)c3cc5nc(-c6ccccc6)oc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5nc(-c6ccccc6)oc5c5c6ccccc6n4c35)cc21 ; CC1(C)c2ccccc2-c2ccc(N(c3ccc4c(c3)C(C)(C)c3ccccc3-4)c3ccc4c(c3)c3cc5nc(-c6ccccc6)oc5c5c6ccccc6n4c35)cc21] 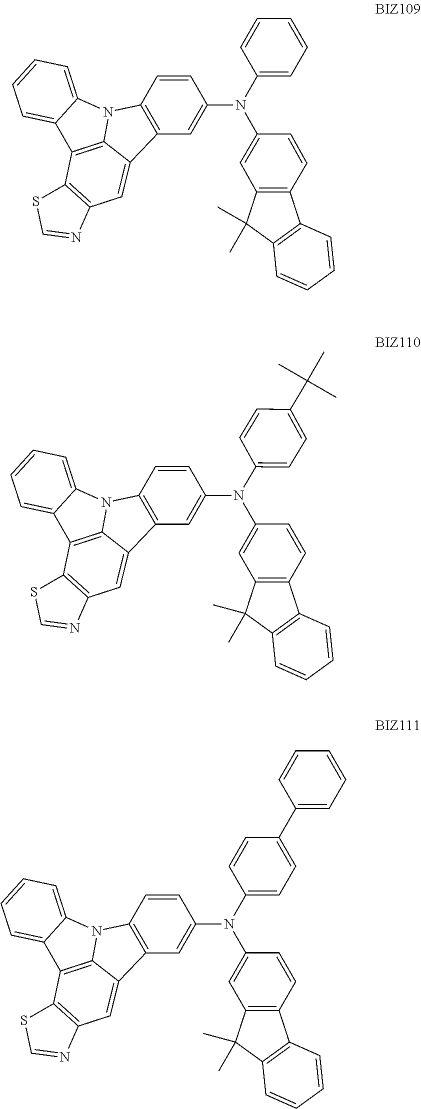 [SMILES: CC1(C)c2ccccc2-c2ccc(N(c3ccccc3)c3ccc4c(c3)c3cc5ncsc5c5c6ccccc6n4c35)cc21 ; CC(C)(C)c1ccc(N(c2ccc3c(c2)C(C)(C)c2ccccc2-3)c2ccc3c(c2)c2cc4ncsc4c4c5ccccc5n3c24)cc1 ; CC1(C)c2ccccc2-c2ccc(N(c3ccc(-c4ccccc4)cc3)c3ccc4c(c3)c3cc5ncsc5c5c6ccccc6n4c35)cc21] 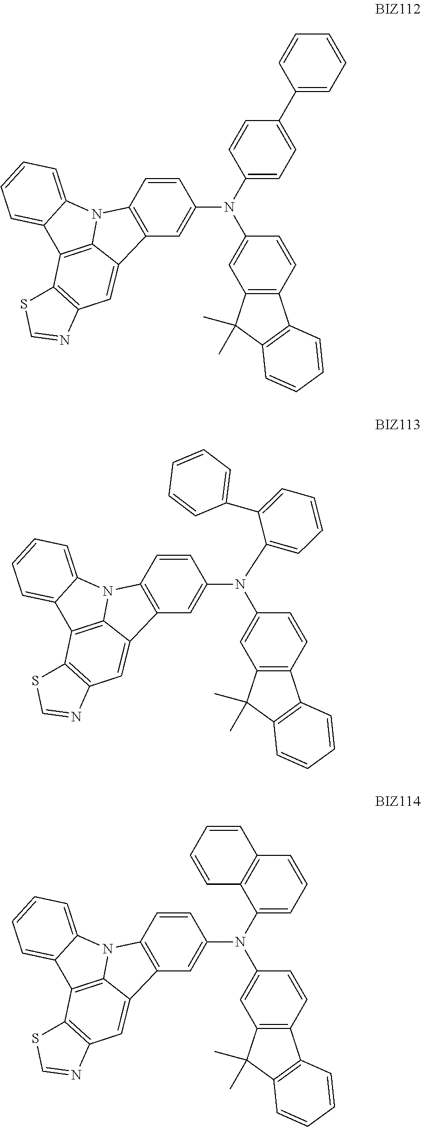 [SMILES: CC1(C)c2ccccc2-c2ccc(N(c3ccc(-c4ccccc4)cc3)c3ccc4c(c3)c3cc5ncsc5c5c6ccccc6n4c35)cc21 ; CC1(C)c2ccccc2-c2ccc(N(c3ccc4c(c3)c3cc5ncsc5c5c6ccccc6n4c35)c3ccccc3-c3ccccc3)cc21 ; CC1(C)c2ccccc2-c2ccc(N(c3ccc4c(c3)c3cc5ncsc5c5c6ccccc6n4c35)c3cccc4ccccc34)cc21] 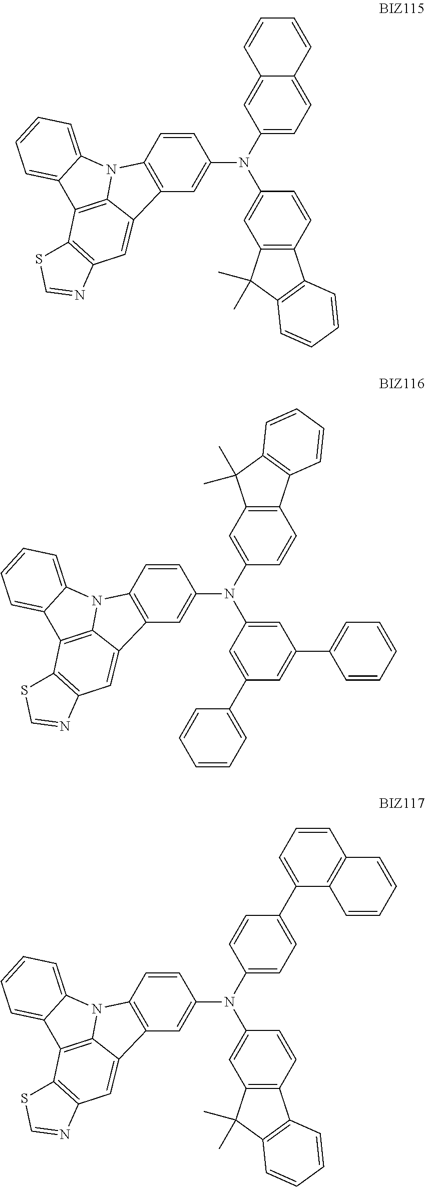 [SMILES: CC1(C)c2ccccc2-c2ccc(N(c3ccc4ccccc4c3)c3ccc4c(c3)c3cc5ncsc5c5c6ccccc6n4c35)cc21 ; CC1(C)c2ccccc2-c2ccc(N(c3cc(-c4ccccc4)cc(-c4ccccc4)c3)c3ccc4c(c3)c3cc5ncsc5c5c6ccccc6n4c35)cc21 ; CC1(C)c2ccccc2-c2ccc(N(c3ccc(-c4cccc5ccccc45)cc3)c3ccc4c(c3)c3cc5ncsc5c5c6ccccc6n4c35)cc21] 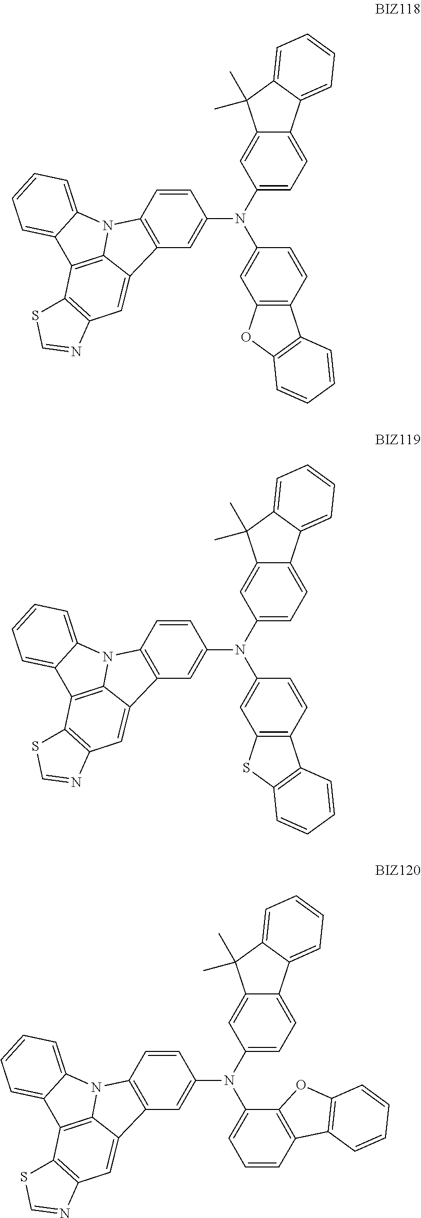 [SMILES: CC1(C)c2ccccc2-c2ccc(N(c3ccc4c(c3)oc3ccccc34)c3ccc4c(c3)c3cc5ncsc5c5c6ccccc6n4c35)cc21 ; CC1(C)c2ccccc2-c2ccc(N(c3ccc4c(c3)sc3ccccc34)c3ccc4c(c3)c3cc5ncsc5c5c6ccccc6n4c35)cc21 ; CC1(C)c2ccccc2-c2ccc(N(c3ccc4c(c3)c3cc5ncsc5c5c6ccccc6n4c35)c3cccc4c3oc3ccccc34)cc21] 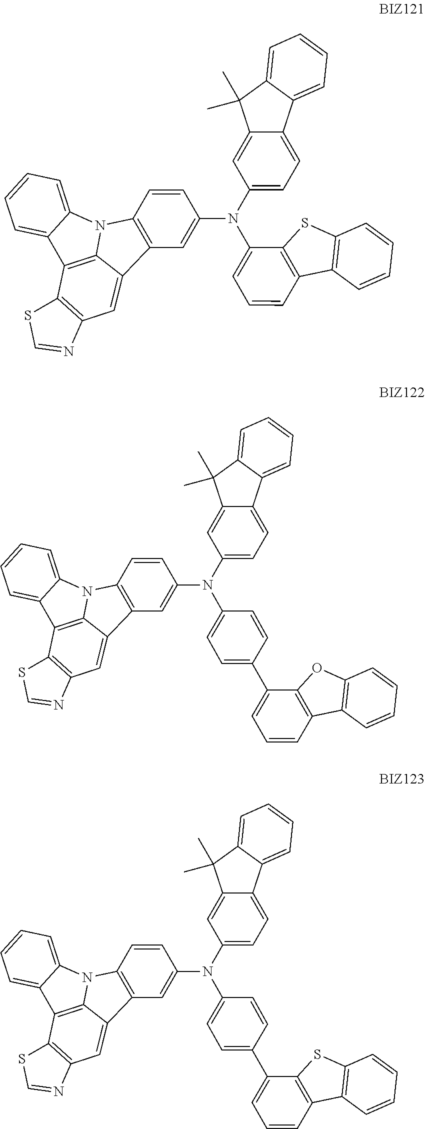 [SMILES: CC1(C)c2ccccc2-c2ccc(N(c3ccc4c(c3)c3cc5ncsc5c5c6ccccc6n4c35)c3cccc4c3sc3ccccc34)cc21 ; CC1(C)c2ccccc2-c2ccc(N(c3ccc(-c4cccc5c4oc4ccccc45)cc3)c3ccc4c(c3)c3cc5ncsc5c5c6ccccc6n4c35)cc21 ; CC1(C)c2ccccc2-c2ccc(N(c3ccc(-c4cccc5c4sc4ccccc45)cc3)c3ccc4c(c3)c3cc5ncsc5c5c6ccccc6n4c35)cc21] 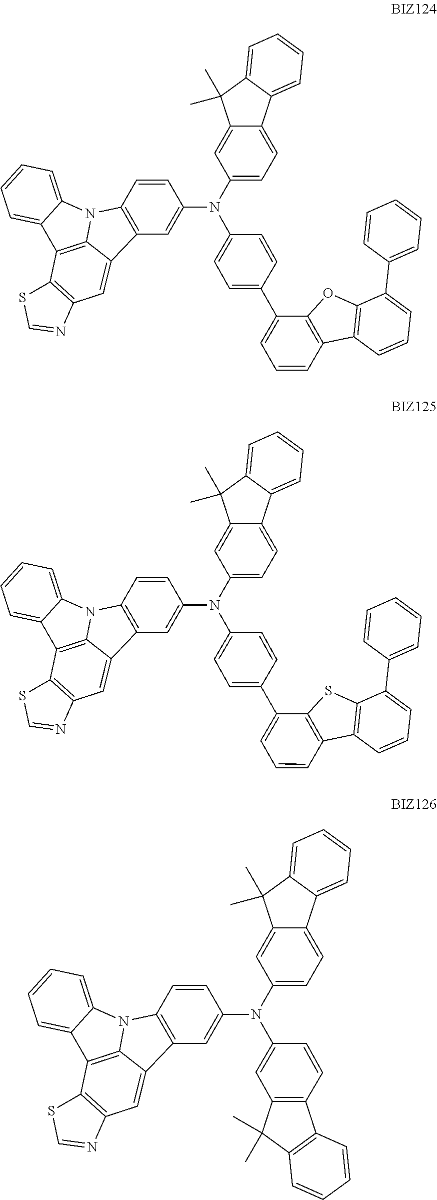 [SMILES: CC1(C)c2ccccc2-c2ccc(N(c3ccc(-c4cccc5c4oc4c(-c6ccccc6)cccc45)cc3)c3ccc4c(c3)c3cc5ncsc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5ncsc5c5c6ccccc6n4c35)cc21 ; CC1(C)c2ccccc2-c2ccc(N(c3ccc4c(c3)C(C)(C)c3ccccc3-4)c3ccc4c(c3)c3cc5ncsc5c5c6ccccc6n4c35)cc21] 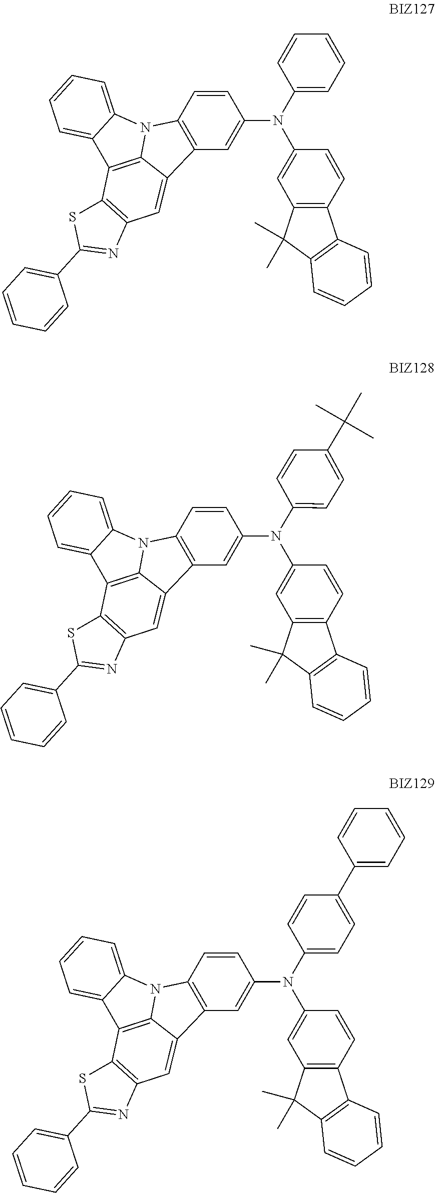 [SMILES: CC1(C)c2ccccc2-c2ccc(N(c3ccccc3)c3ccc4c(c3)c3cc5nc(-c6ccccc6)sc5c5c6ccccc6n4c35)cc21 ; CC(C)(C)c1ccc(N(c2ccc3c(c2)C(C)(C)c2ccccc2-3)c2ccc3c(c2)c2cc4nc(-c5ccccc5)sc4c4c5ccccc5n3c24)cc1 ; CC1(C)c2ccccc2-c2ccc(N(c3ccc(-c4ccccc4)cc3)c3ccc4c(c3)c3cc5nc(-c6ccccc6)sc5c5c6ccccc6n4c35)cc21] 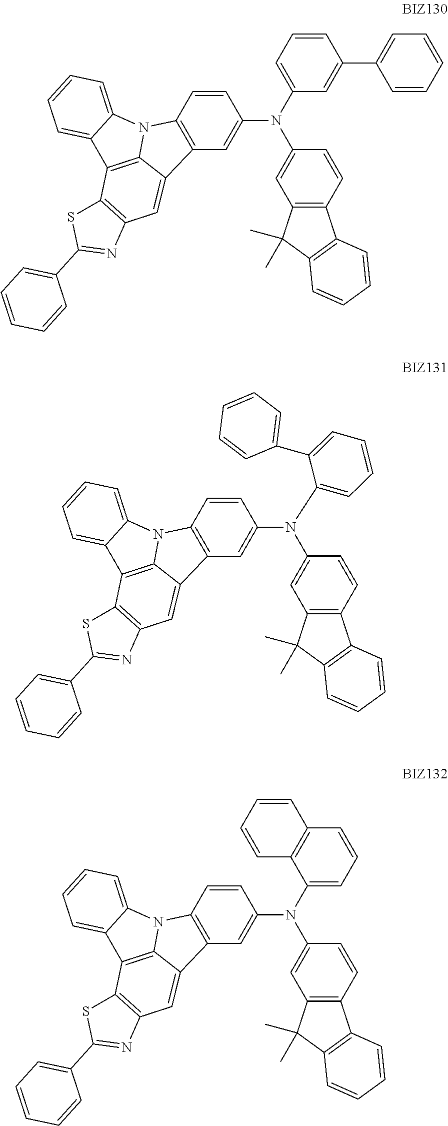 [SMILES: CC1(C)c2ccccc2-c2ccc(N(c3cccc(-c4ccccc4)c3)c3ccc4c(c3)c3cc5nc(-c6ccccc6)sc5c5c6ccccc6n4c35)cc21 ; CC1(C)c2ccccc2-c2ccc(N(c3ccc4c(c3)c3cc5nc(-c6ccccc6)sc5c5c6ccccc6n4c35)c3ccccc3-c3ccccc3)cc21 ; CC1(C)c2ccccc2-c2ccc(N(c3ccc4c(c3)c3cc5nc(-c6ccccc6)sc5c5c6ccccc6n4c35)c3cccc4ccccc34)cc21] 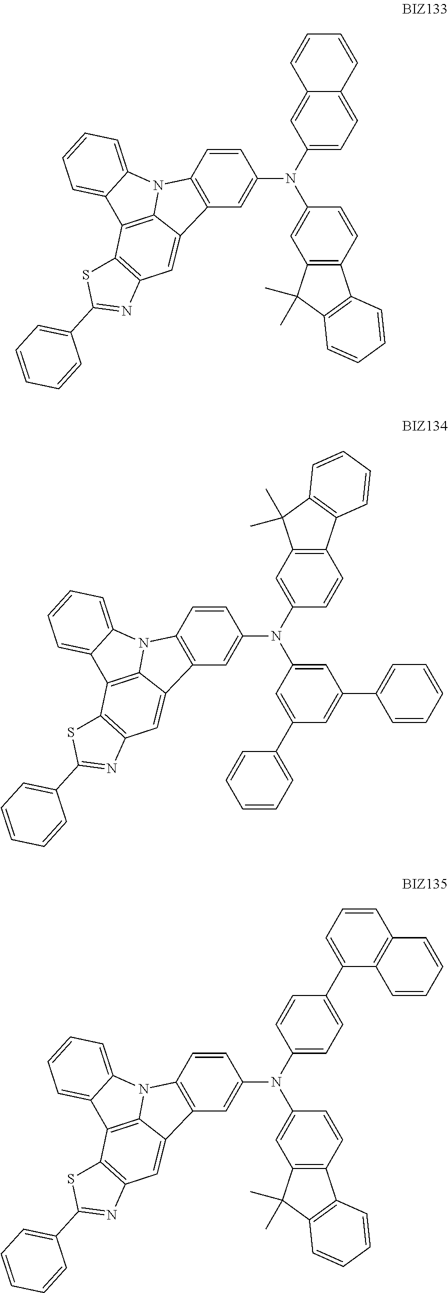 [SMILES: CC1(C)c2ccccc2-c2ccc(N(c3ccc4ccccc4c3)c3ccc4c(c3)c3cc5nc(-c6ccccc6)sc5c5c6ccccc6n4c35)cc21 ; CC1(C)c2ccccc2-c2ccc(N(c3cc(-c4ccccc4)cc(-c4ccccc4)c3)c3ccc4c(c3)c3cc5nc(-c6ccccc6)sc5c5c6ccccc6n4c35)cc21 ; CC1(C)c2ccccc2-c2ccc(N(c3ccc(-c4cccc5ccccc45)cc3)c3ccc4c(c3)c3cc5nc(-c6ccccc6)sc5c5c6ccccc6n4c35)cc21] 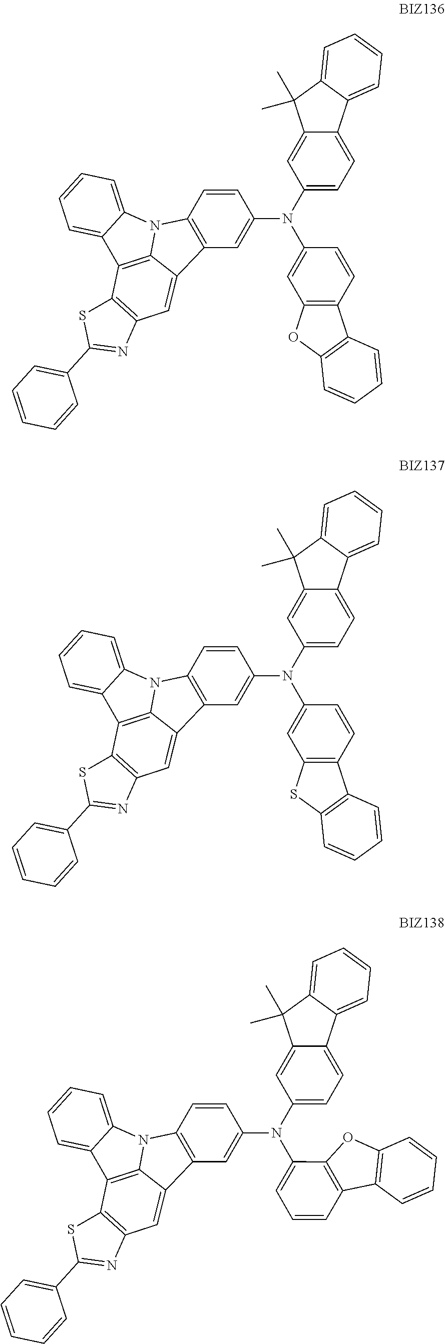 [SMILES: CC1(C)c2ccccc2-c2ccc(N(c3ccc4c(c3)oc3ccccc34)c3ccc4c(c3)c3cc5nc(-c6ccccc6)sc5c5c6ccccc6n4c35)cc21 ; CC1(C)c2ccccc2-c2ccc(N(c3ccc4c(c3)sc3ccccc34)c3ccc4c(c3)c3cc5nc(-c6ccccc6)sc5c5c6ccccc6n4c35)cc21 ; CC1(C)c2ccccc2-c2ccc(N(c3ccc4c(c3)c3cc5nc(-c6ccccc6)sc5c5c6ccccc6n4c35)c3cccc4c3oc3ccccc34)cc21] 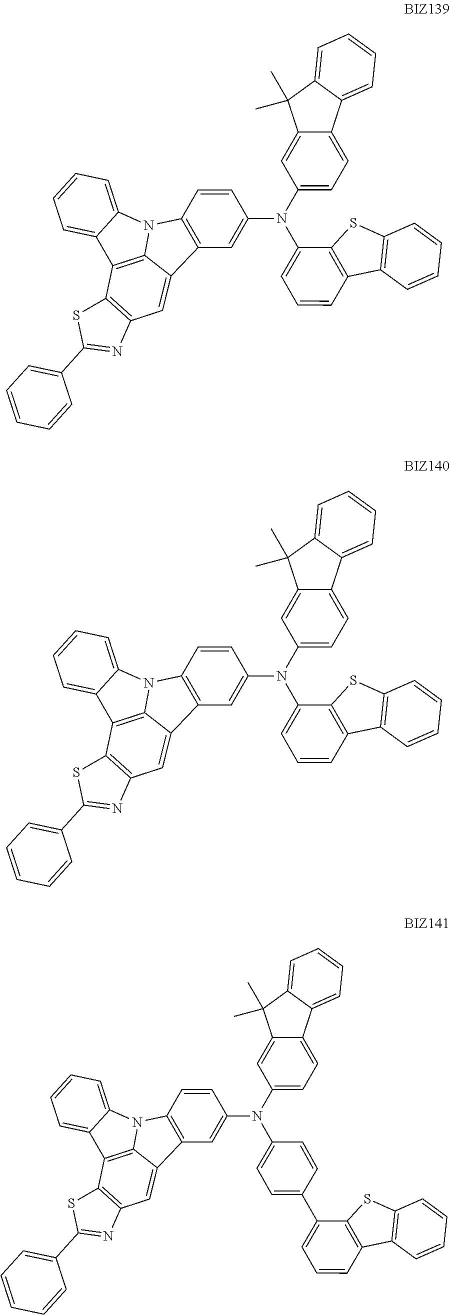 [SMILES: CC1(C)c2ccccc2-c2ccc(N(c3ccc4c(c3)c3cc5nc(-c6ccccc6)sc5c5c6ccccc6n4c35)c3cccc4c3sc3ccccc34)cc21 ; CC1(C)c2ccccc2-c2ccc(N(c3ccc4c(c3)c3cc5nc(-c6ccccc6)sc5c5c6ccccc6n4c35)c3cccc4c3sc3ccccc34)cc21 ; CC1(C)c2ccccc2-c2ccc(N(c3ccc(-c4cccc5c4sc4ccccc45)cc3)c3ccc4c(c3)c3cc5nc(-c6ccccc6)sc5c5c6ccccc6n4c35)cc21] 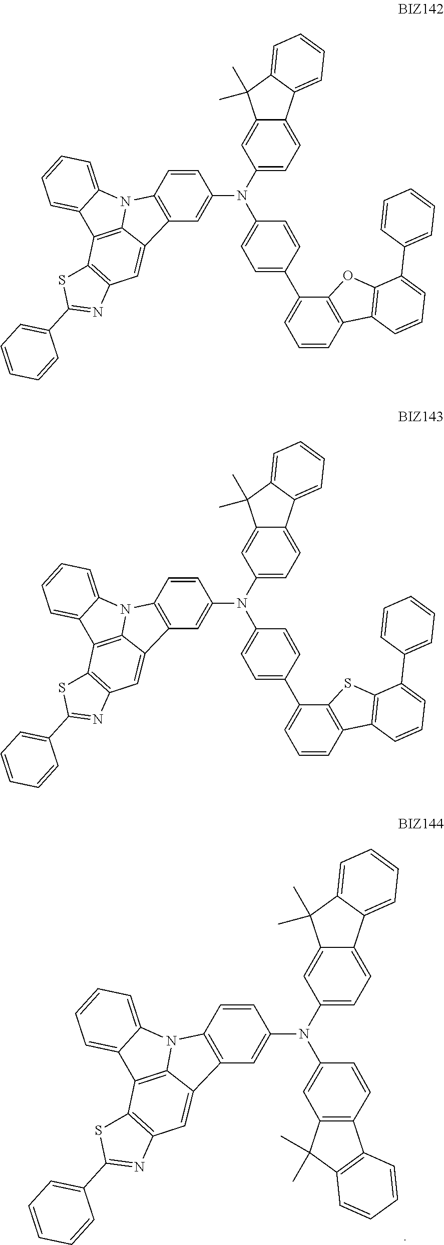 [SMILES: CC1(C)c2ccccc2-c2ccc(N(c3ccc(-c4cccc5c4oc4c(-c6ccccc6)cccc45)cc3)c3ccc4c(c3)c3cc5nc(-c6ccccc6)sc5c5c6ccccc6n4c35)cc21 ; CC1(C)c2ccccc2-c2ccc(N(c3ccc(-c4cccc5c4sc4c(-c6ccccc6)cccc45)cc3)c3ccc4c(c3)c3cc5nc(-c6ccccc6)sc5c5c6ccccc6n4c35)cc21 ; CC1(C)c2ccccc2-c2ccc(N(c3ccc4c(c3)C(C)(C)c3ccccc3-4)c3ccc4c(c3)c3cc5nc(-c6ccccc6)sc5c5c6ccccc6n4c35)cc21]
